(19) United States
(12) Patent Application Publication
(10) Pub. No.: US 2025/0262242 A1
(43) Pub. Date: Aug. 21, 2025

(54) HUMAN T CELL RECEPTORS SPECIFIC FOR ANTIGENIC PEPTIDES DERIVED FROM MITOGEN-ACTIVATED PROTEIN KINASE 8 INTERACTING PROTEIN 2 (MAPK8IP2), EPSTEIN-BARR VIRUS OR HUMAN ENDOGENOUS RETROVIRUS, AND USES THEREOF

(71) Applicant: ImmunoSCAPE Pte. Ltd., Singapore (SG)

(72) Inventors: Daniel MacLeod, La Jolla, CA (US); Katja Fink, Singapore (SG); Michael Fehlings, La Jolla, CA (US); Loan Ping Eng, Singapore (SG); Kan Xing Wu, Singapore (SG); Alessandra Nardin, Singapore (SG); Xin-Zi Tang, Singapore (SG)

(73) Assignee: ImmunoSCAPE Pte. Ltd., Singapore (SG)

(21) Appl. No.: 18/873,284

(22) PCT Filed: Jun. 15, 2023
(86) PCT No.: PCT/EP2023/066121

§ 371 Date: Dec. 9, 2024

(30) Foreign Application Priority Data
Jun. 15, 2022 (SG) 10202250175C
Nov. 7, 2022 (SG) 10202260001W

(51) Int. Cl. A61K 35/17; A61K 40/32; A61K 40/42; A61K 40/46; A61K 45/06; A61P 31/14; C07K 14/725; C07K 16/08; C07K 16/28

(52) U.S. Cl. CPC A61K 35/17; A61K 40/32; A61K 40/421; A61K 40/46; A61K 45/06; A61P 31/14; C07K 14/7051; C07K 16/085; C07K 16/2809; C07K 2317/565

(57) ABSTRACT

The present application describes T cell receptors specifically binding tumor antigen derived peptides, especially derived from Mitogen-Activated Protein Kinase 8 Interacting Protein 2 (MAPK8IP2), Epstein-Barr Virus (EBV) proteins or Human Endogenous Retrovirus (HERV), as well as engineered T cells expressing these receptors, nucleic acids encoding these T cell receptors and methods of using T cells expressing these engineered T cells in adoptive cell transfer to treat diseases in a subject.

This application claims priority from SG 10202250175C filed 15 Jun. 2022, and from SG 10202260001W filed 7 Nov. 2022, the contents and elements of which are herein incorporated by reference for all purposes.

### TECHNICAL FIELD

The present invention relates to the field of immunotherapy for the treatment and prevention of disease, particularly cancers, and in particular, to adoptive T cell therapy or T cell receptor (TCR) gene therapy or TCR fusion protein therapy.

### BACKGROUND OF THE INVENTION

T cells form part of the adaptive immune response. T cells develop in the thymus and are equipped with a unique T cell receptor (TCR) that recognizes peptides derived from cellular or extra-cellular antigens and presented by major histocompatibility complex (MHC) molecules. There are two types of T cells: CD8 T cells, which bind to peptides presented on MHC class I (MHC-I), and CD4 T cells, which bind to peptides presented on MHC class II (MHC-II) peptides. CD8 T cells are equipped with the capacity to induce cytotoxicity in target cells upon specific TCR binding to a peptide presented on MHC-I, leading to the elimination of target cells. CD4 T cells primarily play a role in supporting CD8 T cell function and other function of other immune cells. Conventional CD8 T cells express a TCR that comprises a TCRα and a TCRβ chain on the cell surface. The TCR comprises a C′-terminal constant region and a N′-terminal variable region comprising framework region (FR) 1 to 4, interspersed with complementarity determining regions (CDRs) 1, 2 and 3 in the following sequence: FR1-CDR1-FR2-CDR2-FR3-CDR3-FR4. The CDRs are the main contact point of binding to peptides presented on MHC-I. The specific binding of a TCR to a given MHC-I allele or a group of related MHC-I alleles is called MHC-restriction (Murphy and Weaver Casey; Sidney et al., 2008).

The diversity of the TCR is accomplished by assembling of variable (V), diversity (D) and joining (J) gene segments for the TCRβ chain and V and J gene segments for the TCRα chain during T cell development. Additional diversity is generated with the additional insertion of nucleotides between the V-D and D-J gene segments during T cell development, generating a unique TCR in each developing T cell.

T cells recognize peptides derived from pathogens or from cancer cells and are therefore a crucial component of the immune response to infection and cancer. The ability of T cells to specifically bind to and kill tumor cells has been recognized more than 30 years ago (Topalian et al., J Immunother 12, 203-206 (1992)). Various strategies employing T cells for immunotherapy have been explored (Ellis et al., 2021; Garber, 2018; Rosenberg and Restifo, 2015). The extraction, expansion and re-infusion of tumor-infiltrated T cells has been used successfully as a therapy. However, this procedure is complicated and only applicable for a limited range of cancers. An alternative strategy is the expression of synthetic, exogenous tumor-specific TCRs on T cells from patients or healthy donors. The TCR-engineered T cells made to recognize tumor cells are expanded and adoptively transferred into patients (Shafer et al., 2022). Alternatively, TCRs can be used in other forms for therapeutic application, for example in the form of bi-specific molecules (Strobel, 2022).

The Epstein-Barr virus (a γ herpes virus that stays dormant (latent) for a long period in memory B cells; hereinafter may be abbreviated as EBV) is involved in many malignancies, for example, Burkitt's lymphoma, Hodgkin's disease (HD) and nasopharyngeal carcinoma (NPC), as well as post-transplant lymphoproliferative disorder. In latent infections, viral protein expression is suppressed. All EBV-positive malignant cells exhibit one of the following three latency types. These types are distinguished from each other by the EBV antigen expression patterns. In latency type I, only the EBV nuclear antigen (EBNA) 1 is expressed; in latency type II, latent membrane protein (LMP) 1 and 2 are expressed along with EBNA1; in latency type III, all EBV latent proteins are expressed, meaning EBNA1, EBNA2, EBNA3A, EBNA3B, EBNA3, leader protein (LP), LMP1 and LMP2 (US Patent Application No. 20090305324, incorporated herein by reference in its entirety). Some EBV proteins are known viral oncogenic proteins that can drive the development of cancer and other diseases, causing a high global burden of EBV-driven malignancies, such as nasopharyngeal carcinomas (NPC), gastric carcinoma, Burkitt's lymphoma, Hodgkin's disease, Non-Hodgkin's lymphoma, NK/T cell lymphoma, etc. (Khan and Hashim, Infect Agent Cancer 9 (2014); Thompson and Kurzrock, 2004; US Patent Application No. 20090305324, each incorporated herein by reference in their entirety). In addition, chronic EBV infection is a potentially life-threatening condition in immune-suppressed individuals such as patients undergoing transplantation, as is the case for post-transplant lymphoproliferative disorder. Moreover, chronic or recurrent EBV infection has been linked to several autoimmune disorders, such as systemic lupus erythematosus (SLE), Sjögren's syndrome, multiple sclerosis, and other diseases (Houen and Trier, Front. Immunol. January 2021, Vol. 11, art. 587380).

The immunogenicity of peptides derived from oncogenic EBV proteins makes them very promising targets for T cell mediated therapy in all these EBV-driven diseases. Clinical trials expanding and re-infusing EBV-protein-targeting T cells showed promising results (Bollard et al., 2014; Cho et al., 2015). EBV peptides presented by MHC-I and therefore targetable by CD8+ T cells including BRLF1 peptide YVLDHLIVV (SEQ ID NO:105) and LMP2 peptides CLGGLLTMV (SEQ ID NO:106) and FLYALALLL (SEQ ID NO:107) have been reported in the literature. There is thus an increasing interest in using immunotherapy for EBV-associated diseases, disorders and conditions.

Splicing of pre-mRNA by spliceosomes is a cellular process that removes non-coding introns in transcripts and produces alternative splice forms of proteins. Splicing Factor 3B subunit 1 (SF3B1) and other splicing factors have been reported to be mutated in several types of cancers including uveal melanoma (Bigot et al., 2021; Nguyen et al., 2020). Mutated splice-factor-induced peptides are a promising target for TCR-mediated cancer therapy because of the tumor-specific expression of such peptides, and because of the potential increased immunogenicity. Mutated splice factor-induced peptides, including peptide RLPGVLPRA (SEQ ID NO:147) have been reported in the literature (Bigot et al., 2021). The therapeutic value of TCR-based approaches targeting these peptides, however, is not known. The current invention proposes TCR sequences that can be used for the treatment of diseases associated with mutated forms of protein mitogen-activated protein kinase 8 interacting protein 2 (MAPK8IP2), or other splicing factors including SUGP1 and SF3B1.

About 9% of the human genome consists of genetic information from human endogenous retroviruses (HERVs) that was incorporated into the germline as humans evolved (Jansz and Faulkner, 2021). HERV-K is a group of HERVs with relatively intact open reading frames, making the expression of HERV-K proteins more likely compared to other HERVs (Gao et al., 2021). Since expression of human endogenous retrovirus group K (HERV-K) proteins is preferentially seen in cancer cells, T cell receptor-mediated therapy against HERV-K T cell epitopes, including FLQFKTWWI (SEQ ID NO:148), is an attractive strategy for the treatment of cancer that has not yet been tested clinically.

Adoptive cell therapy (ACT, also referred to as adoptive cell transfer) using chimeric antigen receptor (CAR)-engineered T-cells has been shown to induce durable remissions in subjects with refractory B-lymphoid cancers. Results are however comparatively modest when CAR-engineered T-cells are directed against solid malignancies. Thus, alternative strategies to redirect T-cell specificity and cytolytic function are necessary to ameliorate ACT as a therapeutic regimen against solid tumors, and other cancers. Unlike CARs, T-cell receptors (TCRs) may recognize epitopes derived from any subcellular compartment, such as the membrane, cytoplasm, and nucleus. Furthermore, TCRs efficiently respond to epitope densities many fold smaller than required to activate CAR-signaling. Clinical trials demonstrate that TCR-based ACT mediates regression of solid malignancies, including immune-checkpoint refractory tumors (Chandran and Klebanoff, Immunol. Rev. 290:127-147 (2019)).

Results from clinical trials demonstrated the potential of TCR-based therapies (Shafer et al., 2022). However, the number of clinically validated TCRs is very limited and TCR restriction is almost exclusively to the MHC-I allele Human Leukocyte Antigen (HLA)-A*02:01 (Upadhaya et al., 2020). Individuals who do not express the HLA-A*02 allele cannot benefit from these therapies and TCRs restricted to other alleles need to be developed for therapy. Large datasets with TCR sequences have been published from bulk TCR sequencing experiments, but these datasets often only contain the TCRβ CDR3 sequence, lacking the information of the paired TCRα chain sequence, which is required for the expression of a full TCR.

Accordingly, the provision of new TCRs with improved properties, e.g., antigen specificity, binding properties, stability, expression levels and the like would represent a significant advance in the art.

### SUMMARY OF THE INVENTION

In various embodiments, the present disclosure is directed to compositions and methods for editing the genome of a human T cell such that it expresses a novel T Cell Receptor (TCR). The inventors have discovered that a heterologous TCR can be inserted into the genome of a T cell. The methods and compositions provided herein can be used to produce a human T cell with a heterologous TCR having a desired antigen specificity.

In various embodiments, the present invention further provides isolated TCRs, cells expressing these TCRs, nucleic acids encoding the TCRs, and methods of engineering T cells to express the novel TCRs. Also provided is the use of the disclosed articles (TCRs, antigen-binding molecules, polypeptides, nucleic acids, vectors, cells, compositions, etc.) for therapy, such as in a method of performing adoptive cell transfer on a subject in need of such therapy to prevent, treat or ameliorate a disease state of the subject.

In various embodiments, the isolated TCRs comprise a TCRα chain variable domain and/or a TCRβ chain variable domain that binds to Epstein Barr Virus (EBV)-derived antigenic peptides, e.g. when presented by a major histocompatibility complex (MHC) molecule. In various embodiments, the isolated TCRs comprise a TCRα chain variable domain and/or a TCRβ chain variable domain that binds to a peptide of MAPK8IP2, e.g. a mutant splice-factor-induced peptide of MAPK8IP2, optionally when presented by a major histocompatibility complex (MHC) molecule. In some embodiments, the isolated TCRs comprise a TCRα chain variable domain and/or a TCRβ chain variable domain that binds to a human endogenous retrovirus K (HERV-K) gag protein peptide, optionally when presented by a major histocompatibility complex (MHC) molecule. In some embodiments, the TCRα and/or TCRβ chains each comprises three complementarity determining regions (CDR1, CDR2, and CDR3) of amino acid sequence sharing at least about 95% sequence identity with an amino acid sequence selected from Table 3A.

In some embodiments, the present invention provides methods of using the TCRs, nucleic acids, vectors, cells and/or TCR-expressing cells for therapy, such as in T-cell-based adoptive cell transfer (ACT) as a therapeutic treatment in a subject suffering an EBV-associated condition, disease, disorder, or pathology, e.g., cancer. The present invention further provides TCR as part of a fusion construct, whereby the fusion construct consists of a TCR and a single-chain fragment that binds to a molecule specifically expressed on T cells, including but not limited to CD3 for the treatment of cancer.

Other objects and embodiments of the invention will be apparent from the detailed description that follows.

### BRIEF DESCRIPTION OF THE DRAWINGS

For a further understanding of the various described implementations, reference should be made to the detailed description below, in conjunction with the following drawings in which like reference numerals refer to corresponding parts throughout the figures.

FIG. 1 shows the antigen-specific binding of Jurkat reporter cells transduced with TCR receptors A0001-A0005. Antigen presenting cells (APC) expressing HLA-A*02:01 were incubated with EBV BRLF1-derived antigenic peptide YVLDHLIVV (SEQ ID NO:105) at a range between 0.000005 and 50 μM. Successfully bound Jurkat reporter cells are activated via the TCR and generate a luciferase signal that can be quantified. Jurkats NT: non-transduced Jurkat cells.

FIG. 2 shows the antigen-specific binding of Jurkat reporter cells transduced with TCR receptor A0015. A) APC expressing HLA-A*02:01 were incubated with an EBV LMP2A-derived antigenic peptide pool (Miltenyi PepTivator 130-093-615: LMP2A, Premium grade, human) at a range between 0.00006 and 1 nM. B) APC expressing HLA-A*02:01 were incubated with an EBV LMP2A-derived peptide MGSLEMVPM (SEQ ID NO: 146) at a range between 0.00005 and 50 μM. Successfully binding Jurkat reporter cells are activated via the TCR and generate a luciferase signal that can be quantified. Jurkats NT: non-transduced Jurkat cells.

FIG. 3 shows the antigen-specific binding of Jurkat reporter cells transduced with TCR receptor A0099. A) Peripheral blood mononuclear cells (PBMCs) expressing HLA-A alleles 02:01 and 03:01 and HLA-B alleles 07:02 and 35:01 were used as APC and incubated with an EBV-derived peptide pool (Miltenyi PepTivator EBV Consensus) with a concentration range between 0.000001 nM and 1 nM. The binding was tested at three different ratios of transfected Jurkat cells: PBMCs. B) PBMCs expressing HLA-A alleles 01:01 and 11:01 and HLA-B alleles 08:01 and 35:01 and HLA-C alleles 04:01 and 07:01 were used as APC and incubated with EBV protein BZLF1-derived peptide EPLPQGQLTAY (SEQ ID NO:145) at a range of 0.000005 to 50 μM. Successfully binding Jurkat reporter cells are activated via the TCR and generate a luciferase signal that can be quantified. Jurkats NT: non-transduced Jurkat cells.

FIG. 4 shows the EBV LMP2-specific binding of Jurkat reporter cells transduced with TCR receptors. APC expressing HLA-A*02:01 were incubated with EBV LMP2-derived antigenic peptide CLGGLLTMV (SEQ ID NO: 106) at a range between 0.000005 and 50 μM. Successfully binding Jurkat reporter cells are activated via the TCR and generate a luciferase signal that can be quantified. Jurkats NT: non-transduced Jurkat cells.

FIG. 5 shows the EBV LMP2-antigen-specific binding of Jurkat reporter cells transduced with TCR receptors. APC expressing HLA-A*02:01 were incubated with EBV LMP2-derived antigenic peptide FLYALALLL (SEQ ID NO: 107) at a range between 0.000005 and 50 μM. Successfully binding Jurkat reporter cells are activated via the TCR and generate a luciferase signal that can be quantified. Jurkats NT: non-transduced Jurkat cells.

FIG. 6A and FIG. 6B show the sequences logo for the possible CDR3α sequences (C-A-T-X¹-G-X²-S-G-Y-S-T-L-T-F (SEQ ID NO:181)), and CDR3β sequences (C-A-S-X³-X⁴-Q-G-G-(S)-X⁵-X⁶-G-Y-T-F (SEQ ID NO: 182)), respectively, binding to a HLA-A*02-restricted EBV LMP2-derived antigenic peptide of amino acid sequence FLYALALLL (SEQ ID 107), whereby: X¹ is E or A; X² is D, G, N or S, or any of the following amino acids with related properties: E, A, Q or T; X³ is S or T, or any of the following amino acids with related properties: N or Q; X⁴ is K, R or T, or any of the following amino acids with related properties: H, S; X⁵ is G or A; X⁶ is Y or S, or any of the following amino acids with related properties: F, W, H or T.

FIG. 7A and FIG. 7B show the sequences logo for the possible CDR3α sequences (C-A-X¹-X²-G-A-G-S-Y-Q-L-T-F (SEQ ID NO:183)), and CDR3β sequences (C-A-S-S-X³-E-G-Q-A-S-S-Y-E-Q-Y-F (SEQ ID NO:184)), respectively, binding to a HLA-A*02-restricted EBV LMP2-derived antigenic peptide of amino acid sequence CLGGLLTMV (SEQ ID NO:106), whereby: X¹ is G or V, or any of the following amino acids with related properties: A, I or L; X² is A or S, or any of the following amino acids with related properties: G or T; X³ is L or A, or any of the following amino acids with related properties: I, V or G.

FIG. 8 shows splice variant peptide RLPGVLPRA-specific binding of Jurkat reporter cells transduced with TCRs. APC expressing HLA-A*02:01 were incubated with MAPK8IP2 splice variant-derived peptide RLPGVLPRA (SEQ ID NO:147) at a range between 0.000005 and 50 μM. Successfully binding Jurkat reporter cells are activated via the TCR and generate a luciferase signal that can be quantified. TCR_A0130 and TCR_A0131 were expressed successfully and recognized peptide RLPGVLPRA presented on HLA-A*02:01-expressing APC. Jurkats NT: non-transduced Jurkat cells.

FIG. 9 shows the sequence logo for possible CDR3α sequence (C-A-F-M-X¹-X²-D-S-X³-X⁴-Y-X⁵-X⁶-I-X⁷ (SEQ ID NO:185)), binding to a HLA-A*02-restricted mutant splice factor-induced splice variant MAPK8IP2-derived antigenic peptide of amino acid sequence RLPGVLPRA (SEQ ID NO:147), whereby X¹ is L or I or E, or any of the following amino acids with related properties: V or D. X² is P or I or A, or any of the following amino acids with related properties: V, L or G. X³ is G or N, or any of the following amino acids with related properties: Q, A, C or S. X⁴ is T or no AA at this position, or S as an amino acid with related properties. X⁵ is K or Q, or any of the following amino acids with related properties: R, H or N. X⁶ is L or Y, or any of the following amino acids with related properties: I, V, F, W or H. X⁷ is F or W.

FIG. 10 shows the HERV-K-specific binding of Jurkat reporter cells transduced with TCRs. APC expressing HLA-A*02:01 were incubated with peptide FLQFKTWWI (SEQ ID NO:148) at a range between 0.000005 and 50 μM. Successfully binding Jurkat reporter cells are activated via the TCR and generate a luciferase signal that can be quantified. TCR_A0100 was expressed successfully and recognized peptide FLQFKTWWI presented on HLA-A*02:01-expressing APC. Jurkats NT: non-transduced Jurkat cells.

FIG. 11 shows that TCR_A0100 is functional when transduced into primary T cells and exposed to cognate peptide antigen pulsed onto target cells. Production of the cytokine IFNγ was used as a readout to measure the effector function of T cells transduced with TCR_A0194, which is TCR_A0100 containing modified mouse constant regions.

FIG. 12A-F show that T cells transduced with TCR_A0194 effectively kills cancer cells expressing endogenous levels of the target HERV-K antigen. Cancer cell line 92.1, which expresses HERV-K gag and the HLA allele HLA-A*02 (A, D, E), was used to measure cytolysis at 12 h (A, B), 24 h (C, D), and 48 h (E, F). As a control for HLA-specific killing of target cells, HERV-K gag-expressing but HLA-A*02-negative cell line MEL202 was used (B, D, F).

FIG. 13A-B show the effector functions of TCR-transduced primary T cells. Three TCRs targeting a mutant splice factor-induced peptide of MAPK8IP2, known to be shared across patients with multiple types of cancer, were successfully isolated from a renal cell carcinoma patient. The TCRs are A0130 modified with mouse constant regions, A0191 (black circles), A0131 modified with mouse constant regions, A0192 (up triangles), and A0132 modified with mouse constant regions, A0193 (down triangles). Data shows the reactivity of TCR-expressing primary T cells to peptide-pulsed HLA-A*02-positive cells (A). The supernatant of the experiment in A was used to quantify IFNγ by ELISA (B). The negative control consisted of non-transduced cells (empty circles).

FIG. 14A-B show the isolation and validation of cells expressing TCRs A0358 and A0359 that bind to RLPGVLPRA (SEQ ID NO:147). (14A) Validation of specific T cell expansion after in vitro culture. T cells stimulated with peptide RLPGVLPRA and expanded in the presence of the peptide were tested with HLA-A*02:01 tetramers loaded with peptide RLPGVLPRA using flow cytometry. The population of peptide-specific cells is shown in the lower right of the graph. Cells shown were first gated on single cells, live cells and CD8+ cells. The tetramer positive cells (tet APC, SFz3Bmut (RLPG)) versus CD137 expression is shown. (14B) Sorting of tetramer-binding T cells for the isolation of TCR sequences. Index sorting was used to track the flow cytometry profile of the single cells sorted into wells for RT-PCR to amplify the TCR alpha and beta chains. Cells that showed the same TCR sequence (clonal expansion) are highlighted as filled circles in the flow cytometry analysis graphs. For both graphs cells were first sorted on singlets and live cells. The left graph shows that the clonal cells are CD4 negative and CD8 positive. The right graph shows that the clonal cells bind to RLPGVLPRA-loaded-tetramer.

FIG. 15 shows that TCRs A0358, A0130 (A0362) and A0131 (A0363) bind specifically to peptide RLPGVLPRA. Raji cells expressing HLA-A*02:01 were used as antigen presenting cells and loaded with peptide RLPGVLPRA at a range of concentrations as indicated on the x axis. Jurkat reporter cells transduced with TCRs A0358, A0130 and A0131 were added to the antigen presenting cells, leading to an antigen-specific TCR-mediated induction of luciferase, quantified as RLU (y axis). NT: non-transduced T cells. EC50 values were calculated using the parameters [Agonist] vs. response—Variable slope (four parameters) in Graphpad Prism.

FIG. 16 shows the sequence logo for possible CDR3α sequence (C-A-F-M-X¹-X²-D-S-X³-X⁴-Y-X⁵-X⁶-I-X⁷ (SEQ ID NO:304)), binding to a HLA-A*02-restricted mutant splice factor-induced splice variant MAPK8IP2-derived antigenic peptide of amino acid sequence RLPGVLPRA (SEQ ID NO:147), whereby X¹ is L or I or E or G, or any of the following amino acids with related properties: V or D. X² is P or I or A, or any of the following amino acids with related properties: V, L or G. X³ is G or N, or any of the following amino acids with related properties: Q, A, C or S. X⁴ is T or no AA at this position, or S as an amino acid with related properties. X⁵ is K or Q, or any of the following amino acids with related properties: R, H or N. X⁶ is L or Y, or any of the following amino acids with related properties: I, V, F, W or H. X⁷ is F or W.

FIG. 17 shows the sequence logo for possible CDR3α sequence (C-A-F-M-X¹-X²-D-S-N-Y-Q-L-I-W (SEQ ID NO: 305)), binding to a HLA-A*02-restricted mutant splice factor-induced splice variant MAPK8IP2-derived antigenic peptide of amino acid sequence RLPGVLPRA (SEQ ID NO:147), whereby X¹ is I or E, or any of the following amino acids with related properties: V or D. X² is P or A, or any of the following amino acids with related properties: V, L or G.

FIG. 18 shows the sequence logo for possible CDR3α sequence (C-A-X¹-X²-X³-X⁴-D-S-N-Y-Q-L-I-W (SEQ ID NO: 306)), binding to a HLA-A*02-restricted mutant splice factor-induced splice variant MAPK8IP2-derived antigenic peptide of amino acid sequence RLPGVLPRA (SEQ ID NO:147), whereby X¹ is F or M, or any of the following amino acids with related properties: Y or W. X² is M or R, or any of the following amino acids with related properties: K or H. X³ is I or E, or any of the following amino acids with related properties: V, L or D. X⁴ is P or A, or G as an amino acid with related properties.

### DETAILED DESCRIPTION

### I. Introduction

T cells are the most actively studied cell type in the growing field of adoptive cellular therapeutics. T cells interact specifically with the target of their T cell receptor (TCR), enabling highly specific responses with minimal side effects. These potentially highly effective and specific responses can be engineered towards novel antigens and targets by inserting a new receptor with the desired specificity into a T cell. However, development of entirely new types of receptors is time consuming, expensive, and fails to take advantage of the fact that, through development of the endogenous T cell repertoire, the body naturally produces TCRs that bind almost any possible antigenic target. The ability to obtain human T cells and replace their endogenous TCR with a TCR having a desired antigen specificity could be transformative in the development and application of adoptive T cell therapies.

In various embodiments, the present invention provides human T cell receptors (TCRs) that are capable of binding to antigenic peptides associated with disease, such as EBV-derived antigenic peptides, and nucleic acids encoding the TCRs of the invention. In various embodiments, the present invention also provides human T cell receptors (TCRs) that are capable of binding to tumor-derived or tumor-associated peptides, such as mutant splice-factor-induced peptide of MAPK8IP2 and peptide from HERV-K gag protein. Also provided is a method of transducing a human T cell with a nucleic acid encoding a T cell receptor such that the T cell integrates the nucleic acid into its genome and expresses the encoded TCR. In some embodiments, the invention provides a host cell with the nucleic acid integrated into the host cell genome, and such a T cell expressing the TCR. The instant invention further provides methods of preventing, treating or ameliorating a disease in a subject by administering to a subject in need thereof a cell of the invention.

### II. Definitions

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984; Mullis et al. U.S. Pat. No. 4,683,195); Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, ‘Gene Expression Technology’ (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

It is understood that wherever aspects are described herein with the language ‘comprising,’ otherwise analogous aspects described in terms of ‘consisting of’ and/or ‘consisting essentially of’ are also contemplated. The use of the alternative (e.g., ‘or’) should be understood to mean either one, both, or any combination thereof of the alternatives. As used herein, the indefinite articles ‘a’ or ‘an’ should be understood to refer to ‘one or more’ of any recited or enumerated component. The singular forms ‘a,’ ‘an,’ and ‘the’ include plural referents unless the context clearly dictates otherwise.

The terms ‘about’ or ‘consisting essentially of’ refer to a value or composition that is within an acceptable error range for the particular value or composition as determined by one of ordinary skill in the art, which will depend in part on how the value or composition is measured or determined, i.e., the limitations of the measurement system. For example, in some embodiments, ‘about’ or ‘consisting essentially of’ can mean within 1 or more than 1 standard deviation per the practice in the art. Alternatively, ‘about’ or ‘consisting essentially of’ can mean a range of up to 10% (i.e., +/−10%). The term ‘about’ in relation to a numerical value is optional, and means for example +/−10%. By way of illustration, reference e.g. to ‘about 10%’ is to be construed as 9% to 11%. In instances herein where ‘about’ is recited, the value it precedes is also specifically contemplated. By way of illustration, reference e.g. to ‘about 10%’ also specifically contemplates 10%.

Methods and processes according to the present disclosure may be performed, and products may be present or provided, in vitro, ex vivo or in vivo. The term ‘in vitro’ is intended to encompass procedures performed under, and/or materials present/provided in, laboratory conditions, or in culture. The term ‘in vivo’ is intended to encompass procedures performed with/on, and/or materials present/provided in, intact multi-cellular organisms (e.g. a human or animal body). The term ‘ex vivo’ is intended to encompass procedures performed, and/or materials present/provided, outside of the human or animal body. The relevant materials may have been obtained from the human or animal body, and it may be contemplated to administer the relevant material, and/or products of the procedure, to a human/animal body.

The term ‘T cell receptor’ (TCR), as used herein, refers to a heteromeric cell-surface receptor capable of specifically interacting with a target antigen. Herein, a ‘TCR’ or an antigen-binding fragment thereof may also be referred to as an ‘antigen-binding molecule’. As used herein, ‘TCR’ includes but is not limited to naturally occurring and non-naturally occurring TCRs; full-length TCRs and antigen binding portions thereof, chimeric TCRs; TCR fusion constructs; and synthetic TCRs. In humans, TCRs are expressed on the surface of T cells, and they are responsible for T cell recognition and targeting of antigen presenting cells. Antigen presenting cells (APC) display fragments of foreign or self proteins (antigens) complexed with the major histocompatibility complex (MHC; also referred to herein as complexed with a HLA molecule, e.g., a HLA class I or class II molecule). A TCR recognizes and binds to the antigen:HLA complex and recruits CD3 (expressed by T cells), activating the TCR. The activated TCR initiates downstream signaling and an immune response, including the destruction of the APC.

In general, a TCR can comprise two chains, an alpha chain and a beta chain (or less commonly a gamma chain and a delta chain), interconnected by disulfide bonds. Each chain comprises a variable domain (e.g. alpha chain variable domain and beta chain variable domain) and a constant region (e.g. alpha chain constant region and beta chain constant region). The variable domain is located distal to the cell membrane, and the variable domain interacts with an antigen. A variable domain may also be referred to herein as a ‘variable region’. The constant region is located proximal to the cell membrane. A TCR can further comprise a transmembrane region and a short cytoplasmic tail. As used herein, the term ‘constant region’ encompasses the transmembrane region and the cytoplasmic tail, when present, as well as the traditional ‘constant region.’

The variable domains can be further subdivided into regions of hypervariability, termed complementarity determining regions (CDRs), interspersed with regions that are more conserved, termed framework regions (FR). Each alpha chain variable domain and beta chain variable domain comprises three CDRs and four FRs: FR1, CDR1, FR2, CDR2, FR3, CDR3, FR4. Each variable domain contains a binding domain that interacts with an antigen. Though all three CDRs on each chain are involved in antigen binding, CDR3 is believed to be the primary antigen binding region. CDR1 and CDR2 are believed to primarily recognize the HLA complex.

The term CDR3 used herein describes the CDR3 region including the fixed C′-terminal amino acid C (cysteine) and N′-terminal amino acid F (phenylalanine) or W (Tryptophan), or the respective nucleotide sequence coding for these amino acids. The CDR3 including C′-terminal C and N′-terminal F/W, or the respective codons, is also termed ‘Junction’ in the field.

Where not expressly stated, and unless the context indicates otherwise, the term ‘TCR’ also includes an antigen-binding fragment or an antigen-binding portion of any TCR disclosed herein, and includes a monovalent and a divalent fragment or portion, and a single chain TCR. The term ‘TCR’ is not limited to naturally occurring TCRs bound to the surface of a T cell. As used herein, the term ‘TCR’ further refers to a TCR described herein that is expressed on the surface of a cell other than a T cell (e.g., a cell that naturally expresses or that is modified to express CD3, as described herein), or a TCR described herein that is free from a cell membrane (e.g., an isolated TCR or a soluble TCR).

An ‘antigen binding molecule,’ ‘portion of a TCR,’ or ‘TCR fragment’ may refer to a portion of an TCR less than the whole. An antigen binding molecule can include the antigenic complementarity determining regions (CDRs).

An ‘antigen’ refers to any molecule, e.g., a peptide, that provokes an immune response or is capable of being bound by a TCR. An ‘epitope,’ as used herein, refers to a portion of a polypeptide that provokes an immune response or is capable of being bound by a TCR. The immune response may involve either antibody production, or the activation of specific immunologically competent cells, or both. A person of skill in the art would readily understand that any macromolecule, including virtually all proteins or peptides, can serve as an antigen. An antigen and/or an epitope can be endogenously expressed, i.e. expressed by genomic DNA, or can be recombinantly expressed. An antigen and/or epitope can be of exogenous origin. An antigen and/or epitope can possess modifications to the amino acids comprising the antigen and/or epitope if of polypeptide origin (e.g. phosphorylation, glycosylation, cysteinylation, deamidation, and/or other post-translational modifications to the amino acids within the antigen and/or epitope). An antigen and/or an epitope can be specific to a certain tissue, such as a cancer cell, or it can be broadly expressed. In addition, fragments of larger molecules can act as antigens. In some embodiments, antigens are tumor antigens. An epitope can be present in a longer polypeptide (e.g., in a protein), or an epitope can be present as a fragment of a longer polypeptide. In some embodiments, an epitope is complexed with a major histocompatibility complex (MHC; also referred to herein as a HLA molecule, e.g., a HLA class I or class II molecule).

‘Antigen-derived’, for example ‘EBV-derived’, refers to an immunogenic peptide/epitope being a portion of the antigen/polypeptide from which it has been processed. For example, an antigen is processed in the cell by the proteasome or immunoproteasome and the resulting antigen-derived peptides are presented on the MHC class I or MHC class II complex.

An ‘antigen-binding moiety’ may be any moiety capable of binding to a target antigen. Such moieties include moieties comprising an antibody heavy chain variable region (VH) and an antibody light chain variable region (VL) of an antibody capable of specific binding to a target antigen. Examples of such antigen-binding moieties include Fv regions (e.g. scFvs, which are formed by the VH and VL regions, joined by a linker) and Fab regions, which comprise the VH-CH1 and VL-CL regions of antibodies (e.g. scFvs, which are formed by the VH-CH1 and VL-CL regions, joined by a linker). Further examples of antigen-binding moieties include aptamers capable of binding to the target antigen, e.g. nucleic acid aptamers (reviewed, for example, in Zhou and Rossi Nat Rev Drug Discov. 2017 16(3):181-202). In some embodiments, an antigen-binding moiety according to the present disclosure may be or comprise an antigen-binding polypeptide, an aptamer, an antigen-binding polypeptide complex, or an antibody or an antigen-binding fragment or derivative thereof.

‘Administering’ refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase ‘parenteral administration’ as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

‘Parenteral’ administration of composition of the invention includes, e.g., subcutaneous (s.c.), intravenous (i.v.), intramuscular (i.m.), or intrasternal injection, or infusion techniques.

As used herein, ‘treating’ or ‘treatment’ refers to an approach for obtaining beneficial or desired results, including and preferably clinical results. Treatment can refer to either the amelioration of symptoms of the disease or condition, or the delaying of the progression of the disease or condition.

A ‘therapeutically effective amount,’ ‘effective dose,’ ‘effective amount,’ or ‘therapeutically effective dosage’ of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term ‘autologous’ refers to any material derived from the same individual to which it is later to be re-introduced. For example, an autologous T cell therapy comprises administering to a subject a T cell that was isolated from the same subject. The term ‘allogeneic’ refers to any material derived from one individual which is then introduced to another individual of the same species. For example, an allogeneic T cell transplantation comprises administering to a subject a T cell that was obtained from a donor other than the subject.

A ‘cancer’ refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A ‘cancer’ or ‘cancer tissue’ can include a tumor.

In some embodiments, the engineered cells have an anti-tumor effect, and methods of the present invention can be used to reduce the tumor size of a tumor. The particular cancer can be responsive to chemo- or radiation therapy or the cancer can be refractory. A refractory cancer refers to a cancer that is not amenable to surgical intervention, and the cancer is either initially unresponsive to chemo- or radiation therapy or the cancer becomes unresponsive over time.

An ‘anti-tumor effect’ as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor, e.g., a vaccine.

The term ‘progression-free survival,’ which can be abbreviated as PFS, as used herein refers to the time from the treatment date to the date of disease progression per the revised IWG Response Criteria for Malignant Lymphoma or death from any cause.

‘Disease progression’ or ‘progressive disease,’ which can be abbreviated as PD, as used herein, refers to a worsening of one or more symptom associated with a particular disease. For example, disease progression for a subject afflicted with a cancer can include an increase in the number or size of one or more malignant lesions, tumor metastasis, and death.

The ‘duration of response,’ which can be abbreviated as DOR, as used herein refers to the period of time between a subject's first objective response to the date of confirmed disease progression, per the revised IWG Response Criteria for Malignant Lymphoma, or death.

The term ‘overall survival,’ which can be abbreviated as OS, is defined as the time from the date of treatment to the date of death.

As used herein, an ‘engineered immune cell’ refers to an immune cell that has been genetically modified as compared to a naturally-occurring immune cell. The term ‘genetically engineered’ or ‘engineered’ refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell or a modified cell that expresses CD3, which can either be obtained from a patient or a donor. The cell can be modified (e.g. as described herein) to express an exogenous construct, such as, e.g., a T cell receptor (TCR) disclosed herein, which can be incorporated into the cell's genome. In some embodiments, the cell is modified to express CD3.

As used herein, a ‘substantially purified’ cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cells that have been separated from the cells with which they are naturally associated in their natural state. In some embodiments, the cells are cultured in vitro. In other embodiments, the cells are not cultured in vitro.

An ‘immune response’ refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including antibodies, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term ‘immunotherapy’ or ‘cellular immunotherapy’ refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies, antibody therapy, fusion protein therapy. T cell therapy can include adoptive T cell therapy, tumor-infiltrating lymphocyte (TIL) immunotherapy, autologous cell therapy, engineered autologous cell therapy (eACT), and allogeneic T cell transplantation. (see, e.g., June, C. H., ed., 2001, In: Cancer Chemotherapy and Biotherapy: Principles and Practice, Lippincott Williams & Wilkins, Baltimore; Vonderheide et al., 2003, Immun. Research 27:1-15).

Cells used in immunotherapy described herein can come from any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety. An immunotherapy can also comprise administering a modified cell to a subject, wherein the modified cell expresses CD3 and a TCR disclosed herein. An immunotherapy can comprise administering a nucleic acid to a subject, e.g. using a vector or another type of targeting method, such that a cell is modified in vivo to express the nucleic acid. The nucleic acid can encode a TCR. In some embodiments, the modified cell is not a T cell.

A ‘patient’ as used herein includes any human who is afflicted with a cancer (e.g., a lymphoma or a leukemia, or a solid tumor). The terms ‘subject’ and ‘patient’ are used interchangeably herein.

The term ‘HLA,’ as used herein, refers to the human leukocyte antigen. HLA genes encode the major histocompatibility complex (MHC) proteins in humans. MHC proteins are expressed on the surface of cells and are involved in activation of the immune response. HLA class I genes encode MHC class I molecules, which are expressed on the surface of cells in complex with peptide fragments (antigens) of self or non-self proteins. T cells expressing TCR and CD3 recognize the antigen:MHC class I complex and initiate an immune response to target and destroy antigen presenting cells displaying non-self proteins.

As used herein, an ‘HLA class I molecule’ or ‘MHC class I molecule’ refers to a protein product of a wild-type or variant HLA class I gene encoding an MHC class I molecule. Accordingly, ‘HLA class I molecule’ and ‘MHC class I molecule’ are used interchangeably herein.

The MHC Class I molecule comprises two protein chains: the alpha chain and the β2-microglobulin (β 2m) chain. Human β 2m is encoded by the B2M gene. The amino acid sequence of β 2m is set forth in SEQ ID NO: 144 (Table 1). The alpha chain of the MHC Class I molecule is encoded by the HLA gene complex. The HLA complex is located within the 6p21.3 region on the short arm of human chromosome 6 and contains more than 220 genes of diverse function. The HLA gene are highly variant, with over 20,000 HLA alleles and related alleles, including over 15,000 HLA Class I alleles, known in the art, encoding thousands of HLA proteins, including over 10,000 HLA Class I proteins (see, e.g., hla.alleles.org). There are at least three genes in the HLA complex that encode an MHC Class I alpha chain protein: HLA-A, HLA-B, and HLA-C. In addition, HLA-E, HLA-F, and HLA-G encode proteins that associate with the MHC Class I molecule.

TABLE 1
Amino Acid Sequence of Human β2m SEQ ID NO: 144

| SEQ ID NO: | Human Beta-2-globuline (β2m) |
|---|---|
| 144 | MSRSVALAVLALLSLSGLEAIQRTPKIQVYSRHPAENGKSNFLNCYVSGFHPSDIE |
| | VDLLKNGERIEKVEHSDLSFSKDWSFYLLYYTEFTPTEKDEYACRVNHVTLSQP |
| | KIVKWDRDM |

A ‘cytokine,’ as used herein, refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

As used herein, the term ‘nucleic acid’ refers to a polymer comprising multiple nucleotide monomers (e.g., ribonucleotide monomers or deoxyribonucleotide monomers). ‘Nucleic acid’ includes, for example, genomic DNA, cDNA, RNA, and DNA-RNA hybrid molecules. Nucleic acid molecules can be naturally occurring, recombinant, or synthetic. In addition, nucleic acid molecules can be single-stranded, double-stranded or triple-stranded. In some embodiments, nucleic acid molecules can be modified. In the case of a double-stranded polymer, ‘nucleic acid’ can refer to either or both strands of the molecule.

The term ‘nucleotide sequence,’ in reference to a nucleic acid, refers to a contiguous series of nucleotides that are joined by covalent linkages, such as phosphorus linkages (e.g., phosphodiester, alkyl and aryl-phosphonate, phosphorothioate, phosphotriester bonds), and/or non-phosphorus linkages (e.g., peptide and/or sulfamate bonds). In certain embodiments, the nucleotide sequence encoding, e.g., a target-binding molecule linked to a localizing domain is a heterologous sequence (e.g., a gene that is of a different species or cell type origin). The terms ‘nucleotide’ and ‘nucleotide monomer’ refer to naturally occurring ribonucleotide or deoxyribonucleotide monomers, as well as non-naturally occurring derivatives and analogs thereof. Accordingly, nucleotides can include, for example, nucleotides comprising naturally occurring bases (e.g., adenosine, thymidine, guanosine, cytidine, uridine, inosine, deoxyadenosine, deoxythymidine, deoxyguanosine, or deoxycytidine) and nucleotides comprising modified bases known in the art.

Where a nucleotide sequence is disclosed herein, the reverse complement thereof is also expressly contemplated. Moreover, in each instance wherein a nucleotide sequence is disclosed herein, codon degenerate nucleotide sequences thereof encoding the same amino acid sequence are also expressly contemplated. A ‘codon degenerate nucleotide sequence’ of a reference nucleotide sequence refers to a nucleotide sequence having a non-identical nucleotide sequence to the nucleotide sequence of the reference nucleotide sequence, but encoding the same amino acid sequence as the amino acid sequence encoded by the reference nucleotide sequence, as a consequence of degeneracy of the genetic code.

As will be appreciated by those of skill in the art, in some aspects, a nucleic acid described herein may further comprise a plasmid sequence. The plasmid sequence can include, for example, one or more operatively linked sequences selected from the group consisting of a promoter sequence, a selection marker sequence, and a locus-targeting sequence.

The term ‘sequence identity’ means that two nucleotide or amino acid sequences, when optimally aligned, such as by the programs GAP or BESTFIT using default gap weights, share at least, e.g., at least about 70% sequence identity, at least about 80% sequence identity, at least about 85% sequence identity, at least about 90% sequence identity, at least 95% sequence identity, at least about 99% sequence identity, or more. For sequence comparison, typically one sequence acts as a reference sequence (e.g., parent sequence), to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al. 2000, Current Protocols in Molecular Biology). One example of algorithm that is suitable for determining percent sequence identity and sequence similarity is the BLAST algorithm, which is described in Altschul et al, J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

As used herein, the term ‘operably linked’ may refer to a juxtaposition or arrangement of specified elements that allows them to perform in concert to bring about an effect. For example, a promoter may be operably linked to a coding sequence if it controls the transcription of the coding sequence.

‘Expression vector’ refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

T cell receptors, peptides/polypeptides, peptide/polypeptide complexes, nucleic acids/polynucleotides, vectors, compositions or cells according to the present disclosure may optionally be provided in isolated or purified form. For example, articles according to the present disclosure may be isolated/purified from naturally-occurring biological material.

The term ‘isolated’ refers to a composition, compound, substance, or molecule altered by the hand of man from the natural state. For example, a composition or substance that occurs in nature is isolated if it has been changed or removed from its original environment, or both. For example, a polynucleotide or a polypeptide naturally present in a living animal is not isolated, but the same polynucleotide or polypeptide separated from the coexisting materials of its natural state is isolated, as the term is employed herein.

‘Encoding’ refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a ‘nucleotide sequence encoding an amino acid sequence’ includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

A ‘vector’ is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term ‘vector’ includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

The term ‘promoter’ as used herein is defined as a DNA sequence recognized by the synthetic machinery of the cell, or introduced synthetic machinery, required to initiate the specific transcription of a polynucleotide sequence.

As used herein, the term ‘promoter/regulatory sequence’ means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A ‘constitutive’ promoter is a nucleotide sequence which, when operably linked with a polynucleotide which encodes or specifies a gene product, causes the gene product to be produced in a cell under most or all physiological conditions of the cell.

An ‘inducible’ promoter is a nucleotide sequence which, when operably linked with a polynucleotide which encodes or specifies a gene product, causes the gene product to be produced in a cell substantially only when an inducer which corresponds to the promoter is present in the cell.

A ‘tissue-specific’ promoter is a nucleotide sequence which, when operably linked with a polynucleotide encodes or specified by a gene, causes the gene product to be produced in a cell substantially only if the cell is a cell of the tissue type corresponding to the promoter.

A ‘lentivirus’ as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The terms ‘peptide,’ ‘polypeptide,’ and ‘protein’ are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. ‘Polypeptides’ include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. An exemplary ‘peptide’ of a length of normally between 8 and 12 amino acids, presented on MHC-I, represents the molecular structure recognized by a TCR. A ‘peptide’ can be interchangeably called a ‘T cell epitope’ or ‘epitope’.

As used herein, the term ‘conservative sequence modifications’ is intended to refer to amino acid modifications that do not significantly affect or alter the binding characteristics of the TCR containing the amino acid sequence. Such conservative modifications include amino acid substitutions, additions and deletions. Modifications can be introduced into a TCR of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the TCR can be replaced with other amino acid residues from the same side chain family and the altered TCR can be tested for the ability to bind antigens using recognized functional assays.

The phrase ‘antigenic specificity,’ as used herein, means that the TCR can specifically bind to and immunologically recognize an antigen. Exemplary antigens include, but are not limited to EBV antigens, e.g., BRLF1, or LMP2, and mutant splice factor-induced peptide of MAPK8IP2, or HERV-K gag protein.

The term ‘antigen-presenting cell’, as used herein, designates cells having the capability to present processed antigenic moiety fragments via MHC class I or MHC class II molecules. Most cell types including cancer cells can express MHC class I molecules and present fragments via MHC class I molecules, while MHC class II molecules are expressed on professional antigen presenting cells. Professional antigen-presenting cells may be a B-cell, a monocyte, or a dendritic cell. The antigen presenting cells may be synthetic, or be isolated from peripheral blood mononuclear cells (PBMCs). Artificial APCs are a type of cell line that expresses a HLA molecule of interest for testing of TCR binding. The HLA protein can be endogenously expressed, or the artificial APCs can be engineered to express the HLA molecule of interest. Artificial APCs expressing the HLA allele of interest can be loaded with peptides such that the binding of a TCR to a peptide:HLA class I complex can be tested.

In the context of the present invention, by ‘EBV-associated disease, disorder or condition’ is meant any clinical pathology resulting from infection by an Epstein Barr virus. To this end, EBV-associated disease, disorder or condition can mean any disease caused, directly or indirectly, by EBV as well as diseases which predispose a patient to infection by EBV. Examples of diseases falling into the former category include infectious mononucleosis, nasopharyngeal carcinoma, and Burkitt's lymphoma. Diseases in the latter category (i.e., those which place the patient at risk of EBV infection) include acquired immune deficiency syndrome and, generally, any condition that causes a state of immunosuppression or decreased function of the immune system such as patients who receive organ transplants and certain cancer therapies. In one particular embodiment, the EBV-associated disease, disorder or condition suitably is or comprises multiple sclerosis.

The term ‘transfected’ or ‘transformed’ or ‘transduced’ as used herein refers to a process by which exogenous nucleic acid is transferred or introduced into the host cell. A ‘transfected’ or ‘transformed’ or ‘transduced’ cell is one which has been transfected, transformed or transduced with exogenous nucleic acid. The cell includes the primary subject cell and its progeny.

By the term ‘specifically binds,’ as used herein with respect to a T cell receptor, is meant a T cell receptor which recognizes a specific antigen complexed with an MHC molecule, but does not substantially recognize or bind other antigen:MHC complexes in a sample.

By the term ‘stimulation,’ is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like. A ‘stimulatory molecule,’ as the term is used herein, means a molecule on a T cell that specifically binds with a cognate stimulatory ligand present on an antigen presenting cell.

Throughout this specification and the claims which follow, unless the context requires otherwise, the word ‘comprise’, and variations such as ‘comprises’ and ‘comprising’, will be understood to imply the inclusion of a stated integer or step or group of integers or steps but not the exclusion of any other integer or step or group of integers or steps. When used herein, the term ‘comprising’ can be substituted with the term ‘containing’ or ‘including’ or sometimes when used herein with the term ‘having’.

When used herein ‘consisting of’ excludes any element, step, or ingredient not specified in the claim element. When used herein, ‘consisting essentially of’ does not exclude materials or steps that do not materially affect the basic and novel characteristics of the claim.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

In various embodiments, the invention includes one or more of the features defined hereinabove.

### III. Detailed Description of the Embodiments

In various embodiments, the present invention provides human T cell receptors (TCRs) that are capable of binding to antigenic peptides, and nucleic acids encoding the TCRs described herein. In various embodiments, the present invention provides human T cell receptors (TCRs) that are capable of binding to EBV-derived antigenic peptides. In various embodiments, the present invention also provides human T cell receptors (TCRs) that are capable of binding to tumor-derived or tumor-associated peptides, such as mutant splice-factor-induced peptide of MAPK8IP2 and a peptide from HERV-K gag protein. Also provided is a method of transfecting a human T cell with a nucleic acid encoding a T cell receptor such that the T cell integrates the nucleic acid into its genome and expresses the encoded TCR. The method may be performed in vitro, ex vivo or in vivo In some embodiments, the invention provides a host cell with the nucleic acid integrated into the host cell genome, and such a T cell expressing the TCR. The instant invention further provides methods of preventing, treating or ameliorating a disease in a subject by administering to a subject in need thereof a cell, TCR, polypeptide, nucleic acid, vector and/or composition of the invention.

### A. Therapeutic and Prophylactic Applications

The TCRs, antigen-binding molecules, polypeptides, nucleic acids, expression vectors, cells and compositions described herein find use in therapeutic and prophylactic methods.

The present disclosure provides a TCR, antigen-binding molecule, polypeptide, nucleic acid (or plurality thereof), expression vector (or plurality thereof), cell or composition described herein for use in a method of medical treatment or prophylaxis. Also provided is a TCR, antigen-binding molecule, polypeptide, nucleic acid (or plurality thereof), expression vector (or plurality thereof), cell or composition described herein for use in a method of treating or preventing a disease or condition described herein. Also provided is the use of an antigen-binding molecule, polypeptide, nucleic acid (or plurality thereof), expression vector (or plurality thereof), cell or composition described herein in the manufacture of a medicament for treating or preventing a disease or condition described herein. Also provided is a method of treating or preventing a disease or condition described herein, comprising administering to a subject a therapeutically or prophylactically effective amount of a TCR, antigen-binding molecule, polypeptide, nucleic acid (or plurality thereof), expression vector (or plurality thereof), cell or composition described herein.

The methods may be effective to reduce the development or progression of a disease/condition, alleviation of the symptoms of a disease/condition or reduction in the pathology of a disease/condition. The methods may be effective to prevent progression of the disease/condition, e.g. to prevent worsening of, or to slow the rate of development of, the disease/condition. In some embodiments, the methods may lead to an improvement in the disease/condition, e.g. a reduction in the symptoms of the disease/condition or reduction in some other correlate of the severity/activity of the disease/condition. In some embodiments, the methods may prevent development of the disease/condition to a later stage (e.g. a chronic stage or metastasis).

In accordance with various aspects of the present disclosure, treatment or prevention of a disease/condition may comprise one or more of the following: reducing the number and/or activity of cells presenting the MHC:peptide complex for which the TCR is specific; cell killing of/cytotoxicity to cells presenting the MHC:peptide complex for which the TCR is specific; and anti-cancer activity (e.g. cytotoxicity to cancer cells, tumor growth inhibition, reduction of metastasis, etc.) against cancer comprising cells presenting the MHC:peptide complex for which the TCR is specific.

It will be appreciated that articles of the present disclosure find use in the treatment/prevention of diseases/conditions that would derive therapeutic or prophylactic benefit from a reduction in the number or activity of cells infected with EBV and/or expressing EBV-derived antigenic peptides, e.g. cells of an EBV-associated cancer.

For example, the disease/condition may be a disease/condition in which a cell infected with EBV, a cell comprising an EBV antigen (e.g. an EBV antigen described herein, e.g. selected from BRLF1, LMP2 and BZLF1) or a cell comprising a peptide of an EBV antigen (e.g. a peptide of an EBV antigen described herein, e.g. selected from SEQ ID NO:105, SEQ ID NO:106, SEQ ID NO:107, SEQ ID NO:145 and SEQ ID NO:146) is pathologically implicated. Such diseases/conditions include those in which a cell infected with EBV, a cell comprising an EBV antigen (e.g. an EBV antigen described herein, e.g. selected from BRLF1, LMP2 and BZLF1) or a cell comprising a peptide of an EBV antigen (e.g. a peptide of an EBV antigen described herein, e.g. selected from SEQ ID NO: 105, SEQ ID NO:106, SEQ ID NO:107, SEQ ID NO:145 and SEQ ID NO:146) is positively-associated with the onset, development or progression of the disease/condition, and/or severity of one or more symptoms of the disease/condition, or in which such a cell is a risk factor for the onset, development or progression of the disease/condition.

In some embodiments, the disease/condition to be treated/prevented in accordance with the present disclosure is a disease/condition characterized by the presence of a cell infected with EBV, a cell comprising an EBV antigen (e.g. an EBV antigen described herein, e.g. selected from BRLF1, LMP2 and BZLF1) or a cell comprising a peptide of an EBV antigen (e.g. a peptide of an EBV antigen described herein, e.g. selected from SEQ ID NO: 105, SEQ ID NO: 106, SEQ ID NO:107, SEQ ID NO:145 and SEQ ID NO: 146). In some embodiments, the disease/condition is characterised by an increased number/proportion/activity of such cells as compared to the number/proportion/activity of such cells observed in the absence of the disease/condition (e.g. in a healthy subject, or in equivalent non-diseased tissue). It will also be appreciated that articles of the present disclosure find use in the treatment/prevention of diseases/conditions that would derive therapeutic or prophylactic benefit from a reduction in the number or activity of cells comprising a mutant splice-factor-induced peptide of MAPK8IP2 (e.g. SEQ ID NO:147).

For example, the disease/condition may be a disease/condition in which a cell comprising a mutant splice-factor-induced peptide of MAPK8IP2 (e.g. SEQ ID NO:147) is pathologically implicated. Such diseases/conditions include those in which a cell comprising a mutant splice-factor-induced peptide of MAPK8IP2 (e.g. SEQ ID NO:147) is positively-associated with the onset, development or progression of the disease/condition, and/or severity of one or more symptoms of the disease/condition, or in which such a cell is a risk factor for the onset, development or progression of the disease/condition.

In some embodiments, the disease/condition to be treated/prevented in accordance with the present disclosure is a disease/condition characterized by the presence of a cell comprising a mutant splice-factor-induced peptide of MAPK8IP2 (e.g. SEQ ID NO:147). In some embodiments, the disease/condition is characterised by an increased number/proportion/activity of such cells as compared to the number/proportion/activity of such cells observed in the absence of the disease/condition (e.g. in a healthy subject, or in equivalent non-diseased tissue).

It will also be appreciated that articles of the present disclosure find use in the treatment/prevention of diseases/conditions that would derive therapeutic or prophylactic benefit from a reduction in the number or activity of cells comprising a peptide of HERV-K gag protein (e.g. SEQ ID NO:148).

For example, the disease/condition may be a disease/condition in which a cell comprising HERV-K gag protein or a cell comprising a peptide of HERV-K gag protein (e.g. SEQ ID NO:148) is pathologically implicated. Such diseases/conditions include those in which a cell comprising HERV-K gag protein or a cell comprising a peptide of HERV-K gag protein (e.g. SEQ ID NO:148) is positively-associated with the onset, development or progression of the disease/condition, and/or severity of one or more symptoms of the disease/condition, or in which such a cell is a risk factor for the onset, development or progression of the disease/condition.

In some embodiments, the disease/condition to be treated/prevented in accordance with the present disclosure is a disease/condition characterized by the presence of a cell comprising HERV-K gag protein or a cell comprising a peptide of HERV-K gag protein (e.g. SEQ ID NO:148). In some embodiments, the disease/condition is characterised by an increased number/proportion/activity of such cells as compared to the number/proportion/activity of such cells observed in the absence of the disease/condition (e.g. in a healthy subject, or in equivalent non-diseased tissue).

In some embodiments, the disease to be treated/prevented in accordance with the present disclosure is a cancer. Cancer may refer to any unwanted cell proliferation (or any disease manifesting itself by unwanted cell proliferation), neoplasm or tumor. The cancer may be benign or malignant and may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, and/or white blood cells. Tumors may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, hematologic cancer and sarcoma.

In some embodiments the cancer is selected from the group consisting of: a solid cancer, a hematological cancer, gastric cancer (e.g. gastric carcinoma, gastric adenocarcinoma, gastrointestinal adenocarcinoma), liver cancer (hepatocellular carcinoma, cholangiocarcinoma), head and neck cancer (e.g. head and neck squamous cell carcinoma), oral cavity cancer (e.g. oropharyngeal cancer (e.g. oropharyngeal carcinoma), oral cancer, laryngeal cancer, nasopharyngeal carcinoma, oesophageal cancer), colorectal cancer (e.g. colorectal carcinoma), colon cancer, colon carcinoma, cervical carcinoma, prostate cancer, lung cancer (e.g. NSCLC, small cell lung cancer, lung adenocarcinoma, squamous lung cell carcinoma), bladder cancer, urothelial carcinoma, skin cancer (e.g. melanoma, advanced melanoma), renal cell cancer (e.g. renal cell carcinoma), ovarian cancer (e.g. ovarian carcinoma), mesothelioma, breast cancer, brain cancer (e.g. glioblastoma), prostate cancer, pancreatic cancer, a myeloid hematologic malignancy, a lymphoblastic hematologic malignancy, myelodysplastic syndrome (MDS), acute myeloid leukemia (AML), chronic myeloid leukemia (CML), acute lymphoblastic leukemia (ALL), lymphoma, non-Hodgkin's lymphoma (NHL), thymoma or multiple myeloma (MM).

In some embodiments the cancer is a cancer in which EBV is pathologically implicated. That is, in some embodiments the cancer is a cancer which is caused or exacerbated by infection with EBV, a cancer for which infection with EBV is a risk factor and/or a cancer for which infection with EBV is positively associated with onset, development, progression, severity or metastasis of the cancer.

EBV infection is implicated in several cancers, as reviewed e.g. in Jha et al., Front Microbiol. (2016) 7:1602, which is hereby incorporated by reference in its entirety.

In some embodiments, the cancer to be treated/prevented is an EBV-associated cancer. In some embodiments, the cancer is a cancer which is caused or exacerbated by infection with EBV, a cancer for which infection with EBV is a risk factor and/or a cancer for which infection with EBV is positively associated with onset, development, progression, severity or metastasis of the cancer. The cancer may be characterised by EBV infection, e.g. the cancer may comprise cells infected with EBV. Such cancers may be referred to as EBV-positive cancers.

EBV-associated cancers which may be treated/prevented in accordance with the present disclosure include B cell-associated cancers such as Burkitt's lymphoma, post-transplant lymphoproliferative disease (PTLD), central nervous system lymphoma (CNS lymphoma), Hodgkin's lymphoma, non-Hodgkin's lymphoma, and EBV-associated lymphomas associated with immunodeficiency (including e.g. EBV-positive lymphoma associated with X-linked lymphoproliferative disorder, EBV-positive lymphoma associated with HIV infection/AIDS, and oral hairy leukoplakia), and epithelial cell-related cancers such as nasopharyngeal carcinoma (NPC) and gastric carcinoma (GC).

In some embodiments, the cancer is selected from lymphoma (e.g. EBV-positive lymphoma), head and neck squamous cell carcinoma (HNSCC; e.g. EBV-positive HNSCC), nasopharyngeal carcinoma (NPC; e.g. EBV-positive NPC), and gastric carcinoma (GC; e.g. EBV-positive GC).

EBV-infection is also implicated in the development/progression of a variety of autoimmune diseases, such as multiple sclerosis, rheumatoid arthritis, Sjögren's syndrome, systemic lupus erythematosus (SLE) and systemic scleroderma; see e.g. Ascherio and Munger Curr Top Microbiol Immunol. (2015); 390(Pt 1):365-85; Houen and Trier, Front. Immunol. January 2021, Vol. 11, art. 587380), and EBV antigen EBNA2 has recently been shown to associate with genetic regions implicated as risk factors for the development of SLE, multiple sclerosis, rheumatoid arthritis, inflammatory bowel disease, type 1 diabetes, juvenile idiopathic arthritis and celiac disease (Harley et al., Nat Genet. (2018) 50(5): 699-707).

In some embodiments, the disease/condition to be treated/prevented in accordance with the present disclosure is selected from: an EBV-associated cancer, a cancer comprising cells comprising the peptide of SEQ ID NO:105, a cancer comprising cells comprising the peptide of SEQ ID NO: 106, a cancer comprising cells comprising the peptide of SEQ ID NO:107, a cancer comprising cells comprising the peptide of SEQ ID NO:145, a cancer comprising cells comprising the peptide of SEQ ID NO:146, a hematological cancer, a myeloid hematologic malignancy, a hematopoietic malignancy, a lymphoblastic hematologic malignancy, myelodysplastic syndrome, leukemia, T cell leukemia, acute myeloid leukemia, chronic myeloid leukemia, acute lymphoblastic leukemia, lymphoma, Hodgkin's lymphoma, non-Hodgkin's lymphoma, B cell non-Hodgkin's lymphoma, diffuse large B cell lymphoma, primary mediastinal B cell lymphoma, EBV-associated lymphoma, EBV-positive B cell lymphoma, EBV-positive diffuse large B cell lymphoma, EBV-positive lymphoma associated with X-linked lymphoproliferative disorder, EBV-positive lymphoma associated with HIV infection/AIDS, oral hairy leukoplakia, Burkitt's lymphoma, post-transplant lymphoproliferative disease, central nervous system lymphoma, anaplastic large cell lymphoma, T cell lymphoma, ALK-positive anaplastic T cell lymphoma, ALK-negative anaplastic T cell lymphoma, peripheral T cell lymphoma, cutaneous T cell lymphoma, NK-T cell lymphoma, extra-nodal NK-T cell lymphoma, thymoma, multiple myeloma, a solid cancer, epithelial cell cancer, gastric cancer, gastric carcinoma, gastric adenocarcinoma, gastrointestinal adenocarcinoma, liver cancer, hepatocellular carcinoma, cholangiocarcinoma, head and neck cancer, head and neck squamous cell carcinoma, oral cavity cancer, oropharyngeal cancer, oropharyngeal carcinoma, oral cancer, laryngeal cancer, nasopharyngeal carcinoma, oesophageal cancer, colorectal cancer, colorectal carcinoma, colon cancer, colon carcinoma, cervical carcinoma, prostate cancer, lung cancer, non-small cell lung cancer, small cell lung cancer, lung adenocarcinoma, squamous lung cell carcinoma, bladder cancer, urothelial carcinoma, skin cancer, melanoma, advanced melanoma, renal cell cancer, renal cell carcinoma, ovarian cancer, ovarian carcinoma, mesothelioma, breast cancer, brain cancer, glioblastoma, prostate cancer, pancreatic cancer, mastocytosis, advanced systemic mastocytosis, germ cell tumor, testicular embryonal carcinoma, an autoimmune disease, SLE, systemic scleroderma, multiple sclerosis, Sjögren's syndrome, arthritis, rheumatoid arthritis, juvenile idiopathic arthritis, inflammatory bowel disease, Crohn's disease, ulcerative colitis, diabetes, type 1 diabetes, and celiac disease.

In some embodiments, the disease/condition to be treated/prevented in accordance with the present disclosure is a disease/condition associated with mutation to a gene encoding a splicing factor. In some embodiments, the disease/condition is a disease/condition associated with mutation to a gene encoding a component of the spliceosome. In some embodiments, the disease/condition is a disease/condition associated with mutation to SF3B1. In some embodiments, the disease/condition is a disease/condition associated with mutation to SUGP1.

Diseases/conditions associated with mutation to SF3B1 are described e.g. in Bigot et al., Cancer Discov. (2021) August; 11(8):1938-1951, Nguyen et al., Int J Mol Sci. (2020) 21(24):9546 2020, Oka et al., Genome Biol. (2021) 22(1):9, Leeksma et al., Front Oncol. (2020) 10:609409 and Cheruiyot et al. Cancer Res. (2021) 81(17):4499-4513, all of which are hereby incorporated by reference in their entirety. Such diseases/conditions include uveal melanoma, myelodysplastic syndrome (MDS), non-small cell lung cancer (NSCLC), chronic lymphocytic leukemia, pancreatic cancer, acute myeloid leukemia and chronic myelomonocytic leukemia. Mutations to SUGP1, which encodes an interaction partner for SF3B1, results in similar splicing patterns as seen for mutant forms of SF3B1 (Alsafadi et al., 2020).

In some embodiments the disease/condition to be treated/prevented in accordance with the present disclosure is selected from: a cancer associated with mutation to SF3B1, a cancer associated with mutation to SUGP1, a cancer comprising cells comprising a mutant splice-factor-induced peptide of MAPK8IP2, a cancer comprising cells comprising the peptide of SEQ ID NO: 147, a hematological cancer, a myeloid hematologic malignancy, myelodysplastic syndrome, leukemia, chronic lymphocytic leukemia, pancreatic cancer, acute myeloid leukemia and chronic myelomonocytic leukemia, melanoma, uveal melanoma, lung cancer, non-small cell lung cancer and pancreatic cancer.

In some embodiments, the disease/condition to be treated/prevented in accordance with the present disclosure is a disease/condition associated with HERV protein expression. HERV protein expression is associated with various cancers, including breast cancer, pancreatic cancer, germ cell tumors, leukemia, prostate cancer, bladder cancer, ovarian cancer, lung cancer, hepatocellular carcinoma, lymphoma, choriocarcinoma, colorectal carcinoma, soft tissue sarcoma and Kaposi's sarcoma-see e.g. Gao et al., Oncol Lett. (2021) 21(2): 121 and Jansz and Faulkner, Genome Biology (2021) 22:1 22, 1-22, both of which are hereby incorporated by reference in their entirety.

In some embodiments the disease/condition to be treated/prevented in accordance with the present disclosure is selected from: a cancer comprising cells expressing a HERV protein, a cancer comprising cells expressing a HERV-K protein, a cancer comprising cells comprising a HERV-K gag protein-derived peptide, a cancer comprising cells comprising the peptide of SEQ ID NO: 148, breast cancer, pancreatic cancer, germ cell tumor, a hematological cancer, leukemia, prostate cancer, bladder cancer, ovarian cancer, lung cancer, liver cancer, hepatocellular carcinoma, lymphoma, uterine cancer, choriocarcinoma, colorectal cancer, colorectal carcinoma, sarcoma, soft tissue sarcoma and Kaposi's sarcoma.

### Administration

Administration of the polypeptides, nucleic acids, vectors, cells and compositions of the present disclosure is preferably in a ‘therapeutically-effective’ or ‘prophylactically-effective’ amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (Ed. A. Adejare), 23rd Edition (2020), Academic Press.

Administration of the articles of the present disclosure may be e.g. parenteral, systemic, topical, intracavitary, intravascular, intravenous, intra-arterial, intramuscular, intrathecal, intraocular, intraconjunctival, intratumoral, subcutaneous, intradermal, oral or transdermal. Administration may be by injection, infusion or ingestion.

In some aspects and embodiments, articles of the present disclosure may be administered to a tissue/organ of interest (e.g. a tissue/organ affected by the disease/condition (e.g. a tissue/organ in which symptoms of the disease/condition manifest)). In some aspects and embodiments, articles of the present disclosure may be administered to the blood (i.e. intravenous/intra-arterial administration) by injection or infusion (e.g. via cannula), or may be administered subcutaneously or orally.

In some embodiments, therapeutic or prophylactic intervention according to the present disclosure may further comprise administering another agent for the treatment/prevention of the relevant disease/condition.

Administration of the TCRs, antigen-binding molecules, polypeptides, nucleic acids, vectors, cells and compositions described herein may be alone or in combination with other treatments, either simultaneously or sequentially dependent upon the condition to be treated.

In some embodiments, the TCRs, antigen-binding molecules, polypeptides, nucleic acids, vectors, cells and compositions described herein may be administered in combination with another TCR, antigen-binding molecule, polypeptide, nucleic acid, vector, cell or composition. In some embodiments, the TCRs, antigen-binding molecules, polypeptides, nucleic acids, vectors, cells and compositions described herein may be administered in combination with another TCR, antigen-binding molecule, polypeptide, nucleic acid, vector, cell or composition as described herein. In some embodiments, a subject is administered with a plurality of (e.g. 2, 3, 4, or more) non-identical TCRs, antigen-binding molecules, polypeptides, nucleic acids, vectors, cells and compositions.

In some embodiments, a subject is administered with a plurality of non-identical TCRs/antigen-binding molecules/polypeptides. In some embodiments, the plurality of non-identical TCRs/antigen-binding molecules/polypeptides are each TCRs/antigen-binding molecules/polypeptides described herein.

In some embodiments, a subject is administered with nucleic acid/vector(s) encoding a plurality of non-identical TCRs/antigen-binding molecules/polypeptides. In some embodiments, a subject is administered with cells comprising/expressing a plurality of non-identical TCRs/antigen-binding molecules/polypeptides, or cells comprising nucleic acid/vector(s) encoding a plurality of non-identical TCRs/antigen-binding molecules/polypeptides. In some embodiments, the plurality of non-identical TCRs/antigen-binding molecules/polypeptides are each TCRs/antigen-binding molecules/polypeptides described herein.

Simultaneous administration refers to administration with another therapeutic agent together, for example as a pharmaceutical composition containing both agents (combined preparation), or immediately after each other and optionally via the same route of administration (e.g. to the same tissue, artery, vein or other blood vessel).

Sequential administration refers to administration of one agent followed after a given time interval by separate administration of another agent. It is not required that the two agents are administered by the same route, although this is the case in some embodiments. The time interval may be any time interval.

Multiple doses of the polypeptides, nucleic acids, vectors, cells and compositions of the present disclosure may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

Administration may be alone or in combination with other treatments, either simultaneously or sequentially dependent upon the disease/condition to be treated. The TCR, antigen-binding molecule, nucleic acid, vector cell or composition described herein and another prophylactic/therapeutic agent may be administered simultaneously or sequentially.

In some embodiments, the methods comprise additional therapeutic or prophylactic intervention, e.g. for the treatment/prevention of a cancer. In some embodiments, the therapeutic or prophylactic intervention is selected from chemotherapy, immunotherapy, radiotherapy, surgery, vaccination and/or hormone therapy. In some embodiments, the therapeutic or prophylactic intervention comprises leukapheresis. In some embodiments, the therapeutic or prophylactic intervention comprises a stem cell transplant.

Simultaneous administration refers to administration of the TCR, antigen-binding molecule, nucleic acid, vector cell or composition and therapeutic agent together, for example as a pharmaceutical composition containing both agents (combined preparation), or immediately after each other and optionally via the same route of administration, e.g. to the same artery, vein or other blood vessel. Sequential administration refers to administration of one of the TCR, antigen-binding molecule, nucleic acid, vector cell or composition or therapeutic agent followed after a given time interval by separate administration of the other agent. It is not required that the two agents are administered by the same route, although this is the case in some embodiments. The time interval may be any time interval.

In some embodiments, treatment of cancer further comprises chemotherapy and/or radiotherapy. Chemotherapy and radiotherapy respectively refer to treatment of a cancer with a drug or with ionising radiation (e.g. radiotherapy using X-rays or γ-rays). The drug may be a chemical entity, e.g. small molecule pharmaceutical, antibiotic, DNA intercalator, protein inhibitor (e.g. kinase inhibitor), or a biological agent, e.g. antibody, antibody fragment, aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

Chemotherapy may involve administration of more than one drug. A drug may be administered alone or in combination with other treatments, either simultaneously or sequentially dependent upon the condition to be treated.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a predetermined timetable, plan, scheme or schedule of chemotherapy administration which may be prepared by a physician or medical practitioner and may be tailored to suit the patient requiring treatment. The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs may be selected from: Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calquence (Acalabrutinib), Campath (Alemtuzumab), Camptosar (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil--Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R—CHOP, R—CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib) and Zytiga (Abiraterone Acetate).

In some embodiments, the treatment may comprise administration of a corticosteroid, e.g. dexamethasone and/or prednisone.

In some embodiments, the TCRs, antigen-binding molecules nucleic acids, vectors and compositions described herein are used in T-cell-based ACT. In some embodiments, the engineered TCRs described herein are exogenously expressed on T-cells through genetic engineering methods including, but not limited to, lentiviral transduction, or messenger ribonucleic acid (mRNA) transfection, of nucleic acids encoding for the TCR sequences described herein. In some embodiments, the TCRs used for ACT comprise a TCR sequence fused with a T-cell binding domain, including but not limited to a single-chain fragment binding to CD3. In some embodiments, the TCRs are used in T-cell based ACT in combination with one or more therapeutic agents, e.g., immune modulating agents, including but not limited to cytokines, TLR agonists, RIG-I like receptor (RLR) agonists.

Adoptive cell transfer (ACT) is an immunotherapy involving administration of immune cells with direct anti-cancer activity to a subject in need thereof. Adoptive cell transfer generally refers to a process by which cells (e.g. immune cells) are obtained from a subject, typically by drawing a blood sample from which the cells are isolated. The cells are then typically modified and/or expanded, and then administered either to the same subject (in the case of adoptive transfer of autologous/autogeneic cells) or to a different subject (in the case of adoptive transfer of allogeneic cells). The treatment is typically aimed at providing a population of cells with certain desired characteristics to a subject, or increasing the frequency of such cells with such characteristics in that subject. Adoptive transfer may be performed with the aim of introducing a cell or population of cells into a subject, and/or increasing the frequency of a cell or population of cells in a subject.

Adoptive transfer of immune cells is described, for example, in Kalos and June (2013), Immunity 39(1): 49-60, and Davis et al. (2015), Cancer J. 21(6): 486-491, both of which are hereby incorporated by reference in their entirety. The skilled person is able to determine appropriate reagents and procedures for adoptive transfer of cells according to the present disclosure, for example by reference to Dai et al., 2016 J Nat Cancer Inst 108(7): djv439, which is incorporated by reference in its entirety.

The advantages of ACT over other immunotherapies are multiple. Firstly, the antitumor T-cells can be grown in vitro in large numbers, then selected for high-avidity recognition of the desired tumor antigen, as well as effector functions. Secondly, in vitro activation circumvents the presence of inhibitory factors found in vivo. Thirdly, ACT allows for manipulation of the host before cell transfer to provide a favorable microenvironment supporting antitumor activity (Rosenberg and Restifo, Science 348(6230):62-68 (2015)).

ACT using naturally occurring tumor-reactive T-cells achieves durable, complete regressions in patients with melanoma, as well as other common epithelial cancers (Rosenberg and Restifo, Science 348(6230):62-68 (2015)). In some embodiments, TCR-expressing T-cells are used for T-cell-based adoptive cell transfer (ACT) as a therapeutic treatment in a subject suffering from cancer, including an EBV-associated cancer.

In other embodiments, ACT is used in combination with immune modulating agents, selected from the group of cytokines, TLR agonist, RIG-I like receptor (RLR) agonists, immune checkpoint inhibitors, chemotherapeutic agents, antibodies, radiotherapy and a combination thereof.

The present disclosure provides methods comprising administering antigen-specific immune cells comprising/expressing a TCR/antigen-binding molecule according to the present disclosure, or antigen-specific immune cells comprising/expressing nucleic acid/a vector encoding a TCR/antigen-binding molecule according to the present disclosure, to a subject.

In some embodiments, the methods comprise generating antigen-specific immune cells, or generating/expanding a population of antigen-specific immune cells. In some embodiments, the methods comprise modifying an immune cell to comprise/express a TCR/antigen-binding molecule according to the present disclosure. In some embodiments, the methods comprise modifying an immune cell to comprise/express nucleic acid/a vector encoding a TCR/antigen-binding molecule according to the present disclosure.

In some embodiments, the methods comprise administering to a subject antigen-specific immune cells modified to express/comprise a TCR/antigen-binding molecule according to the present disclosure (or modified to express/comprise a nucleic acid/vector encoding such a TCR/antigen-binding molecule).

In some embodiments, the methods comprise:
(a) modifying an immune cell to express or comprise a TCR/antigen-binding molecule according to the present disclosure, or to express or comprise nucleic acid/a vector encoding a TCR/antigen-binding molecule according to the present disclosure, and
(b) administering the immune cell modified to express or comprise a TCR/antigen-binding molecule according to the present disclosure, or modified to express or comprise nucleic acid/a vector encoding a TCR/antigen-binding molecule according to the present disclosure, to a subject.

In some embodiments, the methods comprise:
(a) isolating or obtaining immune cells;
(b) modifying an immune cell to express or comprise a TCR/antigen-binding molecule according to the present disclosure, or to express or comprise nucleic acid/a vector encoding a TCR/antigen-binding molecule according to the present disclosure, and
(c) administering the immune cell modified to express or comprise a TCR/antigen-binding molecule according to the present disclosure, or modified to express or comprise nucleic acid/a vector encoding a TCR/antigen-binding molecule according to the present disclosure, to a subject.

In some embodiments, the subject from which the immune cells (e.g. PBMCs) are isolated is the same subject to which cells are administered (i.e., adoptive transfer may be of autologous/autogeneic cells). In some embodiments, the subject from which the immune cells (e.g. PBMCs) are isolated is a different subject to the subject to which cells are administered (i.e., adoptive transfer may be of allogeneic cells).

In some embodiments the methods may comprise one or more of:
obtaining a blood sample from a subject;
isolating immune cells (e.g. PBMCs) from a blood sample which has been obtained from a subject;
culturing the immune cells in in vitro or ex vivo cell culture;
modifying an immune cell to express or comprise a TCR/antigen-binding molecule according to the present disclosure, or to express or comprise nucleic acid/a vector encoding a TCR/antigen-binding molecule according to the present disclosure (e.g. by transduction with a viral vector encoding such TCR/antigen-binding molecule according to the present disclosure, or a viral vector comprising such nucleic acid);
culturing immune cells expressing/comprising a TCR/antigen-binding molecule according to the present disclosure, or expressing/comprising a nucleic acid/a vector encoding a TCR/antigen-binding molecule according to the present disclosure in in vitro or ex vivo cell culture;
collecting/isolating immune cells expressing/comprising a TCR/antigen-binding molecule according to the present disclosure, or expressing/comprising a nucleic acid/a vector encoding a TCR/antigen-binding molecule according to the present disclosure;
formulating immune cells expressing/comprising a TCR/antigen-binding molecule according to the present disclosure, or expressing/comprising a nucleic acid/a vector encoding a TCR/antigen-binding molecule according to the present disclosure, to a pharmaceutical composition, e.g. by mixing the cells with a pharmaceutically acceptable adjuvant, diluent, or carrier;
administering immune cells expressing/comprising a TCR/antigen-binding molecule according to the present disclosure, or expressing/comprising a nucleic acid/a vector encoding a TCR/antigen-binding molecule according to the present disclosure, or a pharmaceutical composition comprising such cells, to a subject.

In some embodiments, the methods may additionally comprise treating the cells or subject to induce/enhance expression of the TCR/antigen-binding molecule, and/or to induce/enhance proliferation or survival of immune cells comprising/expressing the TCR/antigen-binding molecule.

In some embodiments, a subject is administered lymphodepleting chemotherapy prior to administration of immune cells expressing/comprising a TCR/antigen-binding molecule described herein (or expressing/comprising nucleic acid/a vector encoding such a TCR/antigen-binding molecule).

That is, in some embodiments, methods of treating/preventing a disease/condition in accordance with the present disclosure comprise: (i) administering a lymphodepleting chemotherapy to a subject, and (ii) subsequently administering an immune cell expressing/comprising a TCR/antigen-binding molecule described herein, or expressing/comprising nucleic acid/a vector encoding encoding a TCR/antigen-binding molecule described herein.

As used herein, ‘lymphodepleting chemotherapy’ refers to treatment with a chemotherapeutic agent which results in depletion of lymphocytes (e.g. T cells, B cells, NK cells, NKT cells or innate lymphoid cell (ILCs), or precursors thereof) within the subject to which the treatment is administered. A ‘lymphodepleting chemotherapeutic agent’ refers to a chemotherapeutic agent which results in depletion of lymphocytes.

Lymphodepleting chemotherapy and its use in methods of treatment by adoptive cell transfer are described e.g. in Klebanoff et al., Trends Immunol. (2005) 26(2):111-7 and Muranski et al., Nat Clin Pract Oncol. (2006) (12):668-81, both of which are hereby incorporated by reference in their entirety. The aim of lymphodepleting chemotherapy is to deplete the recipient subject's endogenous lymphocyte population.

In the context of treatment of disease by adoptive transfer of immune cells, lymphodepleting chemotherapy is typically administered prior to adoptive cell transfer, to condition the recipient subject to receive the adoptively transferred cells. Lymphodepleting chemotherapy is thought to promote the persistence and activity of adoptively transferred cells by creating a permissive environment, e.g. through elimination of cells expressing immunosuppressive cytokines, and creating the ‘lymphoid space’ required for expansion and activity of adoptively transferred lymphoid cells.

Chemotherapeutic agents commonly used in lymphodepleting chemotherapy include e.g. fludarabine, cyclophosphamide, bedamustine and pentostatin.

In some embodiments, therapeutic or prophylactic intervention for the treatment/prevention of a disease/condition in accordance with the present disclosure comprises administration of a nucleic acid/vector, or of a composition comprising a nucleic acid/vector according to the present disclosure. In some embodiments, administration of such an article results in modification of a cell or cells to comprise/express a nucleic acid/vector, and/or to comprise/express TCR/antigen-binding molecule/polypeptide(s) according to the present disclosure. That is, in some embodiments the nucleic acid/vector/composition is employed as a gene therapy.

In some aspects and embodiments in accordance with the present disclosure there may be targeted delivery of articles of the present disclosure, i.e. wherein the concentration of the relevant agent in the subject is increased in a given tissue(s)/organ(s) relative to other parts of the body. In some embodiments, the methods comprise intravascular (e.g. intravenous or intra-arterial), intramuscular or subcutaneous administration and wherein the relevant article is formulated in a targeted agent delivery system (e.g. as described herein). Suitable targeted delivery systems include, for example, nanoparticles, liposomes, micelles, beads, polymers, metal particles, dendrimers, antibodies, aptamers, nanotubes or micro-sized silica rods. Such systems may comprise a magnetic element to direct the agent to the desired organ or tissue. Suitable nanocarriers and delivery systems will be apparent to one skilled in the art.

### B. CDRs, TCRα Chain Variable Domains and TCRβ Chain Variable Domains

Complementarity determining regions (CDRs) are regions of high variability present in the variable domain of TCRs, CARs, single chain fragments and antibodies. These highly variable CDRs are interspaced by relatively constant sequences termed framework regions (FR). In the case of TCRs, the 3 CDR regions of the TCRα chain variable domain are paired with the 3 CDRs of the TCRβ chain variable domain. Together, the 6 CDRs form the antigen binding site of the TCR, thus conferring onto each TCR its specificity (Schroeder and Cavacini, J Allergy Clin Immunol 125(202):S41-S52 (2010); Bhati et al., Protein Science 23:260-272 (2014)).

In some embodiments, there is provided an isolated T cell receptor (TCR) comprising a TCRα chain and a TCRβ chain that binds to Epstein Barr Virus (EBV)-derived antigenic peptides, such as when presented by a major histocompatibility complex (MHC) molecule. In some embodiments, the TCRα chain and the TCRβ chain each comprises three complementarity determining regions (CDR1, CDR2, and CDR3), each comprising an amino acid sequence sharing at least about 95% sequence identity with an amino acid sequence selected from Table 3A. In some embodiments, the TCRα chain and the TCRβ chain CDR1 amino acid sequences share at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with an amino acid sequence selected from: SEQ ID NOs: 1; 2; 3; 4; 5; 6; 136; 25; 26; 27; 28; 29; 30; 31; and 32, and combinations thereof, as set forth in Table 3A. In some embodiments, the TCRα chain and the TCRβ chain CDR2 amino acid sequences share at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with the TCRα chain and the TCRβ chain CDR2 amino acid sequences selected from: SEQ ID NOs: 7; 8; 9; 10; 11; 12; 13; 137; 33; 34; 35; 36; 37; 38; 39; 40; and 41, and combinations thereof, as set forth in Table 3A.

In some embodiments the TCRα chain comprises a complementary determining region CDR3 as set forth in SEQ ID NO:181 and/or the TCRβ chain comprises a complementary determining region CDR3 as set forth in SEQ ID NO:182. In some embodiments the TCRα chain comprises a complementary determining region CDR3 as set forth in SEQ ID NO:183 and/or the TCRβ chain comprises a complementary determining region CDR3 as set forth in SEQ ID NO:184.

In some embodiments the TCRα chain comprises a complementary determining region CDR3 as set forth in SEQ ID NO:15; 16; 17; 18; 19; 20; 21; 22; 23; 24; and 138 and/or the TCRβ chain comprises a complementary determining region CDR3 as set forth in SEQ ID NO:182. In some embodiments the TCRα chain comprises a complementary determining region CDR3 as set forth in SEQ ID NO:181 and/or the TCRβ chain comprises a complementary determining region CDR3 as set forth in SEQ ID NO:43; 44; 45; 46; 47; 48; 49; 50; 51; 52; 53; 54; and 139.

In some embodiments the TCRα chain comprises a complementary determining region CDR3 as set forth in SEQ ID NO: 15; 16; 17; 18; 19; 20; 21; 22; 23; 24; and 138 and/or the TCRβ chain comprises a complementary determining region CDR3 as set forth in SEQ ID NO:184. In some embodiments the TCRα chain comprises a complementary determining region CDR3 as set forth in SEQ ID NO:183 and/or the TCRβ chain comprises a complementary determining region CDR3 as set forth in SEQ ID NO:43; 44; 45; 46; 47; 48; 49; 50; 51; 52; 53; 54; and 139.

In some embodiments, the TCRα chain comprises a complementary determining region CDR3 as set forth in Table 3A sharing at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with a member selected from SEQ ID NOs: 15; 16; 17; 18; 19; 20; 21; 22; 23; 24; and 138; in combination with the TCRβ chain, which comprises a complementary determining region CDR3 as set forth in Table 3A sharing at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with a member selected from SEQ ID NOs: 43; 44; 45; 46; 47; 48; 49; 50; 51; 52; 53; 54; and 139. In some embodiments, the TCR comprises a variable domain comprising the TCRα chain CDR3 and TCRβ chain CDR3 of polypeptide SEQ ID NO pairs selected from the group consisting of: SEQ ID NOs: 15 and 43; SEQ ID NOs: 16 and 44; SEQ ID NOs: 15 and 45; SEQ ID NOs: 17 and 46; SEQ ID NOs: 18 and 47; SEQ ID NOs: 19 and 48; SEQ ID NOs: 20 and 49; SEQ ID NO:21 and 50; SEQ ID NOs: 22 and 50; SEQ ID NOs: 21 and 51; SEQ ID NOs: 23 and 52; SEQ ID NOs: 23 and 53; SEQ ID NOs: 24 and 54; and SEQ ID NOs: 138 and 139. In some embodiments, there is provided an isolated T cell receptor (TCR) comprising a TCRα chain and a TCRβ chain that binds to Mutant splice factor-induced peptide of MAPK8IP2-derived antigenic peptide, such as when presented by a major histocompatibility complex (MHC) molecule. In some embodiments, the TCRα chain and the TCRβ chain each comprises three complementarity determining regions (CDR1, CDR2, and CDR3), each comprising an amino acid sequence sharing at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with an amino acid sequence selected from Table 3A. In some embodiments, the TCRα chain and the TCRβ chain CDR1 amino acid sequences share at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with an amino acid sequence selected from: SEQ ID NOs: 2, 25, 31, 32, 149, 154, 165, and 197, and combinations thereof, as set forth in Table 3A. In some embodiments, the TCRα chain and the TCRβ chain CDR2 amino acid sequences share at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with the TCRα chain and the TCRβ chain CDR2 amino acid sequences selected from: SEQ ID NOs: 8, 33, 40, 150, 156, 157, and 198, and combinations thereof, as set forth in Table 3A.

In some embodiments the TCRα chain comprises a complementary determining region CDR3 as set forth in SEQ ID NO:185, 304, 305 or 306. In some embodiments the TCRα chain comprises a complementary determining region CDR3 as set forth in SEQ ID NO:185, 304, 305 or 306 and/or the TCRβ chain comprises a complementary determining region CDR3 as set forth in SEQ ID NO:42, 159, 160, 195 or 199.

In some embodiments, as in Table 3A, the TCRα chain comprises a complementary determining region CDR3 sharing at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with a member selected from SEQ ID NOs: 14, 151, 152, 194 and 196; in combination with the TCRβ chain, which comprises a complementary determining region CDR3 sharing at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with a member selected from SEQ ID NOs: 42, 159, 160, 195 and 199. In some embodiments, the TCR comprises a variable domain comprising the TCRα chain CDR3 and TCRβ chain CDR3 of polypeptide SEQ ID NO pairs selected from the group consisting of: SEQ ID NOs: 14 and 42; SEQ ID NOs: 151 and 159; SEQ ID NOs: 152 and 160, SEQ ID NOs: 194 and 195, and SEQ ID NOs: 196 and 199.

In some embodiments, there is provided an isolated T cell receptor (TCR) comprising a TCRα chain and a TCRβ chain that binds to HERV-K gag protein-derived antigenic peptide, such as when presented by a major histocompatibility complex (MHC) molecule. In some embodiments, the TCRα chain and the TCRβ chain each comprises three complementarity determining regions (CDR1, CDR2, and CDR3), each comprising an amino acid sequence sharing at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with an amino acid sequence selected from Table 3A. In some embodiments, the TCRα chain and the TCRβ chain CDR1 amino acid sequences share at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with an amino acid sequence selected from: SEQ ID NOs: 4, and 155, as set forth in Table 3A. In some embodiments, the TCRα chain and the TCRβ chain CDR2 amino acid sequences share at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with the TCRα chain and the TCRβ chain CDR2 amino acid sequences selected from: SEQ ID NOs: 10, and 158, as set forth in Table 3A.

In some embodiments, the TCRα chain comprises a complementary determining region CDR3 sharing at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with SEQ ID NO: 153, in combination with the TCRβ chain, which comprises a complementary determining region CDR3 sharing at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with SEQ ID NO:161. In some embodiments, the TCR comprises a variable domain comprising the TCRα chain CDR3 and TCRβ chain CDR3 of polypeptide SEQ ID NO pair SEQ ID NO:153 and 161.

In some embodiments, a TCR/antigen-binding molecule according to the present disclosure comprises a TCRα chain variable domain according to one of the following:
(1) [A0002, A0004] a TCRα chain variable domain incorporating the following CDRs:
   CDR1α having the amino acid sequence of SEQ ID NO:1
   CDR2α having the amino acid sequence of SEQ ID NO:7
   CDR3α having the amino acid sequence of SEQ ID NO:15,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.
(2) [A0003] a TCRα chain variable domain incorporating the following CDRs:
   CDR1α having the amino acid sequence of SEQ ID NO:1
   CDR2α having the amino acid sequence of SEQ ID NO:7
   CDR3α having the amino acid sequence of SEQ ID NO:16,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.
(3) [A0005] a TCRα chain variable domain incorporating the following CDRs:
   CDR1α having the amino acid sequence of SEQ ID NO:2
   CDR2α having the amino acid sequence of SEQ ID NO:8
   CDR3α having the amino acid sequence of SEQ ID NO:17,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.

In some embodiments, a TCR/antigen-binding molecule according to the present disclosure comprises:
(4) a TCRα chain variable domain comprising FR1, FR2, FR3 and FR4 according to one of rows 1, 2, 3 or 4 of column A of Table 3B.

In some embodiments, a TCR/antigen-binding molecule according to the present disclosure comprises:
(5) a TCRα chain variable domain comprising the CDRs of one of (1) to (3) above, and comprising FR1, FR2, FR3 and FR4 according to one of rows 1, 2, 3 or 4 of column A of Table 3B.

In some embodiments, a TCR/antigen-binding molecule according to the present disclosure comprises a TCRα chain variable domain according to one of:
(6) a TCRα chain variable domain comprising the CDRs according to (1) above, and FR1, FR2, FR3 and FR4 according to row 1 or 3 of column A of Table 3B.
(7) a TCRα chain variable domain comprising the CDRs according to (2) above, and FR1, FR2, FR3 and FR4 according to row 2 of column A of Table 3B.
(8) a TCRα chain variable domain comprising the CDRs according to (3) above, and FR1, FR2, FR3 and FR4 according to row 4 of column A of Table 3B.

In some embodiments, the TCR/antigen-binding molecule according to the present disclosure comprises a TCRβ chain variable domain according to one of the following:
(9) [A0002] a TCRβ chain variable domain incorporating the following CDRs:
   CDR1β having the amino acid sequence of SEQ ID NO:26
   CDR2β having the amino acid sequence of SEQ ID NO:34
   CDR3β having the amino acid sequence of SEQ ID NO:43,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(10) [A0003] a TCRβ chain variable domain incorporating the following CDRs:
   CDR1β having the amino acid sequence of SEQ ID NO:27
   CDR2β having the amino acid sequence of SEQ ID NO:35
   CDR3β having the amino acid sequence of SEQ ID NO:44,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(11) [A0004] a TCRβ chain variable domain incorporating the following CDRs:
   CDR1β having the amino acid sequence of SEQ ID NO:25
   CDR2β having the amino acid sequence of SEQ ID NO:33
   CDR3β having the amino acid sequence of SEQ ID NO:45,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(12) [A0005] a TCRβ chain variable domain incorporating the following CDRs:
   CDR1β having the amino acid sequence of SEQ ID NO:28
   CDR2β having the amino acid sequence of SEQ ID NO:36
   CDR3β having the amino acid sequence of SEQ ID NO:46,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.

In some embodiments, a TCR/antigen-binding molecule according to the present disclosure comprises:
(13) a TCRβ chain variable domain comprising FR1, FR2, FR3 and FR4 according to one of rows 1, 2, 3 or 4 of column B of Table 3B.

In some embodiments, a TCR/antigen-binding molecule according to the present disclosure comprises:
(14) a TCRβ chain variable domain comprising the CDRs of one of (9) to (12) above, and comprising FR1, FR2, FR3 and FR4 according to one of rows 1, 2, 3 or 4 of column B of Table 3B.

In some embodiments, a TCR/antigen-binding molecule according to the present disclosure comprises a TCRβ chain variable domain according to one of:
(15) a TCRβ chain variable domain comprising the CDRs according to (9) above, and FR1, FR2, FR3 and FR4 according to row 1 of column B of Table 3B.
(16) a TCRβ chain variable domain comprising the CDRs according to (10) above, and FR1, FR2, FR3 and FR4 according to row 2 of column B of Table 3B.
(17) a TCRβ chain variable domain comprising the CDRs according to (11) above, and FR1, FR2, FR3 and FR4 according to row 3 of column B of Table 3B.
(18) a TCRβ chain variable domain comprising the CDRs according to (12) above, and FR1, FR2, FR3 and FR4 according to row 4 of column B of Table 3B.

In some embodiments, a TCR/antigen-binding molecule according to the present disclosure comprises a TCRα chain variable domain comprising the CDRs according to one of (1) to (3) above, and a TCRβ chain variable domain comprising the CDRs according to one of (9) to (12) above.

In some embodiments, a TCR/antigen-binding molecule according to the present disclosure comprises:
a TCRα chain variable domain comprising the CDRs according to (1), and a TCRβ chain variable domain comprising the CDRs according to (9);
a TCRα chain variable domain comprising the CDRs according to (1), and a TCRβ chain variable domain comprising the CDRs according to (11);
a TCRα chain variable domain comprising the CDRs according to (2), and a TCRβ chain variable domain comprising the CDRs according to (10); or
a TCRα chain variable domain comprising the CDRs according to (3), and a TCRβ chain variable domain comprising the CDRs according to (12).

In some embodiments, a TCR/antigen-binding molecule according to the present disclosure comprises a TCRα chain variable domain according to one of (1) to (8) above, and a TCRβ chain variable domain according to one of (9) to (18) above.

In some embodiments, the TCR/antigen-binding molecule according to the present disclosure comprises a TCRα chain variable domain according to:
(19) [A0015] a TCRα chain variable domain incorporating the following CDRs:
   CDR1α having the amino acid sequence of SEQ ID NO:3
   CDR2α having the amino acid sequence of SEQ ID NO:9
   CDR3α having the amino acid sequence of SEQ ID NO:18,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.

In some embodiments, a TCR/antigen-binding molecule according to the present disclosure comprises:
(20) a TCRα chain variable domain comprising FR1, FR2, FR3 and FR4 according to row 5 of column A of Table 3B.

In some embodiments, a TCR/antigen-binding molecule according to the present disclosure comprises:
(21) a TCRα chain variable domain comprising the CDRs of (19) above, and comprising FR1, FR2, FR3 and FR4 according to row 5 of column A of Table 3B.

In some embodiments, the TCR/antigen-binding molecule according to the present disclosure comprises a TCRβ chain variable domain according to:
(22) [A0015] a TCRβ chain variable domain incorporating the following CDRs:
   CDR1β having the amino acid sequence of SEQ ID NO:29
   CDR2β having the amino acid sequence of SEQ ID NO:37
   CDR3β having the amino acid sequence of SEQ ID NO:47,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.

In some embodiments, a TCR/antigen-binding molecule according to the present disclosure comprises:
(23) a TCRβ chain variable domain comprising FR1, FR2, FR3 and FR4 according to row 5 of column B of Table 3B.

In some embodiments, a TCR/antigen-binding molecule according to the present disclosure comprises:
(24) a TCRβ chain variable domain comprising the CDRs of (22) above, and comprising FR1, FR2, FR3 and FR4 according to row 5 of column B of Table 3B.

In some embodiments, a TCR/antigen-binding molecule according to the present disclosure comprises a TCRα chain variable domain comprising the CDRs according to (19) above, and a TCRβ chain variable domain comprising the CDRs according to (22) above.

In some embodiments, a TCR/antigen-binding molecule according to the present disclosure comprises a TCRα chain variable domain according to one of (19) to (21) above, and a TCRβ chain variable domain according to one of (22) to (24) above.

In some embodiments, a TCR/antigen-binding molecule according to the present disclosure comprises a TCRα chain variable domain according to one of the following:
(25) [A0061, A0064, A0065, A0066 consensus] a TCRα chain variable domain incorporating the following CDRs:
   CDR1α having the amino acid sequence of SEQ ID NO:4 or 6
   CDR2α having the amino acid sequence of SEQ ID NO:10 or 12
   CDR3α having the amino acid sequence of SEQ ID NO:183, 19, 21 or 22,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.
(26) [A0061] a TCRα chain variable domain incorporating the following CDRs:
   CDR1α having the amino acid sequence of SEQ ID NO:4
   CDR2α having the amino acid sequence of SEQ ID NO:10
   CDR3α having the amino acid sequence of SEQ ID NO:183 or 19,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.
(27) [A0064, A0066] a TCRα chain variable domain incorporating the following CDRs:
   CDR1α having the amino acid sequence of SEQ ID NO:6
   CDR2α having the amino acid sequence of SEQ ID NO:12
   CDR3α having the amino acid sequence of SEQ ID NO:183 or 21,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.
(28) [A0065] a TCRα chain variable domain incorporating the following CDRs:
   CDR1α having the amino acid sequence of SEQ ID NO:6
   CDR2α having the amino acid sequence of SEQ ID NO:12
   CDR3α having the amino acid sequence of SEQ ID NO:183 or 22,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.

In some embodiments, a TCR/antigen-binding molecule according to the present disclosure comprises:
(29) a TCRα chain variable domain comprising FR1, FR2, FR3 and FR4 according to one of rows 6, 8, 9 or 10 of column A of Table 3B.

In some embodiments, a TCR/antigen-binding molecule according to the present disclosure comprises:
(30) a TCRα chain variable domain comprising the CDRs of one of (25) to (28) above, and comprising FR1, FR2, FR3 and FR4 according to one of rows 6, 8, 9 or 10 of column A of Table 3B.

In some embodiments, a TCR/antigen-binding molecule according to the present disclosure comprises a TCRα chain variable domain according to one of:
(31) a TCRα chain variable domain comprising the CDRs according to (25) above, and FR1, FR2, FR3 and FR4 according to one of rows 6, 8, 9 or 10 of column A of Table 3B.
(32) a TCRα chain variable domain comprising the CDRs according to (26) above, and FR1, FR2, FR3 and FR4 according to row 6 of column A of Table 3B.
(33) a TCRα chain variable domain comprising the CDRs according to (27) above, and FR1, FR2, FR3 and FR4 according to row 8 of column A of Table 3B.
(34) a TCRα chain variable domain comprising the CDRs according to (28) above, and FR1, FR2, FR3 and FR4 according to row 9 of column A of Table 3B.
(35) a TCRα chain variable domain comprising the CDRs according to (27) above, and FR1, FR2, FR3 and FR4 according to row 10 of column A of Table 3B.

In some embodiments, the TCR/antigen-binding molecule according to the present disclosure comprises a TCRβ chain variable domain according to one of the following:
(36) [A0061, A0064, A0065, A0066 consensus] a TCRβ chain variable domain incorporating the following CDRs:
   CDR1β having the amino acid sequence of SEQ ID NO:30 or 32
   CDR2B having the amino acid sequence of SEQ ID NO:38 or 40
   CDR3β having the amino acid sequence of SEQ ID NO:184, 48, 50 or 51,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(37) [A0061] a TCRβ chain variable domain incorporating the following CDRs:
   CDR1β having the amino acid sequence of SEQ ID NO:30
   CDR2β having the amino acid sequence of SEQ ID NO:38
   CDR3β having the amino acid sequence of SEQ ID NO:184 or 48,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(38) [A0064, A0065] a TCRβ chain variable domain incorporating the following CDRs:
   CDR1β having the amino acid sequence of SEQ ID NO:32
   CDR2β having the amino acid sequence of SEQ ID NO:40
   CDR3β having the amino acid sequence of SEQ ID NO:184 or 50,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(39) [A0066] a TCRβ chain variable domain incorporating the following CDRs:
   CDR1β having the amino acid sequence of SEQ ID NO:32
   CDR2B having the amino acid sequence of SEQ ID NO:40
   CDR3β having the amino acid sequence of SEQ ID NO:184 or 51,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.

In some embodiments, a TCR/antigen-binding molecule according to the present disclosure comprises:
(40) a TCRβ chain variable domain comprising FR1, FR2, FR3 and FR4 according to one of rows 6, 8, 9 or 10 of column B of Table 3B.

In some embodiments, a TCR/antigen-binding molecule according to the present disclosure comprises:
(41) a TCRβ chain variable domain comprising the CDRs of one of (36) to (39) above, and comprising FR1, FR2, FR3 and FR4 according to one of rows 6, 8, 9 or 10 of column B of Table 3B.

In some embodiments, a TCR/antigen-binding molecule according to the present disclosure comprises a TCRβ chain variable domain according to one of:
(42) a TCRβ chain variable domain comprising the CDRs according to (36) above, and FR1, FR2, FR3 and FR4 according to row 6, 8, 9 or 10 of column B of Table 3B.
(43) a TCRβ chain variable domain comprising the CDRs according to (37) above, and FR1, FR2, FR3 and FR4 according to row 6 of column B of Table 3B.
(44) a TCRβ chain variable domain comprising the CDRs according to (38) above, and FR1, FR2, FR3 and FR4 according to row 8 of column B of Table 3B.
(45) a TCRβ chain variable domain comprising the CDRs according to (38) above, and FR1, FR2, FR3 and FR4 according to row 9 of column B of Table 3B.
(46) a TCRβ chain variable domain comprising the CDRs according to (39) above, and FR1, FR2, FR3 and FR4 according to row 10 of column B of Table 3B.

In some embodiments, a TCR/antigen-binding molecule according to the present disclosure comprises a TCRα chain variable domain comprising the CDRs according to one of (25) to (28) above, and a TCRβ chain variable domain comprising the CDRs according to one of (36) to (39) above.

In some embodiments, a TCR/antigen-binding molecule according to the present disclosure comprises:
a TCRα chain variable domain comprising the CDRs according to (25), and a TCRβ chain variable domain comprising the CDRs according to (36);
a TCRα chain variable domain comprising the CDRs according to (26), and a TCRβ chain variable domain comprising the CDRs according to (37);
a TCRα chain variable domain comprising the CDRs according to (27), and a TCRβ chain variable domain comprising the CDRs according to (38);
a TCRα chain variable domain comprising the CDRs according to (27), and a TCRβ chain variable domain comprising the CDRs according to (39); or
a TCRα chain variable domain comprising the CDRs according to (28), and a TCRβ chain variable domain comprising the CDRs according to (38).

In some embodiments, a TCR/antigen-binding molecule according to the present disclosure comprises a TCRα chain variable domain according to one of (25) to (35) above, and a TCRβ chain variable domain according to one of (36) to (46) above.

In some embodiments, a TCR/antigen-binding molecule according to the present disclosure comprises a TCRα chain variable domain according to one of the following:
(47) [A0062, A0068, A0069, A0070 consensus] a TCRα chain variable domain incorporating the following CDRs:
   CDR1α having the amino acid sequence of SEQ ID NO:5
   CDR2α having the amino acid sequence of SEQ ID NO:11 or 13
   CDR3α having the amino acid sequence of SEQ ID NO:181, 20, 23 or 24,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.
(48) [A0062] a TCRα chain variable domain incorporating the following CDRs:
   CDR1α having the amino acid sequence of SEQ ID NO:5
   CDR2α having the amino acid sequence of SEQ ID NO:11
   CDR3α having the amino acid sequence of SEQ ID NO:181 or 20,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.
(49) [A0068, A0069] a TCRα chain variable domain incorporating the following CDRs:
   CDR1α having the amino acid sequence of SEQ ID NO:5
   CDR2α having the amino acid sequence of SEQ ID NO:13
   CDR3α having the amino acid sequence of SEQ ID NO:181 or 23,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.
(50) [A0070] a TCRα chain variable domain incorporating the following CDRs:
   CDR1α having the amino acid sequence of SEQ ID NO:5
   CDR2α having the amino acid sequence of SEQ ID NO:13
   CDR3α having the amino acid sequence of SEQ ID NO:181 or 24,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.

In some embodiments, a TCR/antigen-binding molecule according to the present disclosure comprises:
(51) a TCRα chain variable domain comprising FR1, FR2, FR3 and FR4 according to one of rows 7, 11, 12 or 13 of column A of Table 3B.

In some embodiments, a TCR/antigen-binding molecule according to the present disclosure comprises:
(52) a TCRα chain variable domain comprising the CDRs of one of (47) to (50) above, and comprising FR1, FR2, FR3 and FR4 according to one of rows 7, 11, 12 or 13 of column A of Table 3B.

In some embodiments, a TCR/antigen-binding molecule according to the present disclosure comprises a TCRα chain variable domain according to one of:
(53) a TCRα chain variable domain comprising the CDRs according to (47) above, and FR1, FR2, FR3 and FR4 according to one of rows 7, 11, 12 or 13 of column A of Table 3B.
(54) a TCRα chain variable domain comprising the CDRs according to (48) above, and FR1, FR2, FR3 and FR4 according to row 7 of column A of Table 3B.
(55) a TCRα chain variable domain comprising the CDRs according to (49) above, and FR1, FR2, FR3 and FR4 according to row 11 of column A of Table 3B.
(56) a TCRα chain variable domain comprising the CDRs according to (49) above, and FR1, FR2, FR3 and FR4 according to row 12 of column A of Table 3B.
(57) a TCRα chain variable domain comprising the CDRs according to (50) above, and FR1, FR2, FR3 and FR4 according to row 13 of column A of Table 3B.

In some embodiments, the TCR/antigen-binding molecule according to the present disclosure comprises a TCRβ chain variable domain according to one of the following:
(58) [A0062, A0068, A0069, A0070 consensus] a TCRβ chain variable domain incorporating the following CDRs:
   CDR1β having the amino acid sequence of SEQ ID NO:31
   CDR2β having the amino acid sequence of SEQ ID NO:39 or 41
   CDR3β having the amino acid sequence of SEQ ID NO: 182, 49, 52, 53 or 54 or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(59) [A0062] a TCRβ chain variable domain incorporating the following CDRs:
   CDR1β having the amino acid sequence of SEQ ID NO:31
   CDR2β having the amino acid sequence of SEQ ID NO:39
   CDR3β having the amino acid sequence of SEQ ID NO: 182 or 49,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(60) [A0068] a TCRβ chain variable domain incorporating the following CDRs:
   CDR1β having the amino acid sequence of SEQ ID NO:31
   CDR2β having the amino acid sequence of SEQ ID NO:41
   CDR3β having the amino acid sequence of SEQ ID NO:182 or 52,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(61) [A0069] a TCRβ chain variable domain incorporating the following CDRs:
   CDR1β having the amino acid sequence of SEQ ID NO:31
   CDR2β having the amino acid sequence of SEQ ID NO:41
   CDR3β having the amino acid sequence of SEQ ID NO: 182 or 53,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(62) [A0070] a TCRβ chain variable domain incorporating the following CDRs:
   CDR1β having the amino acid sequence of SEQ ID NO:31
   CDR2β having the amino acid sequence of SEQ ID NO:41
   CDR3β having the amino acid sequence of SEQ ID NO: 182 or 54,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.

In some embodiments, a TCR/antigen-binding molecule according to the present disclosure comprises:
(63) a TCRβ chain variable domain comprising FR1, FR2, FR3 and FR4 according to one of rows 7, 11, 12 or 13 of column B of Table 3B.

In some embodiments, a TCR/antigen-binding molecule according to the present disclosure comprises:
(64) a TCRβ chain variable domain comprising the CDRs of one of (58) to (62) above, and comprising FR1, FR2, FR3 and FR4 according to one of rows 7, 11, 12 or 13 of column B of Table 3B.

In some embodiments, a TCR/antigen-binding molecule according to the present disclosure comprises a TCRβ chain variable domain according to one of:
(65) a TCRβ chain variable domain comprising the CDRs according to (58) above, and FR1, FR2, FR3 and FR4 according to one of rows 7, 11, 12 or 13 of column B of Table 3B.
(66) a TCRβ chain variable domain comprising the CDRs according to (59) above, and FR1, FR2, FR3 and FR4 according to row 7 of column B of Table 3B.
(67) a TCRβ chain variable domain comprising the CDRs according to (60) above, and FR1, FR2, FR3 and FR4 according to row 11 of column B of Table 3B.
(68) a TCRβ chain variable domain comprising the CDRs according to (61) above, and FR1, FR2, FR3 and FR4 according to row 12 of column B of Table 3B.
(69) a TCRβ chain variable domain comprising the CDRs according to (62) above, and FR1, FR2, FR3 and FR4 according to row 13 of column B of Table 3B.

In some embodiments, a TCR/antigen-binding molecule according to the present disclosure comprises a TCRα chain variable domain comprising the CDRs according to one of (47) to (50) above, and a TCRβ chain variable domain comprising the CDRs according to one of (58) to (62) above.

In some embodiments, a TCR/antigen-binding molecule according to the present disclosure comprises:
a TCRα chain variable domain comprising the CDRs according to (47), and a TCRβ chain variable domain comprising the CDRs according to (58);
a TCRα chain variable domain comprising the CDRs according to (48), and a TCRβ chain variable domain comprising the CDRs according to (59);
a TCRα chain variable domain comprising the CDRs according to (49), and a TCRβ chain variable domain comprising the CDRs according to (60);
a TCRα chain variable domain comprising the CDRs according to (49), and a TCRβ chain variable domain comprising the CDRs according to (61); or
a TCRα chain variable domain comprising the CDRs according to (50), and a TCRβ chain variable domain comprising the CDRs according to (62).

In some embodiments, a TCR/antigen-binding molecule according to the present disclosure comprises a TCRα chain variable domain according to one of (47) to (57) above, and a TCRβ chain variable domain according to one of (58) to (69) above.

In some embodiments, the TCR/antigen-binding molecule according to the present disclosure comprises a TCRα chain variable domain according to:
(70) [A0099] a TCRα chain variable domain incorporating the following CDRs:
   CDR1α having the amino acid sequence of SEQ ID NO:136
   CDR2α having the amino acid sequence of SEQ ID NO:137
   CDR3α having the amino acid sequence of SEQ ID NO:138,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.

In some embodiments, a TCR/antigen-binding molecule according to the present disclosure comprises:
(71) a TCRα chain variable domain comprising FR1, FR2, FR3 and FR4 according to row 14 of column A of Table 3B.

In some embodiments, a TCR/antigen-binding molecule according to the present disclosure comprises:
(72) a TCRα chain variable domain comprising the CDRs of (70) above, and comprising FR1, FR2, FR3 and FR4 according to row 14 of column A of Table 3B.

In some embodiments, the TCR/antigen-binding molecule according to the present disclosure comprises a TCRβ chain variable domain according to:
(73) [A0099] a TCRβ chain variable domain incorporating the following CDRs:
   CDR1β having the amino acid sequence of SEQ ID NO:27
   CDR2β having the amino acid sequence of SEQ ID NO:35
   CDR3β having the amino acid sequence of SEQ ID NO:139,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.

In some embodiments, a TCR/antigen-binding molecule according to the present disclosure comprises:
(74) a TCRβ chain variable domain comprising FR1, FR2, FR3 and FR4 according to row 14 of column B of Table 3B.

In some embodiments, a TCR/antigen-binding molecule according to the present disclosure comprises:
(75) a TCRβ chain variable domain comprising the CDRs of (73) above, and comprising FR1, FR2, FR3 and FR4 according to row 14 of column B of Table 3B.

In some embodiments, a TCR/antigen-binding molecule according to the present disclosure comprises a TCRα chain variable domain comprising the CDRs according to (70) above, and a TCRβ chain variable domain comprising the CDRs according to (73) above.

In some embodiments, a TCR/antigen-binding molecule according to the present disclosure comprises a TCRα chain variable domain according to one of (70) to (72) above, and a TCRβ chain variable domain according to one of (73) to (75) above.

In some embodiments, the TCR/antigen-binding molecule according to the present disclosure comprises a TCRα chain variable domain according to:
(76) [A0100] a TCRα chain variable domain incorporating the following CDRs:
   CDR1α having the amino acid sequence of SEQ ID NO:4
   CDR2α having the amino acid sequence of SEQ ID NO:10
   CDR3α having the amino acid sequence of SEQ ID NO:153,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.

In some embodiments, a TCR/antigen-binding molecule according to the present disclosure comprises:
(77) a TCRα chain variable domain comprising FR1, FR2, FR3 and FR4 according to row 17 of column A of Table 3B.

In some embodiments, a TCR/antigen-binding molecule according to the present disclosure comprises:
(78) a TCRα chain variable domain comprising the CDRs of (76) above, and comprising FR1, FR2, FR3 and FR4 according to row 17 of column A of Table 3B.

In some embodiments, the TCR/antigen-binding molecule according to the present disclosure comprises a TCRβ chain variable domain according to:
(79) [A0100] a TCRβ chain variable domain incorporating the following CDRs:
   CDR1β having the amino acid sequence of SEQ ID NO:155
   CDR2β having the amino acid sequence of SEQ ID NO:158
   CDR3β having the amino acid sequence of SEQ ID NO:161,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.

In some embodiments, a TCR/antigen-binding molecule according to the present disclosure comprises:
(80) a TCRβ chain variable domain comprising FR1, FR2, FR3 and FR4 according to row 17 of column B of Table 3B.

In some embodiments, a TCR/antigen-binding molecule according to the present disclosure comprises:
(81) a TCRβ chain variable domain comprising the CDRs of (79) above, and comprising FR1, FR2, FR3 and FR4 according to row 17 of column B of Table 3B.

In some embodiments, a TCR/antigen-binding molecule according to the present disclosure comprises a TCRα chain variable domain comprising the CDRs according to (76) above, and a TCRβ chain variable domain comprising the CDRs according to (79) above.

In some embodiments, a TCR/antigen-binding molecule according to the present disclosure comprises a TCRα chain variable domain according to one of (76) to (78) above, and a TCRβ chain variable domain according to one of (79) to (81) above.

In some embodiments, a TCR/antigen-binding molecule according to the present disclosure comprises a TCRα chain variable domain according to one of the following:
(82) [A0130, A0131, A0132, A0358, A0359 consensus] a TCRα chain variable domain incorporating the following CDRs:
   CDR1α having the amino acid sequence of SEQ ID NO:149, 165 or 2
   CDR2α having the amino acid sequence of SEQ ID NO:150 or 8
   CDR3α having the amino acid sequence of SEQ ID NO:185, 304, 305, 306, 151, 152, 14, 194 or 196
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.
(83) [A0130] a TCRα chain variable domain incorporating the following CDRs:
   CDR1α having the amino acid sequence of SEQ ID NO:149
   CDR2α having the amino acid sequence of SEQ ID NO:150
   CDR3α having the amino acid sequence of SEQ ID NO:185, 304, 305, 306 or 151,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.
(84) [A0131] a TCRα chain variable domain incorporating the following CDRs:
   CDR1α having the amino acid sequence of SEQ ID NO:165
   CDR2α having the amino acid sequence of SEQ ID NO:150
   CDR3α having the amino acid sequence of SEQ ID NO:185, 304, 305, 306 or 152,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.
(85) [A0132] a TCRα chain variable domain incorporating the following CDRs:
   CDR1α having the amino acid sequence of SEQ ID NO:149
   CDR2α having the amino acid sequence of SEQ ID NO:150
   CDR3α having the amino acid sequence of SEQ ID NO:185, 304, 305, 306 or 14,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.
(86) [A0358] a TCRα chain variable domain incorporating the following CDRs:
   CDR1α having the amino acid sequence of SEQ ID NO:165
   CDR2α having the amino acid sequence of SEQ ID NO:150
   CDR3α having the amino acid sequence of SEQ ID NO:185, 304, 305, 306 or 194,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.
(87) [A0359] a TCRα chain variable domain incorporating the following CDRs:
   CDR1α having the amino acid sequence of SEQ ID NO:2
   CDR2α having the amino acid sequence of SEQ ID NO:8
   CDR3α having the amino acid sequence of SEQ ID NO:185, 304, 305, 306 or 196,
   or a variant thereof in which 1 or 2 or 3 amino acids in CDR1α, and/or in which 1 or 2 or 3 amino acids in CDR2α, and/or in which 1 or 2 or 3 amino acids in CDR3α are substituted with another amino acid.

In some embodiments, a TCR/antigen-binding molecule according to the present disclosure comprises:
(88) a TCRα chain variable domain comprising FR1, FR2, FR3 and FR4 according to one of rows 15, 16, 18, 19 or 20 of column A of Table 3B.

In some embodiments, a TCR/antigen-binding molecule according to the present disclosure comprises:
(89) a TCRα chain variable domain comprising the CDRs of one of (82) to (87) above, and comprising FR1, FR2, FR3 and FR4 according to one of rows 15, 16, 18, 19 or 20 of column A of Table 3B.

In some embodiments, a TCR/antigen-binding molecule according to the present disclosure comprises a TCRα chain variable domain according to one of:
(90) a TCRα chain variable domain comprising the CDRs according to (82) above, and FR1, FR2, FR3 and FR4 according to one of rows 15, 16, 18, 19 or 20 of column A of Table 3B.
(91) a TCRα chain variable domain comprising the CDRs according to (83) above, and FR1, FR2, FR3 and FR4 according to row 15 of column A of Table 3B.
(92) a TCRα chain variable domain comprising the CDRs according to (84) above, and FR1, FR2, FR3 and FR4 according to row 16 of column A of Table 3B.
(93) a TCRα chain variable domain comprising the CDRs according to (85) above, and FR1, FR2, FR3 and FR4 according to row 18 of column A of Table 3B.
(94) a TCRα chain variable domain comprising the CDRs according to (86) above, and FR1, FR2, FR3 and FR4 according to row 19 of column A of Table 3B.
(95) a TCRα chain variable domain comprising the CDRs according to (87) above, and FR1, FR2, FR3 and FR4 according to row 20 of column A of Table 3B.

In some embodiments, the TCR/antigen-binding molecule according to the present disclosure comprises a TCRβ chain variable domain according to one of the following:
(96) [A0130, A0131, A0132, A0358, A0359 consensus] a TCRβ chain variable domain incorporating the following CDRs:
   CDR1β having the amino acid sequence of SEQ ID NO:154, 31, 32, 25 or 197
   CDR2β having the amino acid sequence of SEQ ID NO:156, 157, 40, 33 or 198
   CDR3β having the amino acid sequence of SEQ ID NO:159, 160, 42, 195 or 199,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(97) [A0130] a TCRβ chain variable domain incorporating the following CDRs:
   CDR1β having the amino acid sequence of SEQ ID NO:154
   CDR2β having the amino acid sequence of SEQ ID NO:156
   CDR3β having the amino acid sequence of SEQ ID NO:159,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(98) [A0131] a TCRβ chain variable domain incorporating the following CDRs:
   CDR1β having the amino acid sequence of SEQ ID NO:31
   CDR2β having the amino acid sequence of SEQ ID NO:157
   CDR3β having the amino acid sequence of SEQ ID NO:160,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(99) [A0132] a TCRβ chain variable domain incorporating the following CDRs:
   CDR1β having the amino acid sequence of SEQ ID NO:32
   CDR2β having the amino acid sequence of SEQ ID NO:40
   CDR3β having the amino acid sequence of SEQ ID NO:42,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(100) [A0358] a TCRβ chain variable domain incorporating the following CDRs:
   CDR1β having the amino acid sequence of SEQ ID NO:25
   CDR2β having the amino acid sequence of SEQ ID NO:33
   CDR3β having the amino acid sequence of SEQ ID NO:195,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.
(101) [A0359] a TCRβ chain variable domain incorporating the following CDRs:
   CDR1β having the amino acid sequence of SEQ ID NO:197
   CDR2β having the amino acid sequence of SEQ ID NO:198
   CDR3β having the amino acid sequence of SEQ ID NO:199,
   or a variant thereof in which 1 or 2 or 3 amino acids in CDR1β, and/or in which 1 or 2 or 3 amino acids in CDR2β, and/or in which 1 or 2 or 3 amino acids in CDR3β are substituted with another amino acid.

In some embodiments, a TCR/antigen-binding molecule according to the present disclosure comprises:
(102) a TCRβ chain variable domain comprising FR1, FR2, FR3 and FR4 according to one of rows 15, 16, 18, 19 or 20 of column B of Table 3B.

In some embodiments, a TCR/antigen-binding molecule according to the present disclosure comprises:
(103) a TCRβ chain variable domain comprising the CDRs of one of (96) to (101) above, and comprising FR1, FR2, FR3 and FR4 according to one of rows 15, 16, 18, 19 or 20 of column B of Table 3B.

In some embodiments, a TCR/antigen-binding molecule according to the present disclosure comprises a TCRβ chain variable domain according to one of:
(104) a TCRβ chain variable domain comprising the CDRs according to (96) above, and FR1, FR2, FR3 and FR4 according to one of rows 15, 16, 18, 19 or 20 of column B of Table 3B.
(105) a TCRβ chain variable domain comprising the CDRs according to (97) above, and FR1, FR2, FR3 and FR4 according to row 15 of column B of Table 3B.
(106) a TCRβ chain variable domain comprising the CDRs according to (98) above, and FR1, FR2, FR3 and FR4 according to row 16 of column B of Table 3B.
(107) a TCRβ chain variable domain comprising the CDRs according to (99) above, and FR1, FR2, FR3 and FR4 according to row 18 of column B of Table 3B.
(108) a TCRβ chain variable domain comprising the CDRs according to (100) above, and FR1, FR2, FR3 and FR4 according to row 19 of column B of Table 3B.
(109) a TCRβ chain variable domain comprising the CDRs according to (101) above, and FR1, FR2, FR3 and FR4 according to row 20 of column B of Table 3B.

In some embodiments, a TCR/antigen-binding molecule according to the present disclosure comprises a TCRα chain variable domain comprising the CDRs according to one of (82) to (87) above, and a TCRβ chain variable domain comprising the CDRs according to one of (96) to (101) above.

In some embodiments, a TCR/antigen-binding molecule according to the present disclosure comprises:
a TCRα chain variable domain comprising the CDRs according to (82), and a TCRβ chain variable domain comprising the CDRs according to (96);
a TCRα chain variable domain comprising the CDRs according to (83), and a TCRβ chain variable domain comprising the CDRs according to (97);
a TCRα chain variable domain comprising the CDRs according to (84), and a TCRβ chain variable domain comprising the CDRs according to (98);
a TCRα chain variable domain comprising the CDRs according to (85), and a TCRβ chain variable domain comprising the CDRs according to (99); or
a TCRα chain variable domain comprising the CDRs according to (86), and a TCRβ chain variable domain comprising the CDRs according to (100).
a TCRα chain variable domain comprising the CDRs according to (87), and a TCRβ chain variable domain comprising the CDRs according to (101).

In some embodiments, a TCR/antigen-binding molecule according to the present disclosure comprises a TCRα chain variable domain according to one of (82) to (95) above, and a TCRβ chain variable domain according to one of (96) to (109) above.

In some embodiments the TCR comprises a TCRα chain variable domain having an amino acid sequence as shown in column A of Table 4. In some embodiments the TCR comprises a TCRβ chain variable domain having an amino acid sequence as shown in column B of Table 4.

In some embodiments the TCR comprises a TCRα chain variable domain having an amino acid sequence as shown in column A of Table 4, and a TCRβ chain variable domain having an amino acid sequence as shown in column B of Table 4, wherein the TCRα chain variable domain sequence and TCRβ chain variable domain sequence are selected from the same row of Table 4.

An exemplary known TCR, peptide used for isolation of TCRs, and target antigen are set forth in Kamga et al., 2019, as follows: TCR_A0001, for target antigen BRLF1, specific for peptide YVLDHLIVV (SEQ ID NO:105). Known exemplary amino acid sequences of CDR1, 2 and 3 regions of TCRα and TCRβ are set forth in Kamga et al., 2019, as follows: TCR_A0001 CDR1α: YGGTVN (SEQ ID NO:1); CDR2α: YFSGDPLV (SEQ ID NO: 7); CDR3α: CAVKDTDKLIF (SEQ ID NO:15); CDR1β: KGHDR (SEQ ID NO:25); CDR2β: SFDVKD (SEQ ID NO:33); and CDR3β:CATSDWDDSTGELFF (SEQ ID NO:192).

TABLE 2
Listing of exemplary TCRs, peptide used for isolation of TCRs, and target antigen

| TCR | | | | |
|---|---|---|---|---|
| NO | TCR ID | Peptide | Target antigen | Reference |
| 1 | TCR_A0001 | YVLDHLIVV | EBV BRLF1 | Reported in |
| | | (SEQ ID NO: 105) | | Kamga et al |
| | | | | 2019 |
| 2 | TCR_A0002 | YVLDHLIVV | EBV BRLF1 | TCRα CDR3 |
| | | (SEQ ID NO: 105) | | as reported in |
| | | | | Kamga et al. |
| 3 | TCR_A0003 | YVLDHLIVV | EBV BRLF1 | |
| | | (SEQ ID NO: 105) | | |
| 4 | TCR_A0004 | YVLDHLIVV | EBV BRLF1 | TCRα CDR3 |
| | | (SEQ ID NO: 105) | | as reported in |
| | | | | Kamga et al. |
| 5 | TCR_A0005 | YVLDHLIVV | EBV BRLF1 | |
| | | (SEQ ID NO: 105) | | |
| 6 | TCR_A0015 | Peptide pool/MGSLEMVPM | EBV LMP2 | |
| | | (SEQ ID NO: 146) | | |
| 7 | TCR_A0061 | CLGGLLTMV | EBV-LMP2 | |
| | | (SEQ ID NO: 106) | | |
| 8 | TCR_A0062 | FLYALALLL | EBV LMP2 | |
| | | (SEQ ID NO: 107) | | |
| 9 | TCR_A0064 | CLGGLLTMV | EBV-LMP2 | |
| | | (SEQ ID NO: 106) | | |
| 10 | TCR_A0065 | CLGGLLTMV | EBV-LMP2 | |
| | | (SEQ ID NO: 106) | | |
| 11 | TCR_A0066 | CLGGLLTMV | EBV-LMP2 | |
| | | (SEQ ID NO: 106) | | |
| 12 | TCR_A0068 | FLYALALLL | EBV LMP2 | |
| | | (SEQ ID NO: 107) | | |
| 13 | TCR_A0069 | FLYALALLL | EBV LMP2 | |
| | | (SEQ ID NO: 107) | | |
| 14 | TCR_A0070 | FLYALALLL | EBV LMP2 | |
| | | (SEQ ID NO: 107) | | |
| 15 | TCR_A0099 | Peptide pool/EPLPQGQLTAY | EBV/BZLF1 | |
| | | (SEQ ID NO: 145) | | |
| 16 | TCR_A0130 | RLPGVLPRA | Mutant splice | |
| | | (SEQ ID NO: 147) | factor-induced | |
| | | | peptide of | |
| | | | MAPK8IP2 | |
| 17 | TCR_A0131 | RLPGVLPRA | Mutant splice | |
| | | (SEQ ID NO: 147) | factor-induced | |
| | | | peptide of | |
| | | | MAPK8IP2 | |
| 18 | TCR_A0100 | FLQFKTWWI | HERV-K gag | |
| | | (SEQ ID NO: 148) | protein | |
| 19 | TCR_A0132 | RLPGVLPRA | Mutant splice | |
| | | (SEQ ID NO: 147) | factor-induced | |
| | | | peptide of | |
| | | | MAPK8IP2 | |
| 20 | TCR_A0358 | RLPGVLPRA | Mutant splice | |
| | | (SEQ ID NO: 147) | factor-induced | |
| | | | peptide of | |
| | | | MAPK8IP2 | |
| 21 | TCR_A0359 | RLPGVLPRA | Mutant splice | |
| | | (SEQ ID NO: 147) | factor-induced | |
| | | | peptide of | |
| | | | MAPK8IP2 | |

TABLE 3A
Amino acid sequences of CDR1, 2 and 3 regions of TCRα and TCRβ

| | Column A | | | | | | Column B | | | | | |
|---|---|---|---|---|---|---|---|---|---|---|---|---|
| | Alpha chain | | | | | | Beta chain | | | | | |
| | SEQ | | SEQ | | SEQ | | SEQ | | SEQ | | SEQ | |
| TCR ID | ID | CDR1α | ID | CDR2α | ID | CDR3α | ID | CDR1β | ID | CDR2β | ID | CDR3β |
| TCR_A0002 | 1 | YGGTVN | 7 | YFSGDPLV | 15 | CAVKDTDKLIF | 26 | LGHDT | 34 | YNNKEL | 43 | CASSPDFNEQFF |
| TCR_A0003 | 1 | YGGTVN | 7 | YFSGDPLV | 16 | CAGGAAGNKLTF | 27 | SGHAT | 35 | FQNNGV | 44 | CASSSPLGGFAGANVLT |
| | | | | | | | | | | | | F |
| TCR_A0004 | 1 | YGGTVN | 7 | YFSGDPLV | 15 | CAVKDTDKLIF | 25 | KGHDR | 33 | SFDVKD | 45 | CATSDFISDTQYF |
| TCR_A0005 | 2 | TSDQSYG | 8 | QGSYDEQN | 17 | CAMREGGNFNKFY | 28 | SQVTM | 36 | ANQGSEA | 46 | CSVGGTSGTLPANEQFF |
| | | | | | | F | | | | | | |
| TCR_A0015 | 3 | SSVSVY | 9 | YLSGSTLV | 18 | CAVSALSYNQGGK | 29 | SGHNS | 37 | FNNNVP | 47 | CASSWTGNEQYF |
| | | | | | | LIF | | | | | | |
| TCR_A0061 | 4 | DSAIYN | 10 | IQSSQRE | 19 | CAVLMDSNYQLIW | 30 | WSHSY | 38 | SAAADI | 48 | CASSSDGMNTEAFF |
| TCR_A0062 | 5 | TSINN | 11 | IRSNERE | 20 | CATEGSSGYSTLTF | 31 | MNHEY | 39 | SVGAGI | 49 | CASSKQGGGYGYTF |
| TCR_A0064 | 6 | TTLSN | 12 | LVKSGEV | 21 | CAGAGAGSYQLTF | 32 | SGHRS | 40 | YFSETQ | 50 | CASSLEGQASSYEQYF |
| TCR_A0065 | 6 | TTLSN | 12 | LVKSGEV | 22 | CAVSGAGSYQLTF | 32 | SGHRS | 40 | YFSETQ | 50 | CASSLEGQASSYEQYF |
| TCR_A0066 | 6 | TTLSN | 12 | LVKSGEV | 21 | CAGAGAGSYQLTF | 32 | SGHRS | 40 | YESETQ | 51 | CASSAEGQASSYEQYF |
| TCR_A0068 | 5 | TSINN | 13 | IRSNERE | 23 | CATEGGSGYSTLTF | 31 | MNHEY | 41 | SVGAGI | 52 | CASSRQGGSGSGYTF |
| TCR_A0069 | 5 | TSINN | 13 | IRSNERE | 23 | CATEGDSGYSTLTF | 31 | MNHEY | 41 | SVGAGI | 53 | CASTTQGGAYGYTF |
| TCR_A0070 | 5 | TSINN | 13 | IRSNERE | 24 | CATAGNSGYSTLTF | 31 | MNHEY | 41 | SVGAGI | 54 | CASTPQGGNEAFF |
| TCR_A0099 | 136 | SSNFYA | 137 | MTLNGDE | 138 | CAVNAGGTSYGKL | 27 | SGHAT | 35 | FQNNGV | 139 | CASSSDWTANNEQFF |
| | | | | | | TF | | | | | | |
| TCR_A0130 | 149 | TSESNYY | 150 | QEAYKQQN | 151 | CAFMIPDSNYQLIW | 154 | LGHNA | 156 | YNFKEQ | 159 | CASSQVGTSGRGGELFF |
| TCR_A0131 | 165 | TSENNYY | 150 | QEAYKQQN | 152 | CAFMLIDSGTYKYI | 31 | MNHEY | 157 | SMNVEV | 160 | CASSLGQGTETQYF |
| | | | | | | F | | | | | | |
| TCR_A0100 | 4 | DSAIYN | 10 | IQSSQRE | 153 | CAVGGNNNDMRF | 155 | PRHDT | 158 | FYEKMQ | 161 | CASSLINTEAFF |
| TCR_A0132 | 149 | TSESNYY | 150 | QEAYKQQN | 14 | CAFMEADSNYQLI | 32 | SGHRS | 40 | YFSETQ | 42 | CASKGRRGPDYNSPLH |
| | | | | | | W | | | | | | F |
| TCR_A0358 | 165 | TSENNYY | 150 | QEAYKQQN | 194 | CAFMGPDSGTYKYI | 25 | KGHDR | 33 | SFDVKD | 195 | CATSDSDRIYGYTF |
| | | | | | | F | | | | | | |
| TCR_A0359 | 2 | TSDQSYG | 8 | QGSYDEQN | 196 | CAMREPDSNYQLI | 197 | SNHLY | 198 | FYNNEI | 199 | CASQKGLEYEQYF |
| | | | | | | W | | | | | | |

TABLE 3B
Amino acid sequences of CDR1, 2 and 3 regions of TCRα and TCRβ

| | | Column A | | | | Column B | | | |
|---|---|---|---|---|---|---|---|---|---|
| | | Alpha chain | | | | Beta chain | | | |
| Row | TCR | FR1 | FR2 | FR3 | FR4 | FR1 | FR2 | FR3 | FR4 |
| 1 | TCR_A0002 | SEQ ID | SEQ ID | SEQ ID | SEQ ID | SEQ ID | SEQ ID | SEQ ID | SEQ ID |
| | | NO: 225 | NO: 226 | NO: 227 | NO: 228 | NO: 259 | NO: 260 | NO: 261 | NO: 262 |
| 2 | TCR_A0003 | SEQ ID | SEQ ID | SEQ ID | SEQ ID | SEQ ID | SEQ ID | SEQ ID | SEQ ID |
| | | NO: 225 | NO: 226 | NO: 227 | NO: 229 | NO: 263 | NO: 264 | NO: 265 | NO: 266 |
| 3 | TCR_A0004 | SEQ ID | SEQ ID | SEQ ID | SEQ ID | SEQ ID | SEQ ID | SEQ ID | SEQ ID |
| | | NO: 225 | NO: 226 | NO: 227 | NO: 228 | NO: 267 | NO: 268 | NO: 269 | NO: 262 |
| 4 | TCR_A0005 | SEQ ID | SEQ ID | SEQ ID | SEQ ID | SEQ ID | SEQ ID | SEQ ID | SEQ ID |
| | | NO: 230 | NO: 231 | NO: 232 | NO: 233 | NO: 270 | NO: 271 | NO: 272 | NO: 262 |
| 5 | TCR_A0015 | SEQ ID | SEQ ID | SEQ ID | SEQ ID | SEQ ID | SEQ ID | SEQ ID | SEQ ID |
| | | NO: 234 | NO: 235 | NO: 236 | NO: 237 | NO: 273 | NO: 274 | NO: 275 | NO: 276 |
| 6 | TCR_A0061 | SEQ ID | SEQ ID | SEQ ID | SEQ ID | SEQ ID | SEQ ID | SEQ ID | SEQ ID |
| | | NO: 238 | NO: 239 | NO: 240 | NO: 241 | NO: 277 | NO: 278 | NO: 279 | NO: 280 |
| 7 | TCR_A0062 | SEQ ID | SEQ ID | SEQ ID | SEQ ID | SEQ ID | SEQ ID | SEQ ID | SEQ ID |
| | | NO: 242 | NO: 243 | NO: 244 | NO: 245 | NO: 281 | NO: 282 | NO: 283 | NO: 284 |
| 8 | TCR_A0064 | SEQ ID | SEQ ID | SEQ ID | SEQ ID | SEQ ID | SEQ ID | SEQ ID | SEQ ID |
| | | NO: 246 | NO: 247 | NO: 248 | NO: 249 | NO: 285 | NO: 286 | NO: 287 | NO: 276 |
| 9 | TCR_A0065 | SEQ ID | SEQ ID | SEQ ID | SEQ ID | SEQ ID | SEQ ID | SEQ ID | SEQ ID |
| | | NO: 246 | NO: 247 | NO: 248 | NO: 249 | NO: 285 | NO: 286 | NO: 287 | NO: 276 |
| 10 | TCR_A0066 | SEQ ID | SEQ ID | SEQ ID | SEQ ID | SEQ ID | SEQ ID | SEQ ID | SEQ ID |
| | | NO: 246 | NO: 247 | NO: 248 | NO: 249 | NO: 285 | NO: 286 | NO: 287 | NO: 276 |
| 11 | TCR_A0068 | SEQ ID | SEQ ID | SEQ ID | SEQ ID | SEQ ID | SEQ ID | SEQ ID | SEQ ID |
| | | NO: 242 | NO: 243 | NO: 244 | NO: 245 | NO: 281 | NO: 282 | NO: 283 | NO: 284 |
| 12 | TCR_A0069 | SEQ ID | SEQ ID | SEQ ID | SEQ ID | SEQ ID | SEQ ID | SEQ ID | SEQ ID |
| | | NO: 242 | NO: 243 | NO: 244 | NO: 245 | NO: 281 | NO: 282 | NO: 283 | NO: 284 |
| 13 | TCR_A0070 | SEQ ID | SEQ ID | SEQ ID | SEQ ID | SEQ ID | SEQ ID | SEQ ID | SEQ ID |
| | | NO: 242 | NO: 243 | NO: 244 | NO: 245 | NO: 281 | NO: 282 | NO: 283 | NO: 280 |
| 14 | TCR_A0099 | SEQ ID | SEQ ID | SEQ ID | SEQ ID | SEQ ID | SEQ ID | SEQ ID | SEQ ID |
| | | NO: 250 | NO: 251 | NO: 252 | NO: 253 | NO: 263 | NO: 264 | NO: 265 | NO: 262 |
| 15 | TCR_A0130 | SEQ ID | SEQ ID | SEQ ID | SEQ ID | SEQ ID | SEQ ID | SEQ ID | SEQ ID |
| | | NO: 254 | NO: 255 | NO: 256 | NO: 241 | NO: 288 | NO: 289 | NO 290 | NO: 291 |
| 16 | TCR_A0131 | SEQ ID | SEQ ID | SEQ ID | SEQ ID | SEQ ID | SEQ ID | SEQ ID | SEQ ID |
| | | NO: 254 | NO: 255 | NO: 256 | NO: 257 | NO: 292 | NO: 293 | NO: 294 | NO: 295 |
| 17 | TCR_A0100 | SEQ ID | SEQ ID | SEQ ID | SEQ ID | SEQ ID | SEQ ID | SEQ ID | SEQ ID |
| | | NO: 238 | NO: 239 | NO: 240 | NO: 258 | NO: 296 | NO: 297 | NO: 298 | NO: 280 |
| 18 | TCR_A0132 | SEQ ID | SEQ ID | SEQ ID | SEQ ID | SEQ ID | SEQ ID | SEQ ID | SEQ ID |
| | | NO: 254 | NO: 255 | NO: 256 | NO: 241 | NO: 285 | NO: 286 | NO: 287 | NO: 299 |
| 19 | TCR_A0358 | SEQ ID | SEQ ID | SEQ ID | SEQ ID | SEQ ID | SEQ ID | SEQ ID | SEQ ID |
| | | NO: 254 | NO: 255 | NO: 256 | NO: 257 | NO: 267 | NO: 300 | NO: 269 | NO: 284 |
| 20 | TCR_A0359 | SEQ ID | SEQ ID | SEQ ID | SEQ ID | SEQ ID | SEQ ID | SEQ ID | SEQ ID |
| | | NO: 230 | NO: 231 | NO: 232 | NO: 241 | NO: 301 | NO: 302 | NO: 303 | NO: 276 |

### C. TCRs

The T cell receptor (TCR) is composed of two chains (αβ or γδ) that pair on the surface of the T cell to form a heterodimeric receptor. The αβ TCR is expressed on most T cells in the body and is known to be involved in the recognition of MHC-restricted antigens. The molecular genetics, structure, and biochemistry of αβ TCRs have now been studied thoroughly. Each α and β chain is composed of two domains: Constant domains (C) that anchor the protein in the cell membrane and that associate with invariant subunits of the CD3 signaling apparatus, and Variable domains (V) that confer antigen recognition through six loops, called complementarity determining regions (CDR). The V domains of each chain have three CDRs. These CDRs interact with a complex between an antigenic peptide bound to a protein encoded by the major histocompatibility complex (pepMHC) (Davis and Bjorkman (1988) Nature, 334, 395-402; Davis et al. (1998) Annu Rev Immunol, 16, 523-544; Murphy (2012), xix, 868 p.).

Provided herein are novel synthetic TCRs comprising the TCRα and TCRβ CDR sequences listed in Table 3A, FR sequences listed in Table 3B, and/or amino acid sequences listed in Table 4, or nucleotide sequences listed in Table 5, or optimized nucleotide sequences listed in Table 6, herein. Provided herein are also synthetic TCRs comprising TCRα and TCRβ human variable regions and mouse constant regions, in order to improve the expression of the TCR, and in order to use the mouse constant region for the tracking of transfected human T cells with an anti-mouse antibody. Provided are human/mouse hybrid TCRs comprising mouse TCRa and TCRb constant region amino acid sequences listed in Table 7. Provided herein are also synthetic TCRs comprising TCRα and TCRβ human variable regions and human constant regions. In some embodiments the human constant region amino acid sequences are listed in Table 8.

In some embodiments, the TCRs bind to EBV-derived antigenic peptides mixes. In some embodiments, the TCRs bind to EBV-derived antigenic single peptides. In some embodiments, the TCRs bind to the EBV BRLF1-derived antigenic peptide with the sequence from SEQ ID NO:105 (YVLDHLIVV). In some embodiments, the TCRs bind to the EBV LMP2-derived antigenic peptide with the sequence from SEQ ID NO:106 (CLGGLLTMV). In some embodiments, the TCRs bind to the EBV LMP2-derived antigenic peptide with the sequence from SEQ ID NO:107 (FLYALALLL). In some embodiments, the TCR binds to BRLF1. In some embodiments, the TCR binds to LMP2. In some embodiments, the TCRs bind to the LMP2A-derived antigenic peptide with the sequence from SEQ ID NO:146 (MGSLEMVPM). In some embodiments, the TCR binds to BZLF1. In some embodiments, the TCRs bind to the BZLF1-derived antigenic peptide with the sequence from SEQ ID NO:145 (EPLPQGQLTAY). In some embodiments, the TCR binds to BMLF1, BALF2, BMRF1, BNRF1, BLLF1, BXLF2, EBNA1, EBNA2, EBNA3, EBNA4, EBNA6, or LMP1. In some embodiments, the TCR binds to a mutant splice factor-induced peptide of MAPK8IP2. In some embodiments, the TCRs bind to the MAPK8IP2-derived antigenic peptide with the sequence from SEQ ID NO:147 (RLPGVLPRA). In some embodiments, the TCR binds to a peptide from HERV-K gag protein. In some embodiments, the TCRs bind to the HERV-K-derived antigenic peptide with the sequence from SEQ ID NO:148 (FLQFKTWWI).

In some embodiments, the TCR comprises the CDRs, FRs and/or the alpha and/or beta chain variable domains of a TCR described herein, or CDRs, FRs and/or alpha and/or beta chain variable domains which are derived from those of a TCR described herein. In some embodiments, a TCR is selected from TCR_A0002, TCR_A0003, TCR_A0004, TCR_A0005, TCR_A0015, TCR_A0061, TCR_A0062, TCR_A0064, TCR_A0065, TCR_A0066, TCR_A0068, TCR_A0069, TCR_A0070, TCR_A0099, TCR_A0130, TCR_A0131, TCR_A0100, TCR_A0132, TCR_A0358, TCR_A0359, TCR_0362 and TCR_0363.

In some embodiments, the TCR of the present disclosure comprises a polypeptide or polypeptides comprising an alpha chain comprising the alpha chain CDRs of a clone shown in Table 3A herein, and a beta chain comprising the beta chain CDRs of a clone shown in Table 3A herein. That is, in some embodiments, the TCR comprises a polypeptide or polypeptides comprising: (i) an alpha chain comprising CDR1α, CDR2α and CDR3α as indicated in column A of Table A, and (ii) a beta chain comprising CDR1β, CDR2β and CDR3β as indicated in column B of Table 3A, wherein the sequences of columns A and B are selected from the same row of Table 3A.

In some embodiments, the TCR of the present disclosure comprises a polypeptide or polypeptides comprising an alpha chain amino acid sequence comprising the alpha chain FRs shown in Table 3B herein, and a beta chain amino acid sequence comprising the beta chain FRs of a TCR shown in Table 3B herein. That is, in some embodiments, the TCR comprises a polypeptide or polypeptides comprising: (i) a FR1, FR2, FR3 and FR4 as indicated in column A of Table 3B, and (ii) a beta chain comprising FR1, FR2, FR3, and FR4 as indicated in column B of Table 3B, wherein the sequences of columns A and B are selected from the same row of Table 3B.

In some embodiments, the TCR of the present disclosure comprises a polypeptide or polypeptides comprising an alpha chain amino acid sequence having at least 70%, preferably one of at least 80%, at least 85%, at least 90%, at least 91%, at least 92%, at least 93%, at least 94%, at least 95%, at least 96%, at least 97%, at least 98%, at least 99% or 100% amino acid sequence identity to an amino acid sequence indicated in column A of Table 4 herein, and a beta chain amino acid sequence having at least 70%, preferably one of at least 80%, at least 85%, at least 90%, at least 91%, at least 92%, at least 93%, at least 94%, at least 95%, at least 96%, at least 97%, at least 98%, at least 99% or 100% amino acid sequence identity to an amino acid sequence indicated in column B of Table 4 herein.

That is, in some embodiments, the TCR comprises a polypeptide or polypeptides comprising: (i) alpha chain amino acid sequence having at least 70%, preferably one of at least 80%, at least 85%, at least 90%, at least 91%, at least 92%, at least 93%, at least 94%, at least 95%, at least 96%, at least 97%, at least 98%, at least 99% or 100% amino acid sequence identity to an amino acid sequence indicated in column A of Table 4, and (ii) a beta chain amino acid sequence having at least 70%, preferably one of at least 80%, at least 85%, at least 90%, at least 91%, at least 92%, at least 93%, at least 94%, at least 95%, at least 96%, at least 97%, at least 98%, at least 99% or 100% amino acid sequence identity to an amino acid sequence indicated in column B of Table 4, wherein the sequences of columns A and B are selected from the same row of Table 4.

In some embodiments, the TCR of the present disclosure comprises a polypeptide or polypeptides comprising an alpha chain of a TCR shown in Table 4 herein, and a beta chain of a TCR shown in Table 4 herein. That is, in some embodiments, the TCR comprises a polypeptide or polypeptides comprising: (i) an alpha chain comprising a sequence as indicated in column A of Table 4, and (ii) a beta chain comprising a sequence as indicated in column B of Table 4, wherein the sequences of columns A and B are selected from the same row of Table 4. In some embodiments, the TCRs comprise a TCRα peptide chain sharing at least 70%, preferably one of at least 80%, at least 85%, at least 90%, at least 91%, at least 92%, at least 93%, at least 94%, at least 95%, at least 96%, at least 97%, at least 98%, at least 99% or 100% amino acid sequence identity with a member selected from SEQ ID NOs: 55; 56; 57; 58; 59; 60; 61; 62; 63; 64; 65; 66; 140; 162; 163; 164; 200; and 202 in combination with: a TCRβ peptide chain sharing at least 70%, preferably one of at least 80%, at least 85%, at least 90%, at least 91%, at least 92%, at least 93%, at least 94%, at least 95%, at least 96%, at least 97%, at least 98%, at least 99% or 100% amino acid sequence identity with a member selected from SEQ ID NOs: 67; 68; 69; 70; 71; 72; 73; 74; 75; 76; 77; 78; 91; 141; 166; 167; 168; 201; and 203. In some embodiments, the TCRs comprise a variable domain comprising a TCRα chain and TCRβ chain of polypeptide SEQ ID NO pairs selected from the group consisting of: SEQ ID NOs: 55 and 67; SEQ ID NOs: 56 and 68; SEQ ID NOs: 55 and 69; SEQ ID NOs: 57 and 70; SEQ ID NOs: 58 and 71; SEQ ID NOs: 59 and 72; SEQ ID NOs: 60 and 73; SEQ ID NOs: 61 and 74; SEQ ID NOs: 62 and 74; SEQ ID NOs: 61 and 75; SEQ ID NOs: 63 and 76; SEQ ID NOs: 64 and 77; SEQ ID NOs: 65 and 78; SEQ ID NOs: 140 and 141; SEQ ID NOs: 162 and 166; SEQ ID NOs: 163 and 167; SEQ ID NOs: 164 and 168; SEQ ID NOs: 66 and 91; SEQ ID NOs: 200 and 201; and SEQ ID NOS 202 and 203.

The TCRs disclosed herein may be encoded by any nucleotide sequence that encodes for the required amino acid sequence(s), taking into account codon degeneracy.

In some embodiments, the TCR of the present disclosure comprises a polypeptide or polypeptides encoded by a nucleic acid described herein. In some embodiments, the TCR of the present disclosure comprises an alpha chain of a TCR encoded by a nucleotide sequence in Table 5 or 6 herein, and a beta chain of a TCR encoded by a nucleotide sequence in Table 5 or 6 herein. That is, in some embodiments, the TCR comprises a polypeptide or polypeptides encoded by: (i) a nucleotide sequence comprising a sequence as indicated in column A of Table 5 or 6, and (ii) a nucleic acid sequence comprising a sequence as indicated in column B of Table 5 or 6, wherein the sequences of columns A and B are selected from the same row of Table 5 or 6.

In some embodiments, the TCRs comprise a TCRα chain variable domain encoded by a nucleic acid sharing at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with a member selected from SEQ ID NOs: 79; 80; 81; 82; 83; 84; 85; 86; 87; 88; 89; 90; 142; 169; 170; 171; 108; 109; 110; 111; 112; 113; 114; 115; 116; 117; 118; 119; 120; 134; 175; 176; 177; 186; 188; 204; 206; 213; 215; 217; 219; and 221 in combination with a TCRβ chain encoded by a nucleic acid sharing at least 70%, preferably one of at least 80%, at least 85%, at least 90%, at least 91%, at least 92%, at least 93%, at least 94%, at least 95%, at least 96%, at least 97%, at least 98%, at least 99% or 100% sequence identity with a member selected from SEQ ID NOs: 92; 93; 94; 95; 96; 97; 98; 99; 100; 101; 102; 103; 104; 143; 172; 173; 174; 121; 122; 123; 124; 125; 126; 127; 128; 129; 130; 131; 132; 133; 135; 178; 179; 180, 187; 189, 205; 207; 214; 216; 218; 220; and 222 as set forth in Table 5 and Table 6. In some embodiments, the TCRs comprise TCRα chain and TCRβ chain of nucleotide SEQ ID NO pairs selected from the group consisting of: SEQ ID NOs: 79 and 92; SEQ ID NOs: 80 and 93; SEQ ID NOs: 79 and 94; SEQ ID NOs: 81 and 95; SEQ ID NOs: 82 and 96; SEQ ID NOs: 83 and 97; SEQ ID NOs: 84 and 98; SEQ ID NOs: 85 and 99; SEQ ID NOs: 86 and 100; SEQ ID NOs: 87 and 101; SEQ ID NOs: 88 and 102; SEQ ID NOs: 89 and 103; SEQ ID NOs: 90 and 104; SEQ ID NOs: 142 and 143; SEQ ID NOs: 169 and 172; SEQ ID NOs: 170 and 173; SEQ ID NOs: 171 and 174; SEQ ID NOs: 186 and 187; SEQ ID NOs: 204 and 205; SEQ ID NOs: 206 and 207; SEQ ID NOs: 108 and 121; SEQ ID NOs: 109 and 122; SEQ ID NOs: 110 and 123; SEQ ID NOs: 111 and 124; SEQ ID NOs: 112 and 125; SEQ ID NOs: 113 and 126; SEQ ID NOs: 114 and 127; SEQ ID NOs: 115 and 128; SEQ ID NOs: 116 and 129; SEQ ID NOs: 117 and 130; SEQ ID NOs: 118 and 131; SEQ ID NOs: 119 and 132; SEQ ID NOs: 120 and 133; SEQ ID NOs: 134 and 135; SEQ ID NOs: 175 and 178; SEQ ID NOs: 176 and 179; SEQ ID NOs: 177 and 180; SEQ ID NOs: 188 and 189; SEQ ID NOs: 213 and 214; SEQ ID NOs: 215 and 216; SEQ ID NOs: 217 and 218; SEQ ID NOs: 219 and 220; and SEQ ID NOs: 221 and 222.

In some embodiments, the TCRs comprise the TCRα chain and the TCRβ chain complete amino acid sequences which share at least about 80%, about 85%, about 90%, or about 95% sequence identity with the sequence combinations set forth in Table 4. In some embodiments, the TCRs are encoded by the TCRα chain and the TCRβ chain complete nucleotide sequences which share at least about 80%, about 85%, about 90%, or about 95% sequence identity with the sequence combinations set forth in Table 5 and Table 6.

TABLE 4
Amino acid sequences of the TCRα and TCRβ variable regions. The CDR3 region is underlined.

| TCR ID | TCRα variable region [column A] | TCRβ variable region [column B] |
|---|---|---|
| TCR_A0002 | AQSVSQHNHHVILSEAASLELGCNYSY | DTAVSQTPKYLVTQMGNDKSIKCEQN |
| | GGTVNLFWYVQYPGQHLQLLLKYFSG | LGHDTMYWYKQDSKKFLKIMFSYNN |
| | DPLVKGIKGFEAEFIKSKFSFNLRKPSV | KELIINETVPNRFSPKSPDKAHLNLHIN |
| | QWSDTAEYFCAVKDTDKLIFGTGTRLQ | SLELGDSAVYFCASSPDFNEQFFGPGT |
| | VFP | RLTVL |
| | (SEQ ID NO: 55) | (SEQ ID NO: 67) |
| TCR_A0003 | AQSVSQHNHHVILSEAASLELGCNYSY | EAGVAQSPRYKIIEKRQSVAFWCNPIS |
| | GGTVNLFWYVQYPGQHLQLLLKYFSG | GHATLYWYQQILGQGPKLLIQFQNNG |
| | DPLVKGIKGFEAEFIKSKFSFNLRKPSV | VVDDSQLPKDRFSAERLKGVDSTLKIQ |
| | QWSDTAEYFCAGGAAGNKLTFGGGTR | PAKLEDSAVYLCASSSPLGGFAGANVL |
| | VLVKP | TFGAGSRLTVL |
| | (SEQ ID NO: 56) | (SEQ ID NO: 68) |
| TCR_A0004 | AQSVSQHNHHVILSEAASLELGCNYSY | DADVTQTPRNRITKTGKRIMLECSQTK |
| | GGTVNLFWYVQYPGQHLQLLLKYFSG | GHDRMYWYRQDPGLGLRLIYYSFDVK |
| | DPLVKGIKGFEAEFIKSKFSFNLRKPSV | DINKGEISDGYSVSRQAQAKFSLSLESA |
| | QWSDTAEYFCAVKDTDKLIFGTGTRLQ | IPNQTALYFCATSDFISDTQYFGPGTRL |
| | VFP | TVL |
| | (SEQ ID NO: 55) | (SEQ ID NO: 69) |
| TCR_A0005 | AQKITQTQPGMFVQEKEAVTLDCTYDT | SAVISQKPSRDICQRGTSLTIQCQVDSQ |
| | SDQSYGLFWYKQPSSGEMIFLIYQGSY | VTMMFWYRQQPGQSLTLIATANQGSE |
| | DEQNATEGRYSLNFQKARKSANLVISA | ATYESGFVIDKFPISRPNLTFSTLTVSN |
| | SQLGDSAMYFCAMREGGNFNKFYFGS | MSPEDSSIYLCSVGGTSGTLPANEQFFG |
| | GTKLNVKP | PGTRLTVL |
| | (SEQ ID NO: 57) | (SEQ ID NO: 70) |
| TCR_A0015 | AQSVTQLDSQVPVFEEAPVELRCNYSS | DAGVIQSPRHEVTEMGQEVTLRCKPIS |
| | SVSVYLFWYVQYPNQGLQLLLKYLSGS | GHNSLFWYRQTMMRGLELLIYFNNNV |
| | TLVKGINGFEAEFNKSQTSFHLRKPSVH | PIDDSGMPEDRFSAKMPNASFSTLKIQP |
| | ISDTAEYFCAVSALSYNQGGKLIFGQGT | SEPRDSAVYFCASSWTGNEQYFGPGTR |
| | ELSVKP | LTVT |
| | (SEQ ID NO: 58) | (SEQ ID NO: 71) |
| TCR_A0061 | KQEVTQIPAALSVPEGENLVLNCSFTDS | DAGITQSPRYKITETGRQVTLMCHQT |
| | AIYNLQWFRQDPGKGLTSLLLIQSSQRE | WSHSYMFWYRQDLGHGLRLIYYSAA |
| | QTSGRLNASLDKSSGRSTLYIAASQPGD | ADITDKGEVPDGYVVSRSKTENFPLTL |
| | SATYLCAVLMDSNYQLIWGAGTKLIIK | ESATRSQTSVYFCASSSDGMNTEAFFG |
| | P | QGTRLTVV |
| | (SEQ ID NO: 59) | (SEQ ID NO: 72) |
| TCR_A0062 | SQQGEEDPQALSIQEGENATMNCSYKT | NAGVTQTPKFQVLKTGQSMTLQCAQD |
| | SINNLQWYRQNSGRGLVHLILIRSNERE | MNHEYMSWYRQDPGMGLRLIHYSVG |
| | KHSGRLRVTLDTSKKSSSLLITASRAAD | AGITDQGEVPNGYNVSRSTTEDFPLRL |
| | TASYFCATEGSSGYSTLTFGKGTMLLV | LSAAPSQTSVYFCASSKQGGGYGYTFG |
| | SP | SGTRLTVV |
| | (SEQ ID NO: 60) | (SEQ ID NO: 73) |
| TCR_A0064 | GQQVMQIPQYQHVQEGEDFTTYCNSST | KAGVTQTPRYLIKTRGQQVTLSCSPISG |
| | TLSNIQWYKQRPGGHPVFLIQLVKSGE | HRSVSWYQQTPGQGLQFLFEYFSETQR |
| | VKKQKRLTFQFGEAKKNSSLHITATQT | NKGNFPGRFSGRQFSNSRSEMNVSTLE |
| | TDVGTYFCAGAGAGSYQLTFGKGTKL | LGDSALYLCASSLEGQASSYEQYFGPG |
| | SVIP | TRLTVT |
| | (SEQ ID NO: 61) | (SEQ ID NO: 74) |
| TCR_A0065 | GQQVMQIPQYQHVQEGEDFTTYCNSST | KAGVTQTPRYLIKTRGQQVTLSCSPISG |
| | TLSNIQWYKQRPGGHPVFLIQLVKSGE | HRSVSWYQQTPGQGLQFLFEYFSETQR |
| | VKKQKRLTFQFGEAKKNSSLHITATQT | NKGNFPGRFSGRQFSNSRSEMNVSTLE |
| | TDVGTYFCAVSGAGSYQLTFGKGTKLS | LGDSALYLCASSLEGQASSYEQYFGPG |
| | VIP | TRLTVT |
| | (SEQ ID NO: 62) | (SEQ ID NO: 74) |
| TCR_A0066 | GQQVMQIPQYQHVQEGEDFTTYCNSST | KAGVTQTPRYLIKTRGQQVTLSCSPISG |
| | TLSNIQWYKQRPGGHPVFLIQLVKSGE | HRSVSWYQQTPGQGLQFLFEYFSETQR |
| | VKKQKRLTFQFGEAKKNSSLHITATQT | NKGNFPGRFSGRQFSNSRSEMNVSTLE |
| | TDVGTYFCAGAGAGSYQLTFGKGTKL | LGDSALYLCASSAEGQASSYEQYFGPG |
| | SVIP | TRLTVT |
| | (SEQ ID NO: 61) | (SEQ ID NO: 75) |
| TCR_A0068 | SQQGEEDPQALSIQEGENATMNCSYKT | NAGVTQTPKFQVLKTGQSMTLQCAQD |
| | SINNLQWYRQNSGRGLVHLILIRSNERE | MNHEYMSWYRQDPGMGLRLIHYSVG |
| | KHSGRLRVTLDTSKKSSSLLITASRAAD | AGITDQGEVPNGYNVSRSTTEDFPLRL |
| | TASYFCATEGGSGYSTLTFGKGTMLLV | LSAAPSQTSVYFCASSRQGGSGSGYTF |
| | SP | GSGTRLTVV |
| | (SEQ ID NO: 63) | (SEQ ID NO: 76) |
| TCR_A0069 | SQQGEEDPQALSIQEGENATMNCSYKT | NAGVTQTPKFQVLKTGQSMTLQCAQD |
| | SINNLQWYRQNSGRGLVHLILIRSNERE | MNHEYMSWYRQDPGMGLRLIHYSVG |
| | KHSGRLRVTLDTSKKSSSLLITASRAAD | AGITDQGEVPNGYNVSRSTTEDFPLRL |
| | TASYFCATEGDSGYSTLTFGKGTMLLV | LSAAPSQTSVYFCASTTQGGAYGYTFG |
| | SP | SGTRLTVV |
| | (SEQ ID NO: 64) | (SEQ ID NO: 77) |
| TCR_A0070 | SQQGEEDPQALSIQEGENATMNCSYKT | NAGVTQTPKFQVLKTGQSMTLQCAQD |
| | SINNLQWYRQNSGRGLVHLILIRSNERE | MNHEYMSWYRQDPGMGLRLIHYSVG |
| | KHSGRLRVTLDTSKKSSSLLITASRAAD | AGITDQGEVPNGYNVSRSTTEDFPLRL |
| | TASYFCATAGNSGYSTLTFGKGTMLLV | LSAAPSQTSVYFCASTPQGGNEAFFGQ |
| | SP | GTRLTVV |
| | (SEQ ID NO: 65) | (SEQ ID NO: 78) |
| TCR_A0099 | ILNVEQSPQSLHVQEGDSTNFTCSFPSS | EAGVAQSPRYKIIEKRQSVAFWCNPIS |
| | NFYALHWYRWETAKSPEALFVMTLNG | GHATLYWYQQILGQGPKLLIQFQNNG |
| | DEKKKGRISATLNTKEGYSYLYIKGSQP | VVDDSQLPKDRFSAERLKGVDSTLKIQ |
| | EDSATYLCAVNAGGTSYGKLTFGQGTI | PAKLEDSAVYLCASSSDWTANNEQFF |
| | LTVHP | GPGTRLTVL |
| | (SEQ ID NO: 140) | (SEQ ID NO: 141) |
| TCR_A0130 | AQTVTQSQPEMSVQEAETVTLSCTYDT | ETGVTQTPRHLVMGMTNKKSLKCEQH |
| | SESNYYLFWYKQPPSRQMILVIRQEAY | LGHNAMYWYKQSAKKPLELMFVYNF |
| | KQQNATENRFSVNFQKAAKSFSLKISD | KEQTENNSVPSRFSPECPNSSHLFLHLH |
| | SQLGDTAMYFCAFMIPDSNYQLIWGAG | TLQPEDSALYLCASSQVGTSGRGGELF |
| | TKLIIKP | FGEGSRLTVL |
| | (SEQ ID NO: 162) | (SEQ ID NO: 166) |
| TCR_A0131 | AQTVTQSQPEMSVQEAETVTLSCTYDT | EAQVTQNPRYLITVTGKKLTVTCSQN |
| | SENNYYLFWYKQPPSRQMILVIRQEAY | MNHEYMSWYRQDPGLGLRQIYYSMN |
| | KQQNATENRFSVNFQKAAKSFSLKISD | VEVTDKGDVPEGYKVSRKEKRNFPLIL |
| | SQLGDTAMYFCAFMLIDSGTYKYIFGT | ESPSPNQTSLYFCASSLGQGTETQYFGP |
| | GTRLKVLA | GTRLLVL |
| | (SEQ ID NO: 163) | (SEQ ID NO: 167) |
| TCR_A0100 | KQEVTQIPAALSVPEGENLVLNCSFTDS | AAGVIQSPRHLIKEKRETATLKCYPIPR |
| | AIYNLQWFRQDPGKGLTSLLLIQSSQRE | HDTVYWYQQGPGQDPQFLISFYEKMQ |
| | QTSGRLNASLDKSSGRSTLYIAASQPGD | SDKGSIPDRFSAQQFSDYHSELNMSSL |
| | SATYLCAVGGNNNDMRFGAGTRLTVK | ELGDSALYFCASSLINTEAFFGQGTRLT |
| | P | VV |
| | SEQ ID NO: 164) | (SEQ ID NO: 168) |
| TCR_A0132 | AQTVTQSQPEMSVQEAETVTLSCTYDT | KAGVTQTPRYLIKTRGQQVTLSCSPISG |
| | SESNYYLFWYKQPPSRQMILVIRQEAY | HRSVSWYQQTPGQGLQFLFEYFSETQR |
| | KQQNATENRFSVNFQKAAKSFSLKISD | NKGNFPGRFSGRQFSNSRSEMNVSTLE |
| | SQLGDTAMYFCAFMEADSNYQLIWGA | LGDSALYLCASKGRRGPDYNSPLHFG |
| | GTKLIIKP | NGTRLTVT |
| | (SEQ ID NO: 66) | (SEQ ID NO: 91) |
| TCR_A0358 | AQTVTQSQPEMSVQEAETVTLSCTYDT | DADVTQTPRNRITKTGKRIMLECSQTK |
| | SENNYYLFWYKQPPSRQMILVIRQEAY | GHDRMYWYRQDPGLGLQLIYYSFDV |
| | KQQNATENRFSVNFQKAAKSFSLKISD | KDINKGEISDGYSVSRQAQAKFSLSLES |
| | SQLGDTAMYFCAFMGPDSGTYKYIFGT | AIPNQTALYFCATSDSDRIYGYTFGSGT |
| | GTRLKVLA | RLTVV |
| | (SEQ ID NO: 200) | (SEQ ID NO: 201) |
| TCR_A0359 | AQKITQTQPGMFVQEKEAVTLDCTYDT | EPEVTQTPSHQVTQMGQEVILRCVPIS |
| | SDQSYGLFWYKQPSSGEMIFLIYQGSY | NHLYFYWYRQILGQKVEFLVSFYNNEI |
| | DEQNATEGRYSLNFQKARKSANLVISA | SEKSEIFDDQFSVERPDGSNFTLKIRST |
| | SQLGDSAMYFCAMREPDSNYQLIWGA | KLEDSAMYFCASQKGLEYEQYFGPGT |
| | GTKLIIKP | RLTVT |
| | (SEQ ID NO: 202) | (SEQ ID NO: 203) |

Exemplary known TCRα and TCRβ variable regions amino acid sequences are set forth in Kamga et al., 2019, as follows: TCR_A0001: TCRα variable region:

(SEQ ID NO: 55)
AQSVSQHNHHVILSEAASLELGCNYSYGGTVNLFWYVQYPGQHLQLLLK
YFSGDPLVKGIKGFEAEFIKSKFSFNLRKPSVQWSDTAEYFCAVKDTDK
LIFGTGTRLQVFP;
TCRβ variable region:
(SEQ ID NO: 193)
DADVTQTPRNRITKTGKRIMLECSQTKGHDRMYWYRQDPGLGLRLIYYS
FDVKDINKGEISDGYSVSRQAQAKFSLSLESAIPNQTALYFCATSDWDD
STGELFFGEGSRLTVL. The CDR3 region is underlined.

In some embodiments, the TCRα chain variable domain nucleotide sequence is selected from the group consisting of SEQ ID NO:79, SEQ ID NO:80, SEQ ID NO:81, SEQ ID NO:82, SEQ ID NO:83, SEQ ID NO: 84, SEQ ID NO: 85, SEQ ID NO:86, SEQ ID NO:87, SEQ ID NO:88, SEQ ID NO: 89, SEQ ID NO:90, SEQ ID NO: 142, SEQ ID NO: 169, SEQ ID NO: 170, SEQ ID NO:171, SEQ ID NO: 186, SEQ ID NO:204 and SEQ ID NO: 206, or a codon degenerate nucleotide sequence thereof encoding the amino acid sequence encoded by the reference sequence. In some embodiments, the TCRα chain variable domain nucleotide sequence shares at least about 80%, or 85%, or 90%, or 95% sequence identity with a member selected from: SEQ ID NO:79, SEQ ID NO: 80, SEQ ID NO:81, SEQ ID NO: 82, SEQ ID NO:83, SEQ ID NO:84, SEQ ID NO:85, SEQ ID NO:86, SEQ ID NO: 87, SEQ ID NO:88 SEQ ID NO:89, SEQ ID NO:90, SEQ ID NO:142, SEQ ID NO: 169, SEQ ID NO: 170, SEQ ID NO:171, SEQ ID NO:186, SEQ ID NO:204 and SEQ ID NO:206, as listed in Table 5, or a codon degenerate nucleotide sequence thereof encoding the amino acid sequence encoded by the reference sequence.

In some embodiments, the TCR β chain variable domain nucleotide sequence is selected from the group consisting of SEQ ID NO:92, SEQ ID NO:93, SEQ ID NO:94, SEQ ID NO:95, SEQ ID NO:96, SEQ ID NO: 97, SEQ ID NO:98, SEQ ID NO:99, SEQ ID NO: 100, SEQ ID NO: 101, SEQ ID NO: 102, SEQ ID NO:103, SEQ ID NO: 104, SEQ ID NO:143, SEQ ID NO:172, SEQ ID NO:173, SEQ ID NO:174, SEQ ID NO: 187, SEQ ID NO: 205 and SEQ ID NO:207, or a codon degenerate nucleotide sequence thereof encoding the amino acid sequence encoded by the reference sequence. In some embodiments, the TCR β chain variable domain nucleotide sequence is at least 80%, or 85%, or 90%, or 95%, or 99% identical to SEQ ID NO:92, SEQ ID NO: 93, SEQ ID NO:94, SEQ ID NO:95, SEQ ID NO:96, SEQ ID NO:97, SEQ ID NO:98, SEQ ID NO:99, SEQ ID NO: 100, SEQ ID NO:101, SEQ ID NO: 102, SEQ ID NO:103, SEQ ID NO: 104, SEQ ID NO:143, SEQ ID NO: 172, SEQ ID NO:173, SEQ ID NO:174, SEQ ID NO:187, SEQ ID NO:205 and SEQ ID NO:207 as listed in Table 5, or a codon degenerate nucleotide sequence thereof encoding the amino acid sequence encoded by the reference sequence.

TABLE 5
Nucleotide sequences of the TCRα and TCRβ variable regions. The CDR3 region is underlined.

| TCR ID | TCRα variable region [column A] | TCRβ variable region [column B] |
|---|---|---|
| TCR_A0002 | GCCCAGTCTGTGAGCCAGCATAACCA | GACACAGCTGTTTCCCAGACTCCAAA |
| | CCACGTAATTCTCTCTGAAGCAGCCT | ATACCTGGTCACACAGATGGGAAAC |
| | CACTGGAGTTGGGATGCAACTATTCC | GACAAGTCCATTAAATGTGAACAAA |
| | TATGGTGGAACTGTTAATCTCTTCTG | ATCTGGGCCATGATACTATGTATTGG |
| | GTATGTCCAGTACCCTGGTCAACACC | TATAAACAGGACTCTAAGAAATTTCT |
| | TTCAGCTTCTCCTCAAGTACTTTTCAG | GAAGATAATGTTTAGCTACAATAATA |
| | GGGATCCACTGGTTAAAGGCATCAA | AGGAGCTCATTATAAATGAAACAGTT |
| | GGGCTTTGAGGCTGAATTTATAAAGA | CCAAATCGCTTCTCACCTAAATCTCC |
| | GTAAATTCTCCTTTAATCTGAGGAAA | AGACAAAGCTCACTTAAATCTTCACA |
| | CCCTCTGTGCAGTGGAGTGACACAGC | TCAATTCCCTGGAGCTTGGTGACTCT |
| | TGAGTACTTCTGTGCCGTGAAGGACA | GCTGTGTATTTCTGTGCCAGCAGCCC |
| | CCGACAAGCTCATCTTTGGGACTGGG | AGACTTCAATGAGCAGTTCTTCGGGC |
| | ACCAGATTACAAGTCTTTCCA | CAGGGACACGGCTCACCGTGCTA |
| | (SEQ ID NO: 79) | (SEQ ID NO: 92) |
| TCR_A0003 | GCCCAGTCTGTGAGCCAGCATAACCA | GAAGCTGGAGTTGCCCAGTCTCCCAG |
| | CCACGTAATTCTCTCTGAAGCAGCCT | ATATAAGATTATAGAGAAAAGGCAG |
| | CACTGGAGTTGGGATGCAACTATTCC | AGTGTGGCTTTTTGGTGCAATCCTAT |
| | TATGGTGGAACTGTTAATCTCTTCTG | ATCTGGCCATGCTACCCTTTACTGGT |
| | GTATGTCCAGTACCCTGGTCAACACC | ACCAGCAGATCCTGGGACAGGGCCC |
| | TTCAGCTTCTCCTCAAGTACTTTTCAG | AAAGCTTCTGATTCAGTTTCAGAATA |
| | GGGATCCACTGGTTAAAGGCATCAA | ACGGTGTAGTGGATGATTCACAGTTG |
| | GGGCTTTGAGGCTGAATTTATAAAGA | CCTAAGGATCGATTTTCTGCAGAGAG |
| | GTAAATTCTCCTTTAATCTGAGGAAA | GCTCAAAGGAGTAGACTCCACTCTCA |
| | CCCTCTGTGCAGTGGAGTGACACAGC | AGATCCAACCTGCAAAGCTTGAGGAC |
| | TGAGTACTTCTGTGCCGGGGGAGCTG | TCGGCCGTGTATCTCTGTGCCAGCAG |
| | CAGGCAACAAGCTAACTTTTGGAGG | TTCACCATTGGGGGGGTTCGCGGGGG |
| | AGGAACCAGGGTGCTAGTTAAACCA | CCAACGTCCTGACTTTCGGGGCCGGC |
| | (SEQ ID NO: 80) | AGCAGGCTGACCGTGCTG |
| | | (SEQ ID NO: 93) |
| TCR_A0004 | GCCCAGTCTGTGAGCCAGCATAACCA | GATGCTGATGTTACCCAGACCCCAAG |
| | CCACGTAATTCTCTCTGAAGCAGCCT | GAATAGGATCACAAAGACAGGAAAG |
| | CACTGGAGTTGGGATGCAACTATTCC | AGGATTATGCTGGAATGTTCTCAGAC |
| | TATGGTGGAACTGTTAATCTCTTCTG | TAAGGGTCATGATAGAATGTACTGGT |
| | GTATGTCCAGTACCCTGGTCAACACC | ATCGACAAGACCCAGGACTGGGCCT |
| | TTCAGCTTCTCCTCAAGTACTTTTCAG | ACGGTTGATCTATTACTCCTTTGATGT |
| | GGGATCCACTGGTTAAAGGCATCAA | CAAAGATATAAACAAAGGAGAGATC |
| | GGGCTTTGAGGCTGAATTTATAAAGA | TCTGATGGATACAGTGTCTCTCGACA |
| | GTAAATTCTCCTTTAATCTGAGGAAA | GGCACAGGCTAAATTCTCCCTGTCCC |
| | CCCTCTGTGCAGTGGAGTGACACAGC | TAGAGTCTGCCATCCCCAACCAGACA |
| | TGAGTACTTCTGTGCCGTGAAGGACA | GCTCTTTACTTCTGTGCCACCAGTGAT |
| | CCGACAAGCTCATCTTTGGGACTGGG | TTCATCTCAGATACGCAGTATTTTGG |
| | ACCAGATTACAAGTCTTTCCA | CCCAGGCACCCGGCTGACAGTGCTC |
| | (SEQ ID NO: 79) | (SEQ ID NO: 94) |
| TCR_A0005 | GCCCAGAAGATAACTCAAACCCAAC | AGTGCTGTCATCTCTCAAAAGCCAAG |
| | CAGGAATGTTCGTGCAGGAAAAGGA | CAGGGATATCTGTCAACGTGGAACCT |
| | GGCTGTGACTCTGGACTGCACATATG | CCCTGACGATCCAGTGTCAAGTCGAT |
| | ACACCAGTGATCAAAGTTATGGTCTA | AGCCAAGTCACCATGATGTTCTGGTA |
| | TTCTGGTACAAGCAGCCCAGCAGTGG | CCGTCAGCAACCTGGACAGAGCCTGA |
| | GGAAATGATTTTTCTTATTTATCAGG | CACTGATCGCAACTGCAAATCAGGGC |
| | GGTCTTATGACGAGCAAAATGCAAC | TCTGAGGCCACATATGAGAGTGGATT |
| | AGAAGGTCGCTACTCATTGAATTTCC | TGTCATTGACAAGTTTCCCATCAGCC |
| | AGAAGGCAAGAAAATCCGCCAACCT | GCCCAAACCTAACATTCTCAACTCTG |
| | TGTCATCTCCGCTTCACAACTGGGGG | ACTGTGAGCAACATGAGCCCTGAAG |
| | ACTCAGCAATGTATTTCTGTGCAATG | ACAGCAGCATATATCTCTGCAGCGTT |
| | AGAGAGGGCGGGAACTTCAACAAAT | GGTGGGACTAGCGGGACTCTCCCTGC |
| | TTTACTTTGGATCTGGGACCAAACTC | CAATGAGCAGTTCTTCGGGCCAGGGA |
| | AATGTAAAACCA | CACGGCTCACCGTGCTA |
| | (SEQ ID NO: 81) | (SEQ ID NO: 95) |
| TCR_A0015 | GCCCAGTCTGTGACCCAGCTTGACAG | GATGCTGGAGTTATCCAGTCACCCCG |
| | CCAAGTCCCTGTCTTTGAAGAAGCCC | CCATGAGGTGACAGAGATGGGACAA |
| | CTGTGGAGCTGAGGTGCAACTACTCA | GAAGTGACTCTGAGATGTAAACCAAT |
| | TCGTCTGTTTCAGTGTATCTCTTCTGG | TTCAGGCCACAACTCCCTTTTCTGGT |
| | TATGTGCAATACCCCAACCAAGGACT | ACAGACAGACCATGATGCGGGGACT |
| | CCAGCTTCTCCTGAAGTATTTATCAG | GGAGTTGCTCATTTACTTTAACAACA |
| | GATCCACCCTGGTTAAAGGCATCAAC | ACGTTCCGATAGATGATTCAGGGATG |
| | GGTTTTGAGGCTGAATTTAACAAGAG | CCCGAGGATCGATTCTCAGCTAAGAT |
| | TCAAACTTCCTTCCACTTGAGGAAAC | GCCTAATGCATCATTCTCCACTCTGA |
| | CCTCAGTCCATATAAGCGACACGGCT | AGATCCAGCCCTCAGAACCCAGGGA |
| | GAGTACTTCTGTGCTGTGAGTGCCCT | CTCAGCTGTGTACTTCTGTGCCAGCA |
| | TTCTTATAACCAGGGAGGAAAGCTTA | GCTGGACAGGGAACGAGCAGTACTT |
| | TCTTCGGACAGGGAACGGAGTTATCT | CGGGCCGGGCACCAGGCTCACGGTC |
| | GTGAAACCC | ACA |
| | (SEQ ID NO: 82) | (SEQ ID NO: 96) |
| TCR_A0061 | AAACAGGAGGTGACACAGATTCCTG | GATGCTGGAATCACCCAGAGCCCAA |
| | CAGCTCTGAGTGTCCCAGAAGGAGA | GATACAAGATCACAGAGACAGGAAG |
| | AAACTTGGTTCTCAACTGCAGTTTCA | GCAGGTGACCTTGATGTGTCACCAGA |
| | CTGATAGCGCTATTTACAACCTCCAG | CTTGGAGCCACAGCTATATGTTCTGG |
| | TGGTTTAGGCAGGACCCTGGGAAAG | TATCGACAAGACCTGGGACATGGGCT |
| | GTCTCACATCTCTGTTGCTTATTCAGT | GAGGCTGATCTATTACTCAGCAGCTG |
| | CAAGTCAGAGAGAGCAAACAAGTGG | CTGATATTACAGATAAAGGAGAAGTC |
| | AAGACTTAATGCCTCGCTGGATAAAT | CCCGATGGCTATGTTGTCTCCAGATC |
| | CATCAGGACGTAGTACTTTATACATT | CAAGACAGAGAATTTCCCCCTCACTC |
| | GCAGCTTCTCAGCCTGGTGACTCAGC | TGGAGTCAGCTACCCGCTCCCAGACA |
| | CACCTACCTCTGTGCTGTCCTTATGG | TCTGTGTATTTCTGCGCCAGCAGCTC |
| | ATAGCAACTATCAGTTAATCTGGGGC | GGACGGGATGAACACTGAAGCTTTCT |
| | GCTGGGACCAAGCTAATTATAAAGCC | TTGGACAAGGCACCAGACTCACAGTT |
| | A | GTA |
| | (SEQ ID NO: 83) | (SEQ ID NO: 97) |
| TCR_A0062 | AGTCAACAGGGAGAAGAGGATCCTC | AATGCTGGTGTCACTCAGACCCCAAA |
| | AGGCCTTGAGCATCCAGGAGGGTGA | ATTCCAGGTCCTGAAGACAGGACAG |
| | AAATGCCACCATGAACTGCAGTTACA | AGCATGACACTGCAGTGTGCCCAGGA |
| | AAACTAGTATAAACAATTTACAGTGG | TATGAACCATGAATACATGTCCTGGT |
| | TATAGACAAAATTCAGGTAGAGGCCT | ATCGACAAGACCCAGGCATGGGGCT |
| | TGTCCACCTAATTTTAATACGTTCAA | GAGGCTGATTCATTACTCAGTTGGTG |
| | ATGAAAGAGAGAAACACAGTGGAAG | CTGGTATCACTGACCAAGGAGAAGTC |
| | ATTAAGAGTCACGCTTGACACTTCCA | CCCAATGGCTACAATGTCTCCAGATC |
| | AGAAAAGCAGTTCCTTGTTGATCACG | AACCACAGAGGATTTCCCGCTCAGGC |
| | GCTTCCCGGGCAGCAGACACTGCTTC | TGCTGTCGGCTGCTCCCTCCCAGACA |
| | TTACTTCTGTGCTACGGAGGGCTCTT | TCTGTGTACTTCTGTGCCAGCAGTAA |
| | CAGGATACAGCACCCTCACCTTTGGG | ACAGGGAGGGGGCTATGGCTACACC |
| | AAGGGGACTATGCTTCTAGTCTCTCC | TTCGGTTCGGGGACCAGGTTAACCGT |
| | A | TGTA |
| | (SEQ ID NO: 84) | (SEQ ID NO: 98) |
| TCR_A0064 | GGACAACAGGTAATGCAAATTCCTCA | AAGGCTGGAGTCACTCAAACTCCAAG |
| | GTACCAGCATGTACAAGAAGGAGAG | ATATCTGATCAAAACGAGAGGACAG |
| | GACTTCACCACGTACTGCAATTCCTC | CAAGTGACACTGAGCTGCTCCCCTAT |
| | AACTACTTTAAGCAATATACAGTGGT | CTCTGGGCATAGGAGTGTATCCTGGT |
| | ATAAGCAAAGGCCTGGTGGACATCC | ACCAACAGACCCCAGGACAGGGCCT |
| | CGTTTTTTTGATACAGTTAGTGAAGA | TCAGTTCCTCTTTGAATACTTCAGTGA |
| | GTGGAGAAGTGAAGAAGCAGAAAAG | GACACAGAGAAACAAAGGAAACTTC |
| | ACTGACATTTCAGTTTGGAGAAGCAA | CCTGGTCGATTCTCAGGGCGCCAGTT |
| | AAAAGAACAGCTCCCTGCACATCAC | CTCTAACTCTCGCTCTGAGATGAATG |
| | AGCCACCCAGACTACAGATGTAGGA | TGAGCACCTTGGAGCTGGGGGACTCG |
| | ACCTACTTCTGTGCAGGAGCTGGGGC | GCCCTTTATCTTTGCGCCAGCAGCCT |
| | TGGGAGTTACCAACTCACTTTCGGGA | CGAGGGACAGGCGAGCTCCTACGAG |
| | AGGGGACCAAACTCTCGGTCATACCA | CAGTACTTCGGGCCGGGCACCAGGCT |
| | (SEQ ID NO: 85) | CACGGTCACA |
| | | (SEQ ID NO: 99) |
| TCR_A0065 | GGACAACAGGTAATGCAAATTCCTCA | AAGGCTGGAGTCACTCAAACTCCAAG |
| | GTACCAGCATGTACAAGAAGGAGAA | ATATCTGATCAAAACGAGAGGACAG |
| | GACTTCACCACGTACTGCAATTCCTC | CAAGTGACACTGAGCTGCTCCCCTAT |
| | AACTACTTTAAGCAATATACAGTGGT | CTCTGGGCATAGGAGTGTATCCTGGT |
| | ATAAGCAAAGGCCTGGTGGACATCC | ACCAACAGACCCCAGGACAGGGCCT |
| | CGTTTTTTTGATACAGTTAGTGAAGA | TCAGTTCCTCTTTGAATACTTCAGTGA |
| | GTGGAGAAGTGAAGAAGCAGAAAAG | GACACAGAGAAACAAAGGAAACTTC |
| | ACTGACATTTCAGTTTGGAGAAGCAA | CCTGGTCGATTCTCAGGGCGCCAGTT |
| | AAAAGAACAGCTCCCTGCACATCAC | CTCTAACTCTCGCTCTGAGATGAATG |
| | AGCCACCCAGACTACAGATGTAGGA | TGAGCACCTTGGAGCTGGGGGACTCG |
| | ACCTACTTCTGTGCAGTCTCTGGGGC | GCCCTTTATCTTTGCGCCAGCAGCTT |
| | TGGGAGTTACCAACTCACTTTCGGGA | GGAGGGGCAGGCCTCCTCCTACGAGC |
| | AGGGGACCAAACTCTCGGTCATACCA | AGTACTTCGGGCCGGGCACCAGGCTC |
| | (SEQ ID NO: 86) | ACGGTCACA |
| | | (SEQ ID NO: 100) |
| TCR_A0066 | GGACAACAGGTAATGCAAATTCCTCA | AAGGCTGGAGTCACTCAAACTCCAAG |
| | GTACCAGCATGTACAAGAAGGAGAG | ATATCTGATCAAAACGAGAGGACAG |
| | GACTTCACCACGTACTGCAATTCCTC | CAAGTGACACTGAGCTGCTCCCCTAT |
| | AACTACTTTAAGCAATATACAGTGGT | CTCTGGGCATAGGAGTGTATCCTGGT |
| | ATAAGCAAAGGCCTGGTGGACATCC | ACCAACAGACCCCAGGACAGGGCCT |
| | CGTTTTTTTGATACAGTTAGTGAAGA | TCAGTTCCTCTTTGAATACTTCAGTGA |
| | GTGGAGAAGTGAAGAAGCAGAAAAG | GACACAGAGAAACAAAGGAAACTTC |
| | ACTGACATTTCAGTTTGGAGAAGCAA | CCTGGTCGATTCTCAGGGCGCCAGTT |
| | AAAAGAACAGCTCCCTGCACATCAC | CTCTAACTCTCGCTCTGAGATGAATG |
| | AGCCACCCAGACTACAGATGTAGGA | TGAGCACCTTGGAGCTGGGGGACTCG |
| | ACCTACTTCTGTGCAGGGGCTGGGGC | GCCCTTTATCTTTGCGCCAGCAGCGC |
| | TGGGAGTTACCAACTCACTTTCGGGA | GGAGGGACAGGCTTCCTCCTACGAGC |
| | AGGGGACCAAACTCTCGGTCATACCA | AGTACTTCGGGCCGGGCACCAGGCTC |
| | (SEQ ID NO: 87) | ACGGTCACA |
| | | (SEQ ID NO: 101) |
| TCR_A0068 | AGTCAACAGGGAGAAGAGGATCCTC | AATGCTGGTGTCACTCAGACCCCAAA |
| | AGGCCTTGAGCATCCAGGAGGGTGA | ATTCCAGGTCCTGAAGACAGGACAG |
| | AAATGCCACCATGAACTGCAGTTACA | AGCATGACACTGCAGTGTGCCCAGGA |
| | AAACTAGTATAAACAATTTACAGTGG | TATGAACCATGAATACATGTCCTGGT |
| | TATAGACAAAATTCAGGTAGAGGCCT | ATCGACAAGACCCAGGCATGGGGCT |
| | TGTCCACCTAATTTTAATACGTTCAA | GAGGCTGATTCATTACTCAGTTGGTG |
| | ATGAAAGAGAGAAACACAGTGGAAG | CTGGTATCACTGACCAAGGAGAAGTC |
| | ATTAAGAGTCACGCTTGACACTTCCA | CCCAATGGCTACAATGTCTCCAGATC |
| | AGAAAAGCAGTTCCTTGTTGATCACG | AACCACAGAGGATTTCCCGCTCAGGC |
| | GCTTCCCGGGCAGCAGACACTGCTTC | TGCTGTCGGCTGCTCCCTCCCAGACA |
| | TTACTTCTGTGCTACTGAGGGCGGTT | TCTGTGTACTTCTGTGCCAGCAGTCG |
| | CAGGATACAGCACCCTCACCTTTGGG | ACAAGGGGGTTCCGGGAGTGGCTAC |
| | AAGGGGACTATGCTTCTAGTCTCTCC | ACCTTCGGTTCGGGGACCAGGTTAAC |
| | A | CGTTGTA |
| | (SEQ ID NO: 88) | (SEQ ID NO: 102) |
| TCR_A0069 | AGTCAACAGGGAGAAGAGGATCCTC | AATGCTGGTGTCACTCAGACCCCAAA |
| | AGGCCTTGAGCATCCAGGAGGGTGA | ATTCCAGGTCCTGAAGACAGGACAG |
| | AAATGCCACCATGAACTGCAGTTACA | AGCATGACACTGCAGTGTGCCCAGGA |
| | AAACTAGTATAAACAATTTACAGTGG | TATGAACCATGAATACATGTCCTGGT |
| | TATAGACAAAATTCAGGTAGAGGCCT | ATCGACAAGACCCAGGCATGGGGCT |
| | TGTCCACCTAATTTTAATACGTTCAA | GAGGCTGATTCATTACTCAGTTGGTG |
| | ATGAAAGAGAGAAACACAGTGGAAG | CTGGTATCACTGACCAAGGAGAAGTC |
| | ATTAAGAGTCACGCTTGACACTTCCA | CCCAATGGCTACAATGTCTCCAGATC |
| | AGAAAAGCAGTTCCTTGTTGATCACG | AACCACAGAGGATTTCCCGCTCAGGC |
| | GCTTCCCGGGCAGCAGACACTGCTTC | TGCTGTCGGCTGCTCCCTCCCAGACA |
| | TTACTTCTGTGCTACGGAGGGGGATT | TCTGTGTACTTCTGTGCCAGCACCAC |
| | CAGGATACAGCACCCTCACCTTTGGG | CCAGGGGGGGGCCTATGGCTACACCT |
| | AAGGGGACTATGCTTCTAGTCTCTCC | TCGGTTCGGGGACCAGGTTAACCGTT |
| | A | GTA |
| | (SEQ ID NO: 89) | (SEQ ID NO: 103) |
| TCR_A0070 | AGTCAACAGGGAGAAGAGGATCCTC | AATGCTGGTGTCACTCAGACCCCAAA |
| | AGGCCTTGAGCATCCAGGAGGGTGA | ATTCCAGGTCCTGAAGACAGGACAG |
| | AAATGCCACCATGAACTGCAGTTACA | AGCATGACACTGCAGTGTGCCCAGGA |
| | AAACTAGTATAAACAATTTACAGTGG | TATGAACCATGAATACATGTCCTGGT |
| | TATAGACAAAATTCAGGTAGAGGCCT | ATCGACAAGACCCAGGCATGGGGCT |
| | TGTCCACCTAATTTTAATACGTTCAA | GAGGCTGATTCATTACTCAGTTGGTG |
| | ATGAAAGAGAGAAACACAGTGGAAG | CTGGTATCACTGACCAAGGAGAAGTC |
| | ATTAAGAGTCACGCTTGACACTTCCA | CCCAATGGCTACAATGTCTCCAGATC |
| | AGAAAAGCAGTTCCTTGTTGATCACG | AACCACAGAGGATTTCCCGCTCAGGC |
| | GCTTCCCGGGCAGCAGACACTGCTTC | TGCTGTCGGCTGCTCCCTCCCAGACA |
| | TTACTTCTGTGCTACGGCCGGTAATT | TCTGTGTACTTCTGTGCCAGCACCCC |
| | CAGGATACAGCACCCTCACCTTTGGG | CCAGGGGGGCAACGAAGCTTTCTTTG |
| | AAGGGGACTATGCTTCTAGTCTCTCC | GACAAGGCACCAGACTCACAGTTGTA |
| | A | (SEQ ID NO: 104) |
| | (SEQ ID NO: 90) | |
| TCR_A0099 | ATACTGAACGTGGAACAAAGTCCTCA | GAAGCTGGAGTTGCCCAGTCTCCCAG |
| | GTCACTGCATGTTCAGGAGGGAGAC | ATATAAGATTATAGAGAAAAGGCAG |
| | AGCACCAATTTCACCTGCAGCTTCCC | AGTGTGGCTTTTTGGTGCAATCCTAT |
| | TTCCAGCAATTTTTATGCCTTACACTG | ATCTGGCCATGCTACCCTTTACTGGT |
| | GTACAGATGGGAAACTGCAAAAAGC | ACCAGCAGATCCTGGGACAGGGCCC |
| | CCCGAGGCCTTGTTTGTAATGACTTT | AAAGCTTCTGATTCAGTTTCAGAATA |
| | AAATGGGGATGAAAAGAAGAAAGGA | ACGGTGTAGTGGATGATTCACAGTTG |
| | CGAATAAGTGCCACTCTTAATACCAA | CCTAAGGATCGATTTTCTGCAGAGAG |
| | GGAGGGTTACAGCTATTTGTACATCA | GCTCAAAGGAGTAGACTCCACTCTCA |
| | AAGGATCCCAGCCTGAAGACTCAGC | AGATCCAACCTGCAAAGCTTGAGGAC |
| | CACATACCTCTGTGCCGTTAATGCTG | TCGGCCGTGTATCTCTGTGCCAGCAG |
| | GTGGTACTAGCTATGGAAAGCTGACA | CTCCGATTGGACAGCGAACAATGAGC |
| | TTTGGACAAGGGACCATCTTGACTGT | AGTTCTTCGGGCCAGGGACACGGCTC |
| | CCATCCA | ACCGTGCTA |
| | (SEQ ID NO: 142) | (SEQ ID NO: 143) |
| TCR_A0130 | GCCCAGACAGTCACTCAGTCTCAACC | GAAACGGGAGTTACGCAGACACCAA |
| | AGAGATGTCTGTGCAGGAGGCAGAG | GACACCTGGTCATGGGAATGACAAAT |
| | ACTGTGACCCTGAGTTGCACATATGA | AAGAAGTCTTTGAAATGTGAACAACA |
| | CACCAGTGAGAGTAATTATTATTTGT | TCTGGGGCATAACGCTATGTATTGGT |
| | TCTGGTACAAACAGCCTCCCAGCAGG | ACAAGCAAAGTGCTAAGAAGCCACT |
| | CAGATGATTCTCGTTATTCGCCAAGA | GGAGCTCATGTTTGTCTACAACTTTA |
| | AGCTTATAAGCAACAGAATGCAACG | AAGAACAGACTGAAAACAACAGTGT |
| | GAGAATCGTTTCTCTGTGAACTTCCA | GCCAAGTCGCTTCTCACCTGAATGCC |
| | GAAAGCAGCCAAATCCTTCAGTCTCA | CCAACAGCTCTCACTTATTCCTTCACC |
| | AGATCTCAGACTCACAGCTGGGGGA | TACACACCCTGCAGCCAGAAGACTCG |
| | CACTGCGATGTATTTCTGTGCTTTCAT | GCCCTGTATCTCTGTGCCAGCAGCCA |
| | GATACCGGATAGCAACTATCAGTTAA | AGTTGGGACTAGCGGGAGGGGCGGG |
| | TCTGGGGCGCTGGGACCAAGCTAATT | GAGCTGTTTTTTGGAGAAGGCTCTAG |
| | ATAAAGCCA | GCTGACCGTACTG |
| | (SEQ ID NO: 169) | (SEQ ID NO: 172) |
| TCR_A0131 | GCCCAGACAGTCACTCAGTCTCAACC | GAAGCCCAAGTGACCCAGAACCCAA |
| | AGAGATGTCTGTGCAGGAGGCAGAG | GATACCTCATCACAGTGACTGGAAAG |
| | ACTGTGACCCTGAGTTGCACATATGA | AAGTTAACAGTGACTTGTTCTCAGAA |
| | CACCAGTGAGAATAATTATTATTTGT | TATGAACCATGAGTATATGTCCTGGT |
| | TCTGGTACAAGCAGCCTCCCAGCAGG | ATCGACAAGACCCAGGGCTGGGCTTA |
| | CAGATGATTCTCGTTATTCGCCAAGA | AGGCAGATCTACTATTCAATGAATGT |
| | AGCTTATAAGCAACAGAATGCAACG | TGAGGTGACTGATAAGGGAGATGTTC |
| | GAGAATCGTTTCTCTGTGAACTTCCA | CTGAAGGGTACAAAGTCTCTCGAAAA |
| | GAAAGCAGCCAAATCCTTCAGTCTCA | GAGAAGAGGAATTTCCCCCTGATCCT |
| | AGATCTCAGACTCACAGCTGGGGGA | GGAGTCGCCCAGCCCCAACCAGACCT |
| | CACTGCGATGTATTTCTGTGCTTTCAT | CTCTGTACTTCTGTGCCAGCAGTCTTG |
| | GTTAATAGACTCAGGAACCTACAAAT | GACAGGGAACAGAGACCCAGTACTT |
| | ACATCTTTGGAACAGGCACCAGGCTG | CGGGCCAGGCACGCGGCTCCTGGTGC |
| | AAGGTTTTAGCA | TC |
| | (SEQ ID NO: 170) | (SEQ ID NO: 173) |
| TCR_A0100 | AAACAGGAGGTGACACAGATTCCTG | GCTGCTGGAGTCATCCAGTCCCCAAG |
| | CAGCTCTGAGTGTCCCAGAAGGAGA | ACATCTGATCAAAGAAAAGAGGGAA |
| | AAACTTGGTTCTCAACTGCAGTTTCA | ACAGCCACTCTGAAATGCTATCCTAT |
| | CTGATAGCGCTATTTACAACCTCCAG | CCCTAGACACGACACTGTCTACTGGT |
| | TGGTTTAGGCAGGACCCTGGGAAAG | ACCAGCAGGGTCCAGGTCAGGACCC |
| | GTCTCACATCTCTGTTGCTTATTCAGT | CCAGTTCCTCATTTCGTTTTATGAAAA |
| | CAAGTCAGAGAGAGCAAACAAGTGG | GATGCAGAGCGATAAAGGAAGCATC |
| | AAGACTTAATGCCTCGCTGGATAAAT | CCTGATCGATTCTCAGCTCAACAGTT |
| | CATCAGGACGTAGTACTTTATACATT | CAGTGACTATCATTCTGAACTGAACA |
| | GCAGCTTCTCAGCCTGGTGACTCAGC | TGAGCTCCTTGGAGCTGGGGGACTCA |
| | CACCTACCTCTGTGCTGTGGGAGGCA | GCCCTGTACTTCTGTGCCAGCAGCTT |
| | ATAACAATGACATGCGCTTTGGAGCA | AATTAACACTGAAGCTTTCTTTGGAC |
| | GGGACCAGACTGACAGTAAAACCA | AAGGCACCAGACTCACAGTTGTA |
| | (SEQ ID NO: 171) | (SEQ ID NO: 174) |
| TCR_A0132 | GCCCAGACAGTCACTCAGTCTCAACC | AAGGCTGGAGTCACTCAAACTCCAAG |
| | AGAGATGTCTGTGCAGGAGGCAGAG | ATATCTGATCAAAACGAGAGGACAG |
| | ACTGTGACCCTGAGTTGCACATATGA | CAAGTGACACTGAGCTGCTCCCCTAT |
| | CACCAGTGAGAGTAATTATTATTTGT | CTCTGGGCATAGGAGTGTATCCTGGT |
| | TCTGGTACAAACAGCCTCCCAGCAGG | ACCAACAGACCCCAGGACAGGGCCT |
| | CAGATGATTCTCGTTATTCGCCAAGA | TCAGTTCCTCTTTGAATACTTCAGTGA |
| | AGCTTATAAGCAACAGAATGCAACG | GACACAGAGAAACAAAGGAAACTTC |
| | GAGAATCGTTTCTCTGTGAACTTCCA | CCTGGTCGATTCTCAGGGCGCCAGTT |
| | GAAAGCAGCCAAATCCTTCAGTCTCA | CTCTAACTCTCGCTCTGAGATGAATG |
| | AGATCTCAGACTCACAGCTGGGGGA | TGAGCACCTTGGAGCTGGGGGACTCG |
| | CACTGCGATGTATTTCTGTGCTTTCAT | GCCCTTTATCTTTGCGCCAGCAAGGG |
| | GGAGGCGGATAGCAACTATCAGTTA | CAGGCGGGGGCCGGACTATAATTCAC |
| | ATCTGGGGCGCTGGGACCAAGCTAAT | CCCTCCACTTTGGGAACGGGACCAGG |
| | TATAAAGCCA | CTCACTGTGACA |
| | (SEQ ID NO: 186) | (SEQ ID NO: 187) |
| TCR_A0358 | GCCCAGACAGTCACTCAGTCTCAACC | GATGCTGATGTTACCCAGACCCCAAG |
| | AGAGATGTCTGTGCAGGAGGCAGAG | GAATAGGATCACAAAGACAGGAAAG |
| | ACTGTGACCCTGAGTTGCACATATGA | AGGATTATGCTGGAATGTTCTCAGAC |
| | CACCAGTGAGAATAATTATTATTTGT | TAAGGGTCATGATAGAATGTACTGGT |
| | TCTGGTACAAGCAGCCTCCCAGCAGG | ATCGACAAGACCCAGGACTGGGCCT |
| | CAGATGATTCTCGTTATTCGCCAAGA | ACAGTTGATCTATTACTCCTTTGATGT |
| | AGCTTATAAGCAACAGAATGCAACG | CAAAGATATAAACAAAGGAGAGATC |
| | GAGAATCGTTTCTCTGTGAACTTCCA | TCTGATGGATACAGTGTCTCTCGACA |
| | GAAAGCAGCCAAATCCTTCAGTCTCA | GGCACAGGCTAAATTCTCCCTGTCCC |
| | AGATCTCAGACTCACAGCTGGGGGA | TAGAGTCTGCCATCCCCAACCAGACA |
| | CACTGCGATGTATTTCTGTGCTTTCAT | GCTCTTTACTTCTGTGCCACCAGTGAT |
| | GGGACCTGACTCAGGAACCTACAAA | TCCGACAGAATCTATGGCTACACCTT |
| | TACATCTTTGGAACAGGCACCAGGCT | CGGTTCGGGGACCAGGTTAACCGTTG |
| | GAAGGTTTTAGCA | TA |
| | (SEQ ID NO: 204) | (SEQ ID NO: 205) |
| TCR_A0359 | GCCCAGAAGATAACTCAAACCCAAC | GAACCTGAAGTCACCCAGACTCCCAG |
| | CAGGAATGTTCGTGCAGGAAAAGGA | CCATCAGGTCACACAGATGGGACAG |
| | GGCTGTGACTCTGGACTGCACATATG | GAAGTGATCTTGCGCTGTGTCCCCAT |
| | ACACCAGTGATCAAAGTTATGGTCTA | CTCTAATCACTTATACTTCTATTGGTA |
| | TTCTGGTACAAGCAGCCCAGCAGTGG | CAGACAAATCTTGGGGCAGAAAGTC |
| | GGAAATGATTTTTCTTATTTATCAGG | GAGTTTCTGGTTTCCTTTTATAATAAT |
| | GGTCTTATGACGAGCAAAATGCAAC | GAAATCTCAGAGAAGTCTGAAATATT |
| | AGAAGGTCGCTACTCATTGAATTTCC | CGATGATCAATTCTCAGTTGAAAGGC |
| | AGAAGGCAAGAAAATCCGCCAACCT | CTGATGGATCAAATTTCACTCTGAAG |
| | TGTCATCTCCGCTTCACAACTGGGGG | ATCCGGTCCACAAAGCTGGAGGACTC |
| | ACTCAGCAATGTATTTCTGTGCAATG | AGCCATGTACTTCTGTGCCAGCCAAA |
| | AGAGAGCCCGATAGCAACTATCAGTT | AGGGACTAGAGTACGAGCAGTACTTC |
| | AATCTGGGGCGCTGGGACCAAGCTA | GGGCCGGGCACCAGGCTCACGGTCA |
| | ATTATAAAGCCA | CAG |
| | (SEQ ID NO: 206) | (SEQ ID NO: 207) |

The exemplary TCR_A0001 nucleotide sequences set forth in Kamga et al. 2019, are detailed as follows: TCRα variable region:

(SEQ ID NO: 307)
GCCCAGTCTGTGAGCCAGCATAACCACCACGTAATTCTCTCTGAAGCAG
CCTCACTGGAGTTGGGATGCAACTATTCCTATGGTGGAACTGTTAATCT
CTTCTGGTATGTCCAGTACCCTGGTCAACACCTTCAGCTTCTCCTCAAG
TACTTTTCAGGGGATCCACTGGTTAAAGGCATCAAGGGCTTTGAGGCTG
AATTTATAAAGAGTAAATTCTCCTTTAATCTGAGGAAACCCTCTGTGCA
GTGGAGTGACACAGCTGAGTACTTCTGTGCCGTGAAAGACACCGACAAG
CTCATCTTTGGGACTGGGACCAGATTACAAGTCTTTCCAA;
and
TCRβ variable region:
(SEQ ID NO: 308)
GATGCTGATGTTACCCAGACCCCAAGGAATAGGATCACAAAGACAGGAA
AGAGGATTATGCTGGAATGTTCTCAGACTAAGGGTCATGATAGAATGTA
CTGGTATCGACAAGACCCAGGACTGGGCCTACGGTTGATCTATTACTCC
TTTGATGTCAAAGATATAAACAAAGGAGAGATCTCTGATGGATACAGTG
TCTCTCGACAGGCACAGGCTAAATTCTCCCTGTCCCTAGAGTCTGCCAT
CCCCAACCAGACAGCTCTTTACTTCTGTGCCACCAGTGATTGGGACGAC
AGCACCGGGGAGCTGTTTTTTGGAGAAGGCTCTAGGCTGACCGTACTG
G. The CDR3 regions are underlined.

In some embodiments, the TCRα chain variable domain codon-optimized nucleotide sequence is selected from the group consisting of SEQ ID NO: 108, SEQ ID NO: 109, SEQ ID NO: 110, SEQ ID NO:111, SEQ ID NO:112, SEQ ID NO:113, SEQ ID NO:114, SEQ ID NO:115, SEQ ID NO:116, SEQ ID NO: 117, SEQ ID NO: 118, SEQ ID NO: 119, SEQ ID NO: 120, SEQ ID NO: 134, SEQ ID NO:175, SEQ ID NO: 176, SEQ ID NO: 177, SEQ ID NO: 188, SEQ ID NO:213, SEQ ID NO:215, SEQ ID NO:217, SEQ ID NO:219 and SEQ ID NO:221, or a codon degenerate nucleotide sequence thereof encoding the amino acid sequence encoded by the reference sequence. In some embodiments, the TCRα chain variable domain codon-optimized nucleotide sequence shares at least about 80%, about 85%, about 90%, or about 95% sequence identity with a member selected from: SEQ ID NO: 108, SEQ ID NO: 109, SEQ ID NO:110, SEQ ID NO:111, SEQ ID NO:112, SEQ ID NO:113, SEQ ID NO: 114, SEQ ID NO: 115, SEQ ID NO:116, SEQ ID NO:117 SEQ ID NO:118, SEQ ID NO:119, SEQ ID NO: 120, SEQ ID NO: 134, SEQ ID NO: 175, SEQ ID NO:176, SEQ ID NO: 177, SEQ ID NO:188, SEQ ID NO: 213, SEQ ID NO:215, SEQ ID NO:217, SEQ ID NO:219 and SEQ ID NO:221, as listed in Table 6, or a codon degenerate nucleotide sequence thereof encoding the amino acid sequence encoded by the reference sequence.

In some embodiments, the TCR β chain variable domain codon-optimized nucleotide sequence selected from the group consisting of SEQ ID NO:121, SEQ ID NO: 122, SEQ ID NO: 123, SEQ ID NO:124, SEQ ID NO:125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 135, SEQ ID NO: 178, SEQ ID NO: 179, SEQ ID NO: 180, SEQ ID NO: 189, SEQ ID NO:214, SEQ ID NO:216, SEQ ID NO:218, SEQ ID NO:220 and SEQ ID NO:222, or a codon degenerate nucleotide sequence thereof encoding the amino acid sequence encoded by the reference sequence. In some embodiments, the TCR β chain variable domain codon-optimized nucleotide sequence shares at least about 80%, about 85%, about 90%, or about 95% sequence identity with a member selected from: SEQ ID NO: 121, SEQ ID NO: 122, SEQ ID NO: 123, SEQ ID NO: 124, SEQ ID NO:125, SEQ ID NO:126, SEQ ID NO: 127, SEQ ID NO: 128, SEQ ID NO: 129, SEQ ID NO: 130, SEQ ID NO: 131, SEQ ID NO:132, SEQ ID NO: 133, SEQ ID NO: 135, SEQ ID NO: 178, SEQ ID NO: 179, SEQ ID NO: 180, SEQ ID NO:189, SEQ ID NO: 214, SEQ ID NO:216, SEQ ID NO:218, SEQ ID NO:220 and SEQ ID NO:222, as listed in Table 6, or a codon degenerate nucleotide sequence thereof encoding the amino acid sequence encoded by the reference sequence.

TABLE 6
Codon-optimized nucleotide sequences of the TCRα and TCRβ variable regions. The CDR3
region is underlined.

| TCR ID | TCRα variable region [column A] | TCRβ variable region [column B] |
|---|---|---|
| TCR_A0002 | >COATCR_A0002 | >COBTCR_A0002 |
| | GCGCAGAGCGTTTCGCAACACAACCA | GACACCGCCGTTTCCCAGACACCGA |
| | CCACGTCATCCTGTCCGAGGCTGCTT | AGTACCTGGTGACCCAGATGGGCAA |
| | CTCTGGAGCTGGGGTGCAACTACAGC | CGACAAGAGCATCAAGTGCGAGCAG |
| | TACGGTGGCACGGTCAATCTATTTTG | AACCTGGGCCATGACACGATGTATTG |
| | GTACGTGCAGTATCCAGGACAGCATC | GTACAAGCAGGACTCCAAGAAATTT |
| | TCCAGCTGCTGCTCAAGTACTTTTCAG | CTGAAGATCATGTTTAGCTACAACAA |
| | GGGACCCGTTGGTGAAAGGCATCAAG | CAAGGAGCTCATCATTAACGAGACC |
| | GGCTTCGAAGCAGAGTTCATTAAGTC | GTGCCCAACCGCTTCTCACCAAAGTC |
| | GAAATTTTCCTTCAACCTGCGTAAGC | GCCCGACAAAGCGCACTTGAATCTA |
| | CTTCCGTGCAGTGGTCTGATACTGCC | CACATCAATTCTCTGGAGCTGGGTGA |
| | GAGTACTTCTGTGCCGTGAAGGACAC | TTCTGCCGTGTACTTCTGTGCTTCCTC |
| | CGACAAGCTTATCTTCGGTACCGGCA | GCCGGATTTCAACGAACAGTTCTTCG |
| | CCCGCCTGCAGGTGTTCCCC | GCCCTGGGACTCGTCTGACCGTCCTT |
| | (SEQ ID NO: 108) | (SEQ ID NO: 121) |
| TCR_A0003 | >COATCR_A0003 | >COBTCR_A0003 |
| | GCGCAGAGCGTGTCCCAACACAACCA | GAAGCAGGGGTGGCTCAGAGCCCGC |
| | CCACGTCATCCTGTCCGAGGCTGCTT | GCTACAAGATTATTGAGAAGCGCCA |
| | CCCTGGAGCTGGGGTGCAACTACAGC | GTCCGTGGCGTTCTGGTGCAATCCCA |
| | TACGGCGGCACCGTCAATTTGTTCTG | TCTCTGGCCACGCCACTCTTTATTGG |
| | GTACGTGCAGTATCCGGGACAGCATC | TACCAACAGATCCTGGGACAGGGCC |
| | TCCAGCTGCTGCTCAAGTACTTTAGT | CTAAATTGCTCATCCAGTTCCAGAAC |
| | GGTGATCCACTTGTTAAAGGCATCAA | AACGGTGTGGTCGATGACAGCCAGC |
| | GGGCTTCGAAGCCGAGTTCATTAAGT | TGCCCAAGGACAGGTTTTCAGCCGA |
| | CGAAATTTTCATTCAACCTGCGCAAG | GCGCCTGAAGGGCGTCGACTCGACC |
| | CCCTCTGTGCAGTGGTCTGACACCGC | CTCAAAATCCAGCCAGCCAAGCTGG |
| | AGAGTACTTCTGTGCCGGGGGCGCGG | AGGACAGTGCGGTGTACCTGTGCGC |
| | CCGGCAACAAGCTGACCTTCGGAGGC | CTCCTCCTCTCCTCTGGGCGGCTTCG |
| | GGTACTCGTGTGCTGGTGAAGCCT | CGGGGGCCAACGTGCTGACCTTCGGT |
| | (SEQ ID NO: 109) | GCTGGCTCCCGTCTGACCGTTCTA |
| | | (SEQ ID NO: 122) |
| TCR_A0004 | >COATCR_A0004 | >COBTCR_A0004 |
| | GCGCAGAGCGTGTCCCAACACAACCA | GATGCGGACGTGACCCAGACTCCCC |
| | CCACGTTATCCTGTCCGAGGCTGCAT | GCAACCGCATCACCAAGACCGGCAA |
| | CCCTGGAGCTGGGGTGCAACTACAGC | GCGCATCATGTTGGAGTGCTCTCAAA |
| | TACGGTGGCACCGTCAATCTATTTTG | CAAAGGGCCACGACAGGATGTACTG |
| | GTACGTGCAGTATCCTGGACAGCATT | GTACCGGCAGGACCCGGGGCTGGGC |
| | TGCAGCTGCTGCTCAAGTACTTCAGT | CTCCGTCTTATCTACTACTCCTTCGAC |
| | GGGGACCCGCTGGTGAAAGGCATCA | GTGAAGGACATCAATAAGGGTGAGA |
| | AGGGCTTCGAAGCTGAGTTCATTAAG | TCAGCGATGGCTACTCCGTGTCGCGA |
| | TCGAAATTTTCATTCAACCTGCGCAA | CAGGCTCAGGCCAAATTTTCACTATC |
| | GCCCTCTGTGCAGTGGTCTGATACGG | TCTGGAGTCCGCCATCCCCAACCAGA |
| | CCGAGTACTTTTGTGCCGTGAAGGAC | CGGCACTGTACTTCTGTGCCACCTCC |
| | ACCGACAAGCTTATCTTCGGTACCGG | GACTTCATTAGTGACACCCAGTATTT |
| | CACTCGTCTCCAGGTCTTCCCC | CGGACCTGGTACTCGCCTGACCGTGC |
| | (SEQ ID NO: 110) | TG |
| | | (SEQ ID NO: 123) |
| TCR_A0005 | >COATCR_A0005 | >COBTCR_A0005 |
| | GCACAGAAGATCACCCAGACTCAACC | TCGGCAGTGATTAGCCAGAAGCCCTC |
| | TGGTATGTTTGTGCAGGAGAAGGAGG | TCGGGACATCTGCCAGCGTGGTACA |
| | CTGTCACATTGGACTGTACCTACGAC | AGTTTGACCATCCAGTGCCAAGTTGA |
| | ACCTCCGACCAGAGCTACGGCCTTTT | TTCTCAGGTCACCATGATGTTTTGGT |
| | CTGGTACAAGCAGCCGAGTTCGGGGG | ACCGCCAGCAGCCGGGACAGAGCCT |
| | AGATGATCTTCCTGATCTATCAGGGC | AACTCTTATCGCGACGGCCAACCAG |
| | TCCTACGACGAACAGAACGCGACGG | GGCTCCGAGGCTACCTACGAGAGCG |
| | AGGGCCGCTACTCCCTCAACTTCCAG | GCTTCGTCATTGACAAGTTTCCCATC |
| | AAGGCCCGCAAAAGCGCGAACCTGG | TCCCGCCCTAACCTGACCTTCTCGAC |
| | TGATTTCTGCTTCCCAGCTGGGTGATT | TCTCACCGTGTCCAATATGTCTCCTG |
| | CTGCCATGTATTTCTGCGCCATGCGTG | AAGACAGCTCCATCTATCTGTGCTCC |
| | AGGGCGGCAACTTCAACAAGTTCTAC | GTGGGCGGCACCTCCGGCACCCTGCC |
| | TTCGGATCAGGGACCAAGCTGAATGT | AGCCAACGAGCAGTTCTTCGGTCCCG |
| | GAAGCCC | GGACCCGCCTGACCGTGCTG |
| | (SEQ ID NO: 111) | (SEQ ID NO: 124) |
| TCR_A0015 | >COATCR_A0015 | >COBTCR_A0015 |
| | GCGCAGAGCGTCACCCAGTTGGATTC | GACGCAGGGGTGATCCAGAGCCCGC |
| | TCAGGTCCCAGTGTTCGAGGAGGCTC | GCCATGAAGTCACCGAGATGGGCCA |
| | CGGTGGAGCTTCGTTGTAACTACTCC | GGAGGTGACTCTTAGGTGTAAACCC |
| | TCGTCAGTATCCGTGTACCTCTTTTGG | ATCTCTGGCCACAACTCCCTCTTTTG |
| | TACGTGCAGTATCCCAACCAGGGTCT | GTACCGCCAGACTATGATGCGTGGTC |
| | GCAGCTGCTGCTCAAGTACCTGTCTG | TGGAGCTGCTGATTTACTTCAACAAC |
| | GCTCCACTCTGGTGAAGGGCATTAAT | AATGTGCCCATCGATGACTCTGGTAT |
| | GGCTTCGAAGCAGAGTTCAACAAGTC | GCCTGAGGACCGCTTTTCAGCCAAGA |
| | GCAGACCTCCTTCCATCTGCGCAAGC | TGCCCAACGCGTCCTTCTCGACCCTG |
| | CCTCTGTCCACATCTCCGACACCGCC | AAGATCCAGCCGTCCGAGCCACGGG |
| | GAGTACTTTTGCGCCGTGTCCGCCCT | ACAGCGCCGTGTACTTCTGCGCTTCC |
| | GAGCTACAACCAAGGGGGTAAGCTA | AGTTGGACCGGCAACGAGCAGTATT |
| | ATCTTCGGACAGGGCACCGAGCTGAG | TCGGACCTGGCACCCGCTTGACGGTT |
| | TGTTAAACCT | ACA |
| | (SEQ ID NO: 112) | (SEQ ID NO: 125) |
| TCR_A0061 | >COATCR_A0061 | >COBTCR_A0061 |
| | AAGCAGGAGGTGACACAAATTCCCGC | GATGCAGGCATCACCCAGAGCCCGC |
| | CGCCCTGTCCGTCCCCGAGGGCGAGA | GCTACAAGATTACAGAGACCGGCCG |
| | ATCTGGTGCTCAACTGCTCTTTTACCG | CCAGGTCACCCTGATGTGCCACCAGA |
| | ACAGTGCCATCTACAACCTGCAGTGG | CCTGGTCTCATAGTTACATGTTTTGG |
| | TTCCGCCAGGACCCGGGCAAGGGTCT | TACAGGCAGGACCTGGGCCACGGTC |
| | GACCTCCCTGCTGCTCATCCAGAGCT | TCCGTCTTATCTACTACTCCGCTGCT |
| | CACAGCGGGAACAGACTTCCGGCCGC | GCCGACATCACCGACAAAGGGGAGG |
| | CTGAACGCGTCTTTGGACAAAAGCTC | TGCCCGACGGCTACGTGGTGTCGCGG |
| | CGGGCGCTCGACCCTGTACATCGCCG | TCCAAGACTGAGAACTTCCCTTTGAC |
| | CTTCCCAGCCAGGTGATTCTGCTACCT | TCTGGAGAGCGCCACTCGCTCGCAA |
| | ACCTGTGCGCCGTGCTGATGGACAGC | ACGTCCGTTTATTTCTGCGCCTCCTCT |
| | AACTATCAGCTTATTTGGGGCGCCGG | AGCGATGGTATGAACACGGAAGCGT |
| | CACCAAGCTGATCATCAAGCCT | TCTTCGGACAGGGCACCCGCCTGACC |
| | (SEQ ID NO: 113) | GTGGTC |
| | | (SEQ ID NO: 126) |
| TCR_A0062 | >COATCR_A0062 | >COBTCR_A0062 |
| | TCACAACAGGGCGAGGAAGATCCTCA | AATGCAGGTGTCACCCAGACTCCGA |
| | GGCCCTCAGCATCCAGGAGGGGGAG | AATTTCAGGTCCTGAAGACCGGTCAG |
| | AATGCAACAATGAACTGCTCTTACAA | AGCATGACTTTGCAGTGCGCCCAGG |
| | GACCAGCATTAACAACCTGCAGTGGT | ACATGAACCATGAGTACATGAGTTG |
| | ACCGCCAGAACTCCGGTCGTGGTTTG | GTACCGCCAGGATCCAGGAATGGGC |
| | GTGCATTTGATCCTGATCCGCAGCAA | CTTCGGCTCATTCACTACTCCGTGGG |
| | CGAGAGGGAGAAGCACAGTGGACGC | GGCCGGCATCACCGACCAGGGGGAG |
| | CTGCGGGTCACCCTGGACACCTCCAA | GTGCCTAACGGCTACAACGTGTCCCG |
| | GAAGTCGTCCTCTCTGCTCATCACCG | CTCGACCACAGAGGACTTCCCCCTGC |
| | CTTCCCGCGCCGCGGACACTGCTAGC | GTCTGCTGTCCGCCGCCCCCTCTCAA |
| | TATTTTTGTGCCACCGAGGGCTCCTCT | ACGAGCGTGTACTTCTGTGCTTCTTC |
| | GGCTACTCCACTCTTACCTTCGGCAA | CAAGCAGGGCGGTGGATACGGCTAT |
| | AGGCACCATGCTGCTGGTGTCGCCC | ACCTTCGGCTCCGGCACCCGCCTGAC |
| | (SEQ ID NO: 114) | CGTGGTT |
| | | (SEQ ID NO: 127) |
| TCR_A0064 | >COATCR_A0064 | >COBTCR_A0064 |
| | GGACAGCAGGTCATGCAAATTCCTCA | AAAGCCGGCGTGACCCAGACTCCGC |
| | GTACCAGCATGTCCAGGAGGGCGAG | GCTACCTAATTAAGACTCGTGGTCAA |
| | GACTTCACCACTTACTGCAATAGCTC | CAGGTCACCCTGAGCTGTTCGCCCAT |
| | GACCACTTTGAGCAACATCCAGTGGT | CTCTGGCCACCGGTCCGTTAGTTGGT |
| | ACAAGCAGCGTCCGGGCGGCCACCCC | ACCAGCAGACGCCAGGACAGGGCCT |
| | GTGTTCCTGATCCAGCTGGTGAAGTC | CCAGTTCCTGTTCGAGTACTTCTCCG |
| | TGGGGAAGTTAAGAAACAGAAGCGC | AGACCCAGCGCAACAAGGGCAACTT |
| | CTGACCTTCCAGTTTGGAGAGGCCAA | TCCTGGGCGATTCTCTGGTCGCCAGT |
| | GAAGAACTCCTCTCTTCACATCACCG | TTTCAAATTCCAGGTCGGAGATGAAC |
| | CCACCCAGACAACCGATGTGGGGACC | GTGTCCACCCTTGAGCTGGGGGACA |
| | TACTTTTGTGCTGGTGCGGGTGCAGG | GCGCGCTGTACCTGTGCGCTTCCTCT |
| | CTCCTATCAGCTCACCTTCGGCAAAG | TTGGAGGGCCAGGCCAGCAGCTACG |
| | GCACCAAGCTGTCCGTGATCCCA | AACAGTATTTCGGACCCGGCACCCGC |
| | (SEQ ID NO: 115) | CTGACCGTGACA |
| | | (SEQ ID NO: 128) |
| TCR_A0065 | >COATCR_A0065 | >COBTCR_A0065 |
| | GGACAGCAGGTCATGCAAATTCCTCA | AAAGCCGGCGTGACCCAGACGCCAC |
| | GTATCAGCATGTGCAGGAGGGCGAA | GATACCTAATTAAGACCCGTGGTCAA |
| | GACTTCACCACTTACTGTAATTCCTCG | CAGGTCACCCTTTCATGCTCTCCCAT |
| | ACCACCTTGAGCAACATCCAGTGGTA | CTCGGGCCACCGGTCCGTGAGTTGGT |
| | CAAGCAGCGTCCCGGCGGCCACCCCG | ACCAGCAGACTCCGGGACAGGGCCT |
| | TGTTCCTGATCCAGCTGGTGAAATCT | CCAGTTCCTGTTCGAGTACTTCTCCG |
| | GGCGAGGTGAAGAAACAGAAGCGCC | AGACCCAGCGCAACAAGGGCAACTT |
| | TGACCTTCCAGTTTGGGGAGGCCAAG | TCCCGGGCGCTTCTCTGGACGCCAGT |
| | AAGAACTCCAGCCTTCACATCACCGC | TTTCCAATTCCAGGTCGGAGATGAAC |
| | CACCCAGACTACAGATGTGGGGACCT | GTGTCCACTCTGGAGCTGGGGGACA |
| | ACTTTTGCGCTGTTTCTGGTGCGGGCT | GCGCGCTGTACCTGTGCGCCTCCTCT |
| | CCTACCAGCTCACCTTCGGCAAGGGC | TTGGAGGGCCAGGCCAGCAGCTACG |
| | ACCAAGCTGAGTGTCATCCCG | AACAGTATTTCGGTCCTGGCACCCGC |
| | (SEQ ID NO: 116) | CTGACCGTTACA |
| | | (SEQ ID NO: 129) |
| TCR_A0066 | >COATCR_A0066 | >COBTCR_A0066 |
| | GGACAGCAGGTCATGCAAATTCCTCA | AAAGCCGGTGTTACCCAGACTCCGC |
| | GTACCAGCATGTGCAGGAGGGCGAA | GCTACCTCATTAAGACCAGAGGACA |
| | GACTTCACCACTTACTGTAATTCCTCG | ACAGGTCACTCTTTCATGCTCTCCCA |
| | ACCACACTCAGCAACATCCAGTGGTA | TCTCTGGCCACCGGTCCGTGAGTTGG |
| | CAAGCAGCGTCCCGGCGGCCACCCCG | TACCAGCAGACGCCAGGACAGGGCT |
| | TGTTCCTGATCCAGCTGGTGAAGTCT | TGCAGTTCCTGTTCGAGTACTTCTCC |
| | GGGGAGGTTAAGAAACAGAAGCGCC | GAGACCCAGCGCAACAAGGGCAACT |
| | TGACCTTTCAGTTCGGAGAGGCCAAG | TTCCCGGGCGTTTCTCTGGTCGCCAG |
| | AAGAACTCCTCTTTGCACATCACCGC | TTTTCAAATTCCAGGTCGGAGATGAA |
| | CACCCAGACGACTGATGTGGGGACCT | CGTGTCGACCCTGGAGCTAGGGGAC |
| | ACTTTTGCGCTGGTGCAGGTGCGGGC | AGCGCGCTGTACCTGTGCGCCTCCAG |
| | TCCTATCAGCTTACCTTCGGCAAAGG | CGCAGAGGGCCAGGCCTCCAGCTAC |
| | CACCAAGCTGAGCGTCATCCCG | GAACAGTATTTCGGCCCTGGCACCCG |
| | (SEQ ID NO: 117) | CCTGACCGTGACA |
| | | (SEQ ID NO: 130) |
| TCR_A0068 | >COATCR_A0068 | >COBTCR_A0068 |
| | TCACAGCAAGGCGAGGAAGATCCTCA | AACGCGGGTGTCACCCAGACTCCGA |
| | GGCCCTCAGCATCCAGGAGGGGGAG | AGTTTCAGGTCCTGAAGACCGGTCAG |
| | AATGCAACTATGAACTGCAGCTACAA | AGCATGACTTTGCAGTGCGCCCAGG |
| | GACCAGTATTAACAACCTGCAGTGGT | ACATGAATCATGAGTACATGAGTTG |
| | ACCGCCAGAACTCCGGACGTGGTCTA | GTACAGGCAGGATCCAGGAATGGGC |
| | GTGCATTTGATCCTGATCCGCAGCAA | CTCCGTCTTATTCACTACTCCGTTGG |
| | CGAGAGGGAGAAGCACTCGGGTCGC | GGCCGGCATCACCGACCAGGGGGAG |
| | CTGCGGGTCACCCTGGACACCTCCAA | GTGCCTAACGGCTACAACGTGTCCCG |
| | GAAGTCTTCTTCTCTGCTCATCACTGC | CTCGACCACAGAGGACTTCCCCCTGC |
| | TTCCCGCGCCGCGGACACAGCTAGCT | GGCTGCTGAGCGCAGCTCCCTCTCAA |
| | ATTTTTGTGCCACCGAGGGCGGCTCC | ACGTCCGTGTACTTCTGTGCTTCTAG |
| | GGCTACTCCACCCTTACCTTCGGCAA | CCGCCAGGGCGGTTCAGGCTCCGGCT |
| | AGGCACCATGCTGCTGGTGTCGCCC | ATACCTTCGGCTCGGGCACCCGCCTG |
| | (SEQ ID NO: 118) | ACCGTGGTG |
| | | (SEQ ID NO: 131) |
| TCR_A0069 | >COATCR_A0069 | >COBTCR_A0069 |
| | TCACAACAGGGCGAGGAGGACCCTC | AACGCGGGTGTCACCCAGACTCCTA |
| | AGGCCCTCTCTATCCAGGAGGGCGAG | AGTTTCAGGTCTTGAAGACTGGACAG |
| | AATGCAACAATGAACTGCAGCTACAA | AGCATGACACTGCAGTGTGCCCAGG |
| | GACCAGCATTAACAACCTGCAGTGGT | ACATGAACCATGAGTACATGAGTTG |
| | ACCGGCAGAACTCCGGCCGTGGTTTG | GTACCGCCAGGACCCGGGAATGGGC |
| | GTGCATCTAATCCTGATCCGCAGCAA | CTCCGTCTTATTCACTACTCCGTGGG |
| | CGAGAGGGAGAAGCACAGTGGGCGC | TGCTGGCATCACCGACCAGGGGGAG |
| | CTGCGCGTCACCCTGGACACCTCCAA | GTGCCAAATGGCTACAACGTGTCCCG |
| | GAAGTCGTCCTCTCTGCTCATCACCG | CTCAACGACCGAGGATTTCCCCCTGC |
| | CTTCCCGCGCCGCGGACACTGCTAGC | GGCTGCTGTCTGCAGCTCCCTCTCAA |
| | TATTTTTGTGCCACTGAAGGTGATTCT | ACTAGCGTGTACTTCTGCGCCTCGAC |
| | GGCTACTCCACCCTTACCTTCGGCAA | CACCCAGGGCGGGGCCTACGGCTAT |
| | AGGCACCATGCTGCTGGTGTCGCCC | ACCTTCGGCTCCGGCACCCGCCTGAC |
| | (SEQ ID NO: 119) | CGTGGTT |
| | | (SEQ ID NO: 132) |
| TCR_A0070 | >COATCR_A0070 | >COBTCR_A0070 |
| | TCACAACAGGGAGAGGAGGACCCTC | AACGCAGGTGTCACCCAGACTCCGA |
| | AGGCCCTCAGCATCCAGGAGGGCGA | AGTTTCAGGTCTTGAAGACCGGCCAG |
| | GAATGCCACTATGAACTGCTCTTACA | AGCATGACGCTGCAGTGCGCCCAGG |
| | AGACCAGCATTAACAACCTGCAGTGG | ACATGAATCATGAGTACATGAGTTG |
| | TACCGCCAGAACAGTGGGCGTGGTTT | GTACCGCCAGGATCCAGGTATGGGC |
| | GGTGCATCTCATCCTGATCCGCAGCA | CTTCGTCTCATTCACTACTCCGTGGG |
| | ACGAGCGCGAAAAGCACTCGGGTCG | GGCCGGCATCACCGACCAGGGGGAG |
| | CCTGCGGGTCACCTTGGATACCTCCA | GTGCCTAACGGCTACAACGTGTCCCG |
| | AGAAGTCCTCTTCTCTGCTGATCACTG | GTCGACCACAGAAGACTTCCCCCTGC |
| | CTTCCAGGGCGGCGGACACCGCAAGC | GCCTGCTGTCCGCCGCCCCCTCTCAA |
| | TATTTTTGTGCTACAGCCGGCAACTCC | ACGTCTGTTTATTTCTGTGCTTCCACT |
| | GGCTACTCCACCCTGACCTTCGGCAA | CCTCAGGGAGGCAACGAGGCGTTCT |
| | AGGCACCATGCTTCTGGTGTCGCCC | TCGGACAGGGCACCCGCCTGACCGT |
| | (SEQ ID NO: 120) | GGTG |
| | | (SEQ ID NO: 133) |
| TCR_A0099 | ATACTGAACGTGGAACAAAGTCCTCA | GAAGCTGGAGTTGCCCAGTCTCCCAG |
| | GTCACTGCATGTTCAGGAGGGAGACA | ATATAAGATTATAGAGAAAAGGCAG |
| | GCACCAATTTCACCTGCAGCTTCCCTT | AGTGTGGCTTTTTGGTGCAATCCTAT |
| | CCAGCAATTTTTATGCCTTACACTGGT | ATCTGGCCATGCTACCCTTTACTGGT |
| | ACAGATGGGAAACTGCAAAAAGCCC | ACCAGCAGATCCTGGGACAGGGCCC |
| | CGAGGCCTTGTTTGTAATGACTTTAA | AAAGCTTCTGATTCAGTTTCAGAATA |
| | ATGGGGATGAAAAGAAGAAAGGACG | ACGGTGTAGTGGATGATTCACAGTTG |
| | AATAAGTGCCACTCTTAATACCAAGG | CCTAAGGATCGATTTTCTGCAGAGAG |
| | AGGGTTACAGCTATTTGTACATCAAA | GCTCAAAGGAGTAGACTCCACTCTCA |
| | GGATCCCAGCCTGAAGACTCAGCCAC | AGATCCAACCTGCAAAGCTTGAGGA |
| | ATACCTCTGTGCCGTTAATGCTGGTG | CTCGGCCGTGTATCTCTGTGCCAGCA |
| | GTACTAGCTATGGAAAGCTGACATTT | GCTCCGATTGGACAGCGAACAATGA |
| | GGACAAGGGACCATCTTGACTGTCCA | GCAGTTCTTCGGGCCAGGGACACGG |
| | TCCA | CTCACCGTGCTA |
| | (SEQ ID NO: 134) | (SEQ ID NO: 135) |
| TCR_A0130 | GCACAGACGGTCACCCAGAGCCAGCC | GAGACTGGCGTCACCCAGACTCCGC |
| | GGAGATGTCTGTGCAGGAGGCTGAAA | GCCACCTGGTGATGGGAATGACCAA |
| | CCGTGACCTTGTCATGCACTTACGAC | CAAGAAATCTCTTAAATGCGAGCAA |
| | ACCTCCGAGAGCAACTACTACCTGTT | CATCTAGGCCACAACGCCATGTATTG |
| | TTGGTACAAGCAGCCACCCTCTCGTC | GTACAAGCAGAGCGCCAAGAAGCCC |
| | AGATGATCCTGGTGATTCGCCAGGAG | CTGGAGCTGATGTTCGTGTACAACTT |
| | GCCTACAAGCAACAGAACGCGACTG | CAAGGAGCAGACGGAGAACAACTCC |
| | AGAACCGCTTCTCCGTTAATTTCCAG | GTGCCCTCTCGGTTCAGCCCTGAATG |
| | AAGGCCGCCAAATCGTTTTCCCTCAA | CCCAAATTCGAGTCACTTGTTCCTGC |
| | AATCTCCGACAGTCAGCTGGGTGATA | ACTTGCATACACTCCAGCCGGAGGA |
| | CAGCCATGTACTTCTGTGCGTTCATG | CAGCGCGCTGTACCTGTGCGCCTCCT |
| | ATCCCCGACAGCAACTATCAGCTTAT | CACAGGTTGGCACCTCCGGTCGTGGT |
| | TTGGGGCGCTGGCACCAAGCTGATCA | GGGGAGCTCTTTTTCGGCGAGGGCTC |
| | TCAAGCCT | CCGCCTGACCGTGCTG |
| | (SEQ ID NO: 175) | (SEQ ID NO: 178) |
| TCR_A0131 | GCGCAGACGGTTACCCAGAGCCAACC | GAAGCTCAGGTCACCCAGAATCCAC |
| | TGAGATGTCCGTGCAGGAGGCTGAAA | GTTATCTCATCACAGTCACCGGCAAG |
| | CCGTGACCTTGTCATGCACTTACGAC | AAGCTCACGGTTACCTGCTCTCAGAA |
| | ACCTCCGAGAACAACTATTACCTGTT | CATGAACCACGAGTACATGAGTTGG |
| | TTGGTACAAGCAGCCGCCCTCTCGTC | TACAGGCAGGACCCGGGCCTTGGCTT |
| | AGATGATCCTGGTGATCCGCCAGGAG | GCGGCAAATTTACTACTCCATGAACG |
| | GCCTACAAACAGCAGAACGCAACCG | TGGAGGTGACCGACAAAGGTGATGT |
| | AGAATCGGTTTTCGGTCAACTTCCAG | GCCTGAGGGCTACAAGGTGTCCCGC |
| | AAGGCTGCCAAATCCTTCTCCCTCAA | AAGGAGAAGCGCAACTTTCCCCTGA |
| | GATCAGCGATTCTCAGCTGGGCGACA | TCCTGGAGAGCCCTTCCCCCAACCAG |
| | CGGCCATGTATTTCTGTGCGTTCATGC | ACTTCTCTGTACTTCTGTGCCAGCTC |
| | TTATTGACAGTGGCACCTACAAGTAC | GCTAGGACAGGGCACCGAGACCCAG |
| | ATCTTCGGGACAGGTACTCGCCTGAA | TATTTCGGTCCCGGGACTCGCCTGCT |
| | GGTGCTGGCC | GGTGCTG |
| | (SEQ ID NO: 176) | (SEQ ID NO: 179) |
| TCR_A0100 | AAACAGGAGGTGACACAGATTCCTGC | GCTGCTGGAGTCATCCAGTCCCCAAG |
| | AGCTCTGAGTGTCCCAGAAGGAGAAA | ACATCTGATCAAAGAAAAGAGGGAA |
| | ACTTGGTTCTCAACTGCAGTTTCACTG | ACAGCCACTCTGAAATGCTATCCTAT |
| | ATAGCGCTATTTACAACCTCCAGTGG | CCCTAGACACGACACTGTCTACTGGT |
| | TTTAGGCAGGACCCTGGGAAAGGTCT | ACCAGCAGGGTCCAGGTCAGGACCC |
| | CACATCTCTGTTGCTTATTCAGTCAAG | CCAGTTCCTCATTTCGTTTTATGAAA |
| | TCAGAGAGAGCAAACAAGTGGAAGA | AGATGCAGAGCGATAAAGGAAGCAT |
| | CTTAATGCCTCGCTGGATAAATCATC | CCCTGATCGATTCTCAGCTCAACAGT |
| | AGGACGTAGTACTTTATACATTGCAG | TCAGTGACTATCATTCTGAACTGAAC |
| | CTTCTCAGCCTGGTGACTCAGCCACC | ATGAGCTCCTTGGAGCTGGGGGACTC |
| | TACCTCTGTGCTGTGGGAGGCAATAA | AGCCCTGTACTTCTGTGCCAGCAGCT |
| | CAATGACATGCGCTTTGGAGCAGGGA | TAATTAACACTGAAGCTTTCTTTGGA |
| | CCAGACTGACAGTAAAACCA | CAAGGCACCAGACTCACAGTTGTA |
| | (SEQ ID NO: 177) | (SEQ ID NO: 180) |
| TCR_A0132 | GCGCAGACGGTGACCCAGAGCCAGC | AAGGCCGGCGTTACCCAGACGCCTC |
| | CGGAGATGTCCGTGCAGGAGGCTGA | GTTATCTTATTAAGACCCGAGGACAG |
| | GACCGTCACCCTGTCGTGCACTTACG | CAGGTCACACTATCTTGCTCTCCCAT |
| | ACACCTCCGAGAGCAACTACTACCTG | CTCTGGCCACCGCTCCGTGAGTTGGT |
| | TTTTGGTACAAGCAGCCACCCTCTCG | ACCAACAGACTCCGGGTCAGGGCCT |
| | CCAGATGATCCTGGTGATTCGTCAGG | CCAGTTCCTGTTCGAGTACTTCAGCG |
| | AGGCCTACAAACAGCAGAACGCGAC | AAACCCAGCGCAACAAGGGCAACTT |
| | AGAGAACCGCTTCTCGGTTAATTTCC | CCCAGGGCGCTTCAGCGGACGCCAG |
| | AGAAGGCAGCCAAGTCCTTCTCCCTC | TTTTCAAATTCCAGGTCGGAGATGAA |
| | AAAATTAGCGATTCTCAATTGGGTGA | CGTGTCGACCCTGGAGCTGGGTGATA |
| | CACTGCCATGTACTTCTGTGCTTTTAT | GCGCGCTGTACCTGTGCGCCTCCAAA |
| | GGAAGCGGACAGTAACTATCAGCTTA | GGCCGGCGTGGGCCCGACTACAACT |
| | TCTGGGGCGCCGGCACCAAGCTGATC | CCCCTTTGCATTTTGGCAACGGCACC |
| | ATCAAGCCT | CGCCTGACCGTGACT |
| | (SEQ ID NO: 188) | (SEQ ID NO: 189) |
| TCR_A0358 | GCACAGACGGTCACCCAGAGCCAGC | GACGCGGACGTGACCCAGACACCCC |
| CO_1 | CGGAGATGTCCGTGCAGGAGGCCGA | GCAACCGCATCACCAAGACCGGCAA |
| | GACCGTGACTCTTTCATGCACTTACG | GCGTATCATGCTTGAGTGCTCTCAAA |
| | ACACCTCCGAGAACAACTACTACCTC | CTAAGGGCCACGATCGAATGTATTG |
| | TTTTGGTACAAGCAACCTCCCTCTCG | GTACAGGCAGGACCCGGGTCTGGGT |
| | GCAGATGATCCTGGTGATCCGTCAGG | CTCCAGCTGATTTACTACTCCTTCGA |
| | AGGCTTATAAACAGCAGAACGCGAC | CGTGAAGGACATTAATAAGGGAGAG |
| | AGAAAACCGCTTCTCGGTCAATTTCC | ATCTCGGACGGCTATTCCGTGTCCCG |
| | AGAAGGCTGCCAAGTCCTTTTCTTTG | CCAGGCTCAGGCAAAATTTTCATTGA |
| | AAAATTAGTGACAGCCAGCTGGGAG | GCCTGGAGAGCGCCATCCCTAACCA |
| | ATACGGCCATGTATTTCTGTGCGTTC | GACTGCTCTGTACTTCTGTGCCACCA |
| | ATGGGGCCCGACTCCGGCACCTACAA | GCGATTCTGATCGGATCTACGGCTAC |
| | GTACATCTTCGGTACCGGCACCCGCC | ACCTTCGGCTCCGGGACCCGCCTGAC |
| | TGAAGGTGCTGGCC | CGTGGTT |
| | (SEQ ID NO: 213) | (SEQ ID NO: 214) |
| TCR_A0358 | GCTCAAACAGTGACCCAGAGCCAGCC | GATGCCGACGTGACCCAGACCCCTA |
| CO_2 | CGAGATGAGCGTGCAGGAAGCTGAA | GAAATAGAATTACAAAGACCGGCAA |
| | ACCGTCACCCTGTCTTGTACCTACGA | GCGGATCATGCTGGAATGTAGCCAG |
| | CACCAGCGAGAACAACTACTACCTGT | ACCAAAGGCCACGACCGGATGTACT |
| | TTTGGTATAAGCAGCCACCTAGCAGA | GGTACCGGCAGGACCCCGGACTGGG |
| | CAGATGATCCTGGTGATCCGGCAGGA | CCTCCAGCTGATCTACTACTCTTTTG |
| | GGCCTACAAACAGCAGAACGCCACA | ATGTCAAGGACATCAACAAGGGCGA |
| | GAGAATAGATTCTCTGTGAACTTCCA | GATCAGCGACGGCTACTCCGTGTCCA |
| | GAAGGCCGCCAAGTCCTTCAGCCTGA | GACAAGCTCAGGCCAAGTTCAGCCT |
| | AGATCAGCGACAGCCAACTGGGCGA | GTCTCTGGAGAGCGCCATCCCTAACC |
| | CACCGCCATGTACTTCTGCGCCTTTAT | AGACCGCCCTGTACTTCTGCGCCACC |
| | GGGACCTGATTCCGGCACATACAAGT | AGCGACAGCGATAGAATCTACGGCT |
| | ACATCTTCGGCACAGGCACCAGACTG | ATACATTCGGCAGCGGAACAAGACT |
| | AAAGTGCTGGCC | GACCGTGGTG |
| | (SEQ ID NO: 215) | (SEQ ID NO: 216) |
| TCR_A0359 | GCCCAGAAAATCACACAGACCCAGC | GAGCCTGAGGTGACCCAGACCCCTA |
| | CCGGCATGTTCGTGCAGGAGAAGGA | GCCACCAGGTGACCCAAATGGGCCA |
| | AGCCGTGACCCTGGACTGTACCTACG | GGAGGTCATCCTCAGATGTGTGCCCA |
| | ACACCAGCGACCAGAGCTACGGCCTG | TCAGCAACCACCTGTACTTTTACTGG |
| | TTTTGGTACAAACAGCCTAGCAGCGG | TATAGACAGATCCTGGGCCAGAAAG |
| | CGAGATGATCTTCCTGATCTACCAAG | TGGAATTCCTGGTGTCCTTCTACAAC |
| | GATCTTATGATGAGCAGAACGCCACA | AACGAGATTAGCGAGAAGTCCGAGA |
| | GAGGGAAGATACAGCCTGAACTTCCA | TCTTCGACGACCAGTTCAGCGTGGAA |
| | GAAGGCCAGAAAGTCCGCTAATCTGG | CGGCCTGACGGATCTAATTTCACCCT |
| | TGATCAGCGCTTCTCAGCTGGGCGAC | GAAGATCCGGAGCACAAAGCTGGAA |
| | TCCGCCATGTACTTCTGCGCCATGCG | GATAGCGCCATGTACTTCTGCGCCTC |
| | GGAACCTGATAGCAACTACCAACTGA | TCAGAAGGGCCTGGAATACGAGCAG |
| | TCTGGGGCGCCGGCACCAAGCTCATT | TACTTTGGCCCCGGCACCAGACTGAC |
| | ATCAAGCCA | AGTGACA |
| | (SEQ ID NO: 217) | (SEQ ID NO: 218) |
| TCR_A0130 | GCCCAGACCGTCACCCAGTCCCAGCC | GAAACCGGCGTGACCCAGACCCCTA |
| CO_2 | TGAGATGAGCGTGCAGGAGGCCGAG | GACACCTGGTCATGGGCATGACCAA |
| | ACAGTGACCCTGAGCTGTACCTACGA | CAAAAAGTCCCTGAAGTGCGAGCAG |
| | CACATCTGAAAACAACTACTATCTCT | CACCTGGGCCACAACGCCATGTACTG |
| | TCTGGTACAAACAACCTCCCAGCCGG | GTATAAGCAGAGCGCCAAGAAACCA |
| | CAGATGATCCTGGTGATCAGACAAGA | CTGGAACTGATGTTCGTGTACAACTT |
| | AGCCTACAAGCAGCAGAACGCCACA | CAAGGAACAAACAGAGAACAACAGC |
| | GAGAATAGATTCTCCGTGAACTTCCA | GTGCCCAGCCGGTTCAGCCCCGAGTG |
| | GAAAGCCGCTAAGAGCTTTAGCCTGA | TCCTAATAGCTCCCACCTGTTCCTGC |
| | AGATCTCTGATAGCCAGCTGGGCGAC | ACCTCCATACACTGCAGCCTGAGGAC |
| | ACCGCCATGTACTTCTGCGCCTTCAT | AGCGCTCTGTACCTGTGCGCCTCTAG |
| | GCTGATCGACAGCGGCACCTACAAGT | CCAGGTGGGCACAAGCGGCAGAGGC |
| | ACATCTTTGGAACCGGCACAAGACTG | GGAGAGCTGTTTTTCGGCGAGGGATC |
| | AAGGTGCTGGCT | TAGACTGACCGTGCTG (SEQ ID |
| | (SEQ ID NO: 219) | NO: 220) |
| TCR_A0131 | GCCCAGACCGTCACCCAGTCCCAGCC | GAGGCCCAGGTGACCCAAAATCCTA |
| CO_2 | TGAGATGAGCGTGCAGGAGGCCGAG | GATACCTGATCACCGTCACAGGCAA |
| | ACAGTGACCCTGAGCTGTACCTACGA | GAAACTGACCGTGACATGTAGCCAG |
| | CACATCTGAAAACAACTACTATCTCT | AACATGAACCACGAGTACATGAGCT |
| | TCTGGTACAAACAACCTCCCAGCCGG | GGTATAGACAGGACCCCGGCCTGGG |
| | CAGATGATCCTGGTGATCAGACAAGA | ACTGCGGCAGATCTACTACAGCATG |
| | AGCCTACAAGCAGCAGAACGCCACA | AACGTGGAAGTGACCGATAAGGGCG |
| | GAGAATAGATTCTCCGTGAACTTCCA | ACGTGCCAGAGGGCTACAAGGTGTC |
| | GAAAGCCGCTAAGAGCTTTAGCCTGA | CAGAAAGGAAAAGCGGAACTTCCCT |
| | AGATCTCTGATAGCCAGCTGGGCGAC | CTGATCCTGGAATCTCCTAGCCCCAA |
| | ACCGCCATGTACTTCTGCGCCTTCAT | CCAGACCAGCCTCTACTTCTGCGCCT |
| | GCTGATCGACAGCGGCACCTACAAGT | CTAGCCTGGGCCAGGGCACCGAGAC |
| | ACATCTTTGGAACCGGCACAAGACTG | ACAGTACTTTGGCCCTGGAACCAGAC |
| | AAGGTGCTGGCT | TGCTGGTGCTG |
| | (SEQ ID NO: 221) | (SEQ ID NO: 222) |

In some embodiments, any TCR described herein may be expressed as a hybrid TCR construct comprising a human TCRα variable region amino acid sequence and a human TCRβ variable region amino acid sequence, along with a mouse TCR constant region comprising TCRα constant region of SEQ ID NO:190 and TCRβ constant region of SEQ ID NO: 191 as listed in Table 7.

In some exemplary embodiments, TCRs A0100, A0130, A0131 or A0132 are expressed as a hybrid TCR construct, comprising a human TCRα variable region amino acid sequence selected from the group consisting of SEQ ID NO:162, 163, 164 or 66 in combination with a TCRβ variable region amino acid sequence selected from the group consisting of SEQ ID NO: 166, 167, 168 or 91, whereby the constant part of the TCR comprises a mouse constant region comprising TCRα constant region of SEQ ID NO:190 and TCRβ constant region of SEQ ID NO:191 as listed in Table 7.

TABLE 7
Mouse TCR constant region amino acid sequences

| SEQ ID | Sequence name | Sequence |
|---|---|---|
| (SEQ ID | mTRAC | NIQNPEPAVYQLKDPRSQDSTLCLFTDFDSQINVPKTMESGTFI |
| NO: 190) | | TDKCVLDMKAMDSKSNGAIAWSNQTSFTCQDIFKETNATYPS |
| | | SDVPCDATLTEKSFETDMNLNFQNLLVIVLRILLLKVAGENLL |
| | | MTLRLWSS |
| (SEQ ID | mTRBC | EDLRNVTPPKVSLFEPSKAEIANKQKATLVCLARGFFPDHVELS |
| NO: 191) | | WWVNGKEVHSGVCTDPQAYKESNYSYCLSSRLRVSATFWHN |
| | | PRNHFRCQVQFHGLSEEDKWPEGSPKPVTQNISAEAWGRADC |
| | | GITSASYQQGVLSATILYEILLGKATLYAVLVSTLVVMAMVKR |
| | | KNS |

In some embodiments, any TCR described herein may be expressed as a TCR construct comprising a human TCRα variable region amino acid sequence and a human TCRβ variable region amino acid sequence, along with a human TCR constant region comprising TCRα constant region of SEQ ID NO:208 and a TCRβ constant region of SEQ ID NO:209 or 210 as listed in Table 8.

In some embodiments, any TCR described herein may be expressed as a TCR construct comprising a human TCRα variable region amino acid sequence and a human TCRβ variable region amino acid sequence, along with a human TCR constant region comprising a mutated version of the human TCRα constant region of SEQ ID NO: 211 and a mutated version of human TCRβ constant region of SEQ ID NO:212 as listed in Table 8. The mutation comprises the introduction of a Cys in both the alpha and beta chains of the TCR to create a stabilizing disulfide bridge between the two chains. TCR chains were modified by mutagenesis of residue 48 in the Cα region from Thr to Cys and residue 57 of the Cβ region from Ser to Cys. The method has been described previously in Kuball et al, Blood. 2007 Mar. 15; 109(6): 2331-2338, which is hereby incorporated by reference in its entirety. The mutation promotes stable expression and pairing of the transduced TCR in human T cells in which the endogenous TCR is not knocked out.

TABLE 8
Human TCR constant region amino acid sequences

| SEQ ID NO | Sequence name | Sequence |
|---|---|---|
| 208 | Human TRAC | IQNPDPAVYQLRDSKSSDKSVCLFTDFDSQTNVSQSKDSDVYIT |
| | | DKTVLDMRSMDFKSNSAVAWSNKSDFACANAFNNSIIPEDTFF |
| | | PSPESSCDVKLVEKSFETDTNLNFQNLSVIGFRILLLKVAGENLL |
| | | MTLRLWSS |
| 209 | Human TRBC1 | DLNKVFPPEVAVFEPSEAEISHTQKATLVCLATGFFPDHVELSW |
| | | WVNGKEVHSGVSTDPQPLKEQPALNDSRYCLSSRLRVSATFW |
| | | QNPRNHFRCQVQFYGLSENDEWTQDRAKPVTQIVSAEAWGR |
| | | ADCGFTSVSYQQGVLSATILYEILLGKATLYAVLVSALVLMAM |
| | | VKRKDF |
| 210 | Human TRBC2 | DLKNVFPPKVAVFEPSEAEISHTQKATLVCLATGFYPDHVELS |
| | | WWVNGKEVHSGVSTDPQPLKEQPALNDSRYCLSSRLRVSATF |
| | | WQNPRNHFRCQVQFYGLSENDEWTQDRAKPVTQIVSAEAWG |
| | | RADCGFTSESYQQGVLSATILYEILLGKATLYAVLVSALVLMA |
| | | MVKRKDSRG |
| 211 | Human TRAC with | NIQNPDPAVYQLRDSKSSDKSVCLFTDFDSQTNVSQSKDSDVY |
| | T48C mutation | ITDKCVLDMRSMDFKSNSAVAWSNKSDFACANAFNNSIIPEDT |
| | (bold) | FFPSPESSCDVKLVEKSFETDTNLNFQNLSVIGFRILLLKVAGFN |
| | | LLMTLRLWSS* |
| 212 | Human TRBC2 | EDLKNVFPPEVAVFEPSEAEISHTQKATLVCLATGFYPDHVELS |
| | with S57C mutation | WWVNGKEVHSGVCTDPQPLKEQPALNDSRYCLSSRLRVSATF |
| | (bold) | WQNPRNHFRCQVQFYGLSENDEWTQDRAKPVTQIVSAEAWG |
| | | RADCGFTSESYQQGVLSATILYEILLGKATLYAVLVSALVLMA |
| | | MVKRKDSRG |

In some embodiments, the TCR comprises a TCRα chain comprising a TCRα constant region having at least 80%, 85%, 90%, or 95% sequence identity to an amino acid sequence selected from: SEQ ID NO:211, 208 and 190; and a TCRβ chain comprising a TCRβ constant region having at least 80%, 85%, 90%, or 95% sequence identity to an amino acid sequence selected from: SEQ ID NO:212, 210, 209 and 191.

In some embodiments, the TCR comprises a TCRα chain comprising a TCRα constant region having at least 80%, 85%, 90%, or 95% sequence identity to SEQ ID NO:211 and 208; and a TCRβ chain comprising a TCRβ constant region having at least 80%, 85%, 90%, or 95% sequence identity to an amino acid sequence selected from: SEQ ID NO:212, 210 and 209. In some embodiments, the TCR comprises a TCRα chain comprising a TCRα constant region having at least 80%, 85%, 90%, or 95% sequence identity to SEQ ID NO:211; and a TCRβ chain comprising a TCRβ constant region having at least 80%, 85%, 90%, or 95% sequence identity to an amino acid sequence selected from: SEQ ID NO:212. In some embodiments, the TCR comprises a TCRα chain comprising a TCRα constant region having at least 80%, 85%, 90%, or 95% sequence identity to SEQ ID NO: 211; and a TCRβ chain comprising a TCRβ constant region having at least 80%, 85%, 90%, or 95% sequence identity to an amino acid sequence selected from: SEQ ID NO:210. In some embodiments, the TCR comprises a TCRα chain comprising a TCRα constant region having at least 80%, 85%, 90%, or 95% sequence identity to SEQ ID NO:211; and a TCRβ chain comprising a TCRβ constant region having at least 80%, 85%, 90%, or 95% sequence identity to an amino acid sequence selected from: SEQ ID NO:209. In some embodiments, the TCR comprises a TCRα chain comprising a TCRα constant region having at least 80%, 85%, 90%, or 95% sequence identity to SEQ ID NO:208; and a TCRβ chain comprising a TCRβ constant region having at least 80%, 85%, 90%, or 95% sequence identity to an amino acid sequence selected from: SEQ ID NO: 212. In some embodiments, the TCR comprises a TCRα chain comprising a TCRα constant region having at least 80%, 85%, 90%, or 95% sequence identity to SEQ ID NO:208; and a TCRβ chain comprising a TCRβ constant region having at least 80%, 85%, 90%, or 95% sequence identity to an amino acid sequence selected from: SEQ ID NO:210. In some embodiments, the TCR comprises a TCRα chain comprising a TCRα constant region having at least 80%, 85%, 90%, or 95% sequence identity to SEQ ID NO:208; and a TCRβ chain comprising a TCRβ constant region having at least 80%, 85%, 90%, or 95% sequence identity to an amino acid sequence selected from: SEQ ID NO:209.

In some embodiments, the TCR comprises a TCRα chain comprising a TCRα constant region having at least 80%, 85%, 90%, or 95% sequence identity to SEQ ID NO:190; and a TCRβ chain comprising a TCRβ constant region having at least 80%, 85%, 90%, or 95% sequence identity to an amino acid sequence selected from: SEQ ID NO: 191.

In an exemplary embodiment, there is provided human TCRs binding to EBV-derived antigenic peptides, as listed in Table 2. In an exemplary embodiment, the human TCRs bind to EBV-derived antigenic peptides presented on HLA-A*2:01. In other embodiments, the human TCRs bind to EBV-derived antigenic peptides from a peptide pool presented on PBMCs expressing 01:01, 02:01, 03:01 or 11:01 and HLA-B alleles 07:02, 08:01 or 35:01 and HLA-C alleles 04:01 and 07:01.

In various embodiments, the human T cell receptors bind to EBV-derived peptides comprising the amino acid sequence YVLDHLIVV (SEQ ID NO:105) derived from BRLF1, or amino acid sequences CLGGLLTMV (SEQ ID NO:106), FLYALALLL (SEQ ID NO:107), or MGSLEMVPM (SEQ ID NO:146) derived from LMP2, or EPLPQGQLTAY (SEQ ID NO:145) derived from BZLF1. In various embodiments, said antigenic peptides are presented on HLA-A*2:01 or HLA-B*35:01.

In other embodiments, the human T cell receptor binds to a splice variant-derived peptide, comprising the amino acid sequence RLPGVLPRA (SEQ ID NO:147) derived from mutant splice factor-induced peptide of MAPK8IP2. In some embodiments, said antigenic peptide is presented on HLA-A*2:01.

In other embodiments, the human T cell receptor binds to HERV-K-derived peptide FLQFKTWWI (SEQ ID NO: 148) derived from HERV-K gag protein. In some embodiments, said antigenic peptide is presented on HLA-A*2:01.

In various embodiments, the invention provides a T cell receptor (TCR) binding to a peptide comprising amino acid sequence RLPGVLPRA (SEQ ID NO:147) presented on HLA-A*02, comprising a TCR alpha chain variable domain comprising a complementarity determining region (CDR)3 selected from sequences SEQ ID NOs: 151 and 152.

In various embodiments, the invention provides a TCR binding to a peptide comprising amino acid sequence RLPGVLPRA (SEQ ID NO:147) presented on HLA-A*02, comprising a TCR beta chain variable domain comprising a CDR3 selected from sequences SEQ ID NOs: 159 and 160.

In various embodiments, the invention provides a TCR comprising a variable domain comprising a member selected from the TCR alpha chain and TCR beta chain CDR3 pairs of SEQ ID NO:162 and 166, and SEQ ID NO: 163 and 167.

The invention provides in various embodiments, a TCR binding to a peptide comprising amino acid sequence RLPGVLPRA (SEQ ID NO:147) presented on HLA-A*02, comprising a TCR alpha chain variable domain comprising a CDR3 of a sequence selected from SEQ ID NO:14, 151, and 152 in combination with a TCR beta chain variable domain comprising a CDR3 selected from SEQ ID NO:42, 159, and 160.

The invention provides in various embodiments, TCR binding to a peptide comprising amino acid sequence RLPGVLPRA (SEQ ID NO:147) presented on HLA-A*02, comprising a TCR alpha chain with the variable region amino acid sequence selected from SEQ ID NO:66, 162, and 163 as set forth in Table 4, in combination with a TCR beta chain with the variable region amino acid sequence selected from SEQ ID NO:92, 166 and 167.

In various embodiments, there is provided a TCR binding to a peptide comprising amino acid sequence RLPGVLPRA (SEQ ID NO:147) presented on HLA-A*02, comprising a TCRα chain with the variable region nucleotide sequence selected from SEQ ID NOs: 169, 170, 175, 176, 186, and 188 in combination with a TCRβ chain with the variable region amino acid sequence selected from SEQ ID NOs: 172, 173, 178, 179, 187 and 189.

The invention provides in various embodiments, a TCR binding to an HERV-K-derived peptide comprising amino acid sequence FLQFKTWWI (SEQ ID NO:148) presented on HLA-A*02, comprising a TCR alpha chain variable domain comprising a CDR3 of sequence SEQ ID NO:153.

The invention provides in various embodiments, a TCR binding to an HERV-K-derived peptide comprising amino acid sequence FLQFKTWWI (SEQ ID NO:148) presented on HLA-A*02, comprising a TCR beta chain variable domain comprising a CDR3 of sequence SEQ ID NO:161.

The invention provides in various embodiments, a TCR comprising a variable domain comprising the TCR alpha chain CDR3 and TCR beta chain CDR3 of SEQ ID NOs: 153 and 161.

Exemplary T cell receptors comprise a TCRα chain variable domain comprising a complementarity determining region (CDR)3 (CDR3) selected from the group consisting of SEQ ID NO:14, SEQ ID NO:15, SEQ ID NO:16, SEQ ID NO:17, SEQ ID NO: 18, SEQ ID NO: 19, SEQ ID NO:20, SEQ ID NO:21, SEQ ID NO:22, SEQ ID NO: 23, SEQ ID NO:24, SEQ ID NO:138, SEQ ID NO: 151, SEQ ID NO:152, and SEQ ID NO: 153, combined with a TCR β chain CDR3 selected from the group consisting of SEQ ID NO:42, SEQ ID NO:43, SEQ ID NO: 44, SEQ ID NO:45, SEQ ID NO:46, SEQ ID NO:47, SEQ ID NO:48, SEQ ID NO:49, SEQ ID NO:50, SEQ ID NO: 51, SEQ ID NO:52, SEQ ID NO:53, SEQ ID NO:54, SEQ ID NO:139, SEQ ID NO: 159, SEQ ID NO: 160, and SEQ ID NO:161, and a combination thereof, as listed in Table 3A.

In some embodiments, the TCR alpha chain variable domain comprises a CDR3 alpha motif selected from the group consisting of SEQ ID NO: 181, and SEQ ID NO:183, combined with a TCR beta chain CDR3 beta motif selected from the group consisting of SEQ ID NO: 182 and SEQ ID NO: 184. In some embodiments, the TCR alpha chain variable domain comprises a CDR3 alpha motif of SEQ ID NO:185, combined with a TCR beta chain CDR3 selected from the group consisting of SEQ ID NO:159, 160 or 42.

In some embodiments, the TCRα chain variable domain is selected from the group of amino acid sequences: SEQ ID NO:55, SEQ ID NO:56, SEQ ID NO:57, SEQ ID NO:58, SEQ ID NO:59, SEQ ID NO:60, SEQ ID NO: 61, SEQ ID NO:62, SEQ ID NO:63, SEQ ID NO:64, SEQ ID NO:65, SEQ ID NO:66, SEQ ID NO: 140, SEQ ID NO:162, SEQ ID NO:163, and SEQ ID NO:164, combined with a TCR β chain variable domain selected from the group consisting of SEQ ID NO:67, SEQ ID NO:68, SEQ ID NO:69, SEQ ID NO:70, SEQ ID NO: 71, SEQ ID NO:72, SEQ ID NO:73, SEQ ID NO:74, SEQ ID NO:75, SEQ ID NO:76, SEQ ID NO:77, SEQ ID NO: 78, SEQ ID NO:91, SEQ ID NO: 141, SEQ ID NO: 166, SEQ ID NO: 167, and SEQ ID NO:168, and a combination thereof, as listed in Table 4.

In some embodiments, the TCRα chain variable domain amino acid sequence is at least 80%, or 85%, or 90%, or 95%, or 99% identical to a sequence selected from SEQ ID NO:55, SEQ ID NO:56, SEQ ID NO:57, SEQ ID NO: 58, SEQ ID NO:59, SEQ ID NO:60, SEQ ID NO:61, SEQ ID NO:62, SEQ ID NO:63, SEQ ID NO:64, SEQ ID NO: 65, SEQ ID NO:66, SEQ ID NO: 140, SEQ ID NO: 162, SEQ ID NO:163, or SEQ ID NO: 164, and a combination thereof, as listed in Table 4.

In some embodiments, the TCRα chain variable domain is combined with a TCR β chain variable domain at least 80%, or 85%, or 90%, or 95%, or 99% identical to an amino acid sequence selected from SEQ ID NO:67, SEQ ID NO:68, SEQ ID NO:69, SEQ ID NO:70, SEQ ID NO:71, SEQ ID NO:72, SEQ ID NO:73, SEQ ID NO: 74, SEQ ID NO: 75, SEQ ID NO:76, SEQ ID NO:77, SEQ ID NO: 141, SEQ ID NO: 166, SEQ ID NO:167, or SEQ ID NO:168, and a combination thereof, as listed in Table 4.

Aspects of the present disclosure relate to multispecific antigen-binding molecules. By ‘multispecific’ it is meant that the antigen-binding molecule displays specific binding to more than one target. A multispecific antigen-binding molecule may be e.g. bispecific, trispecific, etc.

In some embodiments, the TCR of the present disclosure comprises a TCRα chain, a TCRβ, and further comprises an antigen-binding moiety (i.e. in addition to its TCRα and TCRβ chains). In some embodiments, the antigen-binding moiety is specific for an antigen other than the peptide:MHC complex to which the TCR binds. That is, in some embodiments, the target antigen for the antigen-binding moiety is non-identical to the target of the TCR.

In some embodiments, the target antigen for the antigen-binding moiety is an immune cell surface molecule. An immune cell surface molecule is any molecule which is expressed in or at the cell membrane of an immune cell. In some embodiments, the part of the immune cell surface molecule which is bound by the antigen-binding moiety is on the external surface of the immune cell (i.e. is extracellular). In some embodiments, the immune cell may be a cell of hematopoietic origin, e.g. a neutrophil, eosinophil, basophil, dendritic cell, lymphocyte, or monocyte. The lymphocyte may be e.g. a T cell, B cell, natural killer (NK) cell, NKT cell or innate lymphoid cell (ILC), or a precursor thereof (e.g. a thymocyte or pre-B cell). In some embodiments, the immune cell is a T cell, e.g. a CD3+ T cell.

In some embodiments, an immune cell surface molecule may be a CD3-TCR complex polypeptide, e.g. TCRα, TCRβ, TCRγ, TCRδ, TRAC, TRBC1, TRBC2, TRGC1, TRGC2, TRDC, CD3ε, CD3δ, CD3γ, CD3ζ or CD3η. In some embodiments, an immune cell surface molecule is a CD3 polypeptide (e.g. CD3ε, CD3δ, CD3γ, CD3ζ or CD3η), CD8, CD4 or CD28. In some embodiments, an immune cell surface molecule is a checkpoint molecule (e.g. PD-1, CTLA-4, LAG-3, TIM-3, VISTA, TIGIT or BTLA), or a ligand for a checkpoint molecule (e.g. PD-L1, PD-L2, CD80, CD86, MHC class I, MHC Class II, Galectin 9, VSIG3, VSIG8, LRIG1, PSGL1, CD155 or HVEM). In some embodiments the immune cell surface molecule is a costimulatory molecule (e.g. CD28, OX40, 4-1BB, ICOS or CD27), or a ligand for a costimulatory molecule (e.g. CD86, CD80, OX40L 4-1BBL, ICOSL or CD70). In preferred embodiments, an immune cell surface molecule is a CD3 polypeptide.

### D. Antigen-Presenting Cells

Antigen-presenting cells (APCs) are cells that express MHC molecules (e.g. MHC class I and/or MHC class II molecules), and are capable of presenting MHC:peptide complexes. APCs may be professional APCs. Professional APCs are specialised for presenting antigens to T cells; they are efficient at processing and presenting MHC-peptide complexes at the cell surface, and express high levels of costimulatory molecules. Professional APCs include dendritic cells (DCs), macrophages, and B cells. Non-professional APCs are other cells capable of presenting MHC-peptide complexes to T cells, in particular MHC Class I-peptide complexes to CD8+ T cells. In various embodiments, the APCs are cells of the T2 cell line which is transporter associated with antigen processing (TAP) protein deficient and expresses a low amount of HLA-A*02:01. T2 cells can only present exogenous peptides. In some embodiments, the APCs are peripheral blood mononuclear cells (PBMCs) expressing HLA-A alleles 01:01, 02:01, 03:01 or 11:01 and HLA-B alleles 07:02, 08:01 or 35:01 and HLA-C alleles 04:01 and 07:01. In some embodiments, the APCs are cells of the Raji cell line stably expressing HLA-A*02:01.

### E. Effector Cells

Cytotoxic T lymphocytes (cytotoxic T cells, CTLs) are an immune effector cell population that can mediate specific immune responses against cancer. Based on this concept, tumor immunotherapy protocols have been developed using adoptive transfer of in vitro-expanded autologous T cells that can kill cancer cells (Vignali and Kallikourdis, (2017) Cytokine Growth Factor Rev 36:107-116). Effector functions of cytotoxic T lymphocytes include but are not limited to cytokine production and cytolysis of target cells.

In some embodiments, peripheral blood mononuclear cells (PBMCs) from healthy donors were used as a source of primary T cells. CD4+ and CD8+ T cells were positively enriched using CD4 and CD8 microbeads, LS columns and magnets from Miltenyi. Cells were resuspended in AIM V medium with 10% heat-inactivated human AB serum and 10 ng/mL IL-15. For T cell activation, cells were incubated with TransAct™ beads (Miltenyi). On the same or the next day, Lentiviruses encoding for the TCR of interest were added to the activated T cells and incubated for three days. Half of the cells were not transduced with lentivirus and were used as control cells. Transduction efficiency was verified by flow cytometry, using an anti-mouse TCR antibody to verify that at least 50% of T cells express the transduced TCR.

### F. Cytokines

In some embodiments, the functionality of TCRs is assessed by quantification of cytokine secretion in the cell culture media.

It is well known that cytokines and their signaling pathways exert potent effects on T cell activation, differentiation, and function. Interferon gamma (IFNγ) is crucial for Th1 differentiation and induction of IFN-γ release. In other subsets, IFN-γ inhibits the differentiation of Th2 and Th17 cells but has been shown to promote tReg and antigen-specific memory T cell generation (Bishop et al. Front Immunol. 2021 Apr. 13). IFNγ is a key moderator of cell-mediated immunity with diverse, mainly pro-inflammatory actions on immunocytes and target tissue. Recent studies have shown it may enhance anti-tumor and antiviral effects of CD8 T cells. IFNγ is released in large amounts by macrophages, activated CD8 T cells, natural killer T cells, and Th1 CD4 T cells (Bhat et al. Cell Death Dis. 2017 Jun. 1; 8(6):e2836).

### G. HLA/MHC

MHC class I molecules are non-covalent heterodimers of an alpha (α) chain and a beta (β) 2-microglobulin (B2M). The α-chain has three domains designated α1, α2 and α3. The α1 and α2 domains together form the groove to which the peptide presented by the MHC class I molecule binds, to form the peptide:MHC complex.

MHC class I α-chains are polymorphic, and different α-chains are capable of binding and presenting different peptides. Genes encoding MHC class I α polypeptides are highly variable, with the result that cells from different subjects often express different MHC class I molecules.

In an exemplary embodiment, the invention provides an engineered T cell expressing a TCR of the invention in which the TCR is specifically bound to a cell expressing an EBV antigen, which is presented by a MHC molecule.

Unlike antibodies, which are closely related proteins that recognize intact protein antigens, TCRs bind, via their CDR loops, to peptides presented by molecules of the major histocompatibility complex (MHC). This TCR-MHC interaction is crucially important in cell mediated immunity, with the specificity in the cellular immune response being attributable to MHC polymorphism, an extensive TCR repertoire, and a variable peptide cargo. The conventional T-cell response is mediated by TCR recognition of short peptide fragments bound to MHC class I or MHC class II molecules. Generally, MHC-I present peptides derives from endogenous protein that are recognized by cytotoxic T-cells, whereas MHC-II present exogenously-derived peptides to T helper cells (Bhati et al., Protein Science, 23:260-272 (2014)).

In humans, MHCs are encoded by the human leukocyte antigen (HLA) locus on chromosome 6. There are three major HLA gene loci (HLA-A, HLA-B and HLA-C) and three minor loci (HLA-E, HLA-F and HLA-G). This locus is highly polymorphic, spans over 5 mega bases and covers over 200 genes, with more than 7000 HLA allelic sequences identified to date. Individual subjects normally express 6 different classical MHC-I and 6 MHC-II molecules that can differ from each other by a single amino acid, or by more than 30 amino acids. These polymorphisms mostly affect the MHC binding cleft, and thus dictate the diversity of peptides presented by each MHC molecule (Bhati et al., Protein Science, 23:260-272 (2014)). For this reason, HLA allele frequencies is a subject of intense research, and can vary according to ethnic background, geographical location, as well as individual variations. The frequency distribution of most common HLA alleles is publicly available (http://www.allelefrequencies.net/top10dist.asp).

In some embodiments, the TCR binds to an EBV-derived antigenic peptide presented by an MHC class I molecule comprising an MHC class I α chain polypeptide encoded by a HLA-A*02 allele. For conciseness, hereinbelow a peptide that is presented by an MHC class I molecule comprising an MHC class I α chain polypeptide encoded by a given HLA allele or a HLA allele within a given genus of HLA alleles may be referred to simply as being presented ‘through’ or ‘on’ the relevant allele. For example, a TCR that binds to an EBV-derived antigenic peptide presented by an MHC class I molecule comprising an MHC class I α chain polypeptide encoded by a HLA-A*02 allele may be described as a TCR that binds to an EBV-derived antigenic peptide presented through/on a HLA-A*02 allele.

In some embodiments, the TCR binds to an EBV-derived antigenic peptide presented through the HLA-A*02:01 allele. In some embodiments, the TCR specifically binds to an EBV-derived antigenic peptide presented through another HLA-A*02 allele, including but not restricted to: HLA-A*02:02, HLA-A*02:03, HLA-A*02:04, HLA-A*02:05, HLA-A*02:06, HLA-A*02:07, HLA-A*02:11, HLA-A*02:12, HLA-A*02:19, HLA-A*02:24, HLA-A*02:264, or HLA-A*02:52. In some embodiments, the TCR specifically binds to a BRLF1-derived antigenic peptide presented through a HLA-A*02 allele. In some embodiments, the TCR specifically binds to a BRLF1-derived antigenic peptide presented through the HLA-A*02:01 allele. In some embodiments, the TCR specifically binds to an BRLF1-derived antigenic peptide presented through another HLA-A*02 allele, including but not restricted to: HLA-A*02:02, HLA-A*02:03, HLA-A*02:04, HLA-A*02:05, HLA-A*02:06, HLA-A*02:07, HLA-A*02:11, HLA-A*02:12, HLA-A*02:19, HLA-A*02:24, HLA-A*02:264, or HLA-A*02:52. In some embodiments, the TCR specifically binds to a LMP2-derived antigenic peptide presented through a HLA-A*02 allele. In some embodiments, the TCR specifically binds to an LMP2-derived antigenic peptide presented through the HLA-A*02:01 allele. In some embodiments, the TCR specifically binds to an LMP2-derived antigenic peptide presented through another HLA-A*02 allele, including but not restricted to: HLA-A*02:02, HLA-A*02:03, HLA-A*02:04, HLA-A*02:05, HLA-A*02:06, HLA-A*02:07, HLA-A*02:11, HLA-A*02:12, HLA-A*02:19, HLA-A*02:24, HLA-A*02:264, or HLA-A*02:52.

In some embodiments, the TCR specifically binds to an EBV-derived antigenic peptide presented through another HLA allele, including but not restricted to HLA-B*35:01. In some embodiments, the TCR specifically binds to a BZLF1-derived antigenic peptide presented through a HLA-B*35 allele. In some embodiments, the TCR specifically binds to a BZLF1-derived antigenic peptide presented through HLA-B*35:01.

In some embodiments, the TCR specifically binds to a splice variant of MAPK8IP2-derived peptide presented through a HLA-A*02 allele. In some embodiments, the TCR specifically binds to a splice variant of MAPK8IP2-derived peptide presented on HLA-A*02:01. In some embodiments, the TCR specifically binds to an splice variant of MAPK8IP2-derived antigenic peptide presented through another HLA-A*02 allele, including but not restricted to: HLA-A*02:02, HLA-A*02:03, HLA-A*02:04, HLA-A*02:05, HLA-A*02:06, HLA-A*02:07, HLA-A*02:11, HLA-A*02:12, HLA-A*02:19, HLA-A*02:24, HLA-A*02:264, or HLA-A*02:52.

In some embodiments, the TCR specifically binds to HERV-K gag protein-derived peptide presented through a HLA-A*02 allele. In some embodiments, the TCR specifically binds to HERV-K gag protein-derived peptide presented on HLA-A*02:01. In some embodiments, the TCR specifically binds to an HERV-K gag protein-derived antigenic peptide presented through another HLA-A*02 allele, including but not restricted to: HLA-A*02:02, HLA-A*02:03, HLA-A*02:04, HLA-A*02:05, HLA-A*02:06, HLA-A*02:07, HLA-A*02:11, HLA-A*02:12, HLA-A*02:19, HLA-A*02:24, HLA-A*02:264, or HLA-A*02:52.

### H. EBV

EBV virology is described e.g. in Stanfield and Luftiq, F1000Res. (2017) 6:386 and Odumade et al., Clin Microbiol Rev (2011) 24(1):193-209, both of which are hereby incorporated by reference in their entirety.

EBV infects epithelial cells via binding of viral protein BMFR2 to β1 integrins, and binding of viral protein gH/gL with integrins avβ6 and avβ8. EBV infects B cells through interaction of viral glycoprotein gp350 with CD21 and/or CD35, followed by interaction of viral gp42 with MHC class II. These interactions trigger fusion of the viral envelope with the cell membrane, allowing the virus to enter the cell. Once inside, the viral capsid dissolves and the viral genome is transported to the nucleus.

EBV has two modes of replication; latent and lytic. The latent cycle does not result in production of virions, and can take place in B cells and epithelial cells. The EBV genomic circular DNA resides in the cell nucleus as an episome and is copied by the host cell's DNA polymerase. In latency, only a fraction of EBV's genes are expressed, in one of three different patterns known as latency programs, which produce distinct sets of viral proteins and RNAs. The latent cycle is described e.g. in Amon and Farrell, Reviews in Medical Virology (2004) 15(3): 149-56, which is hereby incorporated by reference in its entirety.

EBNA1 protein and non-coding RNA EBER are expressed in each of latency programs I-III. Latency programs II and III further involve expression of EBNALP, LMP1, LMP2A and LMP2B proteins, and latency program III further involves expression of EBNA2, EBNA3A, EBNA3B and EBNA3C.

EBNA1 is multifunctional, and has roles in gene regulation, extrachromosomal replication, and maintenance of the EBV episomal genome through positive and negative regulation of viral promoters (Duellman et al., J Gen Virol. (2009); 90(Pt 9): 2251-2259). EBNA2 is involved in the regulation of latent viral transcription and contributes to the immortalization of cells infected with EBV (Kempkes and Ling, Curr Top Microbiol Immunol. (2015) 391:35-59). EBNA-LP is required for transformation of native B cells, and recruits transcription factors for viral replication (Szymula et al., PLOS Pathog. (2018); 14(2):e1006890). EBNA3A, 3B and 3C interact with RBPJ to influence gene expression, contributing to survival and growth of infected cells (Wang et al., J Virol. (2016) 90(6):2906-2919). LMP1 regulates expression of genes involved in B cell activation (Chang et al., J. Biomed. Sci. (2003) 10(5): 490-504). LMP2A and LMP2B inhibit normal B cell signal transduction by mimicking the activated B cell receptor (Portis and Longnecker, Oncogene (2004) 23(53): 8619-8628). EBERs form ribonucleoprotein complexes with host cell proteins and are proposed to have roles in cell transformation.

The latent cycle can progress according to any of latency programs I to III in B cells, and usually progresses from III to II to I. Upon infection of a resting naïve B cell, EBV enters latency program III. Expression of latency III genes activates the B cell, which becomes a proliferating blast. EBV then typically progresses to latency II by restricting expression to a subset of genes, which cause differentiation of the blast to a memory B cell. Further restriction of gene expression causes EBV to enter latency I. EBNA1 expression allows EBV to replicate when the memory B cell divides. In epithelial cells, only latency II occurs.

In primary infection, EBV replicates in oropharyngeal epithelial cells and establishes Latency III, II, and I infections in B-lymphocytes. EBV latent infection of B-lymphocytes is necessary for virus persistence, subsequent replication in epithelial cells, and release of infectious virus into saliva. EBV Latency III and II infections of B-lymphocytes, Latency II infection of oral epithelial cells, and Latency II infection of NK- or T cell can result in malignancies, marked by uniform EBV genome presence and gene expression.

Latent EBV in B cells can be reactivated to switch to lytic replication. The lytic cycle results in the production of infectious virions and can take place in place B cells and epithelial cells, and is reviewed e.g. by Kenney in Chapter 25 of Arvin et al., Human Herpesviruses: Biology, Therapy and Immunoprophylaxis; Cambridge University Press (2007), which is hereby incorporated by reference in its entirety.

Lytic replication requires the EBV genome to be linear. The latent EBV genome is episomal, and so it must be linearised for lytic reactivation. In B cells, lytic replication normally only takes place after reactivation from latency.

Immediate-early lytic gene products such as BZLF1 and BRLF1 act as transactivators, enhancing their own expression, and the expression of later lytic cycle genes.

Early lytic gene products have roles in viral replication (e.g. EBV DNA polymerase catalytic component BALF5; DNA polymerase processivity factor BMRF1, DNA binding protein BALF2, helicase BBLF4, primase BSLF1, and primase-associated protein BBLF2/3) and deoxynucleotide metabolism (e.g. thymidine kinase BXLF1, dUTPase BORF2). Other early lytic gene products act as transcription factors (e.g. BMRF1, BRRF1), have roles in RNA stability and processing (e.g. BMLF1), or are involved in immune evasion (e.g. BHRF1, which inhibits apoptosis).

Late lytic gene products are traditionally classed as those expressed after the onset of viral replication. They generally encode structural components of the virion such as nucleocapsid proteins, as well as glycoproteins which mediate EBV binding and fusion (e.g. gp350/220, gp85, gp42, gp25). Other late lytic gene products have roles in immune evasion; BCLFI encodes a viral homologue of IL-10, and BALF1 encodes a protein with homology to the anti-apoptotic protein Bcl2.

The present compositions and methods are of great value, because Epstein-Barr virus (EBV) is a WHO class I carcinogen, and is estimated to cause 1-2% of all tumors in humans. Epithelial cancers such as nasopharyngeal carcinoma (NPC), and the 10% of gastric carcinomas associated to EBV outnumber in incidence the EBV-associated lymphomes, which include Burkitt's lymphoma, Hodgkin's lymphoma, diffuse large B cell lymphoma, natural killer (NK)/T cell lymphoma, and primary effusion lymphoma. B cell lymphomas emerge spontaneously or during immune suppression. Thus, EBV causes various tumors owing to failing immune control, some of which can be restored by adoptive transfer of blocking of inhibitory receptors (Munz, Nature Rev 17:691-700 (2019)).

In contrast, other EBV-associated pathologies seem to result from excessive immune responses, but still fail to clear the virus. Such immunopathologies include symptomatic primary EBV infection or mononucleosis, EBV-associated haemophagocytic lymphohistocytosis, and a growing body of evidence also points at the autoimmune disease multiple sclerosis (MS). The symptoms of these conditions could be related to stimulation of T cell-mediated cytokine production by latently EBV-infected B cells. In MS, adoptive transfer of EBV-specific T cells has shown promising initial results (Munz, Nature Rev 17:691-700 (2019)).

EBV replication occurs in 2 ways: infected B cell proliferation, or lytic virion production. EBV persists in latently infected B cells, that initially express no EBV protein (latency 0). During homeostatic proliferation of infected memory B cells, the EBNA1 viral protein is transiently expressed (latency I), soon followed LMP1 and LMP2 (latency II). The virus then infects B cells in secondary lymphoid tissues, that additionally express EBNA2, EBNA3A-EBNA3C (latency III) (Munz, Nature Rev 17:691-700 (2019)). Each of these latency phases are associated to different diseases: nasopharyngeal carcinomas (NPC; associated to type II latency EBV proteins), gastric cancers (associated to type I latency EBV proteins), Burkitt's lymphoma (associated to type I latency EBV proteins), Hodgkin's disease (associated to type II latency EBV proteins), Non-Hodgkin's lymphoma (associated to latency type II EBV proteins), NK/T cell lymphoma (associated to latency type II EBV proteins), etc. (Khan and Hashim, 2014; Thompson and Kurzrock, 2004; US Patent Application No. 20090305324, incorporated here by reference).

Other EBV proteins are expressed during the lytic replication phase of the virus. The expression of the early lytic EBV protein BZLF1 appears to play a trigger role in this process, often co-expressed with the early transcription factor BRLF1. These two early lytic proteins are believed to play a crucial role in EBV-associated tumor formation (Munz, Nature Rev 17:691-700 (2019)).

### 1. BRLF1

BRLF1 is an early lytic transcription factor expressed during the lytic replication phase of EBV. Thus far, clinical trials directing BRLF1-specific TCR transgenic T cells has been limited, underscoring the need for new constructs and/or strategies (Munz, Cells 9:1400 (2020)).

In some embodiments, a TCR, e.g., A0002, A0003, A0004 and A0005, specific for EBV lytic gene product BRLF1, is isolated, optionally modified and cloned into a vector (e.g., a viral vector, e.g. a lentivirus vector) for expression in T cells.

The inventors have performed the above process using Jurkat luciferase reporter cells as a model for T cells. Jurkat cells transduced with the lentiviral vector and successfully expressing the novel TCRs TCR_A0002, TCR_A0003, TCR_A0004 and TCR_A0005 were further tested in a specificity assay. Antigen presenting cells (APCs) expressing HLA-A*02:01 are incubated with BRLF1-derived antigenic peptide YVLDHLIVV (SEQ ID NO: 105) and mixed with the said Jurkat cells. Jurkat cells specifically activated by peptide YVLDHLIVV (SEQ ID NO: 105) via the TCR produce luciferase. Luciferin, the substrate for luciferase, is then added along with additional reagents enabling a chemical reaction producing light. Expression of luciferase following TCR activation can thus be quantified as relative light units (RLU). An increasing response with increasing amount of peptide added to the cells is expected until reaching saturation in the system.

In some embodiments, the TCR binding to a HLA-A*02:01-restricted EBV BRLF1-derived antigenic peptide YVLDHLIVV (SEQ ID NO:105), comprises a TCRα chain variable domain CDR3 amino acid sequence selected from the group consisting of: SEQ ID NO:15; SEQ ID NO:16; and SEQ ID NO:17, in combination with a TCRβ chain variable domain CDR3 amino acid sequence selected from the group consisting of: SEQ ID NO: 43; SEQ ID NO:44; SEQ ID NO:45; and SEQ ID NO:46. In some embodiments, the TCR binding to a HLA-A*02:01-restricted EBV BRLF1-derived antigenic peptide YVLDHLIVV (SEQ ID NO:105), comprises a TCRα chain variable domain CDR3 amino acid sequence shares at least about 95% sequence identity with the amino acid sequence selected from the group consisting of: SEQ ID NO:15; SEQ ID NO: 16; and SEQ ID NO: 17, in combination with a TCRβ chain variable domain CDR3 amino acid sequence shares at least about 95% sequence identity to the amino acid sequence with the amino acid sequence selected from the group consisting of: SEQ ID NO:43; SEQ ID NO:44; SEQ ID NO:45; and SEQ ID NO:46.

In some embodiments, a TCR binding to a HLA-A*02:01-restricted EBV BRLF1-derived antigenic peptide YVLDHLIVV (SEQ ID NO:105), comprises a TCRα chain CDR3α and TCRβ chain CDR3β pair of amino acid sequences selected from the group consisting of: SEQ ID NO: 15 and SEQ ID NO:43; SEQ ID NO: 16 and SEQ ID NO: 44; SEQ ID NO:15 and SEQ ID NO:45; and; SEQ ID NO:17 and SEQ ID NO:46. In some embodiments, the TCR binding to a HLA-A*02:01-restricted EBV BRLF1-derived antigenic peptide YVLDHLIVV (SEQ ID NO:105), comprises a TCRα chain CDR3α and TCRβ chain CDR3 amino acid sequence pairs which shares at least about 95% sequence identity with an amino acid sequences selected from the group consisting of: SEQ ID NO: 15 and SEQ ID NO:43; SEQ ID NO:16 and SEQ ID NO:44; SEQ ID NO: 15 and SEQ ID NO:45; and; SEQ ID NO:17 and SEQ ID NO:46.

In some embodiments, the TCR binding to a HLA-A*02:01-restricted EBV BRLF1-derived antigenic peptide YVLDHLIVV (SEQ ID NO:105), comprises a TCRα chain amino acid sequence selected from the group consisting of: SEQ ID NO:55; SEQ ID NO:56; and SEQ ID NO:57, in combination with a TCRβ chain amino acid sequence selected from the group consisting of: SEQ ID NO:67; SEQ ID NO:68; SEQ ID NO:69; and SEQ ID NO: 70. In some embodiments, the TCR binding to a HLA-A*02:01-restricted EBV BRLF1-derived antigenic peptide YVLDHLIVV (SEQ ID NO:105), comprises a TCRα chain variable domain amino acid sequence which shares at least about 80%, about 85%, about 90%, or about 95% sequence identity with an amino acid sequence selected from the group consisting of: SEQ ID NO:55; SEQ ID NO:56; and SEQ ID NO: 57, in combination with a TCRβ chain variable domain amino acid sequence which shares at least about 80%, about 85%, about 90%, or about 95% sequence identity with a member selected from: SEQ ID NO:67; SEQ ID NO:68; SEQ ID NO:69; and SEQ ID NO:70. In some embodiments, a TCR binding to a HLA-A*02:01-restricted EBV BRLF1-derived antigenic peptide YVLDHLIVV (SEQ ID NO:105), comprises a TCRα chain and TCRβ chain amino acid sequence pair selected from the group consisting of: SEQ ID NO:55 and SEQ ID NO: 67; SEQ I DNO: 56 and SEQ ID NO: 68; SEQ ID NO:55 and SEQ ID NO:69; and; SEQ ID NO:57 and SEQ ID NO: 70. In some embodiments, the TCR binding to a HLA-A*02:01-restricted EBV BRLF1-derived antigenic peptide YVLDHLIVV (SEQ ID NO:105), comprises a TCRα chain and TCRβ chain amino acid sequence pair sharing at least about 80%, about 85%, about 90%, or about 95% sequence identity with a member selected from: SEQ ID NO:55 and SEQ ID NO:67; SEQ ID NO: 56 and SEQ ID NO:68; SEQ ID NO: 55 and SEQ ID NO:69; and; SEQ ID NO:57 and SEQ ID NO:70.

In some embodiments, the TCR binding to a HLA-A*02:01-restricted EBV BRLF1-derived antigenic peptide YVLDHLIVV (SEQ ID NO:105), comprises a TCRα chain encoded by a nucleotide sequence selected from the group consisting of: SEQ ID NO: 79; SEQ ID NO: 80; SEQ ID NO:81; SEQ ID NO: 108; SEQ ID NO: 109; SEQ ID NO: 110; and SEQ ID NO:111, in combination with a TCRβ chain encoded by a nucleotide sequence selected from the group consisting of: SEQ ID NO:92; SEQ ID NO:93; SEQ ID NO:94; SEQ ID NO:95; SEQ ID NO: 121; SEQ ID NO:122; SEQ ID NO:123; and SEQ ID NO:124. In some embodiments, the TCR binding to a HLA-A*02:01-restricted EBV BRLF1-derived antigenic peptide YVLDHLIVV (SEQ ID NO:105), comprises a TCRα chain encoded by a nucleotide sequence sharing at least about 80%, about 85%, about 90%, or about 95% sequence identity with a member selected from: SEQ ID NO:79; SEQ ID NO:80; SEQ ID NO:81; SEQ ID NO: 108; SEQ ID NO:109; SEQ ID NO:110; and SEQ ID NO:111, in combination with a TCRβ chain encoded by a nucleotide sequence sharing at least about 80%, about 85%, about 90%, or about 95% sequence identity to a member selected from: SEQ ID NO:92; SEQ ID NO:93; SEQ ID NO:94; SEQ ID NO:95; SEQ ID NO:121; SEQ ID NO: 122; SEQ ID NO:123; and SEQ ID NO:124.

In some embodiments, the TCR binding to an HLA-A*02:01-restricted EBV BRLF1-derived antigenic peptide YVLDHLIVV (SEQ ID NO:105), comprises a TCRα chain CDR3α and TCRβ chain CDR3β encoded by nucleotide sequence pairs selected from the group consisting of: the nucleotide sequences underscored in SEQ ID NO: 79 and SEQ ID NO:92; SEQ ID NO:80 and SEQ ID NO:93; SEQ ID NO:79 and SEQ ID NO:94; and; SEQ ID NO:81 and SEQ ID NO:95. In some embodiments, the TCR binding to a HLA-A*02:01-restricted EBV BRLF1-derived antigenic peptide YVLDHLIVV (SEQ ID NO:105), comprises a TCRα chain CDR3α and TCRβ chain CDR3β encoded by a nucleotide sequence pair sharing at least about 95% sequence identity to a member selected from: the nucleotide sequences underscored in SEQ ID NO:79 and SEQ ID NO:92; SEQ ID NO: 80 and SEQ ID NO:93; SEQ ID NO: 79 and SEQ ID NO:94; and; SEQ ID NO:81 and SEQ ID NO:95.

### 2. LMP2

In exemplary embodiments, the TCR specifically binds to an EBV-derived antigen. In exemplary embodiments, the TCR specifically binds to an EBV-derived antigen expressed by a cell (e.g., on a cell surface). In some embodiments, the TCR specifically binds to an EBV-derived antigen expressed by a cell in vivo, e.g., a cell which is part of a subject suffering from a disease related to the expression of the EBV-derived antigen. In various embodiments, a TCR specific for EBV latent gene product LMP2 is isolated, optionally modified and cloned into a vector (e.g., a viral vector, e.g. a lentivirus vector) for expression in T cells.

TCR_A0015 was predicted in silico to bind to an EBV protein. Exemplary methods for designing and/or engineering TCRs are provided in commonly-owned applications: Singapore Patent Application No.: 10202109992T; ‘Systems and Methods for the Identification of Target-Specific T cells and Their Receptor Sequences Using Machine Learning’; Applicant(s): IMMUNOSCAPE PTE. LTD.; Filing Date: 10 Sep. 2021; and Singapore Patent Application No. 10202204588Y; ‘Systems and Methods for Identification of Target-Specific T cells and Their Receptor Sequences Using Machine Learning’; Applicant(s): IMMUNOSCAPE PTE. LTD.; Filing Date: 28 Apr. 2022. The disclosures of these applications are incorporated herein by reference in their entirety for all purposes.

This TCR was isolated, modified and cloned into a lentivirus vector for expression in Jurkat luciferase reporter cells. Jurkat cells transduced with the lentiviral vector and successfully expressing this novel TCR were further tested in a specificity assay. Therefore, APCs expressing HLA-A*02:01 were incubated with a pool of antigenic peptides derived from EBV LMP2 and mixed with said Jurkat cells. In some embodiments, overlapping peptides including peptide MGSLEMVPM (SEQ ID NO:146) from EBV LMP2 were tested. Therefore, APCs expressing HLA-A*02:01 were incubated peptide MGSLEMVPM (SEQ ID NO:146) and mixed with said Jurkat cells. Jurkat cells that are specifically activated by the peptide via the TCR produce luciferase. Luciferin, the substrate for luciferase, is then added along with additional reagents enabling a chemical reaction producing light. Expression of luciferase following TCR activation can thus be quantified as relative light units (RLU). An increasing response with increasing amount of peptide added to the cells is expected until reaching saturation in the system.

TCRs specific for EBV latent gene product LMP2 were isolated, modified and cloned into a lentivirus vector for expression in Jurkat luciferase reporter cells. Jurkat cells transduced with the lentiviral vector and successfully expressing the novel TCRs TCR_A0061, TCR_A0062, TCR_A0064, TCR_A0065, TCR_A0066, TCR_A0068, TCR_A0069 and TCR_A0070 were further tested in a specificity assay. APCs expressing HLA-A*02:01 were incubated with LMP2-derived antigenic peptides CLGGLLTMV (SEQ ID NO:106) or FLYALALLL (SEQ ID NO: 107) and mixed with the Jurkat cells. Jurkat cells specifically activated by peptide CLGGLLTMV (SEQ ID NO: 106) or FLYALALLL (SEQ ID NO:107) via the TCR produce luciferase. Luciferin, the substrate for luciferase, is then added along with additional reagents enabling a chemical reaction producing light. Expression of luciferase following TCR activation can thus be quantified as relative light units (RLU). An increasing response with increasing amount of peptide added to the cells is expected until reaching saturation in the system. In some embodiments, the TCR binding a HLA-A*02:01-restricted EBV LMP2-derived antigenic peptide MGSLEMVPM (SEQ ID NO:146) from a peptide pool, comprises a TCRα chain CDR3α and TCRβ chain CDR3β amino acid sequence pair of SEQ ID NO:18 and SEQ ID NO:47. In some embodiments, the TCR binding a HLA-A*02:01-restricted unidentified EBV LMP2-derived antigenic peptide from a peptide pool, comprises a TCRα chain CDR3α and TCRβ chain CDR3β amino acid sequence pair at least 80%, or at least 85%, or at least 90%, or at least 95% identical to the amino acid sequence pair of SEQ ID NO:18 and SEQ ID NO: 47.

In some embodiments, the TCR binding a HLA-A*02:01-restricted EBV LMP2-derived antigenic peptide MGSLEMVPM (SEQ ID NO:146) from a peptide pool, comprises a TCRα chain and TCRβ chain amino acid sequence pair of SEQ ID NO:58 and SEQ ID NO:71. In some embodiments, the TCR binding a HLA-A*02:01-restricted unidentified EBV LMP2-derived antigenic peptide from a peptide pool, comprises a TCRα chain and TCRβ chain amino acid sequence pair at least 80%, or at least 85%, or at least 90%, or at least 95% identical to the amino acid sequence pair of SEQ ID NO:58 and SEQ ID NO:71.

In some embodiments, the TCR binding a HLA-A*02:01-restricted EBV LMP2-derived antigenic peptide MGSLEMVPM (SEQ ID NO:146) from a peptide pool, comprises a TCRα chain CDR3α and TCRβ chain CDR3β encoded by the nucleotide sequence pair underscored in SEQ ID NO:82 and SEQ ID NO:96; and in SEQ ID NO:112 and SEQ ID NO:125. In some embodiments, the TCR binding a HLA-A*02:01-restricted EBV LMP2-derived antigenic peptide MGSLEMVPM (SEQ ID NO:146) from a peptide pool, comprises the TCRα chain CDR3α and TCRβ chain CDR3β encoded by a nucleotide sequence pair at least 80%, or at least 85%, or at least 90%, or at least 95% identical to the nucleotide sequence pair underscored in SEQ ID NO:82 and SEQ ID NO: 96; and SEQ ID NO:112 and SEQ ID NO:125.

In some embodiments, the TCR binding a HLA-A*02:01-restricted EBV LMP2-derived antigenic peptide MGSLEMVPM (SEQ ID NO:146) from a peptide pool, comprises a TCRα chain and TCRβ chain encoded by the nucleotide sequence pair of SEQ ID NO:82 and SEQ ID NO:96; and of SEQ ID NO:112 and SEQ ID NO: 125. In some embodiments, the TCR binding a HLA-A*02:01-restricted EBV LMP2-derived antigenic peptide MGSLEMVPM (SEQ ID NO:146) from a peptide pool, comprises the TCRα chain and TCRβ chain encoded by a nucleotide sequence pair at least 80%, or at least 85%, or at least 90%, or at least 95% identical to the nucleotide sequence pair of SEQ ID NO:82 and SEQ ID NO:96; and SEQ ID NO:112 and SEQ ID NO:125.

In some embodiments, the TCR binding a HLA-A*02:01-restricted EBV LMP2-derived antigenic peptide CLGGLLTMV (SEQ ID NO:106), comprises a TCRα chain variable domain CDR3 amino acid sequence selected from the group consisting of: SEQ ID NO:19; SEQ ID NO:21, and; SEQ ID NO:22, in combination with a TCRβ chain variable domain CDR3 amino acid sequence selected from the group consisting of: SEQ ID NO: 48; SEQ ID NO:50; and SEQ ID NO:51. In some embodiments, the TCR binding a HLA-A*02:01-restricted EBV LMP2-derived antigenic peptide CLGGLLTMV (SEQ ID NO:106), comprises a TCRα chain variable domain CDR3 amino acid sequence at least 80%, or at least 85%, or at least 90%, or at least 95% identical to an amino acid sequence selected from the group consisting of: SEQ ID NO: 19; SEQ ID NO:21, and; SEQ ID NO:22, in combination with a TCRβ chain variable domain CDR3 amino acid sequence at least 80%, or at least 85%, or at least 90%, or at least 95% identical to an amino acid sequence selected from the group consisting of: SEQ ID NO:48; SEQ ID NO:50; and SEQ ID NO:51.

In some embodiments, the TCR binding a HLA-A*02:01-restricted EBV LMP2-derived antigenic peptide CLGGLLTMV (SEQ ID NO:106), comprises a TCRα chain CDR3α and a TCRβ chain CDR3β amino acid sequence pair selected from the group consisting of: SEQ ID NO: 19 and SEQ ID NO:48; SEQ ID NO:21 and SEQ ID NO:50; SEQ ID NO:22 and SEQ ID NO:50; and; SEQ ID NO:21 and SEQ ID NO:51. In some embodiments, the TCR binding a HLA-A*02:01-restricted EBV LMP2-derived antigenic peptide CLGGLLTMV (SEQ ID NO:106), comprises a TCRα chain CDR3α and a TCRβ chain CDR3β amino acid sequence pair at least 80%, or at least 85%, or at least 90%, or at least 95% identical to an amino acid sequence pair selected from the group consisting of: SEQ ID NO:19 and SEQ ID NO:48; SEQ ID NO:21 and SEQ ID NO: 50; SEQ ID NO:22 and SEQ ID NO:50; and; SEQ ID NO:21 and SEQ ID NO:51.

In some embodiments, the TCR binding a HLA-A*02:01-restricted EBV LMP2-derived antigenic peptide CLGGLLTMV (SEQ ID NO:106), comprises a TCRα chain CDR3α motif of amino acid sequence C-A-X¹-X². G-A-G-S-Y-Q-L-T-F (SEQ ID NO:183), in combination with a TCRβ chain CDR3β amino acid sequence of amino acid sequence C-A-S-S-X³-E-G-Q-A-S-S-Y-E-Q-Y-F (SEQ ID NO:184), wherein:
i) X¹ is a member selected from G, V, and any of the following amino acids with related properties: A, I and L
ii) X² is a member selected from A, S, and any of the following amino acids with related properties: G and T
iii) X³ is a member selected from L, A, and any of the following amino acids with related properties: I, V and G.

In some embodiments, the TCR binding a HLA-A*02:01-restricted EBV LMP2-derived antigenic peptide CLGGLLTMV (SEQ ID NO:106), comprises a TCRα chain amino acid sequence selected from the group consisting of: SEQ ID NO:59; SEQ ID NO:61; and SEQ ID NO:62, combined with a TCRβ chain amino acid sequence selected from the group consisting of: SEQ ID NO:72; SEQ ID NO:74; and SEQ ID NO:75. In some embodiments, the TCR binding a HLA-A*02:01-restricted EBV LMP2-derived antigenic peptide CLGGLLTMV (SEQ ID NO:106), comprises a TCRα chain amino acid sequence at least 80%, or at least 85%, or at least 90%, or at least 95% identical to an amino acid sequence selected from the group consisting of: SEQ ID NO: 59; SEQ ID NO:61; and SEQ ID NO:62, combined with a TCRβ chain amino acid sequence at least 80%, or at least 85%, or at least 90%, or at least 95% identical to an amino acid sequence selected from the group consisting of: SEQ ID NO:72; SEQ ID NO:74; and SEQ ID NO:75.

In some embodiments, the TCR binding a HLA-A*02:01-restricted EBV LMP2-derived antigenic peptide CLGGLLTMV (SEQ ID NO:106), comprises a TCRα chain CDR3α and a TCRβ chain CDR3β encoded by a nucleotide sequence pair selected from the group consisting of: the nucleotide sequences underscored in SEQ ID NO: 83 and SEQ ID NO:97; SEQ ID NO:85 and SEQ ID NO:99; SEQ ID NO:86 and SEQ ID NO:100; and; SEQ ID NO:87 and SEQ ID NO:101. In some embodiments, the TCR binding a HLA-A*02:01-restricted EBV LMP2-derived antigenic peptide CLGGLLTMV (SEQ ID NO:106), comprises a TCRα chain CDR3α and a TCRβ chain CDR3β encoded by a nucleotide sequence pair at least 80%, or at least 85%, or at least 90%, or at least 95% identical to a nucleotide acid sequence pair selected from the group consisting of: the nucleotide sequences underscored in SEQ ID NO:83 and SEQ ID NO:97; SEQ ID NO:85 and SEQ ID NO:99; SEQ ID NO: 86 and SEQ ID NO:100; and; SEQ ID NO:87 and SEQ ID NO: 101.

In some embodiments, the TCR binding a HLA-A*02:01-restricted EBV LMP2-derived antigenic peptide CLGGLLTMV (SEQ ID NO:106), comprises a TCRα chain variable domain encoded by a nucleotide sequence selected from the group consisting of: SEQ ID NO:83; SEQ ID NO:85; SEQ ID NO:86; SEQ ID NO:87; SEQ ID NO: 113; SEQ ID NO:115; SEQ ID NO:116; and SEQ ID NO:117, in combination with a TCRβ chain variable domain encoded by a nucleotide sequence selected from the group consisting of: SEQ ID NO:97; SEQ ID NO: 99; SEQ ID NO: 100; SEQ ID NO: 101; SEQ ID NO: 126; SEQ ID NO: 128; SEQ ID NO: 129; and SEQ ID NO: 130. In some embodiments, the TCR binding a HLA-A*02:01-restricted EBV LMP2-derived antigenic peptide CLGGLLTMV (SEQ ID NO:106), comprises a TCRα chain variable domain encoded by a nucleotide sequence at least 80%, or at least 85%, or at least 90%, or at least 95% identical to nucleotide sequence selected from the group consisting of: SEQ ID NO:83; SEQ ID NO:85; SEQ ID NO:86; SEQ ID NO:87; SEQ ID NO: 113; SEQ ID NO:115; SEQ ID NO:116; and SEQ ID NO:117, in combination with a TCRβ chain variable domain encoded by a nucleotide sequence at least 80%, or at least 85%, or at least 90%, or at least 95% identical to a nucleotide sequence selected from the group consisting of: SEQ ID NO:97; SEQ ID NO:99; SEQ ID NO: 100; SEQ ID NO: 101; SEQ ID NO: 126; SEQ ID NO: 128; SEQ ID NO: 129; and SEQ ID NO:130.

In some embodiments, the TCR binding a HLA-A*02:01-restricted EBV LMP2-derived antigenic peptide FLYALALLL (SEQ ID NO:107), comprises a TCRα chain CDR3α and a TCRβ chain CDR3β amino acid sequence pair selected from the group consisting of: SEQ ID NO:20 and SEQ ID NO:49; SEQ ID NO:23 and SEQ ID NO:52; SEQ ID NO:23 and SEQ ID NO:53; and; SEQ ID NO:24 and SEQ ID NO:54. In some embodiments, the TCR binding a HLA-A*02:01-restricted EBV LMP2-derived antigenic peptide FLYALALLL (SEQ ID NO:107), comprises a TCRα chain CDR3α and a TCRβ chain CDR3β amino acid sequence pair at least 80%, or at least 85%, or at least 90%, or at least 95% identical to an amino acid sequence pair selected from the group consisting of: SEQ ID NO:20 and SEQ ID NO:49; SEQ ID NO:23 and SEQ ID NO:52; SEQ ID NO: 23 and SEQ ID NO:53; and; SEQ ID NO:24 and SEQ ID NO:54.

In some embodiments, the TCR binding a HLA-A*02:01-restricted EBV LMP2-derived antigenic peptide FLYALALLL (SEQ ID NO:107), comprises a TCRα chain CDR3α of amino acid sequence C-A-T-X¹-G-X²-S-G-Y-S-T-L-T-F (SEQ ID NO:181), in combination with a TCRβ chain CDR3β amino acid of amino acid sequence C-A-S-X³-X⁴-Q-G-G-(S)-X⁵-X⁶-G-Y-T-F (SEQ ID NO:182), whereby(S) is optional, and wherein:
i) X¹ is selected from E and A;
ii) X² is selected from D, G, N, S, and any of the following amino acids with related properties: E, A, Q and T;
iii) X³ is selected from S and T, and any of the following amino acids with related properties: N and Q;
iv) X⁴ is selected from K, R and T, and any of the following amino acids with related properties: H, and S;
v) X⁵ is selected from G and A;
vi) X⁶ is selected from Y and S, and any of the following amino acids with related properties: F, W, H and T.

In some embodiments, the TCR binding a HLA-A*02:01-restricted EBV LMP2-derived antigenic peptide FLYALALLL (SEQ ID NO:107), comprises a TCRα chain CDR3α and a TCRβ chain CDR3β encoded by a nucleotide sequence pair selected from the group consisting of: the nucleotide sequences underscored in SEQ ID NO: 84 and SEQ ID NO:98; the nucleotide sequences underscored in SEQ ID NO:88 and SEQ ID NO: 102; the nucleotide sequences underscored in SEQ ID NO:89 and SEQ ID NO:103; and; the nucleotide sequences underscored in SEQ ID NO:90 and SEQ ID NO: 104. In some embodiments, the TCR binding a HLA-A*02:01-restricted EBV LMP2-derived antigenic peptide FLYALALLL (SEQ ID NO:107), comprises a TCRα chain CDR3α and a TCRβ chain CDR3β encoded by a nucleotide sequence pair at least 80%, or at least 85%, or at least 90%, or at least 95% identical to a nucleotide acid sequence pair selected from the group consisting of: the nucleotide sequences underscored in SEQ ID NO:84 and SEQ ID NO:98; the nucleotide sequences underscored in SEQ ID NO:88 and SEQ ID NO:102; the nucleotide sequences underscored in SEQ ID NO:89 and SEQ ID NO: 103; and; the nucleotide sequences underscored in SEQ ID NO:90 and SEQ ID NO:104.

### 3. BZLF1

In exemplary embodiments, the TCR specifically binds to an EBV-derived antigen. In exemplary embodiments, the TCR specifically binds to an EBV-derived antigen expressed by a cell (e.g., on a cell surface). In some embodiments, the TCR specifically binds to an EBV-derived antigen expressed by a cell in vivo, e.g., a cell which is part of a subject suffering from a disease related to the expression of the EBV-derived antigen. In various embodiments, a TCR specific for EBV immediate-early gene BZLF1 is isolated, optionally modified and cloned into a vector (e.g., a viral vector, e.g. a lentivirus vector) for expression in T cells.

Cancers driven by EBV viral oncogenes include Burkitt's lymphoma, Hodgkin's disease, nasopharyngeal carcinoma (NPC), T/NK lymphomas, and others. EBV latent infection also causes lymphoproliferative disease (LPD) (Gottschalk et al., 2009).

TCR_A0099 was predicted in silico to bind to an EBV protein. Exemplary methods for designing and/or engineering TCRs are provided in commonly-owned applications: Singapore Patent Application No.: 10202109992T; ‘Systems and Methods for the Identification of Target-Specific T cells and Their Receptor Sequences Using Machine Learning’; Applicant(s): IMMUNOSCAPE PTE. LTD.; Filing Date: 10 Sep. 2021; and Singapore Patent Application No. 10202204588Y; ‘Systems and Methods for Identification of Target-Specific T cells and Their Receptor Sequences Using Machine Learning’; Applicant(s): IMMUNOSCAPE PTE. LTD.; Filing Date: 28 Apr. 2022. The disclosures of these applications are incorporated herein by reference in their entirety for all purposes.

In some embodiments, TCR_A0099 predicted to be specific for an EBV-derived antigenic peptide is isolated, optionally modified and cloned into a vector (e.g., a viral vector, e.g., a lentivirus vector) for expression in T cells. As set forth herein, the inventors have performed this process on Jurkat luciferase reporter cells as a model for T cell transduction. Jurkat cells were transduced with a lentiviral vector and successfully expressed the novel TCRs TCR_A0099. These cells were further tested in a specificity assay. Therefore, in an exemplary embodiment, PBMCs expressing HLA-A alleles 02:01 and 03:01 and HLA-B alleles 07:02 and 35:01, are incubated with the unidentified EBV-derived antigenic peptide and mixed with said Jurkat cells. In some embodiments, selected EBV peptides, including BZLF1 peptide EPLPQGQLTAY (SEQ ID NO:145), were tested. Therefore, in an exemplary embodiment, PBMCs expressing 01:01 and 11:01 and HLA-B alleles 08:01 and 35:01 and HLA-C alleles 04:01 and 07:01 were incubated with the peptide EPLPQGQLTAY (SEQ ID NO: 145) and mixed with said Jurkat cells. Peptide EPLPQGQLTAY (SEQ ID NO:145) has been described in the literature to be HLA-B 35:01-restricted, which is in line with the alleles expressed by the PBMCs tested in the embodiments described here. Jurkat cells specifically activated by the antigenic peptide via the TCR produce luciferase. Luciferin, the substrate for luciferase, is then added along with additional reagents enabling a chemical reaction producing light. Expression of luciferase following TCR activation can thus be quantified as relative light units (RLU). An increasing response with increasing amount of peptide added to the cells is expected until reaching saturation in the system.

In some embodiments, the TCR binding a HLA-B*35:01-restricted EBV BZLF1-derived antigenic peptide EPLPQGQLTAY (SEQ ID NO:145) from a peptide pool, comprises a TCRα chain CDR3α and TCRβ chain CDR3β amino acid sequence pair of SEQ ID NO:138 and SEQ ID NO:139. In some embodiments, the TCR binding a HLA-B*35:01-restricted EBV BZLF1-derived antigenic peptide EPLPQGQLTAY (SEQ ID NO:145) from a peptide pool, comprises a TCRα chain CDR3α and TCRβ chain CDR3β amino acid sequence pair sharing at least about 95% sequence identity to the amino acid sequence pair SEQ ID NO:138 and SEQ ID NO: 139.

In some embodiments, the TCR binding a HLA-B*35:01-restricted EBV BZLF1-derived antigenic peptide EPLPQGQLTAY (SEQ ID NO:145) from a peptide pool, comprises a TCRα chain CDR3α and TCRβ chain CDR3β encoded by the nucleotide sequence pair selected from the nucleotide sequences underscored in SEQ ID NO: 142 and SEQ ID NO:143; or in SEQ ID NO: 134 and SEQ ID NO:135. In some embodiments, the TCR binding a HLA-B*35:01-restricted EBV BZLF1-derived antigenic peptide EPLPQGQLTAY (SEQ ID NO:145) from a peptide pool, comprises the TCRα chain CDR3α and TCRβ chain CDR3β encoded by a nucleotide sequence pair sharing at least about 95% sequence identity to the nucleotide sequence pair selected from the nucleotide sequences underscored in SEQ ID NO:142 and SEQ ID NO: 143; or in SEQ ID NO:134 and SEQ ID NO: 135.

In some embodiments, the TCR binding a HLA-B*35:01-restricted EBV BZLF1-derived antigenic peptide EPLPQGQLTAY (SEQ ID NO:145) from a peptide pool, comprises a TCRα chain encoded by an amino acid sequence of SEQ ID NO: 140 in combination with a TCRβ chain encoded by an amino acid sequence of SEQ ID NO: 141. In some embodiments, the TCR binding a HLA-B*35:01-restricted EBV BZLF1-derived antigenic peptide EPLPQGQLTAY (SEQ ID NO:145) from a peptide pool, comprises a TCRα chain encoded by an amino acid sequence sharing at least about 80%, about 85%, about 90%, or about 95% sequence identity with SEQ ID NO:140, in combination with a TCRβ chain encoded by an amino acid sequence sharing at least about 80%, about 85%, about 90%, or about 95% sequence identity with SEQ ID NO:141.

In some embodiments, the TCR binding a HLA-B*35:01-restricted EBV BZLF1-derived antigenic peptide EPLPQGQLTAY (SEQ ID NO:145) from a peptide pool, comprises a TCRα chain and TCRβ chain encoded by the nucleotide sequence pair selected from: SEQ ID NO: 142 and SEQ ID NO: 143; or SEQ ID NO:134 and SEQ ID NO: 135. In some embodiments, the TCR binding a HLA-B*35:01-restricted EBV BZLF1-derived antigenic peptide EPLPQGQLTAY (SEQ ID NO:145) from a peptide pool, comprises the TCRα chain and TCRβ chain encoded by a nucleotide sequence pair sharing at least about 95% sequence identity to the nucleotide sequence pair selected from: SEQ ID NO:142 and SEQ ID NO:143; or SEQ ID NO:134 and SEQ ID NO:135.

### I. Mutant Splice-Factor Associated Splice Variant of MAPK8IP

Splicing of pre-mRNA by spliceosomes is a cellular process that removes non-coding introns in transcripts and produces alternative splice forms of proteins. Splicing by spliceosomes produces mature mRNA consisting of only coding exons, which form the templates for protein translation. Splicing Factor 3B subunit 1 (SF3B1) is part of the major spliceosome that comprises five small nuclear ribonucleoprotein particles (snRNPs) (Nguyen et al., 2020). SF3B1 and other splicing factors have been reported to be mutated in several types of cancers including uveal melanoma (Bigot et al., 2021; Nguyen et al., 2020), myelodysplastic syndrome (MDS), non-small cell lung cancer (NSCLC) (Oka et al., 2021) chronic lymphocytic leukemia, pancreatic cancer (Leeksma et al., 2021) acute myeloid leukemia and chronic myelomonocytic leukemia (Cheruiyot et al., 2021). Mutations in SF3B1 (SF3B1mut) can lead to errors in splicing and, for example, premature translation termination. Resulting incomplete or misfolded proteins are rapidly degraded in cells. This degradation occurs via the proteasome, whereby peptides from the degraded proteins are presented on MHC-I molecules, in which form they can be recognized by T cells that express a TCR that is specific for the peptide presented in the context of an MHC-I molecule.

Genetic alterations of other splicing factors such as SUGP1, which interacts with SF3B1 during the cellular splicing mechanism, can also result in similar splicing patterns as seen for SF3B1mut (Alsafadi et al., 2020). Mutated splice-factor-induced peptides are a promising target for TCR-mediated cancer therapy because of the tumor-specific expression of such peptides, the sharedness of such peptides between patients and between cancer indications, and because of the potential increased immunogenicity.

Mutated splice factor-induced peptides, including peptide RLPGVLPRA (SEQ ID NO:147) have been reported in the literature (Bigot et al., 2021). The therapeutic value of TCR-based approaches targeting these peptides, however, is not known. The current invention proposes TCR sequences that can be used for the treatment of diseases associated with mutated forms of SF3B1, or other splicing factors including SUGP1. The current invention proposes TCRs and TCR sequences for binding peptide RLPGVLPRA that is comprised in a splice-factor-induced altered version of protein mitogen-activated protein kinase 8 interacting protein 2 (MAPK8IP2).

In some embodiments, TCRs TCR_A0130, TCR_A0131, TCR_A0132, TCR_A0358 and TCR_A0359 specific for a mutant splice factor-induced peptide of MAPK8IP are isolated, optionally modified and cloned into a vector (e.g., a viral vector, e.g., a lentivirus vector) for expression in T cells. As set forth herein, the inventors have performed this process on Jurkat luciferase reporter cells as a model for T cell transduction. Jurkat cells were transduced with a lentiviral vector and successfully expressed the novel TCRs TCR_A0130, TCR_A0131, TCR_A0132, TCR_A0358 and TCR_A0359. These cells were further tested in a specificity assay. Therefore, in an exemplary embodiment, PBMCs expressing HLA-A alleles 02:01, are incubated with the mutant splice factor-induced antigenic peptide of MAPK8IP and mixed with said Jurkat cells. In some embodiments, mutant splice factor-induced peptide of MAPK8IP peptide RLPGVLPRA (SEQ ID NO:147), were tested. Jurkat cells specifically activated by the antigenic peptide via the TCR produce luciferase. Luciferin, the substrate for luciferase, is then added along with additional reagents enabling a chemical reaction producing light. Expression of luciferase following TCR activation can thus be quantified as relative light units (RLU). An increasing response with increasing amount of peptide added to the cells is expected until reaching saturation in the system.

In some embodiments, the TCR binding a HLA-A*02:01-restricted mutant splice factor-induced splice variant MAPK8IP-derived antigenic peptide RLPGVLPRA (SEQ ID NO:147), comprises a TCRα chain CDR3α and TCRβ chain CDR3β amino acid sequence pair selected from SEQ ID NO:151 and SEQ ID NO:159; or SEQ ID NO: 14 and SEQ ID NO:42; or SEQ ID NO: 152 and SEQ ID NO: 160; or SEQ ID NO:194 and SEQ ID NO: 195. In some embodiments, the TCR binding a HLA-A*02:01-restricted mutant splice factor-induced splice variant MAPK8IP-derived antigenic peptide RLPGVLPRA (SEQ ID NO:147), comprises a TCRα chain CDR3α and TCRβ chain CDR3 amino acid sequence pair sharing at least about 95% sequence identity to the amino acid sequence pair selected from SEQ ID NO: 151 and SEQ ID NO:159; or SEQ ID NO: 14 and SEQ ID NO:42; or SEQ ID NO: 152 and SEQ ID NO:160; or SEQ ID NO: 194 and SEQ ID NO:195.

In some embodiments, the TCR binding a HLA-A*02:01-restricted mutant splice factor-induced splice variant MAPK8IP-derived antigenic peptide RLPGVLPRA (SEQ ID NO:147), comprises a TCRα chain CDR3α and TCRβ chain CDR3β amino acid sequence pair selected from SEQ ID NO:151 and SEQ ID NO:159; or SEQ ID NO: 14 and SEQ ID NO:42; or SEQ ID NO:196 and SEQ ID NO: 199. In some embodiments, the TCR binding a HLA-A*02:01-restricted mutant splice factor-induced splice variant MAPK8IP-derived antigenic peptide RLPGVLPRA (SEQ ID NO:147), comprises a TCRα chain CDR3α and TCRβ chain CDR3β amino acid sequence pair sharing at least about 95% sequence identity to the amino acid sequence pair selected from SEQ ID NO: 151 and SEQ ID NO: 159; or SEQ ID NO:14 and SEQ ID NO:42; or SEQ ID NO: 196 and SEQ ID NO: 199.

In some embodiments, the TCR binding a HLA-A*02:01-restricted mutant splice factor-induced splice variant MAPK8IP-derived antigenic peptide RLPGVLPRA (SEQ ID NO:147), comprises a TCRα chain CDR3α and TCRβ chain CDR3β amino acid sequence pair selected from SEQ ID NO:151 and SEQ ID NO:159; or SEQ ID NO: 152 and SEQ ID NO:160, or SEQ ID NO:14 and SEQ ID NO:42. In some embodiments, the TCR binding a HLA-A*02:01-restricted mutant splice factor-induced splice variant MAPK8IP-derived antigenic peptide RLPGVLPRA (SEQ ID NO:147), comprises a TCRα chain CDR3α and TCRβ chain CDR3β amino acid sequence pair sharing at least about 95% sequence identity to the amino acid sequence pair selected from SEQ ID NO: 151 and SEQ ID NO: 159; or SEQ ID NO: 152 and SEQ ID NO: 160, or SEQ ID NO:14 and SEQ ID NO: 42.

In some embodiments, the TCR binding a HLA-A*02:01-restricted mutant splice factor-induced splice variant MAPK8IP-derived antigenic peptide RLPGVLPRA (SEQ ID NO:147), comprises a TCRα chain CDR3α and TCRβ chain CDR3β amino acid sequence pair selected from SEQ ID NO:151 and SEQ ID NO:159; or SEQ ID NO: 14 and SEQ ID NO:42. In some embodiments, the TCR binding a HLA-A*02:01-restricted mutant splice factor-induced splice variant MAPK8IP-derived antigenic peptide RLPGVLPRA (SEQ ID NO:147), comprises a TCRα chain CDR3α and TCRβ chain CDR3β amino acid sequence pair sharing at least about 95% sequence identity to the amino acid sequence pair selected from SEQ ID NO:151 and SEQ ID NO:159; or SEQ ID NO:14 and SEQ ID NO:42.

In various embodiments, the invention provides a T cell receptor (TCR) binding to a peptide comprising amino acid sequence RLPGVLPRA (SEQ ID NO:147) presented on HLA-A*02, comprising a TCRα chain variable domain comprising a CDR3 of the following sequence: C-A-F-M-X¹-X²-D-S-X³-X⁴-Y-X³-X⁶-I-X⁷ (SEQ ID NO: 304) in combination with a TCRβ chain variable domain comprising a CDR3 with a sequence selected from SEQ ID NOs: 42, 159, 160 and 195, wherein
i) X¹ is L or I or E or G, or any of the following amino acids with related properties V or D
ii) X² is P or I or A, or any of the following amino acids with related properties: V, L or G
iii) X³ is G or N, or any of the following amino acids with related properties: Q, A, C or S
iv) X⁴ is T or no AA at this position, or S as an amino acid with related properties
v) X⁵ is K or Q, or any of the following amino acids with related properties: R, H or N
vi) X⁶ is L or Y, or any of the following amino acids with related properties: I, V, F, W or H
vii) X⁷ is F or W.

In various embodiments, the invention provides a T cell receptor (TCR) binding to a peptide comprising amino acid sequence RLPGVLPRA (SEQ ID NO:147) presented on HLA-A*02, comprising a TCRα chain variable domain comprising a CDR3 of the following sequence: C-A-X¹-X²-X³-X⁴-D-S-N-Y-Q-L-I-W (SEQ ID NO: 306) in combination with a TCRβ chain variable domain comprising a CDR3 with a sequence selected from SEQ-ID NOs: 42, 159 and 199, wherein
i) X¹ is F or M, or any of the following amino acids with related properties Y or W
ii) X² is M or R, or any of the following amino acids with related properties: K or H
iii) X³ is I or E, or any of the following amino acids with related properties: V, L or D
iv) X⁴ is P or A, or G as an amino acid with related properties.

In various embodiments, the invention provides a T cell receptor (TCR) binding to a peptide comprising amino acid sequence RLPGVLPRA (SEQ ID NO:147) presented on HLA-A*02, comprising a TCRα chain variable domain comprising a CDR3 of the following sequence: C-A-F-M-X¹-X²-D-S-X³-X⁴-Y-X³-X⁶-I-X⁷ (SEQ ID NO: 185) in combination with a TCRβ chain variable domain comprising a CDR3 with a sequence selected from SEQ ID NOs: 42, 159 and 160, wherein
i) X¹ is L or I or E, or any of the following amino acids with related properties V or D
ii) X² is P or I or A, or any of the following amino acids with related properties: V, L or G
iii) X³ is G or N, or any of the following amino acids with related properties: Q, A, C or S
iv) X⁴ is T or no AA at this position, or S as an amino acid with related properties
v) X⁵ is K or Q, or any of the following amino acids with related properties: R, H or N
vi) X⁶ is L or Y, or any of the following amino acids with related properties: I, V, F, W or H
vii) X⁷ is F or W.

In various embodiments, the invention provides a T cell receptor (TCR) binding to a peptide comprising amino acid sequence RLPGVLPRA (SEQ ID NO:147) presented on HLA-A*02, comprising a TCRα chain variable domain comprising a CDR3 of the following sequence: C-A-F-M-X¹-X²-D-S-N-Y-Q-L-I-W (SEQ ID NO:305) in combination with a TCRβ chain variable domain comprising a CDR3 with a sequence selected from SEQ ID NOs: 42 and 159, wherein
i) X¹ is I or E, or any of the following amino acids with related properties V or D
ii) X² is P or A, or any of the following amino acids with related properties: V, L or G.

In some embodiments, the TCR binding a HLA-A*02:01-restricted mutant splice factor-induced peptide of MAPK8IP-derived antigenic peptide RLPGVLPRA (SEQ ID NO:147), comprises a TCRα chain CDR3α and TCRβ chain CDR3β encoded by the nucleotide sequence pair underscored in SEQ ID NO:169 and SEQ ID NO: 172; or SEQ ID NO:170 and SEQ ID NO: 173; or SEQ ID NO:175 and SEQ ID NO: 178; or SEQ ID NO: 219 and SEQ ID NO:220; or SEQ ID NO: 176 and SEQ ID NO: 179; or SEQ ID NO:221 and SEQ ID NO: 222; or SEQ ID NO:186 and SEQ ID NO: 187; or SEQ ID NO: 188 and SEQ ID NO: 189; or SEQ ID NO: 204 and SEQ ID NO:205; or SEQ ID NO:213 and SEQ ID NO:214; or SEQ ID NO:215 and SEQ ID NO: 216. In some embodiments, the TCR binding a HLA-A*02:01-restricted mutant splice factor-induced peptide of MAPK8IP-derived antigenic peptide RLPGVLPRA (SEQ ID NO:147), comprises the TCRα chain CDR3α and TCRβ chain CDR3β encoded by a nucleotide sequence pair sharing at least about 95% sequence identity to the nucleotide sequence pair underscored in SEQ ID NO:169 and SEQ ID NO:172; or SEQ ID NO: 170 and SEQ ID NO:173; or SEQ ID NO: 175 and SEQ ID NO: 178; or SEQ ID NO:219 and SEQ ID NO: 220; or SEQ ID NO:176 and SEQ ID NO: 179; or SEQ ID NO:221 and SEQ ID NO:222; or SEQ ID NO: 186 and SEQ ID NO:187; or SEQ ID NO: 188 and SEQ ID NO:189; or SEQ ID NO:204 and SEQ ID NO: 205; or SEQ ID NO:213 and SEQ ID NO:214; or SEQ ID NO:215 and SEQ ID NO:216.

In some embodiments, the TCR binding a HLA-A*02:01-restricted mutant splice factor-induced peptide of MAPK8IP-derived antigenic peptide RLPGVLPRA (SEQ ID NO:147), comprises a TCRα chain encoded by a nucleotide sequence of SEQ ID NOs: 169, 170, 175, 219, 176, 221, 186, 188, 204, 213 and 215, in combination with a TCRβ chain encoded by a nucleotide sequence of SEQ ID NOs: 172, 173, 178, 220, 179, 222, 187, 189, 205, 214 and 216. In some embodiments, the TCR binding a HLA-A*02:01-restricted mutant splice factor-induced peptide of MAPK8IP-derived antigenic peptide RLPGVLPRA (SEQ ID NO:147), comprises a TCRα chain encoded by a nucleotide sequence sharing at least about 80%, about 85%, about 90%, or about 95% sequence identity with SEQ ID NOs: 169, 170, 175, 219, 176, 221, 186, 188, 204, 213 and 215, in combination with a TCRβ chain encoded by a nucleotide sequence sharing at least about 80%, about 85%, about 90%, or about 95% sequence identity with SEQ ID NOs: 172, 173, 178, 220, 179, 222, 187, 189, 205, 214 and 216.

In some embodiments, the TCR binding a HLA-A*02:01-restricted mutant splice factor-induced peptide of MAPK8IP-derived antigenic peptide RLPGVLPRA (SEQ ID NO:147), comprises a TCRα chain CDR3α and TCRβ chain CDR3β encoded by the nucleotide sequence pair underscored in SEQ ID NO:169 and SEQ ID NO: 172; or SEQ ID NO:175 and SEQ ID NO: 178; or SEQ ID NO:219 and SEQ ID NO:220; or SEQ ID NO: 186 and SEQ ID NO:187; or SEQ ID NO: 188 and SEQ ID NO:189; or SEQ ID NO:206 and SEQ ID NO: 207; or SEQ ID NO:217 and SEQ ID NO:218. In some embodiments, the TCR binding a HLA-A*02:01-restricted mutant splice factor-induced peptide of MAPK8IP-derived antigenic peptide RLPGVLPRA (SEQ ID NO: 147), comprises the TCRα chain CDR3α and TCRβ chain CDR3β encoded by a nucleotide sequence pair sharing at least about 95% sequence identity to the nucleotide sequence pair underscored in SEQ ID NO:169 and SEQ ID NO: 172; or SEQ ID NO: 175 and SEQ ID NO:178; or SEQ ID NO:219 and SEQ ID NO:220; or SEQ ID NO: 186 and SEQ ID NO:187; or SEQ ID NO: 188 and SEQ ID NO:189; or SEQ ID NO:206 and SEQ ID NO: 207; or SEQ ID NO:217 and SEQ ID NO:218.

In some embodiments, the TCR binding a HLA-A*02:01-restricted mutant splice factor-induced peptide of MAPK8IP-derived antigenic peptide RLPGVLPRA (SEQ ID NO:147), comprises a TCRα chain encoded by a nucleotide sequence of SEQ ID NOs: 169, 186, 175, 188, 219, 206 or 217, in combination with a TCRβ chain encoded by a nucleotide sequence of SEQ ID NOs: 172, 187, 189, 178, 220, 207 or 218. In some embodiments, the TCR binding a HLA-A*02:01-restricted mutant splice factor-induced peptide of MAPK8IP-derived antigenic peptide RLPGVLPRA (SEQ ID NO:147), comprises a TCRα chain encoded by a nucleotide sequence sharing at least about 80%, about 85%, about 90%, or about 95% sequence identity with SEQ ID NOs: 169, 186, 175, 188, 219, 206 or 217, in combination with a TCRβ chain encoded by a nucleotide sequence sharing at least about 80%, about 85%, about 90%, or about 95% sequence identity with SEQ ID NOs: 172, 187, 189, 178, 220, 207 or 218.

In some embodiments, the TCR binding a HLA-A*02:01-restricted mutant splice factor-induced peptide of MAPK8IP-derived antigenic peptide RLPGVLPRA (SEQ ID NO:147), comprises a TCRα chain CDR3α and TCRβ chain CDR3β encoded by the nucleotide sequence pair underscored in SEQ ID NO:169 and SEQ ID NO: 172; or SEQ ID NO:170 and SEQ ID NO: 173; or SEQ ID NO:175 and SEQ ID NO: 178; or SEQ ID NO: 219 and SEQ ID NO:220; or SEQ ID NO:176 and SEQ ID NO:179; or SEQ ID NO:221 and SEQ ID NO: 222; or SEQ ID NO:186 and SEQ ID NO:187; or SEQ ID NO:188 and SEQ ID NO: 189. In some embodiments, the TCR binding a HLA-A*02:01-restricted mutant splice factor-induced peptide of MAPK8IP-derived antigenic peptide RLPGVLPRA (SEQ ID NO:147), comprises the TCRα chain CDR3α and TCRβ chain CDR3β encoded by a nucleotide sequence pair sharing at least about 95% sequence identity to the nucleotide sequence pair underscored in SEQ ID NO:169 and SEQ ID NO:172; or SEQ ID NO:170 and SEQ ID NO:173; or SEQ ID NO:175 and SEQ ID NO: 178; or SEQ ID NO:219 and SEQ ID NO:220; or SEQ ID NO:176 and SEQ ID NO:179; or SEQ ID NO:221 and SEQ ID NO:222; or SEQ ID NO: 186 and SEQ ID NO: 187; or SEQ ID NO: 188 and SEQ ID NO:189.

In some embodiments, the TCR binding a HLA-A*02:01-restricted mutant splice factor-induced peptide of MAPK8IP-derived antigenic peptide RLPGVLPRA (SEQ ID NO:147), comprises a TCRα chain encoded by a nucleotide sequence of SEQ ID NOs: 169, 170, 186, 175, 188, 176, 219 or 221, in combination with a TCRβ chain encoded by a nucleotide sequence of SEQ ID NOs: 172, 173, 187, 189, 178, 179, 220 or 222. In some embodiments, the TCR binding a HLA-A*02:01-restricted mutant splice factor-induced peptide of MAPK8IP-derived antigenic peptide RLPGVLPRA (SEQ ID NO:147), comprises a TCRα chain encoded by a nucleotide sequence sharing at least about 80%, about 85%, about 90%, or about 95% sequence identity with SEQ ID NOs: 169, 170, 186, 175, 188, 176, 219 or 221, in combination with a TCRβ chain encoded by a nucleotide sequence sharing at least about 80%, about 85%, about 90%, or about 95% sequence identity with SEQ ID NOs: 172, 173, 187, 178, 189, 179, 220 or 222.

In some embodiments, the TCR binding a HLA-A*02:01-restricted mutant splice factor-induced peptide of MAPK8IP-derived antigenic peptide RLPGVLPRA (SEQ ID NO:147), comprises a TCRα chain CDR3α and TCRβ chain CDR3β encoded by the nucleotide sequence pair underscored in SEQ ID NO:169 and SEQ ID NO: 172; or SEQ ID NO:175 and SEQ ID NO: 178; or SEQ ID NO:219 and SEQ ID NO:220; or SEQ ID NO: 186 and SEQ ID NO: 187; or SEQ ID NO: 188 and SEQ ID NO:189. In some embodiments, the TCR binding a HLA-A*02:01-restricted mutant splice factor-induced peptide of MAPK8IP-derived antigenic peptide RLPGVLPRA (SEQ ID NO:147), comprises the TCRα chain CDR3α and TCRβ chain CDR3β encoded by a nucleotide sequence pair sharing at least about 95% sequence identity to the nucleotide sequence pair underscored in SEQ ID NO: 169 and SEQ ID NO:172; or SEQ ID NO:175 and SEQ ID NO:178; or SEQ ID NO: 219 and SEQ ID NO:220; or SEQ ID NO: 186 and SEQ ID NO:187; or SEQ ID NO: 188 and SEQ ID NO: 189.

In some embodiments, the TCR binding a HLA-A*02:01-restricted mutant splice factor-induced peptide of MAPK8IP-derived antigenic peptide RLPGVLPRA (SEQ ID NO:147), comprises a TCRα chain encoded by a nucleotide sequence of SEQ ID NOs: 169, 186, 175, 188 or 219, in combination with a TCRβ chain encoded by a nucleotide sequence of SEQ ID NOs: 172, 187, 189, 178, or 220. In some embodiments, the TCR binding a HLA-A*02:01-restricted mutant splice factor-induced peptide of MAPK8IP-derived antigenic peptide RLPGVLPRA (SEQ ID NO:147), comprises a TCRα chain encoded by a nucleotide sequence sharing at least about 80%, about 85%, about 90%, or about 95% sequence identity with SEQ ID NOs: 169, 186, 175, 188 or 219, in combination with a TCRβ chain encoded by a nucleotide sequence sharing at least about 80%, about 85%, about 90%, or about 95% sequence identity with SEQ ID NOs: 172, 187, 189, 178, or 220.

### J. Human Endogenous Retroviruses (HERVs)

About 9% of the human genome consists of genetic information from human endogenous retroviruses (HERVs) that was incorporated into the germline as humans evolved (Jansz and Faulkner, 2021). The majority of HERV genetic information is fragmented and/or epigenetically repressed, and no HERV proteins are expressed. However, HERV protein can be expressed in various cancers, including breast cancer, pancreatic cancer, germ cell tumors, leukemia, prostate cancer, bladder cancer, ovarian cancer, lung cancer, hepatocellular carcinoma, lymphoma, choriocarcinoma, colorectal carcinoma, soft tissue sarcoma and Kaposi's sarcoma (Gao et al., 2021; Jansz and Faulkner, 2021). HERV-K is a group of HERVs with relatively intact open reading frames, making the expression of HERV-K proteins more likely compared to other HERVs (Gao et al., 2021). T cell responses to HERV-K proteins have been reported previously, including T cell responses to HERV-K gag protein-derived peptide FLQFKTWWI (SEQ ID NO:148; Rakoff-Nahoum et al., 2006; Saini et al., 2020; Wang-Johanning et al., 2008).

Since expression of HERV-K proteins is preferentially seen in cancer cells, T cell receptor-mediated therapy against HERV-K T cell epitopes, including FLQFKTWWI (SEQ ID NO:148), is an attractive strategy for the treatment of cancer that has not yet been tested clinically.

In some embodiments, TCR_A0100 specific for HERV-K-derived peptide FLQFKTWWI (SEQ ID NO:148) is isolated, optionally modified and cloned into a vector (e.g., a viral vector, e.g., a lentivirus vector) for expression in T cells. As set forth herein, the inventors have performed this process on Jurkat luciferase reporter cells as a model for T cell transduction. Jurkat cells were transduced with a lentiviral vector and successfully expressed the novel TCRs TCR_A0100. These cells were further tested in a specificity assay. Therefore, in an exemplary embodiment, APC expressing HLA-A alleles 02:01, are incubated with the HERV-K-derived antigenic peptide and mixed with the said Jurkat cells. Jurkat cells specifically activated by the antigenic peptide via the TCR produce luciferase. Luciferin, the substrate for luciferase, is then added along with additional reagents enabling a chemical reaction producing light. Expression of luciferase following TCR activation can thus be quantified as relative light units (RLU). An increasing response with increasing amount of peptide added to the cells is expected until reaching saturation in the system.

In some embodiments, the TCR binding HLA-A*02:01-restricted ERV-K-derived peptide FLQFKTWWI (SEQ ID NO: 148), comprises a TCRα chain CDR3α and TCRβ chain CDR3β amino acid sequence pair SEQ ID NO: 153 and SEQ ID NO:161. In some embodiments, the TCR binding a HLA-A*02:01-restricted HERV-K-derived peptide FLQFKTWWI (SEQ ID NO:148), comprises a TCRα chain CDR3α and TCRβ chain CDR3β amino acid sequence pair sharing at least about 95% sequence identity to the amino acid sequence pair SEQ ID NO: 153 and SEQ ID NO:161.

In some embodiments, the TCR binding HLA-A*02:01-restricted HERV-K-derived peptide FLQFKTWWI (SEQ ID NO:148), comprises a TCRα chain CDR3α and TCRβ chain CDR3β encoded by the nucleotide sequence pair underscored in SEQ ID NO:171 and SEQ ID NO:174. In some embodiments, the TCR binding a HLA-A*02:01-restricted HERV-K-derived peptide FLQFKTWWI (SEQ ID NO:148), comprises the TCRα chain CDR3α and TCRβ chain CDR3β encoded by a nucleotide sequence pair sharing at least about 95% sequence identity to the nucleotide sequence pair underscored in SEQ ID NO: 171 and SEQ ID NO:174.

In some embodiments, the TCR binding HLA-A*02:01-restricted HERV-K-derived peptide FLQFKTWWI (SEQ ID NO:148), comprises a TCRα chain encoded by a nucleotide sequence of SEQ ID NOs: 171 or 177, in combination with a TCRβ chain encoded by a nucleotide sequence of SEQ ID NOs: 174, or 180. In some embodiments, the TCR binding HLA-A*02:01-restricted HERV-K-derived peptide FLQFKTWWI (SEQ ID NO: 148), comprises a TCRα chain encoded by a nucleotide sequence sharing at least about 80%, about 85%, about 90%, or about 95% sequence identity with SEQ ID NOs: 171 or 177, in combination with a TCRβ chain encoded by a nucleotide sequence sharing at least about 80%, about 85%, about 90%, or about 95% sequence identity with SEQ ID NOs: 174 or 180.

### K. Plasmids, Lentiviral Vector Production and Transduction of Jurkat Cells

In some embodiments, a TCR nucleotide sequence is cloned into a plasmid for viral transduction. In an exemplary embodiment, a TCR sequence is cloned into a plasmid for lentivirus transduction. In an exemplary embodiment, a TCR sequence is cloned into a plasmid for lentivirus transduction with the TCR transgene flanked by long-terminal repeat sequences to enable packaging into lentiviral vectors. Lentiviral vectors are produced for each TCR by transfection of the transgene plasmid along with packaging plasmids encoding the additional lentiviral components into HEK293 cells. Lentiviral vectors collected from the transfected HEK293 cells are then used to transduce each TCR into the T cells. Transduction efficiency and expression of the introduced TCR are confirmed by flow cytometry.

TCR sequences suitable for expression of an exemplary TCR polypeptide in the form of an exogenous receptor on the surface of T cells or as part of a fusion construct, are presented herein. In some embodiments, the TCRα chain and TCRβ chain pairs comprise a sequence selected from the combinations set forth in Table 5 or Table 6 as part of a fusion construct, whereby said fusion construct consists of a TCR and a single-chain fragment that binds to a molecule specifically expressed on T cells, including but not limited to CD3.

### L. TCRs Binding to Antigenic Peptides

In various embodiments, the TCRs specifically recognize and bind antigenic peptides presented on MHC class I molecules.

The immense diversity of the T-cell receptor (TCR) enables specific antigen recognition. Successful recognition of antigenic peptides bound to Major Histocompatibility Complexes (pepMHCs) requires specific binding of the TCR to these complexes, which in turn modulates the cell's fitness, clonal expansion, and acquisition of effector properties. The affinity of a TCR for a given peptide epitope and the specificity of the binding are governed by the heterodimeric αβ T-cell receptors. Within the TCRβ chain, the complementarity-determining region 1 (CDR1) and CDR2 loops of the TCR contact the MHC alpha-helices while the hypervariable CDR3 interact mainly with the peptide. In both TCRα and TCRβ chains, CDR3 loops have the highest sequence diversity and are the principal determinants of receptor binding specificity. Following specific binding of T cell receptors to viral and bacterial-derived peptides bound to MHC, or from neo-antigens, the appropriate T cells expand, resulting in the increased frequency of T cells carrying such receptors (Springer et al., Front. Immunol. 25 Aug. 2020).

### M. Dose-Response Testing of Transduced Jurkat Cells

In various embodiments, the T cells expressing the TCRs exhibit a dose-responsive effect on cells expressing the target antigen.

For example, in a model system, the cell line T2 (ATCC) was used as APCs and seeded into 96 well plates at 50,000 cells per well. 50,000 transduced or non-transduced Jurkat cells were added to the T2 cells. In a separate plate, peptide dilutions were prepared to the desired range of concentrations and added to the plate containing T2 and Jurkat cells. Plates were incubated at 37° C. in a cell culture incubator for 4 h. To reveal the luciferase signal, Bio-Glo-NL™ reagent was added to all wells, reacted for 5 min and then read on a Spectra plate reader. Peptides used were either purified EBV peptides or mixtures of peptides.

### N. Codon Optimization of TCR Nucleotide Sequences

In various embodiments, the nucleotide sequence encoding one or more polypeptides of the TCR is codon optimized, e.g. for expression in a chosen cell, such as a mammalian cell.

Codon optimization is a common method used to increase the expression of recombinant proteins, especially in the field of biotherapeutics. Its basis lies in the use of synonymous codon mutations in messenger RNA (mRNA) coding regions. Codon optimization is known to maximize protein expression by overcoming expression limitations associated with codon usage. This routine method has been reported to increase protein expression by up to >1000-fold. This method is often applied in order to fine-tune the expression of one of two light chain genes of a bispecific antibody (Mauro, BioDrugs 32; 69-81 (2018)).

Altering codon usage is possible since the 20 amino acids are encoded by 61 codons. Except for methionine and tryptophane, which are encoded by a single codon each, all other amino acids are specified by two to six redundant codons. Synonymous codon usage is not random, as it varies between different organisms, between different tissues of the same organism, and even between different parts of the same gene (Mauro, BioDrugs 32; 69-81 (2018)).

The sequences were codon optimised with an algorithm provided by Genscript (GenSmart Codon Optimisation). The optimisation was done for expression in human T cells (https://www.genscript.com/gensmart-free-gene-codon-optimization.html?page_no=1&position_no=1&sensors=googlesearch). See, e.g., WIPO Pat. Appl. No. WO2020024917A1, incorporated herein by reference in its entirety.

In some embodiments, a TCRα encoding nucleotide sequence selected from SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO: 87, SEQ ID NO: 88, SEQ ID NO:89, SEQ ID NO:90, SEQ ID NO: 142, SEQ ID NO: 169, SEQ ID NO:170, SEQ ID NO:171, and SEQ ID NO:186, (as listed in Table 5) is codon-optimized to produce the TCRα nucleotide sequences SEQ ID NO:108, SEQ ID NO: 109, SEQ ID NO:110, SEQ ID NO:111, SEQ ID NO:112, SEQ ID NO: 113, SEQ ID NO:114, SEQ ID NO:115, SEQ ID NO:116, SEQ ID NO:117, SEQ ID NO: 118, SEQ ID NO: 119, SEQ ID NO:120, SEQ ID NO:134, SEQ ID NO: 175, SEQ ID NO:176, SEQ ID NO:177, SEQ ID NO: 188, SEQ ID NO:219, and SEQ ID NO:221 (as listed in Table 6). In some embodiments, a TCRα encoding nucleotide sequence SEQ ID NO:204 (as listed in Table 5) is codon-optimized to produce the TCRα nucleotide sequences SEQ ID NO:213 and SEQ ID NO:215 (as listed in Table 6). In some embodiments, a TCRα encoding nucleotide sequence SEQ ID NO:206 (as listed in Table 5) is codon-optimized to produce the TCRα nucleotide sequence SEQ ID NO:217 (as listed in Table 6).

In some embodiments, a TCRβ-encoding nucleotide sequence selected from SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO: 100, SEQ ID NO: 101, SEQ ID NO: 102, SEQ ID NO: 103, SEQ ID NO: 104, SEQ ID NO:143, SEQ ID NO: 172, SEQ ID NO:173, SEQ ID NO:174, and SEQ ID NO:187, (as listed in Table 5) is codon-optimized to produce the TCRβ nucleotide sequences SEQ ID NO:121, SEQ ID NO: 122, SEQ ID NO:123, SEQ ID NO: 124, SEQ ID NO: 125, SEQ ID NO:126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 135, SEQ ID NO:178, SEQ ID NO:179, SEQ ID NO: 180, SEQ ID NO:189, SEQ ID NO:220 and SEQ ID NO:222 (as listed in Table 6). In some embodiments, a TCRβ encoding nucleotide sequence SEQ ID NO:205 (as listed in Table 5) is codon-optimized to produce the TCRβ nucleotide sequences SEQ ID NO:214 and SEQ ID NO:216 (as listed in Table 6). In some embodiments, a TCRβ encoding nucleotide sequence SEQ ID NO:207 (as listed in Table 5) is codon-optimized to produce the TCRβ nucleotide sequence SEQ ID NO:218 (as listed in Table 6).

The following examples are offered to illustrate certain embodiments of the invention and should not be taken to limit the scope thereof.

### O. Quantification of Cytotoxicity

In various embodiments, the functionality of TCRs is assessed using primary human T cells transduced with said TCRs.

Peripheral blood mononuclear cells (PBMCs) from healthy donors were used as a source of primary T cells for the preparation of effector cells. CD4+ and CD8+ T cells were positively enriched using CD4 and CD8 microbeads, LS columns and magnets from Miltenyi. Cells were resuspended in AIM V medium with 10% heat-inactivated human AB serum and 10 ng/ml IL-15. For T cell activation, cells were incubated with TransAct™ beads (Miltenyi). On the same or the next day, lentiviruses encoding for the TCR of interest were added to the activated T cells and incubated for three days. Half of the cells were not transduced with lentivirus and were used as control cells. Transduction efficiency was verified by flow cytometry, using an anti-mouse TCR antibody to verify that at least 50% of T cells express the transduced TCR.

### 1. Cytometry-Based Cytotoxicity Assay

In some embodiments, the functionality of TCRs is assessed by quantification of cytotoxicity by flow cytometry assay.

TCR-expressing T cells were used as effector cells and were labelled with Cell trace Violet (CTV) for 30 mins and resuspended in assay buffer (99% RPMI, 1% FBS). T2 cells were used as target cells and seeded in 96 well plates. Effector cells were added at the ratio of effector:target cells indicated in the figures. Target peptide of the tested TCR was added to the wells at a range of concentrations as indicated in the figures, and cells were incubated for 20-24 hours. To analyze the cytotoxicity of effectors, cells were collected after centrifugation in a 96 well round bottom plate. Cell culture supernatant was kept for the analysis of secreted cytokines. Cells were stained with 7AAD for 15 mins and analyzed on an Attune flow cytometer (Thermo Fisher Scientific). The percentage of killing for each peptide concentration and effector cell tested was assessed by quantifying the % of 7AAD+CTV-(target) cells per condition, using FlowJo software.

2. Cytotoxicity with xCelligence Impedance Readout

In other embodiments, the functionality of TCRs is assessed by quantification of cytotoxicity with xCelligence impedance readout.

Cancer cell lines endogenously expressing the target antigen of interest and the HLA allele that tested TCRs are restricted for, were used as target cells. As effector cells, primary T cells transduced with the TCR to be tested were used. Non-TCR transduced T cells were used as control. Target cell lines were seeded in xCelligence 96well E plates at 10,000-25,000 cells per well, depending on the cell line used. Effector cells were added the next day at the Effector:Target ratio indicated in the figure legend, preparing each condition in duplicate. Impedance readings were recorded for up to 90 hours. The % of cytolysis per condition and time point was calculated with xCelligence Immunotherapy software.

### P. Quantification of Cytokine Secretion

In some embodiments, the functionality of TCRs is assessed by quantification of cytokine secretion in the cell culture media. IFNγ secreted by T cells into the culture medium during the cytotoxicity assay was quantified by ELISA methodology. The OptEIA IFNγ Kit from Becton Dickinson (BD) was used.

Q. Sequences

| SEQ | | |
|---|---|---|
| ID | | |
| NO: | DESCRIPTION | SEQUENCE |
| 1 | CDR1α A0001, A0002, | YGGTVN |
| | A0003, A0004 | |
| 2 | CDR1α A0005, A0359 | TSDQSYG |
| 3 | CDR1α A0015 | SSVSVY |
| 4 | CDR1α A0061, A0100 | DSAIYN |
| 5 | CDR1α A0062, A0068, | TSINN |
| | A0069, A0070 | |
| 6 | CDR1α A0064, A0065, | TTLSN |
| | A0066 | |
| 7 | CDR2α A0001, A0002, | YFSGDPLV |
| | A0003, A0004 | |
| 8 | CDR2α A0005, A0359 | QGSYDEQN |
| 9 | CDR2α A0015 | YLSGSTLV |
| 10 | CDR2α A0061, A0100 | IQSSQRE |
| 11 | CDR2α A0062 | IRSNERE |
| 12 | CDR2α A0064, A0065, | LVKSGEV |
| | A0066 | |
| 13 | CDR2α A0068, A0069, | IRSNERE |
| | A0070 | |
| 14 | CDR3α A0132 | CAFMEADSNYQLIW |
| 15 | CDR3α A0001, A0002, | CAVKDTDKLIF |
| | A0004 | |
| 16 | CDR3α A0003 | CAGGAAGNKLTF |
| 17 | CDR3α A0005 | CAMREGGNFNKFYF |
| 18 | CDR3α A0015 | CAVSALSYNQGGKLIF |
| 19 | CDR3α A0061 | CAVLMDSNYQLIW |
| 20 | CDR3α A0062 | CATEGSSGYSTLTF |
| 21 | CDR3α A0064, A0066 | CAGAGAGSYQLTF |
| 22 | CDR3α A0065 | CAVSGAGSYQLTF |
| 23 | CDR3α A0068, A0069 | CATEGGSGYSTLTF |
| 24 | CDR3α A0070 | CATAGNSGYSTLTF |
| 25 | CDR1β A0001, A0004, | KGHDR |
| | A0358 | |
| 26 | CDR1β A0002 | LGHDT |
| 27 | CDR1β A0003, A0099 | SGHAT |
| 28 | CDR1β A0005 | SQVTM |
| 29 | CDR1β A0015 | SGHNS |
| 30 | CDR1β A0061 | WSHSY |
| 31 | CDR1β A0062, A0068, | MNHEY |
| | A0069, A0070, A0131 | |
| 32 | CDR1β A0064, A0065, | SGHRS |
| | A0066, A0132 | |
| 33 | CDR2β A0001, A0004, | SFDVKD |
| | A0358 | |
| 34 | CDR2β A0002 | YNNKEL |
| 35 | CDR2β A0003, A0099 | FQNNGV |
| 36 | CDR2β A0005 | ANQGSEA |
| 37 | CDR2β A0015 | FNNNVP |
| 38 | CDR2β A0061 | SAAADI |
| 39 | CDR2β A0062 | SVGAGI |
| 40 | CDR2β A0064, A0065, | YFSETQ |
| | A0066, A0132 | |
| 41 | CDR2β A0068, A0069, | SVGAGI |
| | A0070 | |
| 42 | CDR3β A0132 | CASKGRRGPDYNSPLHF |
| 43 | CDR3β A0002 | CASSPDFNEQFF |
| 44 | CDR3β A0003 | CASSSPLGGFAGANVLTF |
| 45 | CDR3β A0004 | CATSDFISDTQYF |
| 46 | CDR3β A0005 | CSVGGTSGTLPANEQFF |
| 47 | CDR3β A0015 | CASSWTGNEQYF |
| 48 | CDR3β A0061 | CASSSDGMNTEAFF |
| 49 | CDR3β A0062 | CASSKQGGGYGYTF |
| 50 | CDR3β A0064, A0065 | CASSLEGQASSYEQYF |
| 51 | CDR3β A0066 | CASSAEGQASSYEQYF |
| 52 | CDR3β A0068 | CASSRQGGSGSGYTF |
| 53 | CDR3β A0069 | CASTTQGGAYGYTF |
| 54 | CDR3β A0070 | CASTPQGGNEAFF |
| 55 | TCRα variable region | AQSVSQHNHHVILSEAASLELGCNYSYGGTVNLFWYVQYPGQHLQLLLKY |
| | A0001, A0002, A0004 | FSGDPLVKGIKGFEAEFIKSKFSFNLRKPSVQWSDTAEYFCAVKDTDKLIFGT |
| | | GTRLQVFP |
| 56 | TCRα variable region | AQSVSQHNHHVILSEAASLELGCNYSYGGTVNLFWYVQYPGQHLQLLLKY |
| | A0003 | FSGDPLVKGIKGFEAEFIKSKFSFNLRKPSVQWSDTAEYFCAGGAAGNKLTF |
| | | GGGTRVLVKP |
| 57 | TCRα variable region | AQKITQTQPGMFVQEKEAVTLDCTYDTSDQSYGLFWYKQPSSGEMIFLIYQ |
| | A0005 | GSYDEQNATEGRYSLNFQKARKSANLVISASQLGDSAMYFCAMREGGNFN |
| | | KFYFGSGTKLNVKP |
| 58 | TCRα variable region | AQSVTQLDSQVPVFEEAPVELRCNYSSSVSVYLFWYVQYPNQGLQLLLKYL |
| | A0015 | SGSTLVKGINGFEAEFNKSQTSFHLRKPSVHISDTAEYFCAVSALSYNQGGK |
| | | LIFGQGTELSVKP |
| 59 | TCRα variable region | KQEVTQIPAALSVPEGENLVLNCSFTDSAIYNLQWFRQDPGKGLTSLLLIQSS |
| | A0061 | QREQTSGRLNASLDKSSGRSTLYIAASQPGDSATYLCAVLMDSNYQLIWGA |
| | | GTKLIIKP |
| 60 | TCRα variable region | SQQGEEDPQALSIQEGENATMNCSYKTSINNLQWYRQNSGRGLVHLILIRSN |
| | A0062 | EREKHSGRLRVTLDTSKKSSSLLITASRAADTASYFCATEGSSGYSTLTFGKG |
| | | TMLLVSP |
| 61 | TCRα variable region | GQQVMQIPQYQHVQEGEDFTTYCNSSTTLSNIQWYKQRPGGHPVFLIQLVK |
| | A0064, A0066 | SGEVKKQKRLTFQFGEAKKNSSLHITATQTTDVGTYFCAGAGAGSYQLTFG |
| | | KGTKLSVIP |
| 62 | TCRα variable region | GQQVMQIPQYQHVQEGEDFTTYCNSSTTLSNIQWYKQRPGGHPVFLIQLVK |
| | A0065 | SGEVKKQKRLTFQFGEAKKNSSLHITATQTTDVGTYFCAVSGAGSYQLTFG |
| | | KGTKLSVIP |
| 63 | TCRα variable region | SQQGEEDPQALSIQEGENATMNCSYKTSINNLQWYRQNSGRGLVHLILIRSN |
| | A0068 | EREKHSGRLRVTLDTSKKSSSLLITASRAADTASYFCATEGGSGYSTLTFGK |
| | | GTMLLVSP |
| 64 | TCRα variable region | SQQGEEDPQALSIQEGENATMNCSYKTSINNLQWYRQNSGRGLVHLILIRSN |
| | A0069 | EREKHSGRLRVTLDTSKKSSSLLITASRAADTASYFCATEGDSGYSTLTFGK |
| | | GTMLLVSP |
| 65 | TCRα variable region | SQQGEEDPQALSIQEGENATMNCSYKTSINNLQWYRQNSGRGLVHLILIRSN |
| | A0070 | EREKHSGRLRVTLDTSKKSSSLLITASRAADTASYFCATAGNSGYSTLTFGK |
| | | GTMLLVSP |
| 66 | TCRα variable region | AQTVTQSQPEMSVQEAETVTLSCTYDTSESNYYLFWYKQPPSRQMILVIRQE |
| | A0132 | AYKQQNATENRFSVNFQKAAKSFSLKISDSQLGDTAMYFCAFMEADSNYQ |
| | | LIWGAGTKLIIKP |
| 67 | TCRβ variable region | DTAVSQTPKYLVTQMGNDKSIKCEQNLGHDTMYWYKQDSKKFLKIMFSYN |
| | A0002 | NKELIINETVPNRFSPKSPDKAHLNLHINSLELGDSAVYFCASSPDFNEQFFGP |
| | | GTRLTVL |
| 68 | TCRβ variable region | EAGVAQSPRYKIIEKRQSVAFWCNPISGHATLYWYQQILGQGPKLLIQFQNN |
| | A0003 | GVVDDSQLPKDRFSAERLKGVDSTLKIQPAKLEDSAVYLCASSSPLGGFAG |
| | | ANVLTFGAGSRLTVL |
| 69 | TCRβ variable region | DADVTQTPRNRITKTGKRIMLECSQTKGHDRMYWYRQDPGLGLRLIYYSFD |
| | A0004 | VKDINKGEISDGYSVSRQAQAKFSLSLESAIPNQTALYFCATSDFISDTQYFG |
| | | PGTRLTVL |
| 70 | TCRβ variable region | SAVISQKPSRDICQRGTSLTIQCQVDSQVTMMFWYRQQPGQSLTLIATANQG |
| | A0005 | SEATYESGFVIDKFPISRPNLTFSTLTVSNMSPEDSSIYLCSVGGTSGTLPANE |
| | | QFFGPGTRLTVL |
| 71 | TCRβ variable region | DAGVIQSPRHEVTEMGQEVTLRCKPISGHNSLFWYRQTMMRGLELLIYFNN |
| | A0015 | NVPIDDSGMPEDRFSAKMPNASFSTLKIQPSEPRDSAVYFCASSWTGNEQYF |
| | | GPGTRLTVT |
| 72 | TCRβ variable region | DAGITQSPRYKITETGRQVTLMCHQTWSHSYMFWYRQDLGHGLRLIYYSA |
| | A0061 | AADITDKGEVPDGYVVSRSKTENFPLTLESATRSQTSVYFCASSSDGMNTEA |
| | | FFGQGTRLTVV |
| 73 | TCRβ variable region | NAGVTQTPKFQVLKTGQSMTLQCAQDMNHEYMSWYRQDPGMGLRLIHYS |
| | A0062 | VGAGITDQGEVPNGYNVSRSTTEDFPLRLLSAAPSQTSVYFCASSKQGGGY |
| | | GYTFGSGTRLTVV |
| 74 | TCRβ variable region | KAGVTQTPRYLIKTRGQQVTLSCSPISGHRSVSWYQQTPGQGLQFLFEYFSE |
| | A0064, A0065 | TQRNKGNFPGRFSGRQFSNSRSEMNVSTLELGDSALYLCASSLEGQASSYEQ |
| | | YFGPGTRLTVT |
| 75 | TCRβ variable region | KAGVTQTPRYLIKTRGQQVTLSCSPISGHRSVSWYQQTPGQGLQFLFEYFSE |
| | A0066 | TQRNKGNFPGRFSGRQFSNSRSEMNVSTLELGDSALYLCASSAEGQASSYE |
| | | QYFGPGTRLTVT |
| 76 | TCRβ variable region | NAGVTQTPKFQVLKTGQSMTLQCAQDMNHEYMSWYRQDPGMGLRLIHYS |
| | A0068 | VGAGITDQGEVPNGYNVSRSTTEDFPLRLLSAAPSQTSVYFCASSRQGGSGS |
| | | GYTFGSGTRLTVV |
| 77 | TCRβ variable region | NAGVTQTPKFQVLKTGQSMTLQCAQDMNHEYMSWYRQDPGMGLRLIHYS |
| | A0069 | VGAGITDQGEVPNGYNVSRSTTEDFPLRLLSAAPSQTSVYFCASTTQGGAYG |
| | | YTFGSGTRLTVV |
| 78 | TCRβ variable region | NAGVTQTPKFQVLKTGQSMTLQCAQDMNHEYMSWYRQDPGMGLRLIHYS |
| | A0070 | VGAGITDQGEVPNGYNVSRSTTEDFPLRLLSAAPSQTSVYFCASTPQGGNEA |
| | | FFGQGTRLTVV |
| 79 | TCRα variable region | GCCCAGTCTGTGAGCCAGCATAACCACCACGTAATTCTCTCTGAAGCAG |
| | nucleotide sequence | CCTCACTGGAGTTGGGATGCAACTATTCCTATGGTGGAACTGTTAATCTC |
| | A0002, A0004 | TTCTGGTATGTCCAGTACCCTGGTCAACACCTTCAGCTTCTCCTCAAGTA |
| | | CTTTTCAGGGGATCCACTGGTTAAAGGCATCAAGGGCTTTGAGGCTGAA |
| | | TTTATAAAGAGTAAATTCTCCTTTAATCTGAGGAAACCCTCTGTGCAGTG |
| | | GAGTGACACAGCTGAGTACTTCTGTGCCGTGAAGGACACCGACAAGCTC |
| | | ATCTTTGGGACTGGGACCAGATTACAAGTCTTTCCA |
| 80 | TCRα variable region | GCCCAGTCTGTGAGCCAGCATAACCACCACGTAATTCTCTCTGAAGCAG |
| | nucleotide sequence | CCTCACTGGAGTTGGGATGCAACTATTCCTATGGTGGAACTGTTAATCTC |
| | A0003 | TTCTGGTATGTCCAGTACCCTGGTCAACACCTTCAGCTTCTCCTCAAGTA |
| | | CTTTTCAGGGGATCCACTGGTTAAAGGCATCAAGGGCTTTGAGGCTGAA |
| | | TTTATAAAGAGTAAATTCTCCTTTAATCTGAGGAAACCCTCTGTGCAGTG |
| | | GAGTGACACAGCTGAGTACTTCTGTGCCGGGGGAGCTGCAGGCAACAAG |
| | | CTAACTTTTGGAGGAGGAACCAGGGTGCTAGTTAAACCA |
| 81 | TCRα variable region | GCCCAGAAGATAACTCAAACCCAACCAGGAATGTTCGTGCAGGAAAAG |
| | nucleotide sequence | GAGGCTGTGACTCTGGACTGCACATATGACACCAGTGATCAAAGTTATG |
| | A0005 | GTCTATTCTGGTACAAGCAGCCCAGCAGTGGGGAAATGATTTTTCTTATT |
| | | TATCAGGGGTCTTATGACGAGCAAAATGCAACAGAAGGTCGCTACTCAT |
| | | TGAATTTCCAGAAGGCAAGAAAATCCGCCAACCTTGTCATCTCCGCTTCA |
| | | CAACTGGGGGACTCAGCAATGTATTTCTGTGCAATGAGAGAGGGCGGGA |
| | | ACTTCAACAAATTTTACTTTGGATCTGGGACCAAACTCAATGTAAAACCA |
| 82 | TCRα variable region | GCCCAGTCTGTGACCCAGCTTGACAGCCAAGTCCCTGTCTTTGAAGAAG |
| | nucleotide sequence | CCCCTGTGGAGCTGAGGTGCAACTACTCATCGTCTGTTTCAGTGTATCTC |
| | A0015 | TTCTGGTATGTGCAATACCCCAACCAAGGACTCCAGCTTCTCCTGAAGTA |
| | | TTTATCAGGATCCACCCTGGTTAAAGGCATCAACGGTTTTGAGGCTGAAT |
| | | TTAACAAGAGTCAAACTTCCTTCCACTTGAGGAAACCCTCAGTCCATATA |
| | | AGCGACACGGCTGAGTACTTCTGTGCTGTGAGTGCCCTTTCTTATAACCA |
| | | GGGAGGAAAGCTTATCTTCGGACAGGGAACGGAGTTATCTGTGAAACCC |
| 83 | TCRα variable region | AAACAGGAGGTGACACAGATTCCTGCAGCTCTGAGTGTCCCAGAAGGAG |
| | nucleotide sequence | AAAACTTGGTTCTCAACTGCAGTTTCACTGATAGCGCTATTTACAACCTC |
| | A0061 | CAGTGGTTTAGGCAGGACCCTGGGAAAGGTCTCACATCTCTGTTGCTTAT |
| | | TCAGTCAAGTCAGAGAGAGCAAACAAGTGGAAGACTTAATGCCTCGCTG |
| | | GATAAATCATCAGGACGTAGTACTTTATACATTGCAGCTTCTCAGCCTGG |
| | | TGACTCAGCCACCTACCTCTGTGCTGTCCTTATGGATAGCAACTATCAGT |
| | | TAATCTGGGGCGCTGGGACCAAGCTAATTATAAAGCCA |
| 84 | TCRα variable region | AGTCAACAGGGAGAAGAGGATCCTCAGGCCTTGAGCATCCAGGAGGGT |
| | nucleotide sequence | GAAAATGCCACCATGAACTGCAGTTACAAAACTAGTATAAACAATTTAC |
| | A0062 | AGTGGTATAGACAAAATTCAGGTAGAGGCCTTGTCCACCTAATTTTAAT |
| | | ACGTTCAAATGAAAGAGAGAAACACAGTGGAAGATTAAGAGTCACGCT |
| | | TGACACTTCCAAGAAAAGCAGTTCCTTGTTGATCACGGCTTCCCGGGCA |
| | | GCAGACACTGCTTCTTACTTCTGTGCTACGGAGGGCTCTTCAGGATACAG |
| | | CACCCTCACCTTTGGGAAGGGGACTATGCTTCTAGTCTCTCCA |
| 85 | TCRα variable region | GGACAACAGGTAATGCAAATTCCTCAGTACCAGCATGTACAAGAAGGAG |
| | nucleotide sequence | AGGACTTCACCACGTACTGCAATTCCTCAACTACTTTAAGCAATATACAG |
| | A0064 | TGGTATAAGCAAAGGCCTGGTGGACATCCCGTTTTTTTGATACAGTTAGT |
| | | GAAGAGTGGAGAAGTGAAGAAGCAGAAAAGACTGACATTTCAGTTTGG |
| | | AGAAGCAAAAAAGAACAGCTCCCTGCACATCACAGCCACCCAGACTAC |
| | | AGATGTAGGAACCTACTTCTGTGCAGGAGCTGGGGCTGGGAGTTACCAA |
| | | CTCACTTTCGGGAAGGGGACCAAACTCTCGGTCATACCA |
| 86 | TCRα variable region | GGACAACAGGTAATGCAAATTCCTCAGTACCAGCATGTACAAGAAGGAG |
| | nucleotide sequence | AAGACTTCACCACGTACTGCAATTCCTCAACTACTTTAAGCAATATACAG |
| | A0065 | TGGTATAAGCAAAGGCCTGGTGGACATCCCGTTTTTTTGATACAGTTAGT |
| | | GAAGAGTGGAGAAGTGAAGAAGCAGAAAAGACTGACATTTCAGTTTGG |
| | | AGAAGCAAAAAAGAACAGCTCCCTGCACATCACAGCCACCCAGACTAC |
| | | AGATGTAGGAACCTACTTCTGTGCAGTCTCTGGGGCTGGGAGTTACCAA |
| | | CTCACTTTCGGGAAGGGGACCAAACTCTCGGTCATACCA |
| 87 | TCRα variable region | GGACAACAGGTAATGCAAATTCCTCAGTACCAGCATGTACAAGAAGGAG |
| | nucleotide sequence | AGGACTTCACCACGTACTGCAATTCCTCAACTACTTTAAGCAATATACAG |
| | A0066 | TGGTATAAGCAAAGGCCTGGTGGACATCCCGTTTTTTTGATACAGTTAGT |
| | | GAAGAGTGGAGAAGTGAAGAAGCAGAAAAGACTGACATTTCAGTTTGG |
| | | AGAAGCAAAAAAGAACAGCTCCCTGCACATCACAGCCACCCAGACTAC |
| | | AGATGTAGGAACCTACTTCTGTGCAGGGGCTGGGGCTGGGAGTTACCAA |
| | | CTCACTTTCGGGAAGGGGACCAAACTCTCGGTCATACCA |
| 88 | TCRα variable region | AGTCAACAGGGAGAAGAGGATCCTCAGGCCTTGAGCATCCAGGAGGGT |
| | nucleotide sequence | GAAAATGCCACCATGAACTGCAGTTACAAAACTAGTATAAACAATTTAC |
| | A0068 | AGTGGTATAGACAAAATTCAGGTAGAGGCCTTGTCCACCTAATTTTAAT |
| | | ACGTTCAAATGAAAGAGAGAAACACAGTGGAAGATTAAGAGTCACGCT |
| | | TGACACTTCCAAGAAAAGCAGTTCCTTGTTGATCACGGCTTCCCGGGCA |
| | | GCAGACACTGCTTCTTACTTCTGTGCTACTGAGGGCGGTTCAGGATACAG |
| | | CACCCTCACCTTTGGGAAGGGGACTATGCTTCTAGTCTCTCCA |
| 89 | TCRα variable region | AGTCAACAGGGAGAAGAGGATCCTCAGGCCTTGAGCATCCAGGAGGGT |
| | nucleotide sequence | GAAAATGCCACCATGAACTGCAGTTACAAAACTAGTATAAACAATTTAC |
| | A0069 | AGTGGTATAGACAAAATTCAGGTAGAGGCCTTGTCCACCTAATTTTAAT |
| | | ACGTTCAAATGAAAGAGAGAAACACAGTGGAAGATTAAGAGTCACGCT |
| | | TGACACTTCCAAGAAAAGCAGTTCCTTGTTGATCACGGCTTCCCGGGCA |
| | | GCAGACACTGCTTCTTACTTCTGTGCTACGGAGGGGGATTCAGGATACA |
| | | GCACCCTCACCTTTGGGAAGGGGACTATGCTTCTAGTCTCTCCA |
| 90 | TCRα variable region | AGTCAACAGGGAGAAGAGGATCCTCAGGCCTTGAGCATCCAGGAGGGT |
| | nucleotide sequence | GAAAATGCCACCATGAACTGCAGTTACAAAACTAGTATAAACAATTTAC |
| | A0070 | AGTGGTATAGACAAAATTCAGGTAGAGGCCTTGTCCACCTAATTTTAAT |
| | | ACGTTCAAATGAAAGAGAGAAACACAGTGGAAGATTAAGAGTCACGCT |
| | | TGACACTTCCAAGAAAAGCAGTTCCTTGTTGATCACGGCTTCCCGGGCA |
| | | GCAGACACTGCTTCTTACTTCTGTGCTACGGCCGGTAATTCAGGATACAG |
| | | CACCCTCACCTTTGGGAAGGGGACTATGCTTCTAGTCTCTCCA |
| 91 | TCRβ variable region | KAGVTQTPRYLIKTRGQQVTLSCSPISGHRSVSWYQQTPGQGLQFLFEYFSE |
| | A0132 | TQRNKGNFPGRFSGRQFSNSRSEMNVSTLELGDSALYLCASKGRRGPDYNS |
| | | PLHFGNGTRLTVT |
| 92 | TCRβ variable region | GACACAGCTGTTTCCCAGACTCCAAAATACCTGGTCACACAGATGGGAA |
| | nucleotide sequence | ACGACAAGTCCATTAAATGTGAACAAAATCTGGGCCATGATACTATGTA |
| | A0002 | TTGGTATAAACAGGACTCTAAGAAATTTCTGAAGATAATGTTTAGCTAC |
| | | AATAATAAGGAGCTCATTATAAATGAAACAGTTCCAAATCGCTTCTCAC |
| | | CTAAATCTCCAGACAAAGCTCACTTAAATCTTCACATCAATTCCCTGGAG |
| | | CTTGGTGACTCTGCTGTGTATTTCTGTGCCAGCAGCCCAGACTTCAATGA |
| | | GCAGTTCTTCGGGCCAGGGACACGGCTCACCGTGCTA |
| 93 | TCRβ variable region | GAAGCTGGAGTTGCCCAGTCTCCCAGATATAAGATTATAGAGAAAAGGC |
| | nucleotide sequence | AGAGTGTGGCTTTTTGGTGCAATCCTATATCTGGCCATGCTACCCTTTAC |
| | A0003 | TGGTACCAGCAGATCCTGGGACAGGGCCCAAAGCTTCTGATTCAGTTTC |
| | | AGAATAACGGTGTAGTGGATGATTCACAGTTGCCTAAGGATCGATTTTCT |
| | | GCAGAGAGGCTCAAAGGAGTAGACTCCACTCTCAAGATCCAACCTGCAA |
| | | AGCTTGAGGACTCGGCCGTGTATCTCTGTGCCAGCAGTTCACCATTGGGG |
| | | GGGTTCGCGGGGGCCAACGTCCTGACTTTCGGGGCCGGCAGCAGGCTGA |
| | | CCGTGCTG |
| 94 | TCRβ variable region | GATGCTGATGTTACCCAGACCCCAAGGAATAGGATCACAAAGACAGGA |
| | nucleotide sequence | AAGAGGATTATGCTGGAATGTTCTCAGACTAAGGGTCATGATAGAATGT |
| | A0004 | ACTGGTATCGACAAGACCCAGGACTGGGCCTACGGTTGATCTATTACTC |
| | | CTTTGATGTCAAAGATATAAACAAAGGAGAGATCTCTGATGGATACAGT |
| | | GTCTCTCGACAGGCACAGGCTAAATTCTCCCTGTCCCTAGAGTCTGCCAT |
| | | CCCCAACCAGACAGCTCTTTACTTCTGTGCCACCAGTGATTTCATCTCAG |
| | | ATACGCAGTATTTTGGCCCAGGCACCCGGCTGACAGTGCTC |
| 95 | TCRβ variable region | AGTGCTGTCATCTCTCAAAAGCCAAGCAGGGATATCTGTCAACGTGGAA |
| | nucleotide sequence | CCTCCCTGACGATCCAGTGTCAAGTCGATAGCCAAGTCACCATGATGTTC |
| | A0005 | TGGTACCGTCAGCAACCTGGACAGAGCCTGACACTGATCGCAACTGCAA |
| | | ATCAGGGCTCTGAGGCCACATATGAGAGTGGATTTGTCATTGACAAGTT |
| | | TCCCATCAGCCGCCCAAACCTAACATTCTCAACTCTGACTGTGAGCAACA |
| | | TGAGCCCTGAAGACAGCAGCATATATCTCTGCAGCGTTGGTGGGACTAG |
| | | CGGGACTCTCCCTGCCAATGAGCAGTTCTTCGGGCCAGGGACACGGCTC |
| | | ACCGTGCTA |
| 96 | TCRβ variable region | GATGCTGGAGTTATCCAGTCACCCCGCCATGAGGTGACAGAGATGGGAC |
| | nucleotide sequence | AAGAAGTGACTCTGAGATGTAAACCAATTTCAGGCCACAACTCCCTTTTC |
| | A0015 | TGGTACAGACAGACCATGATGCGGGGACTGGAGTTGCTCATTTACTTTA |
| | | ACAACAACGTTCCGATAGATGATTCAGGGATGCCCGAGGATCGATTCTC |
| | | AGCTAAGATGCCTAATGCATCATTCTCCACTCTGAAGATCCAGCCCTCAG |
| | | AACCCAGGGACTCAGCTGTGTACTTCTGTGCCAGCAGCTGGACAGGGAA |
| | | CGAGCAGTACTTCGGGCCGGGCACCAGGCTCACGGTCACA |
| 97 | TCRβ variable region | GATGCTGGAATCACCCAGAGCCCAAGATACAAGATCACAGAGACAGGA |
| | nucleotide sequence | AGGCAGGTGACCTTGATGTGTCACCAGACTTGGAGCCACAGCTATATGT |
| | A0061 | TCTGGTATCGACAAGACCTGGGACATGGGCTGAGGCTGATCTATTACTC |
| | | AGCAGCTGCTGATATTACAGATAAAGGAGAAGTCCCCGATGGCTATGTT |
| | | GTCTCCAGATCCAAGACAGAGAATTTCCCCCTCACTCTGGAGTCAGCTAC |
| | | CCGCTCCCAGACATCTGTGTATTTCTGCGCCAGCAGCTCGGACGGGATG |
| | | AACACTGAAGCTTTCTTTGGACAAGGCACCAGACTCACAGTTGTA |
| 98 | TCRβ variable region | AATGCTGGTGTCACTCAGACCCCAAAATTCCAGGTCCTGAAGACAGGAC |
| | nucleotide sequence | AGAGCATGACACTGCAGTGTGCCCAGGATATGAACCATGAATACATGTC |
| | A0062 | CTGGTATCGACAAGACCCAGGCATGGGGCTGAGGCTGATTCATTACTCA |
| | | GTTGGTGCTGGTATCACTGACCAAGGAGAAGTCCCCAATGGCTACAATG |
| | | TCTCCAGATCAACCACAGAGGATTTCCCGCTCAGGCTGCTGTCGGCTGCT |
| | | CCCTCCCAGACATCTGTGTACTTCTGTGCCAGCAGTAAACAGGGAGGGG |
| | | GCTATGGCTACACCTTCGGTTCGGGGACCAGGTTAACCGTTGTA |
| 99 | TCRβ variable region | AAGGCTGGAGTCACTCAAACTCCAAGATATCTGATCAAAACGAGAGGAC |
| | nucleotide sequence | AGCAAGTGACACTGAGCTGCTCCCCTATCTCTGGGCATAGGAGTGTATC |
| | A0064 | CTGGTACCAACAGACCCCAGGACAGGGCCTTCAGTTCCTCTTTGAATACT |
| | | TCAGTGAGACACAGAGAAACAAAGGAAACTTCCCTGGTCGATTCTCAGG |
| | | GCGCCAGTTCTCTAACTCTCGCTCTGAGATGAATGTGAGCACCTTGGAGC |
| | | TGGGGGACTCGGCCCTTTATCTTTGCGCCAGCAGCCTCGAGGGACAGGC |
| | | GAGCTCCTACGAGCAGTACTTCGGGCCGGGCACCAGGCTCACGGTCACA |
| 100 | TCRβ variable region | AAGGCTGGAGTCACTCAAACTCCAAGATATCTGATCAAAACGAGAGGAC |
| | nucleotide sequence | AGCAAGTGACACTGAGCTGCTCCCCTATCTCTGGGCATAGGAGTGTATC |
| | A0065 | CTGGTACCAACAGACCCCAGGACAGGGCCTTCAGTTCCTCTTTGAATACT |
| | | TCAGTGAGACACAGAGAAACAAAGGAAACTTCCCTGGTCGATTCTCAGG |
| | | GCGCCAGTTCTCTAACTCTCGCTCTGAGATGAATGTGAGCACCTTGGAGC |
| | | TGGGGGACTCGGCCCTTTATCTTTGCGCCAGCAGCTTGGAGGGGCAGGC |
| | | CTCCTCCTACGAGCAGTACTTCGGGCCGGGCACCAGGCTCACGGTCACA |
| 101 | TCRβ variable region | AAGGCTGGAGTCACTCAAACTCCAAGATATCTGATCAAAACGAGAGGAC |
| | nucleotide sequence | AGCAAGTGACACTGAGCTGCTCCCCTATCTCTGGGCATAGGAGTGTATC |
| | A0066 | CTGGTACCAACAGACCCCAGGACAGGGCCTTCAGTTCCTCTTTGAATACT |
| | | TCAGTGAGACACAGAGAAACAAAGGAAACTTCCCTGGTCGATTCTCAGG |
| | | GCGCCAGTTCTCTAACTCTCGCTCTGAGATGAATGTGAGCACCTTGGAGC |
| | | TGGGGGACTCGGCCCTTTATCTTTGCGCCAGCAGCGCGGAGGGACAGGC |
| | | TTCCTCCTACGAGCAGTACTTCGGGCCGGGCACCAGGCTCACGGTCACA |
| 102 | TCRβ variable region | AATGCTGGTGTCACTCAGACCCCAAAATTCCAGGTCCTGAAGACAGGAC |
| | nucleotide sequence | AGAGCATGACACTGCAGTGTGCCCAGGATATGAACCATGAATACATGTC |
| | A0068 | CTGGTATCGACAAGACCCAGGCATGGGGCTGAGGCTGATTCATTACTCA |
| | | GTTGGTGCTGGTATCACTGACCAAGGAGAAGTCCCCAATGGCTACAATG |
| | | TCTCCAGATCAACCACAGAGGATTTCCCGCTCAGGCTGCTGTCGGCTGCT |
| | | CCCTCCCAGACATCTGTGTACTTCTGTGCCAGCAGTCGACAAGGGGGTTC |
| | | CGGGAGTGGCTACACCTTCGGTTCGGGGACCAGGTTAACCGTTGTA |
| 103 | TCRβ variable region | AATGCTGGTGTCACTCAGACCCCAAAATTCCAGGTCCTGAAGACAGGAC |
| | nucleotide sequence | AGAGCATGACACTGCAGTGTGCCCAGGATATGAACCATGAATACATGTC |
| | A0069 | CTGGTATCGACAAGACCCAGGCATGGGGCTGAGGCTGATTCATTACTCA |
| | | GTTGGTGCTGGTATCACTGACCAAGGAGAAGTCCCCAATGGCTACAATG |
| | | TCTCCAGATCAACCACAGAGGATTTCCCGCTCAGGCTGCTGTCGGCTGCT |
| | | CCCTCCCAGACATCTGTGTACTTCTGTGCCAGCACCACCCAGGGGGGGG |
| | | CCTATGGCTACACCTTCGGTTCGGGGACCAGGTTAACCGTTGTA |
| 104 | TCRβ variable region | AATGCTGGTGTCACTCAGACCCCAAAATTCCAGGTCCTGAAGACAGGAC |
| | nucleotide sequence | AGAGCATGACACTGCAGTGTGCCCAGGATATGAACCATGAATACATGTC |
| | A0070 | CTGGTATCGACAAGACCCAGGCATGGGGCTGAGGCTGATTCATTACTCA |
| | | GTTGGTGCTGGTATCACTGACCAAGGAGAAGTCCCCAATGGCTACAATG |
| | | TCTCCAGATCAACCACAGAGGATTTCCCGCTCAGGCTGCTGTCGGCTGCT |
| | | CCCTCCCAGACATCTGTGTACTTCTGTGCCAGCACCCCCCAGGGGGGCA |
| | | ACGAAGCTTTCTTTGGACAAGGCACCAGACTCACAGTTGTA |
| 105 | BRLF1 peptide | YVLDHLIVV |
| 106 | LMP2 peptide | CLGGLLTMV |
| 107 | LMP2 peptide | FLYALALLL |
| 108 | TCRα variable region | GCGCAGAGCGTTTCGCAACACAACCACCACGTCATCCTGTCCGAGGCTG |
| | codon optimized | CTTCTCTGGAGCTGGGGTGCAACTACAGCTACGGTGGCACGGTCAATCT |
| | nucleotide sequence | ATTTTGGTACGTGCAGTATCCAGGACAGCATCTCCAGCTGCTGCTCAAGT |
| | A0002 | ACTTTTCAGGGGACCCGTTGGTGAAAGGCATCAAGGGCTTCGAAGCAGA |
| | | GTTCATTAAGTCGAAATTTTCCTTCAACCTGCGTAAGCCTTCCGTGCAGT |
| | | GGTCTGATACTGCCGAGTACTTCTGTGCCGTGAAGGACACCGACAAGCT |
| | | TATCTTCGGTACCGGCACCCGCCTGCAGGTGTTCCCC |
| 109 | TCRα variable region | GCGCAGAGCGTGTCCCAACACAACCACCACGTCATCCTGTCCGAGGCTG |
| | codon optimized | CTTCCCTGGAGCTGGGGTGCAACTACAGCTACGGCGGCACCGTCAATTT |
| | nucleotide sequence | GTTCTGGTACGTGCAGTATCCGGGACAGCATCTCCAGCTGCTGCTCAAGT |
| | A0003 | ACTTTAGTGGTGATCCACTTGTTAAAGGCATCAAGGGCTTCGAAGCCGA |
| | | GTTCATTAAGTCGAAATTTTCATTCAACCTGCGCAAGCCCTCTGTGCAGT |
| | | GGTCTGACACCGCAGAGTACTTCTGTGCCGGGGGCGCGGCCGGCAACAA |
| | | GCTGACCTTCGGAGGCGGTACTCGTGTGCTGGTGAAGCCT |
| 110 | TCRα variable region | GCGCAGAGCGTGTCCCAACACAACCACCACGTTATCCTGTCCGAGGCTG |
| | codon optimized | CATCCCTGGAGCTGGGGTGCAACTACAGCTACGGTGGCACCGTCAATCT |
| | nucleotide sequence | ATTTTGGTACGTGCAGTATCCTGGACAGCATTTGCAGCTGCTGCTCAAGT |
| | A0004 | ACTTCAGTGGGGACCCGCTGGTGAAAGGCATCAAGGGCTTCGAAGCTGA |
| | | GTTCATTAAGTCGAAATTTTCATTCAACCTGCGCAAGCCCTCTGTGCAGT |
| | | GGTCTGATACGGCCGAGTACTTTTGTGCCGTGAAGGACACCGACAAGCT |
| | | TATCTTCGGTACCGGCACTCGTCTCCAGGTCTTCCCC |
| 111 | TCRα variable region | GCACAGAAGATCACCCAGACTCAACCTGGTATGTTTGTGCAGGAGAAGG |
| | codon optimized | AGGCTGTCACATTGGACTGTACCTACGACACCTCCGACCAGAGCTACGG |
| | nucleotide sequence | CCTTTTCTGGTACAAGCAGCCGAGTTCGGGGGAGATGATCTTCCTGATCT |
| | A0005 | ATCAGGGCTCCTACGACGAACAGAACGCGACGGAGGGCCGCTACTCCCT |
| | | CAACTTCCAGAAGGCCCGCAAAAGCGCGAACCTGGTGATTTCTGCTTCC |
| | | CAGCTGGGTGATTCTGCCATGTATTTCTGCGCCATGCGTGAGGGCGGCA |
| | | ACTTCAACAAGTTCTACTTCGGATCAGGGACCAAGCTGAATGTGAAGCC |
| | | C |
| 112 | TCRα variable region | GCGCAGAGCGTCACCCAGTTGGATTCTCAGGTCCCAGTGTTCGAGGAGG |
| | codon optimized | CTCCGGTGGAGCTTCGTTGTAACTACTCCTCGTCAGTATCCGTGTACCTC |
| | nucleotide sequence | TTTTGGTACGTGCAGTATCCCAACCAGGGTCTGCAGCTGCTGCTCAAGTA |
| | A0015 | CCTGTCTGGCTCCACTCTGGTGAAGGGCATTAATGGCTTCGAAGCAGAG |
| | | TTCAACAAGTCGCAGACCTCCTTCCATCTGCGCAAGCCCTCTGTCCACAT |
| | | CTCCGACACCGCCGAGTACTTTTGCGCCGTGTCCGCCCTGAGCTACAACC |
| | | AAGGGGGTAAGCTAATCTTCGGACAGGGCACCGAGCTGAGTGTTAAACC |
| | | T |
| 113 | TCRα variable region | AAGCAGGAGGTGACACAAATTCCCGCCGCCCTGTCCGTCCCCGAGGGCG |
| | codon optimized | AGAATCTGGTGCTCAACTGCTCTTTTACCGACAGTGCCATCTACAACCTG |
| | nucleotide sequence | CAGTGGTTCCGCCAGGACCCGGGCAAGGGTCTGACCTCCCTGCTGCTCA |
| | A0061 | TCCAGAGCTCACAGCGGGAACAGACTTCCGGCCGCCTGAACGCGTCTTT |
| | | GGACAAAAGCTCCGGGCGCTCGACCCTGTACATCGCCGCTTCCCAGCCA |
| | | GGTGATTCTGCTACCTACCTGTGCGCCGTGCTGATGGACAGCAACTATCA |
| | | GCTTATTTGGGGCGCCGGCACCAAGCTGATCATCAAGCCT |
| 114 | TCRα variable region | TCACAACAGGGCGAGGAAGATCCTCAGGCCCTCAGCATCCAGGAGGGG |
| | codon optimized | GAGAATGCAACAATGAACTGCTCTTACAAGACCAGCATTAACAACCTGC |
| | nucleotide sequence | AGTGGTACCGCCAGAACTCCGGTCGTGGTTTGGTGCATTTGATCCTGATC |
| | A0062 | CGCAGCAACGAGAGGGAGAAGCACAGTGGACGCCTGCGGGTCACCCTG |
| | | GACACCTCCAAGAAGTCGTCCTCTCTGCTCATCACCGCTTCCCGCGCCGC |
| | | GGACACTGCTAGCTATTTTTGTGCCACCGAGGGCTCCTCTGGCTACTCCA |
| | | CTCTTACCTTCGGCAAAGGCACCATGCTGCTGGTGTCGCCC |
| 115 | TCRα variable region | GGACAGCAGGTCATGCAAATTCCTCAGTACCAGCATGTCCAGGAGGGCG |
| | codon optimized | AGGACTTCACCACTTACTGCAATAGCTCGACCACTTTGAGCAACATCCA |
| | nucleotide sequence | GTGGTACAAGCAGCGTCCGGGCGGCCACCCCGTGTTCCTGATCCAGCTG |
| | A0064 | GTGAAGTCTGGGGAAGTTAAGAAACAGAAGCGCCTGACCTTCCAGTTTG |
| | | GAGAGGCCAAGAAGAACTCCTCTCTTCACATCACCGCCACCCAGACAAC |
| | | CGATGTGGGGACCTACTTTTGTGCTGGTGCGGGTGCAGGCTCCTATCAGC |
| | | TCACCTTCGGCAAAGGCACCAAGCTGTCCGTGATCCCA |
| 116 | TCRα variable region | GGACAGCAGGTCATGCAAATTCCTCAGTATCAGCATGTGCAGGAGGGCG |
| | codon optimized | AAGACTTCACCACTTACTGTAATTCCTCGACCACCTTGAGCAACATCCAG |
| | nucleotide sequence | TGGTACAAGCAGCGTCCCGGCGGCCACCCCGTGTTCCTGATCCAGCTGG |
| | A0065 | TGAAATCTGGCGAGGTGAAGAAACAGAAGCGCCTGACCTTCCAGTTTGG |
| | | GGAGGCCAAGAAGAACTCCAGCCTTCACATCACCGCCACCCAGACTACA |
| | | GATGTGGGGACCTACTTTTGCGCTGTTTCTGGTGCGGGCTCCTACCAGCT |
| | | CACCTTCGGCAAGGGCACCAAGCTGAGTGTCATCCCG |
| 117 | TCRα variable region | GGACAGCAGGTCATGCAAATTCCTCAGTACCAGCATGTGCAGGAGGGCG |
| | codon optimized | AAGACTTCACCACTTACTGTAATTCCTCGACCACACTCAGCAACATCCAG |
| | nucleotide sequence | TGGTACAAGCAGCGTCCCGGCGGCCACCCCGTGTTCCTGATCCAGCTGG |
| | A0066 | TGAAGTCTGGGGAGGTTAAGAAACAGAAGCGCCTGACCTTTCAGTTCGG |
| | | AGAGGCCAAGAAGAACTCCTCTTTGCACATCACCGCCACCCAGACGACT |
| | | GATGTGGGGACCTACTTTTGCGCTGGTGCAGGTGCGGGCTCCTATCAGCT |
| | | TACCTTCGGCAAAGGCACCAAGCTGAGCGTCATCCCG |
| 118 | TCRα variable region | TCACAGCAAGGCGAGGAAGATCCTCAGGCCCTCAGCATCCAGGAGGGG |
| | codon optimized | GAGAATGCAACTATGAACTGCAGCTACAAGACCAGTATTAACAACCTGC |
| | nucleotide sequence | AGTGGTACCGCCAGAACTCCGGACGTGGTCTAGTGCATTTGATCCTGATC |
| | A0068 | CGCAGCAACGAGAGGGAGAAGCACTCGGGTCGCCTGCGGGTCACCCTG |
| | | GACACCTCCAAGAAGTCTTCTTCTCTGCTCATCACTGCTTCCCGCGCCGC |
| | | GGACACAGCTAGCTATTTTTGTGCCACCGAGGGCGGCTCCGGCTACTCC |
| | | ACCCTTACCTTCGGCAAAGGCACCATGCTGCTGGTGTCGCCC |
| 119 | TCRα variable region | TCACAACAGGGCGAGGAGGACCCTCAGGCCCTCTCTATCCAGGAGGGCG |
| | codon optimized | AGAATGCAACAATGAACTGCAGCTACAAGACCAGCATTAACAACCTGCA |
| | nucleotide sequence | GTGGTACCGGCAGAACTCCGGCCGTGGTTTGGTGCATCTAATCCTGATCC |
| | A0069 | GCAGCAACGAGAGGGAGAAGCACAGTGGGCGCCTGCGCGTCACCCTGG |
| | | ACACCTCCAAGAAGTCGTCCTCTCTGCTCATCACCGCTTCCCGCGCCGCG |
| | | GACACTGCTAGCTATTTTTGTGCCACTGAAGGTGATTCTGGCTACTCCAC |
| | | CCTTACCTTCGGCAAAGGCACCATGCTGCTGGTGTCGCCC |
| 120 | TCRα variable region | TCACAACAGGGAGAGGAGGACCCTCAGGCCCTCAGCATCCAGGAGGGC |
| | codon optimized | GAGAATGCCACTATGAACTGCTCTTACAAGACCAGCATTAACAACCTGC |
| | nucleotide sequence | AGTGGTACCGCCAGAACAGTGGGCGTGGTTTGGTGCATCTCATCCTGAT |
| | A0070 | CCGCAGCAACGAGCGCGAAAAGCACTCGGGTCGCCTGCGGGTCACCTTG |
| | | GATACCTCCAAGAAGTCCTCTTCTCTGCTGATCACTGCTTCCAGGGCGGC |
| | | GGACACCGCAAGCTATTTTTGTGCTACAGCCGGCAACTCCGGCTACTCCA |
| | | CCCTGACCTTCGGCAAAGGCACCATGCTTCTGGTGTCGCCC |
| 121 | TCRβ variable region | GACACCGCCGTTTCCCAGACACCGAAGTACCTGGTGACCCAGATGGGCA |
| | codon optimized | ACGACAAGAGCATCAAGTGCGAGCAGAACCTGGGCCATGACACGATGT |
| | nucleotide sequence | ATTGGTACAAGCAGGACTCCAAGAAATTTCTGAAGATCATGTTTAGCTA |
| | A0002 | CAACAACAAGGAGCTCATCATTAACGAGACCGTGCCCAACCGCTTCTCA |
| | | CCAAAGTCGCCCGACAAAGCGCACTTGAATCTACACATCAATTCTCTGG |
| | | AGCTGGGTGATTCTGCCGTGTACTTCTGTGCTTCCTCGCCGGATTTCAAC |
| | | GAACAGTTCTTCGGCCCTGGGACTCGTCTGACCGTCCTT |
| 122 | TCRβ variable region | GAAGCAGGGGTGGCTCAGAGCCCGCGCTACAAGATTATTGAGAAGCGCC |
| | codon optimized | AGTCCGTGGCGTTCTGGTGCAATCCCATCTCTGGCCACGCCACTCTTTAT |
| | nucleotide sequence | TGGTACCAACAGATCCTGGGACAGGGCCCTAAATTGCTCATCCAGTTCC |
| | A0003 | AGAACAACGGTGTGGTCGATGACAGCCAGCTGCCCAAGGACAGGTTTTC |
| | | AGCCGAGCGCCTGAAGGGCGTCGACTCGACCCTCAAAATCCAGCCAGCC |
| | | AAGCTGGAGGACAGTGCGGTGTACCTGTGCGCCTCCTCCTCTCCTCTGGG |
| | | CGGCTTCGCGGGGGCCAACGTGCTGACCTTCGGTGCTGGCTCCCGTCTGA |
| | | CCGTTCTA |
| 123 | TCRβ variable region | GATGCGGACGTGACCCAGACTCCCCGCAACCGCATCACCAAGACCGGCA |
| | codon optimized | AGCGCATCATGTTGGAGTGCTCTCAAACAAAGGGCCACGACAGGATGTA |
| | nucleotide sequence | CTGGTACCGGCAGGACCCGGGGCTGGGCCTCCGTCTTATCTACTACTCCT |
| | A0004 | TCGACGTGAAGGACATCAATAAGGGTGAGATCAGCGATGGCTACTCCGT |
| | | GTCGCGACAGGCTCAGGCCAAATTTTCACTATCTCTGGAGTCCGCCATCC |
| | | CCAACCAGACGGCACTGTACTTCTGTGCCACCTCCGACTTCATTAGTGAC |
| | | ACCCAGTATTTCGGACCTGGTACTCGCCTGACCGTGCTG |
| 124 | TCRβ variable region | TCGGCAGTGATTAGCCAGAAGCCCTCTCGGGACATCTGCCAGCGTGGTA |
| | codon optimized | CAAGTTTGACCATCCAGTGCCAAGTTGATTCTCAGGTCACCATGATGTTT |
| | nucleotide sequence | TGGTACCGCCAGCAGCCGGGACAGAGCCTAACTCTTATCGCGACGGCCA |
| | A0005 | ACCAGGGCTCCGAGGCTACCTACGAGAGCGGCTTCGTCATTGACAAGTT |
| | | TCCCATCTCCCGCCCTAACCTGACCTTCTCGACTCTCACCGTGTCCAATA |
| | | TGTCTCCTGAAGACAGCTCCATCTATCTGTGCTCCGTGGGCGGCACCTCC |
| | | GGCACCCTGCCAGCCAACGAGCAGTTCTTCGGTCCCGGGACCCGCCTGA |
| | | CCGTGCTG |
| 125 | TCRβ variable region | GACGCAGGGGTGATCCAGAGCCCGCGCCATGAAGTCACCGAGATGGGC |
| | codon optimized | CAGGAGGTGACTCTTAGGTGTAAACCCATCTCTGGCCACAACTCCCTCTT |
| | nucleotide sequence | TTGGTACCGCCAGACTATGATGCGTGGTCTGGAGCTGCTGATTTACTTCA |
| | A0015 | ACAACAATGTGCCCATCGATGACTCTGGTATGCCTGAGGACCGCTTTTCA |
| | | GCCAAGATGCCCAACGCGTCCTTCTCGACCCTGAAGATCCAGCCGTCCG |
| | | AGCCACGGGACAGCGCCGTGTACTTCTGCGCTTCCAGTTGGACCGGCAA |
| | | CGAGCAGTATTTCGGACCTGGCACCCGCTTGACGGTTACA |
| 126 | TCRβ variable region | GATGCAGGCATCACCCAGAGCCCGCGCTACAAGATTACAGAGACCGGCC |
| | codon optimized | GCCAGGTCACCCTGATGTGCCACCAGACCTGGTCTCATAGTTACATGTTT |
| | nucleotide sequence | TGGTACAGGCAGGACCTGGGCCACGGTCTCCGTCTTATCTACTACTCCGC |
| | A0061 | TGCTGCCGACATCACCGACAAAGGGGAGGTGCCCGACGGCTACGTGGTG |
| | | TCGCGGTCCAAGACTGAGAACTTCCCTTTGACTCTGGAGAGCGCCACTC |
| | | GCTCGCAAACGTCCGTTTATTTCTGCGCCTCCTCTAGCGATGGTATGAAC |
| | | ACGGAAGCGTTCTTCGGACAGGGCACCCGCCTGACCGTGGTC |
| 127 | TCRβ variable region | AATGCAGGTGTCACCCAGACTCCGAAATTTCAGGTCCTGAAGACCGGTC |
| | codon optimized | AGAGCATGACTTTGCAGTGCGCCCAGGACATGAACCATGAGTACATGAG |
| | nucleotide sequence | TTGGTACCGCCAGGATCCAGGAATGGGCCTTCGGCTCATTCACTACTCCG |
| | A0062 | TGGGGGCCGGCATCACCGACCAGGGGGAGGTGCCTAACGGCTACAACGT |
| | | GTCCCGCTCGACCACAGAGGACTTCCCCCTGCGTCTGCTGTCCGCCGCCC |
| | | CCTCTCAAACGAGCGTGTACTTCTGTGCTTCTTCCAAGCAGGGCGGTGGA |
| | | TACGGCTATACCTTCGGCTCCGGCACCCGCCTGACCGTGGTT |
| 128 | TCRβ variable region | AAAGCCGGCGTGACCCAGACTCCGCGCTACCTAATTAAGACTCGTGGTC |
| | codon optimized | AACAGGTCACCCTGAGCTGTTCGCCCATCTCTGGCCACCGGTCCGTTAGT |
| | nucleotide sequence | TGGTACCAGCAGACGCCAGGACAGGGCCTCCAGTTCCTGTTCGAGTACT |
| | A0064 | TCTCCGAGACCCAGCGCAACAAGGGCAACTTTCCTGGGCGATTCTCTGG |
| | | TCGCCAGTTTTCAAATTCCAGGTCGGAGATGAACGTGTCCACCCTTGAGC |
| | | TGGGGGACAGCGCGCTGTACCTGTGCGCTTCCTCTTTGGAGGGCCAGGC |
| | | CAGCAGCTACGAACAGTATTTCGGACCCGGCACCCGCCTGACCGTGACA |
| 129 | TCRβ variable region | AAAGCCGGCGTGACCCAGACGCCACGATACCTAATTAAGACCCGTGGTC |
| | codon optimized | AACAGGTCACCCTTTCATGCTCTCCCATCTCGGGCCACCGGTCCGTGAGT |
| | nucleotide sequence | TGGTACCAGCAGACTCCGGGACAGGGCCTCCAGTTCCTGTTCGAGTACTT |
| | A0065 | CTCCGAGACCCAGCGCAACAAGGGCAACTTTCCCGGGCGCTTCTCTGGA |
| | | CGCCAGTTTTCCAATTCCAGGTCGGAGATGAACGTGTCCACTCTGGAGCT |
| | | GGGGGACAGCGCGCTGTACCTGTGCGCCTCCTCTTTGGAGGGCCAGGCC |
| | | AGCAGCTACGAACAGTATTTCGGTCCTGGCACCCGCCTGACCGTTACA |
| 130 | TCRβ variable region | AAAGCCGGTGTTACCCAGACTCCGCGCTACCTCATTAAGACCAGAGGAC |
| | codon optimized | AACAGGTCACTCTTTCATGCTCTCCCATCTCTGGCCACCGGTCCGTGAGT |
| | nucleotide sequence | TGGTACCAGCAGACGCCAGGACAGGGCTTGCAGTTCCTGTTCGAGTACT |
| | A0066 | TCTCCGAGACCCAGCGCAACAAGGGCAACTTTCCCGGGCGTTTCTCTGGT |
| | | CGCCAGTTTTCAAATTCCAGGTCGGAGATGAACGTGTCGACCCTGGAGC |
| | | TAGGGGACAGCGCGCTGTACCTGTGCGCCTCCAGCGCAGAGGGCCAGGC |
| | | CTCCAGCTACGAACAGTATTTCGGCCCTGGCACCCGCCTGACCGTGACA |
| 131 | TCRβ variable region | AACGCGGGTGTCACCCAGACTCCGAAGTTTCAGGTCCTGAAGACCGGTC |
| | codon optimized | AGAGCATGACTTTGCAGTGCGCCCAGGACATGAATCATGAGTACATGAG |
| | nucleotide sequence | TTGGTACAGGCAGGATCCAGGAATGGGCCTCCGTCTTATTCACTACTCCG |
| | A0068 | TTGGGGCCGGCATCACCGACCAGGGGGAGGTGCCTAACGGCTACAACGT |
| | | GTCCCGCTCGACCACAGAGGACTTCCCCCTGCGGCTGCTGAGCGCAGCT |
| | | CCCTCTCAAACGTCCGTGTACTTCTGTGCTTCTAGCCGCCAGGGCGGTTC |
| | | AGGCTCCGGCTATACCTTCGGCTCGGGCACCCGCCTGACCGTGGTG |
| 132 | TCRβ variable region | AACGCGGGTGTCACCCAGACTCCTAAGTTTCAGGTCTTGAAGACTGGAC |
| | codon optimized | AGAGCATGACACTGCAGTGTGCCCAGGACATGAACCATGAGTACATGAG |
| | nucleotide sequence | TTGGTACCGCCAGGACCCGGGAATGGGCCTCCGTCTTATTCACTACTCCG |
| | A0069 | TGGGTGCTGGCATCACCGACCAGGGGGAGGTGCCAAATGGCTACAACGT |
| | | GTCCCGCTCAACGACCGAGGATTTCCCCCTGCGGCTGCTGTCTGCAGCTC |
| | | CCTCTCAAACTAGCGTGTACTTCTGCGCCTCGACCACCCAGGGCGGGGC |
| | | CTACGGCTATACCTTCGGCTCCGGCACCCGCCTGACCGTGGTT |
| 133 | TCRβ variable region | AACGCAGGTGTCACCCAGACTCCGAAGTTTCAGGTCTTGAAGACCGGCC |
| | codon optimized | AGAGCATGACGCTGCAGTGCGCCCAGGACATGAATCATGAGTACATGAG |
| | nucleotide sequence | TTGGTACCGCCAGGATCCAGGTATGGGCCTTCGTCTCATTCACTACTCCG |
| | A0070 | TGGGGGCCGGCATCACCGACCAGGGGGAGGTGCCTAACGGCTACAACGT |
| | | GTCCCGGTCGACCACAGAAGACTTCCCCCTGCGCCTGCTGTCCGCCGCCC |
| | | CCTCTCAAACGTCTGTTTATTTCTGTGCTTCCACTCCTCAGGGAGGCAAC |
| | | GAGGCGTTCTTCGGACAGGGCACCCGCCTGACCGTGGTG |
| 134 | TCRα variable region | ATACTGAACGTGGAACAAAGTCCTCAGTCACTGCATGTTCAGGAGGGAG |
| | codon optimized | ACAGCACCAATTTCACCTGCAGCTTCCCTTCCAGCAATTTTTATGCCTTA |
| | nucleotide sequence | CACTGGTACAGATGGGAAACTGCAAAAAGCCCCGAGGCCTTGTTTGTAA |
| | A0099 | TGACTTTAAATGGGGATGAAAAGAAGAAAGGACGAATAAGTGCCACTCT |
| | | TAATACCAAGGAGGGTTACAGCTATTTGTACATCAAAGGATCCCAGCCT |
| | | GAAGACTCAGCCACATACCTCTGTGCCGTTAATGCTGGTGGTACTAGCTA |
| | | TGGAAAGCTGACATTTGGACAAGGGACCATCTTGACTGTCCATCCA |
| 135 | TCRβ variable region | GAAGCTGGAGTTGCCCAGTCTCCCAGATATAAGATTATAGAGAAAAGGC |
| | codon optimized | AGAGTGTGGCTTTTTGGTGCAATCCTATATCTGGCCATGCTACCCTTTAC |
| | nucleotide sequence | TGGTACCAGCAGATCCTGGGACAGGGCCCAAAGCTTCTGATTCAGTTTC |
| | A0099 | AGAATAACGGTGTAGTGGATGATTCACAGTTGCCTAAGGATCGATTTTCT |
| | | GCAGAGAGGCTCAAAGGAGTAGACTCCACTCTCAAGATCCAACCTGCAA |
| | | AGCTTGAGGACTCGGCCGTGTATCTCTGTGCCAGCAGCTCCGATTGGAC |
| | | AGCGAACAATGAGCAGTTCTTCGGGCCAGGGACACGGCTCACCGTGCTA |
| 136 | CDR1α A0099 | SSNFYA |
| 137 | CDR2α A0099 | MTLNGDE |
| 138 | CDR3α A0099 | CAVNAGGTSYGKLTF |
| 139 | CDR3β A0099 | CASSSDWTANNEQFF |
| 140 | TCRα variable region | ILNVEQSPQSLHVQEGDSTNFTCSFPSSNFYALHWYRWETAKSPEALFVMTL |
| | A0099 | NGDEKKKGRISATLNTKEGYSYLYIKGSQPEDSATYLCAVNAGGTSYGKLT |
| | | FGQGTILTVHP |
| 141 | TCRβ variable region | EAGVAQSPRYKIIEKRQSVAFWCNPISGHATLYWYQQILGQGPKLLIQFQNN |
| | A0099 | GVVDDSQLPKDRFSAERLKGVDSTLKIQPAKLEDSAVYLCASSSDWTANNE |
| | | QFFGPGTRLTVL |
| 142 | TCRα variable region | ATACTGAACGTGGAACAAAGTCCTCAGTCACTGCATGTTCAGGAGGGAG |
| | nucleotide sequence | ACAGCACCAATTTCACCTGCAGCTTCCCTTCCAGCAATTTTTATGCCTTA |
| | A0099 | CACTGGTACAGATGGGAAACTGCAAAAAGCCCCGAGGCCTTGTTTGTAA |
| | | TGACTTTAAATGGGGATGAAAAGAAGAAAGGACGAATAAGTGCCACTCT |
| | | TAATACCAAGGAGGGTTACAGCTATTTGTACATCAAAGGATCCCAGCCT |
| | | GAAGACTCAGCCACATACCTCTGTGCCGTTAATGCTGGTGGTACTAGCTA |
| | | TGGAAAGCTGACATTTGGACAAGGGACCATCTTGACTGTCCATCCA |
| 143 | TCRβ variable region | GAAGCTGGAGTTGCCCAGTCTCCCAGATATAAGATTATAGAGAAAAGGC |
| | nucleotide sequence | AGAGTGTGGCTTTTTGGTGCAATCCTATATCTGGCCATGCTACCCTTTAC |
| | A0099 | TGGTACCAGCAGATCCTGGGACAGGGCCCAAAGCTTCTGATTCAGTTTC |
| | | AGAATAACGGTGTAGTGGATGATTCACAGTTGCCTAAGGATCGATTTTCT |
| | | GCAGAGAGGCTCAAAGGAGTAGACTCCACTCTCAAGATCCAACCTGCAA |
| | | AGCTTGAGGACTCGGCCGTGTATCTCTGTGCCAGCAGCTCCGATTGGAC |
| | | AGCGAACAATGAGCAGTTCTTCGGGCCAGGGACACGGCTCACCGTGCTA |
| 144 | Human MHC class I | MSRSVALAVLALLSLSGLEAIQRTPKIQVYSRHPAENGKSNFLNCYVSGFHP |
| | β2-microglobulin chain | SDIEVDLLKNGERIEKVEHSDLSFSKDWSFYLLYYTEFTPTEKDEYACRVNH |
| | | VTLSQPKIVKWDRDM |
| 145 | BZLF1 peptide | EPLPQGQLTAY |
| 146 | LMP2A peptide | MGSLEMVPM |
| 147 | MAPK8IP2 peptide | RLPGVLPRA |
| 148 | HERV-K peptide | FLQFKTWWI |
| 149 | CDR1α A0130, A0132 | TSESNYY |
| 150 | CDR2α A0130, A0131, | QEAYKQQN |
| | A0132, A0358 | |
| 151 | CDR3α A0130 | CAFMIPDSNYQLIW |
| 152 | CDR3α A0131 | CAFMLIDSGTYKYIF |
| 153 | CDR3α A0100 | CAVGGNNNDMRF |
| 154 | CDR1β A0130 | LGHNA |
| 155 | CDR1β A0100 | PRHDT |
| 156 | CDR2β A0130 | YNFKEQ |
| 157 | CDR2β A0131 | SMNVEV |
| 158 | CDR2β A0100 | FYEKMQ |
| 159 | CDR3β A0130 | CASSQVGTSGRGGELFF |
| 160 | CDR3β A0131 | CASSLGQGTETQYF |
| 161 | CDR3β A0100 | CASSLINTEAFF |
| 162 | TCRα variable region | AQTVTQSQPEMSVQEAETVTLSCTYDTSESNYYLFWYKQPPSRQMILVIRQE |
| | A0130 | AYKQQNATENRFSVNFQKAAKSFSLKISDSQLGDTAMYFCAFMIPDSNYQLI |
| | | WGAGTKLIIKP |
| 163 | TCRα variable region | AQTVTQSQPEMSVQEAETVTLSCTYDTSENNYYLFWYKQPPSRQMILVIRQ |
| | A0131 | EAYKQQNATENRFSVNFQKAAKSFSLKISDSQLGDTAMYFCAFMLIDSGTY |
| | | KYIFGTGTRLKVLA |
| 164 | TCRα variable region | KQEVTQIPAALSVPEGENLVLNCSFTDSAIYNLQWFRQDPGKGLTSLLLIQSS |
| | A0100 | QREQTSGRLNASLDKSSGRSTLYIAASQPGDSATYLCAVGGNNNDMRFGAG |
| | | TRLTVKP |
| 165 | CDR1α A0131, A0358 | TSENNYY |
| 166 | TCRβ variable region | ETGVTQTPRHLVMGMTNKKSLKCEQHLGHNAMYWYKQSAKKPLELMFV |
| | A0130 | YNFKEQTENNSVPSRFSPECPNSSHLFLHLHTLQPEDSALYLCASSQVGTSGR |
| | | GGELFFGEGSRLTVL |
| 167 | TCRβ variable region | EAQVTQNPRYLITVTGKKLTVTCSQNMNHEYMSWYRQDPGLGLRQIYYSM |
| | A0131 | NVEVTDKGDVPEGYKVSRKEKRNFPLILESPSPNQTSLYFCASSLGQGTETQ |
| | | YFGPGTRLLVL |
| 168 | TCRβ variable region | AAGVIQSPRHLIKEKRETATLKCYPIPRHDTVYWYQQGPGQDPQFLISFYEK |
| | A0100 | MQSDKGSIPDRFSAQQFSDYHSELNMSSLELGDSALYFCASSLINTEAFFGQ |
| | | GTRLTVV |
| 169 | TCRα variable region | GCCCAGACAGTCACTCAGTCTCAACCAGAGATGTCTGTGCAGGAGGCAG |
| | nucleotide sequence | AGACTGTGACCCTGAGTTGCACATATGACACCAGTGAGAGTAATTATTA |
| | A0130 | TTTGTTCTGGTACAAACAGCCTCCCAGCAGGCAGATGATTCTCGTTATTC |
| | | GCCAAGAAGCTTATAAGCAACAGAATGCAACGGAGAATCGTTTCTCTGT |
| | | GAACTTCCAGAAAGCAGCCAAATCCTTCAGTCTCAAGATCTCAGACTCA |
| | | CAGCTGGGGGACACTGCGATGTATTTCTGTGCTTTCATGATACCGGATAG |
| | | CAACTATCAGTTAATCTGGGGCGCTGGGACCAAGCTAATTATAAAGCCA |
| 170 | TCRα variable region | GCCCAGACAGTCACTCAGTCTCAACCAGAGATGTCTGTGCAGGAGGCAG |
| | nucleotide sequence | AGACTGTGACCCTGAGTTGCACATATGACACCAGTGAGAATAATTATTA |
| | A0131 | TTTGTTCTGGTACAAGCAGCCTCCCAGCAGGCAGATGATTCTCGTTATTC |
| | | GCCAAGAAGCTTATAAGCAACAGAATGCAACGGAGAATCGTTTCTCTGT |
| | | GAACTTCCAGAAAGCAGCCAAATCCTTCAGTCTCAAGATCTCAGACTCA |
| | | CAGCTGGGGGACACTGCGATGTATTTCTGTGCTTTCATGTTAATAGACTC |
| | | AGGAACCTACAAATACATCTTTGGAACAGGCACCAGGCTGAAGGTTTTA |
| | | GCA |
| 171 | TCRα variable region | AAACAGGAGGTGACACAGATTCCTGCAGCTCTGAGTGTCCCAGAAGGAG |
| | nucleotide sequence | AAAACTTGGTTCTCAACTGCAGTTTCACTGATAGCGCTATTTACAACCTC |
| | A0100 | CAGTGGTTTAGGCAGGACCCTGGGAAAGGTCTCACATCTCTGTTGCTTAT |
| | | TCAGTCAAGTCAGAGAGAGCAAACAAGTGGAAGACTTAATGCCTCGCTG |
| | | GATAAATCATCAGGACGTAGTACTTTATACATTGCAGCTTCTCAGCCTGG |
| | | TGACTCAGCCACCTACCTCTGTGCTGTGGGAGGCAATAACAATGACATG |
| | | CGCTTTGGAGCAGGGACCAGACTGACAGTAAAACCA |
| 172 | TCRβ variable region | GAAACGGGAGTTACGCAGACACCAAGACACCTGGTCATGGGAATGACA |
| | nucleotide sequence | AATAAGAAGTCTTTGAAATGTGAACAACATCTGGGGCATAACGCTATGT |
| | A0130 | ATTGGTACAAGCAAAGTGCTAAGAAGCCACTGGAGCTCATGTTTGTCTA |
| | | CAACTTTAAAGAACAGACTGAAAACAACAGTGTGCCAAGTCGCTTCTCA |
| | | CCTGAATGCCCCAACAGCTCTCACTTATTCCTTCACCTACACACCCTGCA |
| | | GCCAGAAGACTCGGCCCTGTATCTCTGTGCCAGCAGCCAAGTTGGGACT |
| | | AGCGGGAGGGGCGGGGAGCTGTTTTTTGGAGAAGGCTCTAGGCTGACCG |
| | | TACTG |
| 173 | TCRβ variable region | GAAGCCCAAGTGACCCAGAACCCAAGATACCTCATCACAGTGACTGGAA |
| | nucleotide sequence | AGAAGTTAACAGTGACTTGTTCTCAGAATATGAACCATGAGTATATGTC |
| | A0131 | CTGGTATCGACAAGACCCAGGGCTGGGCTTAAGGCAGATCTACTATTCA |
| | | ATGAATGTTGAGGTGACTGATAAGGGAGATGTTCCTGAAGGGTACAAAG |
| | | TCTCTCGAAAAGAGAAGAGGAATTTCCCCCTGATCCTGGAGTCGCCCAG |
| | | CCCCAACCAGACCTCTCTGTACTTCTGTGCCAGCAGTCTTGGACAGGGAA |
| | | CAGAGACCCAGTACTTCGGGCCAGGCACGCGGCTCCTGGTGCTC |
| 174 | TCRβ variable region | GCTGCTGGAGTCATCCAGTCCCCAAGACATCTGATCAAAGAAAAGAGGG |
| | nucleotide sequence | AAACAGCCACTCTGAAATGCTATCCTATCCCTAGACACGACACTGTCTAC |
| | A0100 | TGGTACCAGCAGGGTCCAGGTCAGGACCCCCAGTTCCTCATTTCGTTTTA |
| | | TGAAAAGATGCAGAGCGATAAAGGAAGCATCCCTGATCGATTCTCAGCT |
| | | CAACAGTTCAGTGACTATCATTCTGAACTGAACATGAGCTCCTTGGAGCT |
| | | GGGGGACTCAGCCCTGTACTTCTGTGCCAGCAGCTTAATTAACACTGAA |
| | | GCTTTCTTTGGACAAGGCACCAGACTCACAGTTGTA |
| 175 | TCRα variable region | GCACAGACGGTCACCCAGAGCCAGCCGGAGATGTCTGTGCAGGAGGCTG |
| | codon optimized | AAACCGTGACCTTGTCATGCACTTACGACACCTCCGAGAGCAACTACTA |
| | nucleotide sequence | CCTGTTTTGGTACAAGCAGCCACCCTCTCGTCAGATGATCCTGGTGATTC |
| | A0130 | GCCAGGAGGCCTACAAGCAACAGAACGCGACTGAGAACCGCTTCTCCGT |
| | | TAATTTCCAGAAGGCCGCCAAATCGTTTTCCCTCAAAATCTCCGACAGTC |
| | | AGCTGGGTGATACAGCCATGTACTTCTGTGCGTTCATGATCCCCGACAGC |
| | | AACTATCAGCTTATTTGGGGCGCTGGCACCAAGCTGATCATCAAGCCT |
| 176 | TCRα variable region | GCGCAGACGGTTACCCAGAGCCAACCTGAGATGTCCGTGCAGGAGGCTG |
| | codon optimized | AAACCGTGACCTTGTCATGCACTTACGACACCTCCGAGAACAACTATTA |
| | nucleotide sequence | CCTGTTTTGGTACAAGCAGCCGCCCTCTCGTCAGATGATCCTGGTGATCC |
| | A0131 | GCCAGGAGGCCTACAAACAGCAGAACGCAACCGAGAATCGGTTTTCGGT |
| | | CAACTTCCAGAAGGCTGCCAAATCCTTCTCCCTCAAGATCAGCGATTCTC |
| | | AGCTGGGCGACACGGCCATGTATTTCTGTGCGTTCATGCTTATTGACAGT |
| | | GGCACCTACAAGTACATCTTCGGGACAGGTACTCGCCTGAAGGTGCTGG |
| | | CC |
| 177 | TCRα variable region | AAACAGGAGGTGACACAGATTCCTGCAGCTCTGAGTGTCCCAGAAGGAG |
| | codon optimized | AAAACTTGGTTCTCAACTGCAGTTTCACTGATAGCGCTATTTACAACCTC |
| | nucleotide sequence | CAGTGGTTTAGGCAGGACCCTGGGAAAGGTCTCACATCTCTGTTGCTTAT |
| | A0100 | TCAGTCAAGTCAGAGAGAGCAAACAAGTGGAAGACTTAATGCCTCGCTG |
| | | GATAAATCATCAGGACGTAGTACTTTATACATTGCAGCTTCTCAGCCTGG |
| | | TGACTCAGCCACCTACCTCTGTGCTGTGGGAGGCAATAACAATGACATG |
| | | CGCTTTGGAGCAGGGACCAGACTGACAGTAAAACCA |
| 178 | TCRβ variable region | GAGACTGGCGTCACCCAGACTCCGCGCCACCTGGTGATGGGAATGACCA |
| | codon optimized | ACAAGAAATCTCTTAAATGCGAGCAACATCTAGGCCACAACGCCATGTA |
| | nucleotide sequence | TTGGTACAAGCAGAGCGCCAAGAAGCCCCTGGAGCTGATGTTCGTGTAC |
| | A0130 | AACTTCAAGGAGCAGACGGAGAACAACTCCGTGCCCTCTCGGTTCAGCC |
| | | CTGAATGCCCAAATTCGAGTCACTTGTTCCTGCACTTGCATACACTCCAG |
| | | CCGGAGGACAGCGCGCTGTACCTGTGCGCCTCCTCACAGGTTGGCACCT |
| | | CCGGTCGTGGTGGGGAGCTCTTTTTCGGCGAGGGCTCCCGCCTGACCGTG |
| | | CTG |
| 179 | TCRβ variable region | GAAGCTCAGGTCACCCAGAATCCACGTTATCTCATCACAGTCACCGGCA |
| | codon optimized | AGAAGCTCACGGTTACCTGCTCTCAGAACATGAACCACGAGTACATGAG |
| | nucleotide sequence | TTGGTACAGGCAGGACCCGGGCCTTGGCTTGCGGCAAATTTACTACTCC |
| | A0131 | ATGAACGTGGAGGTGACCGACAAAGGTGATGTGCCTGAGGGCTACAAG |
| | | GTGTCCCGCAAGGAGAAGCGCAACTTTCCCCTGATCCTGGAGAGCCCTT |
| | | CCCCCAACCAGACTTCTCTGTACTTCTGTGCCAGCTCGCTAGGACAGGGC |
| | | ACCGAGACCCAGTATTTCGGTCCCGGGACTCGCCTGCTGGTGCTG |
| 180 | TCRβ variable region | GCTGCTGGAGTCATCCAGTCCCCAAGACATCTGATCAAAGAAAAGAGGG |
| | codon optimized | AAACAGCCACTCTGAAATGCTATCCTATCCCTAGACACGACACTGTCTAC |
| | nucleotide sequence | TGGTACCAGCAGGGTCCAGGTCAGGACCCCCAGTTCCTCATTTCGTTTTA |
| | A0100 | TGAAAAGATGCAGAGCGATAAAGGAAGCATCCCTGATCGATTCTCAGCT |
| | | CAACAGTTCAGTGACTATCATTCTGAACTGAACATGAGCTCCTTGGAGCT |
| | | GGGGGACTCAGCCCTGTACTTCTGTGCCAGCAGCTTAATTAACACTGAA |
| | | GCTTTCTTTGGACAAGGCACCAGACTCACAGTTGTA |
| 181 | CDR3α consensus | C-A-T-X¹-G-X²-S-G-Y-S-T-L-T-F |
| | sequence for binding to | whereby: X¹ is E or A; X² is D, G, N or S, or any of the |
| | HLA-A*02-restricted | following amino acids with related properties: E, A, Q |
| | EBV LMP2-derived | or T |
| | peptide of SEQ ID | |
| | NO: 107 | |
| 182 | CDR3β consensus | C-A-S-X³-X⁴-Q-G-G-(S)-X⁵-X⁶-G-Y-T-F |
| | sequence for binding to | whereby: X³ is S or T, or any of the following amino acids |
| | HLA-A*02-restricted | with related properties: N or Q; X⁴ is K, R or T, or any of |
| | EBV LMP2-derived | the following amino acids with related properties: H, S; X⁵ |
| | peptide of SEQ ID | is G or A; X⁶ is Y or S, or any of the following amino acids |
| | NO: 107 | with related properties: F, W, H or T |
| 183 | CDR3α consensus | C-A-X¹-X²-G-A-G-S-Y-Q-L-T-F |
| | sequence for binding to | whereby: X¹ is G or V, or any of the following amino acids |
| | HLA-A*02-restricted | with related properties: A, I or L; X² is A or S, or any of |
| | EBV LMP2-derived | the following amino acids with related properties: G or T; |
| | peptide of SEQ ID | |
| | NO: 106 | |
| 184 | CDR3β consensus | C-A-S-S-X³-E-G-Q-A-S-S-Y-E-Q-Y-F |
| | sequence for binding to | whereby: X³ is L or A, or any of the following amino acids |
| | HLA-A*02-restricted | with related properties: I, V or G |
| | EBV LMP2-derived | |
| | peptide of SEQ ID | |
| | NO: 106 | |
| 185 | CDR3α consensus | C-A-F-M-X¹-X²-D-S-X³-X⁴-Y-X⁵-X⁶-I-X⁷ |
| | sequence for binding to | whereby X¹ is L or I or E, or any of the following amino |
| | HLA-A*02-restricted | acids with related properties: V or D. X² is P or I or A, |
| | MAPK8IP2-derived | or any of the following amino acids with related properties: |
| | peptide of SEQ ID | V, L or G. X³ is Gor N, or any of the following amino acids |
| | NO: 147 | with related properties: Q, A, C or S. X⁴ is T or no AA |
| | | at this position, or S as an amino acid with related |
| | | properties. X⁵ is K or Q, or any of the following amino |
| | | acids with related properties: R, H or N. X⁶ is L or Y, or |
| | | any of the following amino acids with related |
| | | properties: I, V, F, W or H. X⁷ is F or W. |
| 186 | TCRα variable region | GCCCAGACAGTCACTCAGTCTCAACCAGAGATGTCTGTGCAGGAGGCAG |
| | nucleotide sequence | AGACTGTGACCCTGAGTTGCACATATGACACCAGTGAGAGTAATTATTA |
| | A0132 | TTTGTTCTGGTACAAACAGCCTCCCAGCAGGCAGATGATTCTCGTTATTC |
| | | GCCAAGAAGCTTATAAGCAACAGAATGCAACGGAGAATCGTTTCTCTGT |
| | | GAACTTCCAGAAAGCAGCCAAATCCTTCAGTCTCAAGATCTCAGACTCA |
| | | CAGCTGGGGGACACTGCGATGTATTTCTGTGCTTTCATGGAGGCGGATA |
| | | GCAACTATCAGTTAATCTGGGGCGCTGGGACCAAGCTAATTATAAAGCC |
| | | A |
| 187 | TCRβ variable region | AAGGCTGGAGTCACTCAAACTCCAAGATATCTGATCAAAACGAGAGGAC |
| | nucleotide sequence | AGCAAGTGACACTGAGCTGCTCCCCTATCTCTGGGCATAGGAGTGTATC |
| | A0132 | CTGGTACCAACAGACCCCAGGACAGGGCCTTCAGTTCCTCTTTGAATACT |
| | | TCAGTGAGACACAGAGAAACAAAGGAAACTTCCCTGGTCGATTCTCAGG |
| | | GCGCCAGTTCTCTAACTCTCGCTCTGAGATGAATGTGAGCACCTTGGAGC |
| | | TGGGGGACTCGGCCCTTTATCTTTGCGCCAGCAAGGGCAGGCGGGGGCC |
| | | GGACTATAATTCACCCCTCCACTTTGGGAACGGGACCAGGCTCACTGTG |
| | | ACA |
| 188 | TCRα variable region | GCGCAGACGGTGACCCAGAGCCAGCCGGAGATGTCCGTGCAGGAGGCT |
| | codon optimized | GAGACCGTCACCCTGTCGTGCACTTACGACACCTCCGAGAGCAACTACT |
| | nucleotide sequence | ACCTGTTTTGGTACAAGCAGCCACCCTCTCGCCAGATGATCCTGGTGATT |
| | A0132 | CGTCAGGAGGCCTACAAACAGCAGAACGCGACAGAGAACCGCTTCTCG |
| | | GTTAATTTCCAGAAGGCAGCCAAGTCCTTCTCCCTCAAAATTAGCGATTC |
| | | TCAATTGGGTGACACTGCCATGTACTTCTGTGCTTTTATGGAAGCGGACA |
| | | GTAACTATCAGCTTATCTGGGGCGCCGGCACCAAGCTGATCATCAAGCC |
| | | T |
| 189 | TCRβ variable region | AAGGCCGGCGTTACCCAGACGCCTCGTTATCTTATTAAGACCCGAGGAC |
| | codon optimized | AGCAGGTCACACTATCTTGCTCTCCCATCTCTGGCCACCGCTCCGTGAGT |
| | nucleotide sequence | TGGTACCAACAGACTCCGGGTCAGGGCCTCCAGTTCCTGTTCGAGTACTT |
| | A0132 | CAGCGAAACCCAGCGCAACAAGGGCAACTTCCCAGGGCGCTTCAGCGG |
| | | ACGCCAGTTTTCAAATTCCAGGTCGGAGATGAACGTGTCGACCCTGGAG |
| | | CTGGGTGATAGCGCGCTGTACCTGTGCGCCTCCAAAGGCCGGCGTGGGC |
| | | CCGACTACAACTCCCCTTTGCATTTTGGCAACGGCACCCGCCTGACCGTG |
| | | ACT |
| 190 | mTRAC | NIQNPEPAVYQLKDPRSQDSTLCLFTDFDSQINVPKTMESGTFITDKCVLDM |
| | | KAMDSKSNGAIAWSNQTSFTCQDIFKETNATYPSSDVPCDATLTEKSFETD |
| | | MNLNFQNLLVIVLRILLLKVAGFNLLMTLRLWSS |
| 191 | mTRBC | EDLRNVTPPKVSLFEPSKAEIANKQKATLVCLARGFFPDHVELSWWVNGKE |
| | | VHSGVCTDPQAYKESNYSYCLSSRLRVSATFWHNPRNHFRCQVQFHGLSEE |
| | | DKWPEGSPKPVTQNISAEAWGRADCGITSASYQQGVLSATILYEILLGKATL |
| | | YAVLVSTLVVMAMVKRKNS |
| 192 | CDR3β A0001 | CATSDWDDSTGELFF |
| 193 | TCRβ variable region | DADVTQTPRNRITKTGKRIMLECSQTKGHDRMYWYRQDPGLGLRLIYYSFD |
| | A0001 | VKDINKGEISDGYSVSRQAQAKFSLSLESAIPNQTALYFCATSDWDDSTGEL |
| | | FFGEGSRLTVL |
| 194 | CDR3α A0358 | CAFMGPDSGTYKYIF |
| 195 | CDR3β A0358 | CATSDSDRIYGYTF |
| 196 | CDR3α A0359 | CAMREPDSNYQLIW |
| 197 | CDR1β A0359 | SNHLY |
| 198 | CDR2β A0359 | FYNNEI |
| 199 | CDR3β A0359 | CASQKGLEYEQYF |
| 200 | TCRα variable region | AQTVTQSQPEMSVQEAETVTLSCTYDTSENNYYLFWYKQPPSRQMILVIRQ |
| | A0358 | EAYKQQNATENRFSVNFQKAAKSFSLKISDSQLGDTAMYFCAFMGPDSGTY |
| | | KYIFGTGTRLKVLA |
| 201 | TCRβ variable region | DADVTQTPRNRITKTGKRIMLECSQTKGHDRMYWYRQDPGLGLQLIYYSFD |
| | A0358 | VKDINKGEISDGYSVSRQAQAKFSLSLESAIPNQTALYFCATSDSDRIYGYTF |
| | | GSGTRLTVV |
| 202 | TCRα variable region | AQKITQTQPGMFVQEKEAVTLDCTYDTSDQSYGLFWYKQPSSGEMIFLIYQ |
| | A0359 | GSYDEQNATEGRYSLNFQKARKSANLVISASQLGDSAMYFCAMREPDSNY |
| | | QLIWGAGTKLIIKP |
| 203 | TCRβ variable region | EPEVTQTPSHQVTQMGQEVILRCVPISNHLYFYWYRQILGQKVEFLVSFYNN |
| | A0359 | EISEKSEIFDDQFSVERPDGSNFTLKIRSTKLEDSAMYFCASQKGLEYEQYFG |
| | | PGTRLTVT |
| 204 | TCRα variable region | GCCCAGACAGTCACTCAGTCTCAACCAGAGATGTCTGTGCAGGAGGCAG |
| | nucleotide sequence | AGACTGTGACCCTGAGTTGCACATATGACACCAGTGAGAATAATTATTA |
| | A0358 | TTTGTTCTGGTACAAGCAGCCTCCCAGCAGGCAGATGATTCTCGTTATTC |
| | | GCCAAGAAGCTTATAAGCAACAGAATGCAACGGAGAATCGTTTCTCTGT |
| | | GAACTTCCAGAAAGCAGCCAAATCCTTCAGTCTCAAGATCTCAGACTCA |
| | | CAGCTGGGGGACACTGCGATGTATTTCTGTGCTTTCATGGGACCTGACTC |
| | | AGGAACCTACAAATACATCTTTGGAACAGGCACCAGGCTGAAGGTTTTA |
| | | GCA |
| 205 | TCRβ variable region | GATGCTGATGTTACCCAGACCCCAAGGAATAGGATCACAAAGACAGGA |
| | nucleotide sequence | AAGAGGATTATGCTGGAATGTTCTCAGACTAAGGGTCATGATAGAATGT |
| | A0358 | ACTGGTATCGACAAGACCCAGGACTGGGCCTACAGTTGATCTATTACTC |
| | | CTTTGATGTCAAAGATATAAACAAAGGAGAGATCTCTGATGGATACAGT |
| | | GTCTCTCGACAGGCACAGGCTAAATTCTCCCTGTCCCTAGAGTCTGCCAT |
| | | CCCCAACCAGACAGCTCTTTACTTCTGTGCCACCAGTGATTCCGACAGAA |
| | | TCTATGGCTACACCTTCGGTTCGGGGACCAGGTTAACCGTTGTA |
| 206 | TCRα variable region | GCCCAGAAGATAACTCAAACCCAACCAGGAATGTTCGTGCAGGAAAAG |
| | nucleotide sequence | GAGGCTGTGACTCTGGACTGCACATATGACACCAGTGATCAAAGTTATG |
| | A0359 | GTCTATTCTGGTACAAGCAGCCCAGCAGTGGGGAAATGATTTTTCTTATT |
| | | TATCAGGGGTCTTATGACGAGCAAAATGCAACAGAAGGTCGCTACTCAT |
| | | TGAATTTCCAGAAGGCAAGAAAATCCGCCAACCTTGTCATCTCCGCTTCA |
| | | CAACTGGGGGACTCAGCAATGTATTTCTGTGCAATGAGAGAGCCCGATA |
| | | GCAACTATCAGTTAATCTGGGGCGCTGGGACCAAGCTAATTATAAAGCC |
| | | A |
| 207 | TCRβ variable region | GAACCTGAAGTCACCCAGACTCCCAGCCATCAGGTCACACAGATGGGAC |
| | nucleotide sequence | AGGAAGTGATCTTGCGCTGTGTCCCCATCTCTAATCACTTATACTTCTAT |
| | A0359 | TGGTACAGACAAATCTTGGGGCAGAAAGTCGAGTTTCTGGTTTCCTTTTA |
| | | TAATAATGAAATCTCAGAGAAGTCTGAAATATTCGATGATCAATTCTCA |
| | | GTTGAAAGGCCTGATGGATCAAATTTCACTCTGAAGATCCGGTCCACAA |
| | | AGCTGGAGGACTCAGCCATGTACTTCTGTGCCAGCCAAAAGGGACTAGA |
| | | GTACGAGCAGTACTTCGGGCCGGGCACCAGGCTCACGGTCACAG |
| 208 | Human TRAC; Uniprot | IQNPDPAVYQLRDSKSSDKSVCLFTDFDSQTNVSQSKDSDVYITDKTVLDM |
| | P01848, entry version | RSMDFKSNSAVAWSNKSDFACANAFNNSIIPEDTFFPSPESSCDVKLVEKSFE |
| | 179, sequence version 2 | TDTNLNFQNLSVIGFRILLLKVAGFNLLMTLRLWSS |
| 209 | Human TRBC1; | DLNKVFPPEVAVFEPSEAEISHTQKATLVCLATGFFPDHVELSWWVNGKEV |
| | Uniprot P01850, entry | HSGVSTDPQPLKEQPALNDSRYCLSSRLRVSATFWQNPRNHFRCQVQFYGL |
| | version 170, sequence | SENDEWTQDRAKPVTQIVSAEAWGRADCGFTSVSYQQGVLSATILYEILLG |
| | version 4 | KATLYAVLVSALVLMAMVKRKDF |
| 210 | Human TRBC2; | DLKNVFPPKVAVFEPSEAEISHTQKATLVCLATGFYPDHVELSWWVNGKEV |
| | Uniprot A0A5B9, entry | HSGVSTDPQPLKEQPALNDSRYCLSSRLRVSATFWQNPRNHFRCQVQFYGL |
| | version 108, sequence | SENDEWTQDRAKPVTQIVSAEAWGRADCGFTSESYQQGVLSATILYEILLG |
| | version 2 | KATLYAVLVSALVLMAMVKRKDSRG |
| 211 | Human TRAC with | NIQNPDPAVYQLRDSKSSDKSVCLFTDFDSQTNVSQSKDSDVYITDKCVLD |
| | T48C mutation | MRSMDFKSNSAVAWSNKSDFACANAFNNSIIPEDTFFPSPESSCDVKLVEKS |
| | | FETDTNLNFQNLSVIGFRILLLKVAGFNLLMTLRLWSS* |
| 212 | Human TRBC2 with | EDLKNVFPPEVAVFEPSEAEISHTQKATLVCLATGFYPDHVELSWWVNGKE |
| | S57C mutation | VHSGVCTDPQPLKEQPALNDSRYCLSSRLRVSATFWQNPRNHFRCQVQFYG |
| | | LSENDEWTQDRAKPVTQIVSAEAWGRADCGFTSESYQQGVLSATILYEILLG |
| | | KATLYAVLVSALVLMAMVKRKDSRG |
| 213 | TCRα variable region | GCACAGACGGTCACCCAGAGCCAGCCGGAGATGTCCGTGCAGGAGGCC |
| | codon optimized | GAGACCGTGACTCTTTCATGCACTTACGACACCTCCGAGAACAACTACT |
| | nucleotide sequence 1 | ACCTCTTTTGGTACAAGCAACCTCCCTCTCGGCAGATGATCCTGGTGATC |
| | A0358 | CGTCAGGAGGCTTATAAACAGCAGAACGCGACAGAAAACCGCTTCTCGG |
| | | TCAATTTCCAGAAGGCTGCCAAGTCCTTTTCTTTGAAAATTAGTGACAGC |
| | | CAGCTGGGAGATACGGCCATGTATTTCTGTGCGTTCATGGGGCCCGACTC |
| | | CGGCACCTACAAGTACATCTTCGGTACCGGCACCCGCCTGAAGGTGCTG |
| | | GCC |
| 214 | TCRβ variable region | GACGCGGACGTGACCCAGACACCCCGCAACCGCATCACCAAGACCGGC |
| | codon optimized | AAGCGTATCATGCTTGAGTGCTCTCAAACTAAGGGCCACGATCGAATGT |
| | nucleotide sequence 1 | ATTGGTACAGGCAGGACCCGGGTCTGGGTCTCCAGCTGATTTACTACTCC |
| | A0358 | TTCGACGTGAAGGACATTAATAAGGGAGAGATCTCGGACGGCTATTCCG |
| | | TGTCCCGCCAGGCTCAGGCAAAATTTTCATTGAGCCTGGAGAGCGCCAT |
| | | CCCTAACCAGACTGCTCTGTACTTCTGTGCCACCAGCGATTCTGATCGGA |
| | | TCTACGGCTACACCTTCGGCTCCGGGACCCGCCTGACCGTGGTT |
| 215 | TCRα variable region | GCTCAAACAGTGACCCAGAGCCAGCCCGAGATGAGCGTGCAGGAAGCT |
| | codon optimized | GAAACCGTCACCCTGTCTTGTACCTACGACACCAGCGAGAACAACTACT |
| | nucleotide sequence 2 | ACCTGTTTTGGTATAAGCAGCCACCTAGCAGACAGATGATCCTGGTGAT |
| | A0358 | CCGGCAGGAGGCCTACAAACAGCAGAACGCCACAGAGAATAGATTCTCT |
| | | GTGAACTTCCAGAAGGCCGCCAAGTCCTTCAGCCTGAAGATCAGCGACA |
| | | GCCAACTGGGCGACACCGCCATGTACTTCTGCGCCTTTATGGGACCTGAT |
| | | TCCGGCACATACAAGTACATCTTCGGCACAGGCACCAGACTGAAAGTGC |
| | | TGGCC |
| 216 | TCRβ variable region | GATGCCGACGTGACCCAGACCCCTAGAAATAGAATTACAAAGACCGGCA |
| | codon optimized | AGCGGATCATGCTGGAATGTAGCCAGACCAAAGGCCACGACCGGATGTA |
| | nucleotide sequence 2 | CTGGTACCGGCAGGACCCCGGACTGGGCCTCCAGCTGATCTACTACTCTT |
| | A0358 | TTGATGTCAAGGACATCAACAAGGGCGAGATCAGCGACGGCTACTCCGT |
| | | GTCCAGACAAGCTCAGGCCAAGTTCAGCCTGTCTCTGGAGAGCGCCATC |
| | | CCTAACCAGACCGCCCTGTACTTCTGCGCCACCAGCGACAGCGATAGAA |
| | | TCTACGGCTATACATTCGGCAGCGGAACAAGACTGACCGTGGTG |
| 217 | TCRα variable region | GCCCAGAAAATCACACAGACCCAGCCCGGCATGTTCGTGCAGGAGAAG |
| | codon optimized | GAAGCCGTGACCCTGGACTGTACCTACGACACCAGCGACCAGAGCTACG |
| | nucleotide sequence | GCCTGTTTTGGTACAAACAGCCTAGCAGCGGCGAGATGATCTTCCTGATC |
| | A0359 | TACCAAGGATCTTATGATGAGCAGAACGCCACAGAGGGAAGATACAGC |
| | | CTGAACTTCCAGAAGGCCAGAAAGTCCGCTAATCTGGTGATCAGCGCTT |
| | | CTCAGCTGGGCGACTCCGCCATGTACTTCTGCGCCATGCGGGAACCTGAT |
| | | AGCAACTACCAACTGATCTGGGGCGCCGGCACCAAGCTCATTATCAAGC |
| | | CA |
| 218 | TCRβ variable region | GAGCCTGAGGTGACCCAGACCCCTAGCCACCAGGTGACCCAAATGGGCC |
| | codon optimized | AGGAGGTCATCCTCAGATGTGTGCCCATCAGCAACCACCTGTACTTTTAC |
| | nucleotide sequence | TGGTATAGACAGATCCTGGGCCAGAAAGTGGAATTCCTGGTGTCCTTCT |
| | A0359 | ACAACAACGAGATTAGCGAGAAGTCCGAGATCTTCGACGACCAGTTCAG |
| | | CGTGGAACGGCCTGACGGATCTAATTTCACCCTGAAGATCCGGAGCACA |
| | | AAGCTGGAAGATAGCGCCATGTACTTCTGCGCCTCTCAGAAGGGCCTGG |
| | | AATACGAGCAGTACTTTGGCCCCGGCACCAGACTGACAGTGACA |
| 219 | TCRα variable region | GCCCAGACCGTCACCCAGTCCCAGCCTGAGATGAGCGTGCAGGAGGCCG |
| | codon optimized | AGACAGTGACCCTGAGCTGTACCTACGACACATCTGAAAACAACTACTA |
| | nucleotide sequence 2 | TCTCTTCTGGTACAAACAACCTCCCAGCCGGCAGATGATCCTGGTGATCA |
| | A0130 | GACAAGAAGCCTACAAGCAGCAGAACGCCACAGAGAATAGATTCTCCG |
| | | TGAACTTCCAGAAAGCCGCTAAGAGCTTTAGCCTGAAGATCTCTGATAG |
| | | CCAGCTGGGCGACACCGCCATGTACTTCTGCGCCTTCATGCTGATCGACA |
| | | GCGGCACCTACAAGTACATCTTTGGAACCGGCACAAGACTGAAGGTGCT |
| | | GGCT |
| 220 | TCRβ variable region | GAAACCGGCGTGACCCAGACCCCTAGACACCTGGTCATGGGCATGACCA |
| | codon optimized | ACAAAAAGTCCCTGAAGTGCGAGCAGCACCTGGGCCACAACGCCATGTA |
| | nucleotide sequence 2 | CTGGTATAAGCAGAGCGCCAAGAAACCACTGGAACTGATGTTCGTGTAC |
| | A0130 | AACTTCAAGGAACAAACAGAGAACAACAGCGTGCCCAGCCGGTTCAGC |
| | | CCCGAGTGTCCTAATAGCTCCCACCTGTTCCTGCACCTCCATACACTGCA |
| | | GCCTGAGGACAGCGCTCTGTACCTGTGCGCCTCTAGCCAGGTGGGCACA |
| | | AGCGGCAGAGGCGGAGAGCTGTTTTTCGGCGAGGGATCTAGACTGACCG |
| | | TGCTG |
| 221 | TCRα variable region | GCCCAGACCGTCACCCAGTCCCAGCCTGAGATGAGCGTGCAGGAGGCCG |
| | codon optimized | AGACAGTGACCCTGAGCTGTACCTACGACACATCTGAAAACAACTACTA |
| | nucleotide sequence 2 | TCTCTTCTGGTACAAACAACCTCCCAGCCGGCAGATGATCCTGGTGATCA |
| | A0131 | GACAAGAAGCCTACAAGCAGCAGAACGCCACAGAGAATAGATTCTCCG |
| | | TGAACTTCCAGAAAGCCGCTAAGAGCTTTAGCCTGAAGATCTCTGATAG |
| | | CCAGCTGGGCGACACCGCCATGTACTTCTGCGCCTTCATGCTGATCGACA |
| | | GCGGCACCTACAAGTACATCTTTGGAACCGGCACAAGACTGAAGGTGCT |
| | | GGCT |
| 222 | TCRβ variable region | GAGGCCCAGGTGACCCAAAATCCTAGATACCTGATCACCGTCACAGGCA |
| | codon optimized | AGAAACTGACCGTGACATGTAGCCAGAACATGAACCACGAGTACATGA |
| | nucleotide sequence 2 | GCTGGTATAGACAGGACCCCGGCCTGGGACTGCGGCAGATCTACTACAG |
| | A0131 | CATGAACGTGGAAGTGACCGATAAGGGCGACGTGCCAGAGGGCTACAA |
| | | GGTGTCCAGAAAGGAAAAGCGGAACTTCCCTCTGATCCTGGAATCTCCT |
| | | AGCCCCAACCAGACCAGCCTCTACTTCTGCGCCTCTAGCCTGGGCCAGG |
| | | GCACCGAGACACAGTACTTTGGCCCTGGAACCAGACTGCTGGTGCTG |
| 223 | Human TRAC | AATATCCAGAATCCTGATCCTGCTGTGTACCAGCTGAGAGATTCTAAAA |
| | nucleotide sequence | GCAGCGACAAATCTGTGTGCCTGTTCACCGACTTCGACAGCCAAACAAA |
| | with T48C mutation | CGTGTCCCAGAGCAAGGACAGCGACGTGTACATCACCGACAAGTGCGTG |
| | | CTGGACATGAGAAGCATGGACTTCAAGAGCAACAGCGCCGTCGCTTGGT |
| | | CCAACAAGTCTGATTTTGCCTGCGCCAACGCCTTCAACAACAGCATCATT |
| | | CCAGAGGACACCTTCTTCCCCAGCCCTGAAAGCTCTTGTGATGTGAAGCT |
| | | GGTGGAAAAGTCCTTCGAGACAGATACAAATCTGAACTTCCAGAACCTG |
| | | AGCGTGATCGGCTTTAGAATCCTGCTGCTCAAGGTGGCCGGCTTCAACCT |
| | | GCTGATGACCCTGCGGCTGTGGAGCAGCTGA |
| 224 | Human TRBC2 | GAAGATCTGAAAAACGTGTTTCCACCTGAGGTGGCCGTGTTCGAGCCTT |
| | nucloetide sequence | CCGAGGCCGAGATCAGCCACACCCAGAAGGCCACACTGGTGTGTCTGGC |
| | with S57C mutation | CACAGGCTTTTACCCCGACCACGTGGAACTGAGCTGGTGGGTTAACGGC |
| | | AAAGAAGTGCATTCTGGAGTGTGCACCGACCCCCAGCCTCTGAAGGAAC |
| | | AGCCTGCCCTGAATGATAGCAGATACTGCCTGAGCAGCCGCCTGAGAGT |
| | | GTCCGCCACCTTCTGGCAGAACCCCAGAAACCACTTCCGGTGCCAGGTG |
| | | CAATTCTACGGCCTGAGCGAGAACGACGAGTGGACCCAAGATAGAGCTA |
| | | AGCCTGTGACCCAGATCGTGTCTGCTGAAGCCTGGGGAAGAGCCGACTG |
| | | CGGCTTCACCAGCGAGAGCTACCAGCAGGGCGTGCTGAGCGCTACAATC |
| | | CTGTACGAGATCCTGCTGGGCAAGGCCACCCTGTATGCCGTGCTGGTGTC |
| | | TGCCCTGGTCCTCATGGCCATGGTGAAGCGGAAGGACAGCCGGGGC |
| 225 | FR1α A0001, A0002, | AQSVSQHNHHVILSEAASLELGCNYS |
| | A0003, A0004 | |
| 226 | FR2α A0001, A0002, | LFWYVQYPGQHLQLLLK |
| | A0003, A0004 | |
| 227 | FR3α A0001, A0002, | KGIKGFEAEFIKSKFSFNLRKPSVQWSDTAEYF |
| | A0003, A0004 | |
| 228 | FR4α A0001, A0002, | GTGTRLQVFP |
| | A0004 | |
| 229 | FR4α A0003 | GGGTRVLVKP |
| 230 | FR1α A0005, A0539 | AQKITQTQPGMFVQEKEAVTLDCTYD |
| 231 | FR2α A0005, A0359 | LFWYKQPSSGEMIFLIY |
| 232 | FR3α A0005, A0359 | ATEGRYSLNFQKARKSANLVISASQLGDSAMYF |
| 233 | FR4α A0005 | GSGTKLNVKP |
| 234 | FR1α A0015 | AQSVTQLDSQVPVFEEAPVELRCNYS |
| 235 | FR2α A0015 | LFWYVQYPNQGLQLLLK |
| 236 | FR3α A0015 | KGINGFEAEFNKSQTSFHLRKPSVHISDTAEYF |
| 237 | FR4α A0015 | GQGTELSVKP |
| 238 | FR1α A0061, A0100 | KQEVTQIPAALSVPEGENLVLNCSFT |
| 239 | FR2α A0061, A0100 | LQWFRQDPGKGLTSLLL |
| 240 | FR3α A0061, A0100 | QTSGRLNASLDKSSGRSTLYIAASQPGDSATYL |
| 241 | FR4α A0061, A0130, | GAGTKLIIKP |
| | A0132, A0359 | |
| 242 | FR1α A0062, A0068, | SQQGEEDPQALSIQEGENATMNCSYK |
| | A0069, A0070 | |
| 243 | FR2α A0062, A0068, | LQWYRQNSGRGLVHLIL |
| | A0069, A0070 | |
| 244 | FR3α A0062, A0068, | KHSGRLRVTLDTSKKSSSLLITASRAADTASYF |
| | A0069, A0070 | |
| 245 | FR4α A0062, A0068, | GKGTMLLVSP |
| | A0069, A0070 | |
| 246 | FR1α A0064, A0065, | GQQVMQIPQYQHVQEGEDFTTYCNSS |
| | A0066 | |
| 247 | FR2α A0064, A0065, | IQWYKQRPGGHPVFLIQ |
| | A0066 | |
| 248 | FR3α A0064, A0065, | KKQKRLTFQFGEAKKNSSLHITATQTTDVGTYF |
| | A0066 | |
| 249 | FR4α A0064, A0065, | GKGTKLSVIP |
| | A0066 | |
| 250 | FR1α A0099 | ILNVEQSPQSLHVQEGDSTNFTCSFP |
| 251 | FR2α A0099 | LHWYRWETAKSPEALFV |
| 252 | FR3α A0099 | KKKGRISATLNTKEGYSYLYIKGSQPEDSATYL |
| 253 | FR4α A0099 | GQGTILTVHP |
| 254 | FR1α A0130, A0131, | AQTVTQSQPEMSVQEAETVTLSCTYD |
| | A0132, A0358 | |
| 255 | FR2α A0130, A0131, | LFWYKQPPSRQMILVIR |
| | A0132, A0358 | |
| 256 | FR3α A0130, A0131, | ATENRFSVNFQKAAKSFSLKISDSQLGDTAMYF |
| | A0132, A0358 | |
| 257 | FR4α A0131, A0358 | GTGTRLKVLA |
| 258 | FR4α A0100 | GAGTRLTVKP |
| 259 | FR1β A0002 | DTAVSQTPKYLVTQMGNDKSIKCEQN |
| 260 | FR2β A0002 | MYWYKQDSKKFLKIMFS |
| 261 | FR3β A0002 | IINETVPNRFSPKSPDKAHLNLHINSLELGDSAVYF |
| 262 | FR3β A0002, A0004, | GPGTRLTVL |
| | A0005, A0099 | |
| 263 | FR1β A0003, A0099 | EAGVAQSPRYKIIEKRQSVAFWCNPI |
| 264 | FR2β A0003, A0099 | LYWYQQILGQGPKLLIQ |
| 265 | FR3β A0003, A0099 | VDDSQLPKDRFSAERLKGVDSTLKIQPAKLEDSAVYL |
| 266 | FR4β A0003 | GAGSRLTVL |
| 267 | FR1β A0001, A0004, | DADVTQTPRNRITKTGKRIMLECSQT |
| | A0358 | |
| 268 | FR2β A0001, A0004 | MYWYRQDPGLGLRLIYY |
| 269 | FR3β A0001, A0004, | INKGEISDGYSVSRQAQAKFSLSLESAIPNQTALYF |
| | A0358 | |
| 270 | FR1β A0005 | SAVISQKPSRDICQRGTSLTIQCQVD |
| 271 | FR2β A0005 | MFWYRQQPGQSLTLIAT |
| 272 | FR3β A0005 | TYESGFVIDKFPISRPNLTFSTLTVSNMSPEDSSIYL |
| 273 | FR1β A0015 | DAGVIQSPRHEVTEMGQEVTLRCKPI |
| 274 | FR2β A0015 | LFWYRQTMMRGLELLIY |
| 275 | FR3β A0015 | IDDSGMPEDRFSAKMPNASFSTLKIQPSEPRDSAVYF |
| 276 | FR4β A0015, A0064, | GPGTRLTVT |
| | A0065, A0066, A0359 | |
| 277 | FR1β A0061 | DAGITQSPRYKITETGRQVTLMCHQT |
| 278 | FR2β A0061 | MFWYRQDLGHGLRLIYY |
| 279 | FR3β A0061 | TDKGEVPDGYVVSRSKTENFPLTLESATRSQTSVYF |
| 280 | FR4β A0061, A0070, | GQGTRLTVV |
| | A0100 | |
| 281 | FR1β A0062, A0068, | NAGVTQTPKFQVLKTGQSMTLQCAQD |
| | A0069, A0070 | |
| 282 | FR2β A0062, A0068, | MSWYRQDPGMGLRLIHY |
| | A0069, A0070 | |
| 283 | FR3β A0062, A0068, | TDQGEVPNGYNVSRSTTEDFPLRLLSAAPSQTSVYF |
| | A0069, A0070 | |
| 284 | FR4β A0062, A0068, | GSGTRLTVV |
| | A0069, A0358 | |
| 285 | FR1β A0064, A0065, | KAGVTQTPRYLIKTRGQQVTLSCSPI |
| | A0066, A0132 | |
| 286 | FR2β A0064, A0065, | VSWYQQTPGQGLQFLFE |
| | A0066, A0132 | |
| 287 | FR3β A0064, A0065, | RNKGNFPGRFSGRQFSNSRSEMNVSTLELGDSALYL |
| | A0066, A0132 | |
| 288 | FR1β A0130 | ETGVTQTPRHLVMGMTNKKSLKCEQH |
| 289 | FR2β A0130 | MYWYKQSAKKPLELMFV |
| 290 | FR3β A0130 | TENNSVPSRFSPECPNSSHLFLHLHTLQPEDSALYL |
| 291 | FR4β A0001, A0130 | GEGSRLTVL |
| 292 | FR1β A0131 | EAQVTQNPRYLITVTGKKLTVTCSQN |
| 293 | FR2β A0131 | MSWYRQDPGLGLRQIYY |
| 294 | FR3β A0131 | TDKGDVPEGYKVSRKEKRNFPLILESPSPNQTSLYF |
| 295 | FR4β A0131 | GPGTRLLVL |
| 296 | FR1β A0100 | AAGVIQSPRHLIKEKRETATLKCYPI |
| 297 | FR2β A0100 | VYWYQQGPGQDPQFLIS |
| 298 | FR3β A0100 | SDKGSIPDRFSAQQFSDYHSELNMSSLELGDSALYF |
| 299 | FR4β A0132 | GNGTRLTVT |
| 300 | FR2β A0001, A0358 | MYWYRQDPGLGLQLIYY |
| 301 | FR1β A0359 | EPEVTQTPSHQVTQMGQEVILRCVPI |
| 302 | FR2β A0359 | FYWYRQILGQKVEFLVS |
| 303 | FR3β A0359 | SEKSEIFDDQFSVERPDGSNFTLKIRSTKLEDSAMYF |
| 304 | CDR3α consensus | C-A-F-M-X¹-X²-D-S-X³-X⁴-Y-X⁵-X⁶-I-X⁷ |
| | sequence v2 for binding | whereby X¹ is L or I or E or G, or any of the following |
| | to HLA-A*02-restricted | amino acids with related properties: V or D. X² is P or I |
| | MAPK8IP2-derived | or A, or any of the following amino acids with related |
| | peptide of SEQ ID | properties: V, L or G. X³ is G or N, or any of the |
| | NO: 147 | following amino acids with related properties: Q, A, C or |
| | | S. X⁴ is T or no AA at this position, or S as an amino |
| | | acid with related properties. X⁵ is K or Q, or any of the |
| | | following amino acids with related properties: R, H or N. |
| | | X⁶ is L or Y, or any of the following amino acids with |
| | | related properties: I, V, F, W or H. X⁷ is F or W. |
| 305 | CDR3α consensus | C-A-F-M-X¹-X²-D-S-N-Y-Q-L-I-W |
| | sequence v3 for binding | whereby X¹ is I or E, or any of the following amino acids |
| | to HLA-A*02-restricted | with related properties: V or D. X² is P or A, or any of the |
| | MAPK8IP2-derived | following amino acids with related properties: V, L or G. |
| | peptide of SEQ ID | |
| | NO: 147 | |
| 306 | CDR3α consensus | C-A-X¹_X²_X³-X⁴-D-S-N-Y-Q-L-I-W |
| | sequence v4 for binding | whereby X¹ is F or M, or any of the following amino acids |
| | to HLA-A*02-restricted | with related properties: Y or W. X² is M or R, or any of the |
| | MAPK8IP2-derived | following amino acids with related properties: K or H. |
| | peptide of SEQ ID | X³ is I or E, or any of the following amino acids with |
| | NO: 147 | related properties: V, L or D. X⁴ is P or A, or G as an |
| | | amino acid with related properties. |
| 307 | TCRα variable region | GCCCAGTCTGTGAGCCAGCATAACCACCACGTAATTCTCTCTGAAGCAG |
| | nucleotide sequence | CCTCACTGGAGTTGGGATGCAACTATTCCTATGGTGGAACTGTTAATCTC |
| | A0001 | TTCTGGTATGTCCAGTACCCTGGTCAACACCTTCAGCTTCTCCTCAAGTA |
| | | CTTTTCAGGGGATCCACTGGTTAAAGGCATCAAGGGCTTTGAGGCTGAA |
| | | TTTATAAAGAGTAAATTCTCCTTTAATCTGAGGAAACCCTCTGTGCAGTG |
| | | GAGTGACACAGCTGAGTACTTCTGTGCCGTGAAAGACACCGACAAGCTC |
| | | ATCTTTGGGACTGGGACCAGATTACAAGTCTTTCCAA |
| 308 | TCRβ variable region | GATGCTGATGTTACCCAGACCCCAAGGAATAGGATCACAAAGACAGGA |
| | nucleotide sequence | AAGAGGATTATGCTGGAATGTTCTCAGACTAAGGGTCATGATAGAATGT |
| | A0001 | ACTGGTATCGACAAGACCCAGGACTGGGCCTACGGTTGATCTATTACTC |
| | | CTTTGATGTCAAAGATATAAACAAAGGAGAGATCTCTGATGGATACAGT |
| | | GTCTCTCGACAGGCACAGGCTAAATTCTCCCTGTCCCTAGAGTCTGCCAT |
| | | CCCCAACCAGACAGCTCTTTACTTCTGTGCCACCAGTGATTGGGACGACA |
| | | GCACCGGGGAGCTGTTTTTTGGAGAAGGCTCTAGGCTGACCGTACTGG |

### R. Peptide:MHC Complexes

It will be appreciated that TCRs according to the present disclosure bind to peptide:MHC polypeptide complexes. In some embodiments, a TCR according to the present disclosure binds to one or more of the following:
(i) a peptide:MHC complex comprising an MHC class I α chain polypeptide encoded by a HLA-A*02 allele (e.g. HLA-A*02:01), and a peptide comprising or consisting of SEQ ID NO:105;
(ii) a peptide:MHC complex comprising an MHC class I α chain polypeptide encoded by a HLA-A*02 allele (e.g. HLA-A*02:01), and a peptide comprising or consisting of SEQ ID NO:106;
(iii) a peptide:MHC complex comprising an MHC class I α chain polypeptide encoded by a HLA-A*02 allele (e.g. HLA-A*02:01), and a peptide comprising or consisting of SEQ ID NO:107;
(iv) a peptide:MHC complex comprising an MHC class I α chain polypeptide encoded by a HLA-B*35 allele (e.g. HLA-B*35:01), and a peptide comprising or consisting of SEQ ID NO:145;
(v) a peptide:MHC complex comprising an MHC class I α chain polypeptide encoded by a HLA-A*02 allele (e.g. HLA-A*02:01), and a peptide comprising or consisting of SEQ ID NO:146;
(vi) a peptide:MHC complex comprising an MHC class I α chain polypeptide encoded by a HLA-A*02 allele (e.g. HLA-A*02:01), and a peptide comprising or consisting of SEQ ID NO:147;
(vi) a peptide:MHC complex comprising an MHC class I α chain polypeptide encoded by a HLA-A*02 allele (e.g. HLA-A*02:01), and a peptide comprising or consisting of SEQ ID NO:148.

### S. Nucleic Acids and Vectors

The present disclosure provides nucleic acids, and pluralities of nucleic acids, encoding the TCRs, antigen-binding molecules, polypeptides and polypeptide complexes according to the present disclosure. In some embodiments, the nucleic acid(s) comprise or consist of DNA and/or RNA. In some embodiments, the nucleic acid is a polynucleotide, e.g. a polydeoxyribonucleotide or a polyribonucleotide.

A TCR, antigen-binding molecule or polypeptide according to the present disclosure may be produced within a cell by translation of RNA encoding the relevant polypeptide(s). A TCR, antigen-binding molecule or polypeptide according to the present disclosure may be produced within a cell by transcription from nucleic acid(s) encoding the relevant polypeptide(s), and subsequent translation of the transcribed RNA. Constituent polypeptides of a TCR or antigen-binding molecule according to the present disclosure may be encoded by different nucleic acids of the plurality of nucleic acids, or by different vectors of the plurality of vectors.

In some embodiments, the nucleic acid(s) may be, or may be comprised/contained in, a vector, or a plurality of vectors. As referred to herein, a ‘vector’ may be a nucleic acid molecule used as a vehicle to transfer exogenous nucleic acid into a cell.

Accordingly, the present disclosure also provides a vector, or plurality of vectors, comprising the nucleic acid or plurality of nucleic acids according to the present disclosure. The vector may facilitate delivery of the nucleic acid(s) encoding a polypeptide according to the present disclosure to a cell. The vector may be an expression vector comprising elements required for expressing a polypeptide according to the present disclosure. The vector may comprise elements facilitating integration of the nucleic acid(s) into the genomic DNA of cell into which the vector is introduced.

Nucleic acids and vectors according to the present disclosure may be provided in purified or isolated form, i.e. from other nucleic acid, or naturally-occurring biological material.

A vector may be a vector for expression of the nucleic acid in the cell (i.e. an expression vector). Such vectors may include a promoter sequence operably linked to a nucleotide sequence encoding a TCR/antigen-binding molecule/polypeptide according to the present disclosure. A vector may also include a termination codon (i.e. 3′ in the nucleotide sequence of the vector to the nucleotide sequence encoding the polypeptide(s)) and expression enhancers. Any suitable vectors, promoters, enhancers and termination codons known in the art may be used to express a peptide or polypeptide from a vector according to the present disclosure.

The term ‘operably linked’ may include the situation where nucleic acid encoding a polypeptide according to the present disclosure and regulatory nucleotide sequence(s) (e.g. a promoter and/or enhancers) are covalently linked in such a way as to place the expression of the nucleic acid encoding a polypeptide under the influence or control of the regulatory nucleotide sequence(s) (thereby forming an expression cassette). Thus, a regulatory sequence is operably linked to the selected nucleotide sequence if the regulatory sequence is capable of effecting transcription of the nucleotide sequence. The resulting transcript(s) may then be translated into the desired polypeptide(s).

Vectors contemplated in connection with the present disclosure include DNA vectors, RNA vectors, plasmids (e.g. conjugative plasmids (e.g. F plasmids), non-conjugative plasmids, R plasmids, col plasmids, episomes), viral vectors (e.g. retroviral vectors, e.g. gammaretroviral vectors (e.g. murine Leukemia virus (MLV)-derived vectors, e.g. SFG vector), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 and Morgan and Boyerinas, Biomedicines (2016) 4:9, which are both hereby incorporated by reference in their entirety. In some embodiments, a vector according to the present disclosure is a lentiviral vector.

In some embodiments, a vector is selected based on tropism for a cell type/tissue/organ to which it is desired to deliver the nucleic acid. In some embodiments, a vector is selected based on tropism for a cell type in which it is desired to express the TCR/antigen-binding molecule/polypeptide(s). For example, it may be desired to deliver the nucleic acid/express the TCR/antigen-binding molecule/polypeptide(s) in an immune cell, e.g. a T cell.

In some embodiments, the nucleic acid is a vector suitable for delivering the nucleic acid encoding the antigen-binding-molecule/TCR as a gene therapy. In some embodiments, the vector is an adeno-associated virus (AAV) vector. Adeno-associated virus vectors and their use to vector gene therapy is reviewed e.g. in Wang et al., Nat. Rev. Drug Discov. (2019) 18:358-378 and Li and Samulski, Nat. Rev. Genet. (2020) 12:255-272, both of which are hereby incorporated by reference in their entirety. In some embodiments, a vector may be an adeno-associated virus vector described in Wang et al., Nat. Rev. Drug Discov. (2019) 18:358-378. In some embodiments, a vector may be an adeno-associated virus vector described in Li and Samulski, Nat. Rev. Genet. (2020) 12:255-272.

In some embodiments, a vector may be an adeno-associated viral vector of one of the following serotypes: AAV1, AAV2, AAV218, AAV5, AAV6, AAV8, AAV9, AAV9.45, AAV10 or AAVrh74.

In some embodiments, the vector may be a eukaryotic vector, i.e. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

In some embodiments a vector comprises modification to increase binding to and/or transduction of a cell-type of interest (i.e. as compared to the level of binding/transduction by the unmodified vector). In some embodiments modification is to a capsid protein.

In some embodiments a vector comprises a capsid protein comprising a cell-targeting peptide. In some embodiments the cell-targeting peptide is a cell-targeting peptide described in Büning and Srivastava, Molecular Therapy: Methods & Clinical Development (2019) 12:248-265, which is hereby incorporated by reference in its entirety, e.g. a cell-targeting peptide shown in Table 1, 2, 3 or 4 thereof.

In some embodiments a vector comprises a capsid protein comprising substitution to one or more tyrosine residues, e.g. one or more surface-exposed tyrosine residues. In some embodiments, one or more tyrosine residues of the capsid protein are substituted with phenylalanine. In some embodiments a vector comprises a capsid protein in which one or more tyrosine residues are substituted with another amino acid as described in Iida et al., Biomed Res Int. (2013) 2013:974819, which is hereby incorporated by reference in its entirety.

In some embodiments, a vector may be an adeno-associated virus vector described in Büning and Srivastava, supra. In some embodiments, a vector may be an adeno-associated virus vector described in lida et al., supra.

In some embodiments the nucleic acid/vector comprises one or more sequences for controlling expression of the nucleic acid. Accordingly, in some embodiments the nucleic acid/vector comprises a control element for inducible expression of the nucleic acid.

A sequence for controlling expression of the nucleic acid may provide for expression of the nucleic acid by cells of a particular type or tissue. For example, expression may be under the control of a cell type- or tissue-specific promoter.

Promoters for cell type- or tissue-specific expression of a nucleic acid in accordance with the present invention can be selected in accordance with the disease to be treated/prevented. For example, the promoter may drive expression in an immune cell.

A sequence for controlling expression of the nucleic acid may provide for expression of the nucleic acid in response to e.g. a given agent/signal. For example, expression may be under the control of inducible promoter. The agent may provide for inducible expression of the nucleic acid in vivo by administration of the agent to a subject having been administered with a modified cell according to the disclosure, or ex vivo/in vitro by administration of the agent to cells in culture ex vivo or in vitro.

In some embodiments a nucleic acid or vector according to the present disclosure may employ a conditional expression system for controlling expression of the nucleic acid encoding the antigen-binding-molecule/TCR by cells comprising the nucleic acid/vector. ‘Conditional expression’ may also be referred to herein as ‘inducible expression’, and refers to expression contingent on certain conditions, e.g. the presence of a particular agent. Conditional expression systems are well known in the art and are reviewed e.g. in Ryding et al. Journal of Endocrinology (2001) 171, 1-14, which is hereby incorporated by reference in its entirety.

### T. Cells

The present disclosure provides cells comprising/expressing T cell receptors (TCRs). TCR-expressing cells may express or comprise a TCR according to the present disclosure. TCR-expressing cells may comprise or express nucleic acid encoding a TCR according to the present disclosure. It will be appreciated that a TCR-expressing cell comprises the TCR it expresses. It will also be appreciated that a cell expressing nucleic acid encoding a TCR also expresses and comprises the TCR encoded by the nucleic acid.

Aspects and embodiments of the present disclosure relate to host cells, and in particular immune cells. It will be appreciated that where cells are referred to herein in the singular (i.e. ‘a/the cell’), pluralities/populations of such cells are also contemplated.

In aspects and embodiments of the present disclosure, the cells are primary cells. That is, in some embodiments, the cells are/were isolated directly from living tissue/a living subject. The cells may be from any animal or human. The cells may be mammalian, more preferably human. The cells may be from a human patient.

In preferred embodiments, the host cell is an immune cell. An ‘immune cell’ may be a cell of hematopoietic origin, e.g. a neutrophil, eosinophil, basophil, dendritic cell, lymphocyte, or monocyte. A lymphocyte may be e.g. a T cell, B cell, NK cell, NKT cell or innate lymphoid cell (ILC), or a precursor thereof. The host cell/immune cell may express e.g. CD3 polypeptides (e.g. CD3γ CD3ε CD3ζ or CD3δ), TCR polypeptides (TCRα or TCRβ), CD27, CD28, CD4 or CD8. In some embodiments, the host cell/immune cell is a T cell, e.g. a CD3+ T cell. In some embodiments, the T cell is a CD3+, CD4+ T cell. In some embodiments, the T cell is a CD3+, CD8+ T cell. In some embodiments, the T cell is a T helper cell (TH cell). In some embodiments, the T cell is a cytotoxic T cell (e.g. a cytotoxic T lymphocyte (CTL)).

An antigen-specific T cell may display certain functional properties of a T cell in response to the antigen/antigenic peptide for which the T cell is specific, or in response a cell comprising/expressing the antigen/antigenic peptide. In some embodiments, the properties are functional properties associated with effector T cells, e.g. cytotoxic T lymphocytes (CTLs).

In some embodiments, an antigen-specific T cell may display one or more of the following properties: cytotoxicity to a cell comprising/expressing the antigen/peptide thereof for which the T cell is specific; proliferation, IFNγ expression, CD107a expression, IL-2 expression, TNFα expression, perforin expression, granzyme expression, granulysin expression, and/or FAS ligand (FASL) expression in response to stimulation with the antigen/peptide thereof for which the T cell is specific, or in response to exposure to a cell comprising/expressing the antigen/peptide thereof for which the T cell is specific.

Antigen-specific T cells according to the present disclosure express/comprise a TCR capable of recognising a peptide of the antigen for which the T cell is specific when presented by the appropriate MHC molecule. In some embodiments, the antigen-specific immune cell is a T cell, e.g. a CD3+ T cell. In some embodiments, the T cell is a CD3+, CD4+ T cell. In some embodiments, the T cell is a CD3+, CD8+ T cell. In some embodiments, the T cell is a T helper cell (TH cell)). In some embodiments, the T cell is a cytotoxic T cell (e.g. a cytotoxic T lymphocyte (CTL)).

In some embodiments, an antigen-specific immune cell (e.g. an antigen-specific T cell) is specific for an antigen of Epstein-Barr virus. Such cells may be referred to as EBV-specific immune cells. An EBV-specific immune cell expresses/comprises a receptor (preferably a T cell receptor) capable of recognising a peptide of an antigen of EBV (e.g. when presented by an MHC molecule). In some embodiments, the EBV-specific immune cell expresses/comprises a TCR specific for a peptide of an EBV antigen presented by MHC class I.

In some embodiments, an antigen-specific immune cell (e.g. an antigen-specific T cell) is specific for the EBV antigen BRLF1. Such cells may be referred to as BRLF1-specific immune cells. A ‘BRLF1-specific immune cell’ as used herein refers to an immune cell which is specific for BRLF1. A BRLF1-specific immune cell expresses/comprises a receptor (preferably a T cell receptor) capable of recognising a peptide of BRLF1 (e.g. when presented by an MHC molecule). In some embodiments, the BRLF1-specific immune cell expresses/comprises a TCR specific for a peptide of BRLF1 presented by MHC class I.

In some embodiments, an antigen-specific immune cell (e.g. an antigen-specific T cell) is specific for the EBV antigen LMP2. Such cells may be referred to as LMP2-specific immune cells. A LMP2-specific immune cell expresses/comprises a receptor (preferably a T cell receptor) capable of recognising a peptide of LMP2 (e.g. when presented by an MHC molecule). In some embodiments, the LMP2-specific immune cell expresses/comprises a TCR specific for a peptide of LMP2 presented by MHC class I.

In some embodiments, an antigen-specific immune cell (e.g. an antigen-specific T cell) is specific for the EBV antigen BZLF1. Such cells may be referred to as BZLF1-specific immune cells. A BZLF1-specific immune cell expresses/comprises a receptor (preferably a T cell receptor) capable of recognising a peptide of BZLF1 (e.g. when presented by an MHC molecule). In some embodiments, the BZLF1-specific immune cell expresses/comprises a TCR specific for a peptide of BZLF1 presented by MHC class I.

In some embodiments, an antigen-specific immune cell (e.g. an antigen-specific T cell) is specific for a mutant splice factor-induced peptide of MAPK8IP2. Such cells express/comprise a receptor (preferably a T cell receptor) capable of recognising a mutant splice factor-induced peptide of MAPK8IP2 (e.g. when presented by an MHC molecule). In some embodiments, such cells express/comprise a TCR specific for the mutant splice factor-induced peptide of MAPK8IP2 presented by MHC class I.

In some embodiments, an antigen-specific immune cell (e.g. an antigen-specific T cell) is specific for the HERV-K antigen gag. Such cells may be referred to as HERV-K gag-specific immune cells. A HERV-K gag-specific immune cell expresses/comprises a receptor (preferably a T cell receptor) capable of recognising a peptide of HERV-K gag (e.g. when presented by an MHC molecule). In some embodiments, the HERV-K gag-specific immune cell expresses/comprises a TCR specific for a peptide of HERV-K gag presented by MHC class I.

An immune cell comprising a TCR/nucleic acid encoding a TCR according to the present disclosure may be characterised by reference to functional properties of the cells. In some embodiments an immune cell comprising a TCR/nucleic acid encoding a TCR according to the present disclosure displays one or more of the following properties:
(a) expression of one or more cytotoxic/effector factors (e.g. IFNγ, granzyme, perforin, granulysin, CD107a, TNFα, FASL) in response to cells presenting the MHC:peptide complex for which the TCR is specific;
(b) proliferation/population expansion, and/or growth factor (e.g. IL-2, GM-CSF) expression in response to cells presenting the MHC:peptide complex for which the TCR is specific;
(c) cytotoxicity to cells presenting the MHC:peptide complex for which the TCR is specific;
(d) no cytotoxicity (i.e. above baseline) to cells which do not present the MHC:peptide complex for which the TCR is specific; and
(e) anti-cancer activity (e.g. cytotoxicity to cancer cells, tumor growth inhibition, reduction of metastasis, etc.) against cancer comprising cells presenting the MHC:peptide complex for which the TCR is specific.

Cell proliferation/population expansion can be investigated by analysing cell division or the number of cells over a period of time. Cell division can be analysed, for example, by in vitro analysis of incorporation of 3H-thymidine or by CFSE dilution assay, e.g. as described in Fulcher and Wong, Immunol Cell Biol (1999) 77 (6): 559-564, hereby incorporated by reference in its entirety. Proliferating cells can also be identified by analysis of incorporation of 5-ethynyl-2′-deoxyuridine (EdU) by an appropriate assay, as described e.g. in Buck et al., Biotechniques. 2008 June; 44(7):927-9, and Sali and Mitchison, PNAS USA 2008 Feb. 19; 105(7): 2415-2420, both hereby incorporated by reference in their entirety.

As used herein, ‘expression’ may be gene or protein expression. Gene expression encompasses transcription of DNA to RNA, and can be measured by various means known to those skilled in the art, for example by measuring levels of mRNA by quantitative real-time PCR (qRT-PCR), or by reporter-based methods. Similarly, protein expression can be measured by various methods well known in the art, e.g. by antibody-based methods, for example by western blot, immunohistochemistry, immunocytochemistry, flow cytometry, ELISA, ELISPOT, or reporter-based methods.

Cytotoxicity and cell killing can be investigated, for example, using any of the methods reviewed in Zaritskaya et al., Expert Rev Vaccines (2011), 9(6):601-616, hereby incorporated by reference in its entirety. Examples of in vitro assays of cytotoxicity/cell killing assays include release assays such as the ⁵¹Cr release assay, the lactate dehydrogenase (LDH) release assay, the 3-(4,5-dimethylthiazol-2-yl)-2,5-diphenyl tetrazolium bromide (MTT) release assay, and the calcein-acetoxymethyl (calcein-AM) release assay. These assays measure cell killing based on the detection of factors released from lysed cells. Cell killing by a given cell type can be analysed e.g. by co-culturing the test cells with the given cell type, and measuring the number/proportion of cells viable/dead test cells after a suitable period of time. Other suitable assays include the xCELLigence real-time cytolytic in vitro potency assay described in Cerignoli et al., PLOS One. (2018) 13(3):e0193498 (hereby incorporated by reference in its entirety).

Cells may be evaluated for anti-cancer activity by analysis in an appropriate in vitro assays or in vivo models of the relevant cancer.

The present disclosure also provides methods for producing cells/populations of cells according to the present disclosure, and the cells/populations of cells obtained or obtainable by such methods.

Methods for producing cells comprising/expressing a TCR of interest are well known to the skilled person, and generally comprise introducing nucleic acid(s)/vector(s) encoding constituent polypeptide(s) of the TCR into the cells.

Such methods may comprise nucleic acid transfer for permanent (i.e. stable) or transient expression of the transferred nucleic acid. In some embodiments, following introduction into a cell, nucleic acid(s) encoding the polypeptide(s) of the TCR may be integrated into or form part of the genomic DNA of the cell. In some embodiments, following introduction into a cell nucleic acid(s) encoding the polypeptide(s) may be maintained extrachromosomally.

Any suitable genetic engineering platform may be used, and include gammaretroviral vectors, lentiviral vectors, adenovirus vectors, DNA transfection, transposon-based gene delivery and RNA transfection, for example as described in Maus et al., Annu Rev Immunol (2014) 32:189-225, hereby incorporated by reference in its entirety. Methods also include those described e.g. in Wang and Rivière Mol Ther Oncolytics. (2016) 3:16015, which is hereby incorporated by reference in its entirety. Suitable methods for introducing nucleic acid(s)/vector(s) into cells include transduction, transfection and electroporation.

Methods for generating/expanding populations of cells comprising/expressing the TCR in vitro/ex vivo are well known to the skilled person. Suitable culture conditions (i.e. cell culture media, additives, stimulations, temperature, gaseous atmosphere), cell numbers, culture periods and methods for introducing nucleic acid(s)/vector(s) encoding polypeptide(s) of interest into cells, etc. can be determined by reference e.g. to WO 2018/177966 A1. In some embodiments, a cell/population of cells according to the present disclosure is prepared under GMP (good manufacturing practice; e.g. as described in the guidelines for good manufacturing practice published by the European Commission (Volume 4 of ‘The rules governing medicinal products in the European Union’ contains guidance for the interpretation of the principles and guidelines of good manufacturing practices for medicinal products for human and veterinary use laid down in Commission Directives 91/356/EEC, as amended by Directive 2003/94/EC, and 91/412/EEC respectively)) conditions.

Conveniently, cultures of cells according to the present disclosure may be maintained at 37° C. in a humidified atmosphere containing 5% CO₂. The cells of cell cultures can be established and/or maintained at any suitable density, as can readily be determined by the skilled person. Cultures can be performed in any vessel suitable for the volume of the culture, e.g. in wells of a cell culture plate, cell culture flasks, a bioreactor, etc. In some embodiments cells are cultured in a bioreactor, e.g. a bioreactor described in Somerville and Dudley, Oncoimmunology (2012) 1(8):1435-1437, which is hereby incorporated by reference in its entirety.

Introducing nucleic acid(s) into a cell may comprise transduction, e.g. lentiviral transduction. Transduction of immune cells with viral vectors is described e.g. in Simmons and Alberola-Ila, Methods Mol Biol. (2016) 1323:99-108, which is hereby incorporated by reference in its entirety.

Agents may be employed to enhance the efficiency of transduction. Hexadimethrine bromide (polybrene) is a cationic polymer which is commonly used to improve transduction, through neutralising charge repulsion between virions and sialic acid residues expressed on the cell surface. Other agents commonly used to enhance transduction include e.g. the poloxamer-based agents such as LentiBOOST (Sirion Biotech), Retronectin (Takara), Vectofusin (Miltenyi Biotech) and also SureENTRY (Qiagen) and ViraDuctin (Cell Biolabs). In some embodiments the methods comprise centrifuging the cells into which it is desired to introduce nucleic acid encoding polypeptide(s) of the TCR in the presence of cell culture medium comprising viral vector comprising the nucleic acid (referred to in the art as ‘spinfection’).

The methods generally comprise introducing a nucleic acid encoding polypeptide(s) of the TCR into a cell, and culturing the cell under conditions suitable for expression of the polypeptide(s) by the cell. In some embodiments, the methods comprise culturing immune cells into which nucleic acid encoding the polypeptide(s) has been introduced, in order to expand their number.

In some embodiments, the methods comprise analysing the cells to confirm successful introduction of the nucleic acid into the cells. In some embodiments, the methods comprise analysing the cells to confirm expression of the polypeptide(s) by the cells (e.g. via evaluation of a detectable entity).

In some embodiments the methods further comprise separating/isolating/purifying/enriching cells expressing the TCR e.g. from other cells (e.g. cells which do not express the TCR). Methods for purifying/isolating immune cells from heterogeneous populations of cells are well known in the art, and may employ e.g. FACS- or MACS-based methods for sorting populations of cells based on the expression of the TCR/constituent polypeptide(s) thereof. In some embodiments, the methods comprise separating/isolating/purifying/enriching cells of a particular type, e.g. CD8+ T cells or CTLs expressing the TCR of interest.

Methods for producing cells according to the present disclosure may comprise modifying the cells to reduce the expression of a CD3-TCR complex polypeptide. In some embodiments, the methods comprise modifying nucleic acid (e.g. endogenous nucleic acid) encoding the CD3-TCR complex polypeptide.

Modification of a given target nucleic acid can be achieved in a variety of ways known to the skilled person, including modification of the target nucleic acid by homologous recombination, and target nucleic acid editing using site-specific nucleases (SSNs).

Suitable methods may employ targeting by homologous recombination, which is reviewed, for example, in Mortensen Curr Protoc Neurosci. (2007) Chapter 4: Unit 4.29 and Vasquez et al., PNAS 2001, 98(15): 8403-8410 both of which are hereby incorporated by reference in their entirety. Targeting by homologous recombination involves the exchange of nucleotide sequence through crossover events guided by homologous sequences. Other suitable techniques include nucleic acid editing using SSNs. Gene editing using SSNs is reviewed e.g. in Eid and Mahfouz, Exp Mol Med. 2016 October; 48(10):e265, which is hereby incorporated by reference in its entirety. Enzymes capable of creating site-specific double strand breaks (DSBs) can be engineered to introduce DSBs to target nucleotide sequence(s) of interest. DSBs may be repaired by either error-prone non-homologous end-joining (NHEJ), in which the two ends of the break are rejoined, often with insertion or deletion of nucleotides. Alternatively, DSBs may be repaired by homology-directed repair (HDR), a high-fidelity mechanism in which a DNA template with ends homologous to the break site is supplied and introduced at the site of the DSB.

SSNs capable of being engineered to generate target nucleotide sequence-specific DSBs include zinc-finger nucleases (ZFNs), transcription activator-like effector nucleases (TALENs) and clustered regularly interspaced palindromic repeats/CRISPR-associated-9 (CRISPR/Cas9) systems. ZFN systems are reviewed e.g. in Umov et al., Nat Rev Genet. (2010) 11 (9): 636-46, which is hereby incorporated by reference in its entirety. ZFNs comprise a programmable Zinc Finger DNA-binding domain and a DNA-cleaving domain (e.g. a FokI endonuclease domain). The DNA-binding domain may be identified by screening a Zinc Finger array capable of binding to the target nucleotide sequence. TALEN systems are reviewed e.g. in Mahfouz et al., Plant Biotechnol J. (2014) 12(8):1006-14, which is hereby incorporated by reference in its entirety. TALENs comprise a programmable DNA-binding TALE domain and a DNA-cleaving domain (e.g. a FokI endonuclease domain). TALEs comprise repeat domains consisting of repeats of 33-39 amino acids, which are identical except for two residues at positions 12 and 13 of each repeat which are repeat variable di-residues (RVDs). Each RVD determines binding of the repeat to a nucleotide in the target DNA sequence according to the following relationship: ‘HD’ binds to C, ‘NI’ binds to A, ‘NG’ binds to T and ‘NN’ or ‘NK’ binds to G (Moscou and Bogdanove, Science (2009) 326(5959):1501.). CRISPR/Cas9 and related systems e.g. CRISPR/Cpf1, CRISPR/C2c1, CRISPR/C2c2 and CRISPR/C2c3 are reviewed e.g. in Nakade et al., Bioengineered (2017) 8 (3): 265-273, which is hereby incorporated by reference in its entirety. These systems comprise an endonuclease (e.g. Cas9, Cpf1 etc.) and the single-guide RNA (sgRNA) molecule. The sgRNA can be engineered to target endonuclease activity to nucleotide sequences of interest.

In some embodiments, modifying nucleic acid (e.g. endogenous nucleic acid) encoding the CD3-TCR complex polypeptide in accordance with the present disclosure employs a site-specific nuclease (SSN) system targeting nucleic acid encoding the CD3-TCR complex polypeptide. The SSN system may be a ZFN system, a TALEN system, CRISPR/Cas9 system, a CRISPR/Cpf1 system, a CRISPR/C2c1 system, a CRISPR/C2c2 system or a CRISPR/C2c3 system.

In some embodiments, a method for producing a cell according to the present disclosure comprises introducing nucleic acid(s) encoding CRISPR/Cas9 system(s) targeting TRAC, TRBC1 and/or TRBC2 (e.g. TRAC and TRBC1) into a cell. In some embodiments, the nucleic acid(s) encode a CRISPR RNA (crRNA) targeting TRAC, TRBC1 and/or TRBC2 (e.g. TRAC and TRBC1; e.g. an exon of TRAC, TRBC1 and/or TRBC2 (e.g. TRAC and TRBC1)) and a trans-activating crRNA (tracrRNA) for processing the crRNA to its mature form.

### U. Compositions

The present disclosure also provides compositions comprising the TCRs, antigen-binding molecules, polypeptides, nucleic acids, vectors and cells described herein.

The polypeptides, polypeptide complexes, nucleic acids, expression vectors and cells described herein may be formulated as pharmaceutical compositions or medicaments for clinical use, and may comprise a pharmaceutically-acceptable carrier, diluent, excipient or adjuvant. In preferred aspects and embodiments, the present disclosure provides a pharmaceutical composition or medicament comprising a cell according to the present disclosure. Thus, the present disclosure also provides a pharmaceutical composition/medicament comprising a polypeptide, polypeptide complex, nucleic acid/plurality, expression vector/plurality or cell described herein. In preferred embodiments, a pharmaceutical composition/medicament according to the present disclosure comprises a nucleic acid/plurality, expression vector/plurality or cell described herein.

The pharmaceutical compositions/medicaments of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilisers, solubilisers, surfactants (e.g., wetting agents), masking agents or colouring agents (e.g. titanium oxide).

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabiliser, solubiliser, surfactant, masking agent, colouring agent, flavouring agent or sweetening agent of a composition according to the present disclosure must also be ‘acceptable’ in the sense of being compatible with the other ingredients of the formulation. Suitable carriers, diluents, excipients, adjuvants, fillers, buffers, preservatives, anti-oxidants, lubricants, binders, stabilisers, solubilisers, surfactants, masking agents, colouring agents, flavouring agents or sweetening agents can be found in standard pharmaceutical texts, for example, Remington's ‘The Science and Practice of Pharmacy’ (Ed. A. Adejare), 23rd Edition (2020), Academic Press.

Pharmaceutical compositions and medicaments of the present disclosure may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration. In some embodiments, a pharmaceutical composition/medicament may be formulated for administration by injection or infusion, or administration by ingestion.

Suitable formulations may comprise the cell provided in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments, the pharmaceutical compositions/medicament is formulated for injection or infusion, e.g. into a blood vessel, tissue/organ of interest, or a tumor.

The present disclosure also provides methods for the production of pharmaceutically useful compositions, such methods of production may comprise one or more steps selected from:
producing a cell described herein;
isolating/purifying a cell described herein; and/or
mixing a cell described herein with a pharmaceutically-acceptable carrier, adjuvant, excipient or diluent.

For example, a further aspect the present disclosure relates to a method of formulating or producing a medicament or pharmaceutical composition for use in the treatment of a disease/condition (e.g. a disease/condition described herein), the method comprising formulating a pharmaceutical composition or medicament by mixing a cell described herein with a pharmaceutically-acceptable carrier, adjuvant, excipient or diluent.

### V. Kits

The present disclosure also provides kits of parts. Aspects and embodiments of the present disclosure relate to kits for producing a cell (e.g. an antigen-specific cell) according to the present disclosure. Aspects and embodiments of the present disclosure relate to kits for performing the methods according to the present disclosure.

In some embodiments, the kit may have at least one container having a predetermined quantity of a TCR, antigen-binding molecule, polypeptide, nucleic acid, vector, cell or composition described herein. The kit may provide the relevant articles together with instructions (e.g. a protocol) as to how to employ them in accordance with a method described herein.

In some embodiments, a kit of parts comprises materials for producing a polypeptide according to the present disclosure. In some embodiments, a kit of parts comprises materials for producing a TCR/antigen-binding molecule according to the present disclosure. In some embodiments, a kit of parts comprises materials for producing a cell according to the present disclosure. In some embodiments, a kit of parts comprises materials for producing a composition according to the present disclosure.

In some embodiments, the kit of parts may comprise a nucleic acid/plurality or an expression vector/plurality according to the present disclosure, and optionally materials for introducing the nucleic acid/plurality or an expression vector/plurality into a cell.

In some embodiments, the kit may comprise materials for producing a TCR, antigen-binding molecule, polypeptide, nucleic acid, vector, cell or composition described herein. In some embodiments, the kit of parts may comprise materials for formulating a TCR, antigen-binding molecule, polypeptide, nucleic acid, vector, cell or composition described herein to a pharmaceutical composition/medicament, e.g. in a composition further comprising a pharmaceutically-acceptable carrier, diluent, excipient or adjuvant.

The kit may provide a TCR, antigen-binding molecule, polypeptide, nucleic acid, vector, cell or composition described herein together with instructions for administration to a patient in order to treat a specified disease/condition (e.g. a disease/condition described herein).

In some embodiments the kit may further comprise at least one container having a predetermined quantity of another therapeutic agent (e.g. as described herein). In such embodiments, the kit may also comprise a second medicament or pharmaceutical composition such that the two medicaments or pharmaceutical compositions may be administered simultaneously or separately such that they provide a combined treatment for the specific disease/condition.

Kits according to the present disclosure may include instructions for use, e.g. in the form of an instruction booklet or leaflet. The instructions may include a protocol for performing any one or more of the methods described herein.

### W. Subjects

The subject in accordance with aspects of the present disclosure may be any animal or human. The subject is preferably mammalian, more preferably human. The subject may be a non-human mammal, but is more preferably human. The subject may be male or female. The subject may be a patient. A subject may have been diagnosed with a disease or condition described herein requiring treatment (e.g. a cancer), may be suspected of having such a disease/condition, or may be at risk of developing/contracting such a disease/condition.

In embodiments according to the present disclosure, the subject is preferably a human subject. In some embodiments, the subject to be treated according to a therapeutic or prophylactic method of the present disclosure is a subject having, or at risk of developing, a disease/condition described herein. In embodiments according to the present disclosure, a subject may be selected for treatment according to the methods based on characterisation for certain markers of such a disease/condition. In some embodiments, a subject may be infected with a virus (e.g. EBV). In some embodiments, a subject may comprise cells comprising/expressing a peptide described herein. In some embodiments, a subject may comprise cells presenting a peptide:MHC complex described herein.

In some embodiments, a subject comprises a HLA allele as described herein. In some embodiments, a subject comprises a HLA-A*02 allele. In some embodiments, a subject comprises HLA-A*02:01. In some embodiments, a subject comprises HLA-A*02:02, HLA-A*02:03, HLA-A*02:04, HLA-A*02:05, HLA-A*02:06, HLA-A*02:07, HLA-A*02:11, HLA-A*02:12, HLA-A*02:19, HLA-A*02:24, HLA-A*02:264, or HLA-A*02:52. In some embodiments, a subject comprises a HLA-B*35 allele. In some embodiments, a subject comprises HLA-B*35:01.

A subject to be administered immune cells in accordance with the present disclosure may be autogeneic/autologous with respect to the subject from which immune cells administered to the subject are derived. A subject to be administered immune cells in accordance with the present disclosure may be genetically identical to the subject from which immune cells administered to the subject are derived. A subject to be administered immune cells in accordance with the present disclosure may be the same subject as the subject from which immune cells administered to the subject are derived. A subject to be treated/prevented in accordance with the present disclosure may be HLA-matched with respect to the subject from which immune cells administered to the subject are derived. A subject to which cells are administered may comprise MHC/HLA genes encoding MHC/HLA molecules which are identical to the MHC/HLA molecules encoded by the MHC/HLA genes of the subject from which immune cells administered to the subject are derived.

A subject to be administered immune cells in accordance with the present disclosure may be allogeneic/non-autologous with respect to the subject from which immune cells administered to the subject are derived. A subject to be administered immune cells in accordance with the present disclosure may be genetically non-identical to the subject from which immune cells administered to the subject are derived. A subject to be administered immune cells in accordance with the present disclosure may be a different subject to the subject from which immune cells administered to the subject are derived. A subject to be treated/prevented in accordance with the present disclosure may be HLA-mismatched with respect to the subject from which immune cells administered to the subject are derived. A subject to which cells are administered may comprise MHC/HLA genes encoding MHC/HLA molecules which are non-identical to the MHC/HLA molecules encoded by the MHC/HLA genes of the subject from which immune cells administered to the subject are derived.

In some embodiments, the subject is a ≥ 4/8 (i.e. 4/8, ⅝, 6/8, ⅞ or 8/8) match across HLA-A, -B, -C, and -DRB1. In some embodiments, the subject is a ≥ 5/10 (i.e. 5/10, 6/10, 7/10, 8/10, 9/10 or 10/10) match across HLA-A, -B, -C, -DRB1 and -DQB1. In some embodiments, the subject is a ≥ 6/12 (i.e. 6/12, 7/12 8/12, 9/12, 10/12, 11/12 or 12/12) match across HLA-A, -B, -C, -DRB1, -DQB1 and -DPB1. In some embodiments, the subject is an 8/8 match across HLA-A, -B, -C, and -DRB1. In some embodiments, the subject is a 10/10 match across HLA-A, -B, -C, -DRB1 and -DQB1. In some embodiments, the subject is a 12/12 match across HLA-A, -B, -C, -DRB1, -DQB1 and -DPB1.

### W. Numbered Statements

The following numbered paragraphs (paras) describe particular aspects and embodiments of the present disclosure:
1A. An isolated T cell receptor (TCR) comprising a TCRα chain and a TCRβ chain binding to Epstein Barr Virus (EBV)-derived antigenic peptides when presented by a major histocompatibility complex (MHC) molecule, wherein said TCRα chain and said TCRβ chain each comprise one, two or three complementarity determining regions selected from CDR1, CDR2, and CDR3, each respectively comprising an amino acid sequence having at least about 95% sequence identity with an amino acid sequence selected from Table 3A.
2A. The TCR according to para 1A, wherein the TCRα chain and the TCRβ chain CDR1 amino acid sequences share at least about 95% sequence identity with an amino acid sequence selected from SEQ ID NOS: 1; 2; 3; 4; 5; 6; 136; 25; 26; 27; 28; 29; 30; 31; and 32, and combinations thereof, as set forth in Table 3A.
3A. The TCR according to para 1A, wherein the TCRα chain and the TCRβ chain CDR2 amino acid sequences share at least about 95% sequence identity with TCRα chain and TCRβ chain CDR2 amino acid sequences selected from SEQ ID NOS: 7; 8; 9; 10; 11; 12; 13; 137; 33; 34; 35; 36; 37; 38; 39; 40; and 41, and combinations thereof, as set forth in Table 3A.
4A. The TCR according to para 1A, wherein:
the TCRα chain comprises a CDR3 sharing at least about 95% sequence identity with a member selected from SEQ ID NOS: 15; 16; 17; 18; 19; 20; 21; 22; 23; 24; and 138, in combination with:
the TCRβ chain, which comprises a CDR3 sharing at least about 95% sequence identity with a member selected from SEQ ID NOS: 43; 44; 45; 46; 47; 48; 49; 50; 51; 52; 53; 54; and 139, and combinations thereof, as set forth in Table 3A.
5A. The TCR according to para 4A comprising a TCRα chain CDR3 and a TCRβ chain CDR3 of polypeptide SEQ ID pairs selected from the group consisting of:
SEQ ID NOS: 15 and 43; SEQ ID NOS: 16 and 44; SEQ ID NOS: 15 and 45; SEQ ID NOS: 17 and 46; SEQ ID NOS: 18 and 47; SEQ ID NOS: 19 and 48; SEQ ID NOS: 20 and 49; SEQ ID NO:21 and 50; SEQ ID NOS: 22 and 50; SEQ ID NOS: 21 and 51; SEQ ID NOS: 23 and 52; SEQ ID NOS: 23 and 53; SEQ ID NOS: 24 and 54; and SEQ ID NOS: 138 and 139.
6A. The TCR according to any one of the paras 1A-5A, comprising:
a TCRα chain sharing at least about 80%, about 85%, about 90%, or about 95% sequence identity with a member selected from SEQ ID NOS: 55; 56; 57; 58; 59; 60; 61; 62; 63; 64; 65; and 140, in combination with:
a TCRβ chain sharing at least about 80%, about 85%, about 90%, or about 95% sequence identity with a member selected from SEQ ID NOS: 67; 68; 69; 70; 71; 72; 73; 74; 75; 76; 77; 78; and 141, as set forth in Table 4.
7A. The TCR according to para 6A comprising a TCRα chain and a TCRβ chain of polypeptide SEQ ID pairs selected from:
SEQ ID NOS: 55 and 67; SEQ ID NOS: 56 and 68; SEQ ID NOS: 55 and 69; SEQ ID NOS: 57 and 70; SEQ ID NOS: 58 and 71; SEQ ID NOS: 59 and 72; SEQ ID NOS: 60 and 73; SEQ ID NOS: 61 and 74; SEQ ID NOS: 62 and 74; SEQ ID NOS: 61 and 75; SEQ ID NOS: 63 and 76; SEQ ID NOS: 64 and 77; SEQ ID NOS: 65 and 78; SEQ ID NOS: and 140 and 141.
8A. A T cell receptor (TCR) according to any one of paras 1A-7A, comprising:
a TCRα chain encoded by a nucleic acid sharing at least about 80%, about 85%, about 90%, or about 95% sequence identity with a member selected from SEQ ID NOS: 79; 80; 81; 82; 83; 84; 85; 86; 87; 88; 89; 90; 142; 108; 109; 110; 111; 112; 113; 114; 115; 116; 117; 118; 119; 120; and 134, in combination with:
a TCRβ chain encoded by a nucleic acid having at least about 80%, about 85%, about 90%, or about 95% sequence identity with a member selected from SEQ ID NOS: 92; 93; 94; 95; 96; 97; 98; 99; 100; 101; 102; 103; 104; 143; 121; 122; 123; 124; 125; 126; 127; 128; 129; 130; 131; 132; 133; and 135.
9A. The TCR according to para 8A comprising a TCRα chain and TCRβ chain of nucleotide SEQ ID pairs selected from:
SEQ ID NOS: 79 and 92; SEQ ID NOS: 80 and 93; SEQ ID NOS: 79 and 94; SEQ ID NOS: 81 and 95; SEQ ID NOS: 82 and 96; SEQ ID NOS: 83 and 97; SEQ ID NOS: 84 and 98; SEQ ID NOS: 85 and 99; SEQ ID NOS: 86 and 100; SEQ ID NOS: 87 and 101; SEQ ID NOS: 88 and 102; SEQ ID NOS: 89 and 103; SEQ ID NOS: 90 and 104; SEQ ID NOS: 142 and 143; SEQ ID NOS: 169 and 172; SEQ ID NOS: 170 and 173; SEQ ID NOS: 171 and 174; SEQ ID NOS: 108 and 121; SEQ ID NOS: 109 and 122; SEQ ID NOS: 110 and 123; SEQ ID NOS: 111 and 124; SEQ ID NOS: 112 and 125; SEQ ID NOS: 113 and 126; SEQ ID NOS: 114 and 127; SEQ ID NOS: 115 and 128; SEQ ID NOS: 116 and 129; SEQ ID NOS: 117 and 130; SEQ ID NOS: 118 and 131; SEQ ID NOS: 119 and 132; SEQ ID NOS: 120 and 133; and SEQ ID NOS: 134 and 135.
10A. The TCR according to any one of the paras 1A-7A, wherein the TCRα chain and the TCRβ chain complete amino acid sequences share at least about 80%, about 85%, about 90%, or about 95% sequence identity with the sequences set forth in Table 4.
11A. The TCR according to para 8A or 9A, wherein the TCRα chain and the TCRβ chain complete nucleotide sequences share at least about 80%, about 85%, about 90%, or about 95% sequence identity with the sequences set forth in Table 5 and Table 6.
12A. The TCR according to any of paras 8A, 9A, or 11A expressed by a T cell.
13A. The TCR according to para 12A, expressed by a human T cell.
14A. The TCR according to any preceding para specifically binding an EBV-derived antigen expressed by a cell.
15A. The TCR according to any preceding para specifically binding an EBV-derived antigen, wherein the TCR is expressed by a T cell and both the T cell and the cell expressing the EBV-derived antigen are present in a single subject.
16A. The TCR according to para 1A, said TCR binding to an HLA-A*02-restricted EBV-derived antigenic peptide having a sequence selected from SEQ ID NO: 105, SEQ ID NO: 106, SEQ ID NO: 107; SEQ ID NO: 145, and SEQ ID NO: 146.
17A. The TCR according to para 16A, wherein the HLA-A*02-restricted EBV BRLF1-derived antigenic peptide has a sequence according to SEQ ID NO: 105 comprising:
a TCRα chain comprising a CDR3 amino acid sequence having at least about 95% sequence identity with a member selected from: SEQ ID NOS: 15, 16, and 17; in combination with:
a TCRβ chain comprising a CDR3 amino acid sequence sharing at least about 95% sequence identity with a member selected from: SEQ ID NOS: 43, 44, 45, and 46.
18A. The TCR according to para 17A comprising a variable domain comprising the TCRα chain CDR3 and TCRβ chain CDR3 of polypeptide SEQ ID pairs selected from:
SEQ ID NOS: 15 and 43; SEQ ID NOS: 16 and 44; SEQ ID NOS: 15 and 45; and SEQ ID NOS: 17 and 46.
19A. The TCR according to para 17A, comprising:
a TCRα chain amino acid sequence sharing at least about 80%, about 85%, about 90%, or about 95% sequence identity with a member selected from SEQ ID NOS: 55; 56; and 57; in combination with:
a TCRβ chain amino acid sequence sharing at least about 80%, about 85%, about 90%, or about 95% sequence identity with a member selected from SEQ ID NOS: 67; 68; 69; and 70.
20A. The TCR according to para 19A comprising a variable domain comprising a TCRα chain and TCRβ chain of polypeptide SEQ ID pairs selected from:
SEQ ID NOS: 55 and 67; SEQ ID NOS: 56 and 68; SEQ ID NOS: 55 and 69; and SEQ ID NOS: 57 and 70.
21A. The TCR according to para 17A, comprising:
a TCRα chain comprising the nucleotide sequence having at least about 80%, about 85%, about 90%, or about 95% sequence identity with a member selected from: SEQ ID NOS: 79; 80; 81; 108; 109; 110; and 111; in combination with:
a TCRβ chain comprising a nucleotide sequence having at least about 80%, about 85%, about 90%, or about 95% sequence identity with a member selected from: SEQ ID NOS: 92; 93; 94; 95; 121; 122; 123; and 124.
22A. The TCR according to para 21A comprising a variable domain comprising a TCRα chain and TCRβ chain of nucleotide SEQ ID pairs selected from the group consisting of:
SEQ ID NOS: 79 and 92; SEQ ID NOS: 80 and 93; SEQ ID NOS: 79 and 94; SEQ ID NOS: 81 and 95; SEQ ID NOS: 108 and 121; SEQ ID NOS: 109 and 122; SEQ ID NOS: 110 and 123; and SEQ ID NOS: 111 and 124.
23A. The TCR according to para 16A, wherein the HLA-A*02-restricted EBV LMP2-derived antigenic peptide has a sequence according to SEQ ID NO:107, comprising:
a TCRα chain variable domain comprising a complementary determining region CDR3 of the following sequence:

(SEQ ID NO: 181)
C-A-T-X¹-G-X²-S-G-Y-S-T-L-T-F,

in combination with:
a TCRβ chain variable domain comprising a complementary determining region (CDR)3 CDR3 of the following sequence: C-A-S-X³-X⁴-Q-G-G-(S)-X⁵-X⁶-G-Y-T-F (SEQ ID NO:182), whereby(S) is optional, and wherein:
i. X¹ is selected from E and A;
ii. X² is selected from D, G, N, S, and any of the following amino acids with related properties: E, A, Q and T;
iii. X³ is selected from S and T, and any of the following amino acids with related properties: N and Q;
iv. X⁴ is selected from K, R and T, and any of the following amino acids with related properties: H, and S;
V. X⁵ is selected from G and A;
vi. X⁶ is selected from Y and S, and any of the following amino acids with related properties: F, W, H and T.
24A. The TCR according to para 23A, comprising:
a TCRα chain variable domain comprising a complementary determining region (CDR)3 CDR3 having at least about 95% sequence identity with a member selected from: SEQ ID NO: 20; SEQ ID NO: 23 and; SEQ ID NO: 24; in combination with:
a TCRβ chain variable domain comprising a complementary determining region (CDR)3 CDR3 having at least about 95% sequence identity with a member selected from: SEQ ID NO: 49; SEQ ID NO: 52; and SEQ ID NO: 53.
25A. The TCR according to para 24A comprising a variable domain comprising the TCRα chain CDR3 and TCRβ chain CDR3 of polypeptide SEQ ID pairs selected from:
SEQ ID NOS: 20 and 49; SEQ ID NO:23 and 52; SEQ ID NOS: 23 and 53
26A. The TCR according to para 23A, comprising:
a TCRα chain amino acid sequence having at least about 80%, about 85%, about 90%, or about 95% sequence identity with a member selected from: SEQ ID NO: 60; SEQ ID NO: 63; SEQ ID NO: 64; and SEQ ID NO: 65; in combination with:
a TCRβ chain amino acid sequence shares at least bout 80%, about 85%, about 90%, or about 95% sequence identity with a member selected from: SEQ ID NO: 73; SEQ ID NO: 76; and SEQ ID NO: 77.
27A. The TCR according to para 26A comprising a variable domain comprising a TCRα chain and TCRβ chain of polypeptide SEQ ID pairs selected from the group consisting of:
SEQ ID NOS: 60 and 73; SEQ ID NOS: 63 and 76; SEQ ID NOS: 64 and 77.
28A. The TCR according to para 23A, comprising:
a TCRα chain encoded by a nucleic acid having at least about 80%, about 85%, about 90%, or about 95% sequence identity with a member selected from: SEQ ID NO: 84; SEQ ID NO: 88; SEQ ID NO: 89; SEQ ID NO: 90; SEQ ID NO: 114; SEQ ID NO: 118; SEQ ID NO: 119; and SEQ ID NO:120; in combination with:
a TCRβ chain variable domain encoded by a nucleic acid having at least about 80%, about 85%, about 90%, or about 95% sequence identity with a member selected from: SEQ ID NO: 98; SEQ ID NO: 102; SEQ ID NO: 103; SEQ ID NO: 104; SEQ ID NO: 127; SEQ ID NO: 132; SEQ ID NO: 132
29A. The TCR according to para 28A comprising a variable domain comprising a TCRα chain and TCRβ chain of nucleotide SEQ ID pairs selected from the group consisting of:
SEQ ID NOS: 84 and 98; SEQ ID NOS: 88 and 102; SEQ ID NOS: 89 and 103; SEQ ID NOS: 114 and 127; SEQ ID NOS: 118 and 131; SEQ ID NOS: 119 and 132.
30A. The TCR according to para 16A, wherein the HLA-A*02-restricted EBV LMP2-derived antigenic peptide has a sequence according to SEQ ID NO:106, comprising:
a TCRα chain variable domain comprising a CDR3 of the following amino acid sequence: C-A-X¹-X²-G-A-G-S-Y-Q-L-T-F (SEQ ID NO:183), in combination with:
a TCRβ chain variable domain comprising a CDR3 of the following amino acid sequence: C-A-S-S-X³-E-G-Q-A-S-S-Y-E-Q-Y-F (SEQ ID NO:184), wherein
i. X¹ is a member selected from G, V, and any of the following amino acids with related properties: A, I and L
ii. X² is a member selected from A, S, and any of the following amino acids with related properties: G and T
iii. X³ is a member selected from L, A, and any of the following amino acids with related properties: I, V and G.
31A. The TCR according to para 30A, comprising:
a TCRα chain comprising a complementary determining region CDR3 amino acid sequence having at least about 95% sequence identity with a member selected from: SEQ ID NOS: 21 and 22; in combination with:
a TCRβ chain comprising a complementary determining region CDR3 amino acid sequence having at least about 95% sequence identity with a member selected from: SEQ ID NOS: 50; and 51.
32A. The TCR according to para 31A consisting of a variable domain comprising the TCRα chain CDR3 and TCRβ chain CDR3 of amino acid pairs selected from:
SEQ ID NOS: 21 and 50; SEQ ID NOS: 22 and 50; and SEQ ID NOS: 21 and 51.
33A. The TCR according to para 30A, comprising:
a TCRα chain amino acid sequence having at least about 80%, about 85, about 90%, or about 95% sequence identity with a member selected from: SEQ ID NOS: 61 and 62; in combination with:
a TCRβ chain amino acid sequence having at least about 80%, about 85, about 90%, or about 95% sequence identity with a member selected from: SEQ ID NOS: 74; and 75.
34A. The TCR according to para 33A comprising a TCRα chain CDR3 and TCRβ chain CDR3 of polypeptide SEQ ID pairs selected from the group consisting of:
SEQ ID NOS: 61 and 74; SEQ ID NO: 62 and 74; and SEQ ID NOS: 61 and 75.
35A. The TCR according to para 30A, comprising:
a TCRα chain encoded by a nucleic acid sharing at least about 80%, about 85%, about 90%, or about 95% sequence identity with a member selected from: SEQ ID NOS: 85; 86; 87; 115; 116; and 117; in combination with:
a TCRβ chain encoded by a nucleic acid sharing at least about 80%, about 85%, about 90%, or about 95% sequence identity with a member selected from: SEQ ID NOS: 99; 100; 101; 128; 129; and 130.
36A. The TCR according to para 35A comprising a TCRα chain and TCRβ chain of nucleotide SEQ ID pairs selected from the group consisting of:
SEQ ID NOS: 85 and 99; SEQ ID NOS: 86 and 102; SEQ ID NOS: 87 and 101; SEQ ID NOS: 115 and 128; SEQ ID NOS: 116 and 129; SEQ ID NOS: 117 and 130.
37A. A TCR according to para 16A, wherein the HLA-A*02-restricted EBV LMP2-derived antigenic peptide has a sequence according to SEQ ID NO: 146, comprising:
a TCRα chain comprising a complementary determining region (CDR)3 CDR3 of amino acid sequence having at least about 95% sequence identity with SEQ ID NO:18; in combination with:
a TCRβ chain comprising a complementary determining region (CDR)3 CDR3 of amino acid sequence sharing at least about 95% sequence identity with SEQ ID NO:47.
38A. The TCR according to para 37A, comprising:
a TCRα chain of amino acid sequence having at least about 80%, about 85%, about 90%, or about 95% sequence identity with SEQ ID NO: 58; in combination with:
a TCRβ chain of amino acid sequence having at least about 80%, about 85%, about 90%, or about 95% sequence identity with SEQ ID NO: 71.
39A. The TCR according to para 37A, comprising:
a TCRα chain nucleotide sequence sharing at least about 80%, about 85%, about 90%, or about 95% sequence identity with a member selected from: SEQ ID NO:82; and SEQ ID NO:112; in combination with:
a TCRβ chain a nucleotide sequence having at least about 80%, about 85%, about 90%, or about 95% sequence identity with a member selected from: SEQ ID NO: 96; and SEQ ID NO: 125.
40A. The TCR according to para 39A comprising a TCRα chain and TCRβ chain of nucleotide SEQ ID pairs selected from:
SEQ ID NOS: 82 and 96; and SEQ ID NOS: 112 and 125.
41A. A TCR according to para 16A, wherein the HLA-B*35-restricted EBV BZLF1-derived antigenic peptide has a sequence according to SEQ ID NO:145, comprising:
a TCRα chain comprising a complementary determining region (CDR)3 CDR3 of amino acid sequence having at least about 95% sequence identity with SEQ ID NO: 138; in combination with:
a TCRβ chain comprising a complementary determining region (CDR)3 CDR3 of amino acid sequence having at least about 95% sequence identity with SEQ ID NO:139.
42A. The TCR according to para 41A, comprising:
a TCRα chain of amino acid sequence having at least about 80%, about 85%, about 90%, or about 95% sequence identity with SEQ ID NO: 140; in combination with:
a TCRβ chain of amino acid sequence SEQ ID NO: 141.
43A. The TCR according to para 41A, comprising:
a TCRα chain of nucleotide sequence sharing at least about 80%, about 85%, about 90%, or about 95% sequence identity with a member selected from: SEQ ID NO: 142; and SEQ ID NO: 134; in combination with:
a TCRβ chain of nucleotide sequence sharing at least about 80%, about 85%, about 90%, or about 95% sequence identity with a member selected from: SEQ ID NO: 143; and SEQ ID NO: 135.
44A. The TCR according to para 43A comprising a TCRα chain and TCRβ chain of nucleotide SEQ ID pairs selected from:
SEQ ID NOS: 142 and 143; and SEQ ID NOS: 134 and 135.
45A. The TCR according to para 1A, said TCR binding to an HLA-A*02-restricted antigenic peptide having a sequence selected from SEQ ID NO:147 and SEQ ID NO:148.
46A. The TCR according to para 45A, wherein the HLA-A*02 MAPK8IP-derived antigenic peptide has a sequence according to SEQ ID NO:147 comprising:
A TCRα chain comprising a CDR3 amino acid sequence sharing at least 95% sequence identity with a member selected from: SEQ ID NOS: 151 and 152; in combination with:
A TCRβ chain comprising a CDR3 amino acid sequence sharing at least about 95% sequence identity with a member selected from: SEQ ID NOS: 159 and 160.
47A. The TCR according to para 46A comprising a variable domain comprising the TCRα chain CDR3 and TCRβ chain CDR3 of polypeptide SEQ ID pairs selected from:
SEQ ID NOS: 151 and 159; and SEQ ID NOS: 152 and 160.
48A. The TCR according to para 46A, comprising:
A TCRα chain amino acid sequence sharing at least about 80%, about 85%, about 90%, or about 95% sequence identity with a member selected from SEQ ID NO:162 and 163; in combination with:
A TCRβ chain amino acid sequence sharing at least about 80%, about 85%, about 90%, or about 95% sequence identity with a member selected from SEQ ID NOS: 166 and 167.
49A. The TCR according to para 48A comprising a variable domain comprising a TCRα chain and a TCRβ chain of polypeptide SEQ ID pairs selected from:
SEQ ID NOS: 162 and 166; and SEQ ID NOS: 163 and 167.
50A. The TCR according to para 46A, comprising:
A TCRα chain comprising the nucleotide sequence sharing at least about 80%, about 85%, about 90%, or about 95% sequence identity with a member selected from: SEQ ID NOS: 169; 170; 175; and 176; in combination with:
A TCRβc chain comprising a nucleotide sequence sharing at least about 80%, about 85%, about 90%, or about 95% sequence identity with a member selected from: SEQ ID NOS: 172; 173; 178; and 179.
51A. The TCR according to para 50A comprising a variable domain comprising a TCRα chain and a TCRβ chain of nucleotide SEQ ID pairs selected from the group consisting of: SEQ ID NOS: 169 and 172; SEQ ID NOS: 170 and 173; SEQ ID NOS: 175 and 178; and SEQ ID NOS: 176 and 179.
52A. A TCR according to para 46A, comprising a TCRα chain variable domain comprising a CDR3 of the following sequence: C-A-F-M-X¹-X²-D-S-X³-X⁴-Y-X⁵-X⁶-I-X⁷ (SEQ ID NO:18185) in combination with a TCRβ chain variable domain comprising a complementary determining region (CDR)3 CDR3 with SEQ ID NOS: 166, or 167;
wherein:
X¹ is L or I, or V as an amino acid with related properties
X² is P or I, or any of the following amino acids with related properties: V and L
X³ is G or N, or any of the following amino acids with related properties: Q, A, C or S
X⁴ is T or no AA at this position, or S as an amino acid with related properties
X⁵ is K or Q, or any of the following amino acids with related properties: R, H or N
X⁶ is L or Y, or any of the following amino acids with related properties: I, V, F, W or H
X⁷ is F or W.
53A. The TCR according to para 52A, comprising a TCRα chain variable domain comprising a CDR3 of SEQ ID NOS: 151, or 152, in combination with a TCRβ chain variable domain comprising a complementary determining region (CDR)3 CDR3 of SEQ ID NOS: 159, or 160.
54A. The TCR according to para 52A, comprising a TCRα chain with the variable region amino acid sequence SEQ ID NOS: 162, or 163, in combination with a TCRβ chain with the variable region amino acid sequence SEQ ID NOS: 166, or 167.
55A. The TCR according to para 52A, comprising a TCRα chain with the variable region nucleotide sequence SEQ ID NOS: 169, 170, 175, or 176, in combination with a TCRβ chain with the variable region amino acid sequence SEQ ID NOS: 172, 173, 178, or 179.
56A. The TCR according to para 45A, wherein the HLA-A*02-restricted HERV-K-derived-antigenic peptide has a sequence according to SEQ ID NO:148, comprising:
A TCRα chain comprising a CDR3 amino acid sequence sharing at least bout 95% sequence identity with SEQ ID NO: 153; in combination with:
A TCRβ chain comprising a CDR3 amino acid sequence sharing at least about 95% sequence identity with SEQ ID NO: 161.
57A. A TCR according to paras 56A comprising of a variable domain comprising the TCRα chain CDR3 and TCRβ chain CDR3 pair of SEQ ID NOS: 153 and 161.
58A. A TCR according to para 56A, comprising a TCRα chain with the variable region amino acid sequence SEQ ID NOS: 164, in combination with a TCRβ chain with the variable region amino acid sequence SEQ ID NOS: 168
59A. The TCR according to para 56A, comprising:
A TCRα chain comprising the nucleotide sequence sharing at least about 80%, about 85%, about 90%, or about 95% sequence identity with a member selected from: SEQ ID NOS: 171 and 177; in combination with:
A TCRβc chain comprising a nucleotide sequence sharing at least about 80%, about 85%, about 90%, or about 95% sequence identity with a member selected from: SEQ ID NOS: 174 and 180.
60A. The TCR according to para 59A comprising a variable domain comprising a TCRα chain and a TCRβ chain of nucleotide SEQ ID pairs selected from the group consisting of: SEQ ID NOS: 171 and 174; and SEQ ID NOS: 177 and 180.
61A. An expression vector comprising a nucleotide sequence according to any one of paras 50A, 51A, 59A and 60A.
62A. A host cell comprising the nucleotide sequence according to para 61A.
63A. The host cell according to para 62A wherein the host cell is an isolated host cell.
64A. The host cell according to para 62A or 63A expressing the TCR.
65A. Use of the TCR-expressing T-cells according to para 64A for T-cell-based adoptive cell transfer (ACT) as a therapeutic treatment in a subject suffering a splice-variant or HERV-K-associated cancer.
66A. The use according to para 65A, wherein the ACT is used in combination with immune modulating agents, selected from the group of cytokines, TLR agonist, RIG-I like receptor (RLR) agonists, immune checkpoint inhibitors, chemotherapeutic agents, antibodies, radiotherapy and a combination thereof.
67A. Use of the TCR-expressing T-cells according to para 65A for T-cell-based adoptive cell transfer (ACT) as a therapeutic treatment in a subject suffering a cancer, condition, disease, disorder, or pathology associated with expression of SF3B1mut- or other genetically altered splice factors.
68A. The use according to para 65A wherein said SF3B1mut- or other genetically altered splice factor-associated condition, disease, disorder, or pathology includes:
SF3B1mut expressing cancers including but not limited to: myelodysplastic syndrome (MDS), non-small cell lung cancer (NSCLC), chronic lymphocytic leukemia, pancreatic cancer, acute myeloid leukemia and chronic myelomonocytic leukemia.
69A. Use of the TCR-expressing T-cells according to para 65A for T-cell-based adoptive cell transfer (ACT) as a therapeutic treatment in a subject suffering a cancer, condition, disease, disorder, or pathology associated with expression of human endogenous retrovirus protein HERV-K.
70A. Use of the TCRα chain and TCRβ chain pairs comprising a sequence selected from the combinations set forth in Table 5 or Table 6 as part of a fusion construct, whereby said fusion construct consists of a TCR and a single-chain fragment that binds to a molecule specifically expressed on T cells, including but not limited to CD3.
1B. An isolated T cell receptor (TCR) comprising a TCRα chain and a TCRβ chain binding to an Epstein-Barr Virus (EBV)-derived antigenic peptide when presented by a major histocompatibility complex (MHC) molecule, wherein said TCRα chain and said TCRβ chain each comprise one, two or three complementarity determining regions selected from CDR1, CDR2, and CDR3, each respectively comprising an amino acid sequence having at least about 95% sequence identity with an amino acid sequence selected from Table 3A.
2B. The TCR according to para 1B, wherein said TCR binds to an HLA-A*02-restricted EBV-derived antigenic peptide having a sequence selected from SEQ ID NO: 105, SEQ ID NO: 106, SEQ ID NO: 107 or SEQ ID NO: 146; or an HLA-B*35-restricted EBV-derived antigenic peptide of SEQ ID NO: 146.
3B. The TCR according to para 2B, wherein the HLA-A*02-restricted EBV BRLF1-derived antigenic peptide has a sequence according to SEQ ID NO: 105 comprising:
a CDR3α amino acid sequence having at least about 95% sequence identity with a member selected from: SEQ ID NOS: 15, 16, and 17; in combination with:
a CDR3β amino acid sequence sharing at least about 95% sequence identity with a member selected from: SEQ ID NOS: 43, 44, 45, and 46.
4B. The TCR according to para 3B, wherein said TCR comprises a variable domain comprising the CDR3α and CDR3β of polypeptide SEQ ID pairs selected from:
SEQ ID NOS: 15 and 43; SEQ ID NOS: 16 and 44; SEQ ID NOS: 15 and 45; and SEQ ID NOS: 17 and 46.
5B. The TCR according to para 3B, comprising:
a TCRα chain amino acid sequence sharing at least about 80%, about 85%, about 90%, or about 95% sequence identity with a member selected from SEQ ID NOS: 55; 56; and 57; in combination with:
a TCRβ chain amino acid sequence sharing at least about 80%, about 85%, about 90%, or about 95% sequence identity with a member selected from SEQ ID NOS: 67; 68; 69; and 70.
6B. The TCR according to para 5B comprising a variable domain comprising a TCRα chain and TCRβ chain of polypeptide SEQ ID pairs selected from:
SEQ ID NOS: 55 and 67; SEQ ID NOS: 56 and 68; SEQ ID NOS: 55 and 69; and SEQ ID NOS: 57 and 70.
7B. The TCR according to para 3B, comprising:
a TCRα chain encoded by a nucleotide sequence having at least about 80%, about 85%, about 90%, or about 95% sequence identity with a member selected from: SEQ ID NOS: 79; 80; 81; 108; 109; 110; and 111; in combination with:
a TCRβ chain encoded by a nucleotide sequence having at least about 80%, about 85%, about 90%, or about 95% sequence identity with a member selected from: SEQ ID NOS: 92; 93; 94; 95; 121; 122; 123; and 124.
8B. The TCR according to para 7B comprising a variable domain comprising a TCRα chain and TCRβ chain of nucleotide SEQ ID pairs selected from the group consisting of:
SEQ ID NOS: 79 and 92; SEQ ID NOS: 80 and 93; SEQ ID NOS: 79 and 94; SEQ ID NOS: 81 and 95; SEQ ID NOS: 108 and 121; SEQ ID NOS: 109 and 122; SEQ ID NOS: 110 and 123; and SEQ ID NOS: 111 and 124.
9B. The TCR according to para 2B, wherein the HLA-A*02-restricted EBV LMP2-derived antigenic peptide has a sequence according to SEQ ID NO:107, comprising:
a TCRα chain variable domain comprising a CDR3α of the following sequence:
   C-A-T-X¹-G-X²-S-G-Y-S-T-L-T-F (SEQ ID NO:181),
in combination with:
a TCRβ chain variable domain comprising a CDR CDR3β of the following sequence: C-A-S-X³-X⁴-Q-G-G-(S)-X⁵-X⁶-G-Y-T-F (SEQ ID NO:182), whereby(S) is optional, and wherein:
i. X¹ is selected from E and A;
ii. X² is selected from D, G, N, S, and any of the following amino acids with related properties: E, A, Q and T;
iii. X³ is selected from S and T, and any of the following amino acids with related properties: N and Q;
iv. X⁴ is selected from K, R and T, and any of the following amino acids with related properties: H, and S;
v. X⁵ is selected from G and A;
vi. X⁶ is selected from Y and S, and any of the following amino acids with related properties: F, W, H and T.
10B. The TCR according to para 9B, comprising:
a CDR3α having at least about 95% sequence identity with a member selected from: SEQ ID NOS: 20; 23; 24; in combination with:
a CDR3β having at least about 95% sequence identity with a member selected from: SEQ ID NOS: 49; 52; 53 and 54.
11B. The TCR according to para 10B comprising a CDR3α and CDR3β of polypeptide SEQ ID pairs selected from:
SEQ ID NOS: 20 and 49; SEQ ID NO:23 and 52; SEQ ID NOS: 23 and 53; and SEQ ID NOS: 24 and 54.
12B. The TCR according to para 9B, comprising:
a TCRα chain amino acid sequence having at least about 80%, about 85%, about 90%, or about 95% sequence identity with a member selected from: SEQ ID NOS: 60; 63; 64; and 65; in combination with:
a TCRβ chain amino acid sequence shares at least bout 80%, about 85%, about 90%, or about 95% sequence identity with a member selected from: SEQ ID NOS: 73; 76; 77; and 78.
13B. The TCR according to para 12B comprising a variable domain comprising a TCRα chain and TCRβ chain of polypeptide SEQ ID pairs selected from the group consisting of:
SEQ ID NOS: 60 and 73; SEQ ID NOS: 63 and 76; SEQ ID NOS: 64 and 77; and SEQ ID NOS: 65 and 78.
14B. The TCR according to para 9B, comprising:
a TCRα chain encoded by a nucleic acid having at least about 80%, about 85%, about 90%, or about 95% sequence identity with a member selected from: SEQ ID NOS: 84; 88; 89; 90; 114; 118; 119; and 120; in combination with:
a TCRβ chain encoded by a nucleic acid having at least about 80%, about 85%, about 90%, or about 95% sequence identity with a member selected from: SEQ ID NOS: 98; 102; 103; 104; 127; 131; 132; and 133.
15B. The TCR according to para 14B comprising a variable domain comprising a TCRα chain and TCRβ chain encoded by a nucleotide SEQ ID pair selected from the group consisting of:
SEQ ID NOS: 84 and 98; SEQ ID NOS: 88 and 102; SEQ ID NOS: 89 and 103; SEQ ID NOS: 90 and 104; SEQ ID NOS: 114 and 127; SEQ ID NOS: 118 and 131; SEQ ID NOS: 119 and 132; and SEQ ID NOS: 120 and 133.
16B. The TCR according to para 2B, wherein the HLA-A*02-restricted EBV LMP2-derived antigenic peptide has a sequence according to SEQ ID NO:106, comprising:
a CDR3α of the following amino acid sequence: C-A-X¹-X²-G-A-G-S-Y-Q-L-T-F (SEQ ID NO:183), in combination with:
a CDR3β of the following amino acid sequence: C-A-S-S-X³-E-G-Q-A-S-S-Y-E-Q-Y-F (SEQ ID NO:184), wherein:
i. X¹ is a member selected from G, V, and any of the following amino acids with related properties: A, I and L
ii. X² is a member selected from A, S, and any of the following amino acids with related properties: G and T
iii. X³ is a member selected from L, A, and any of the following amino acids with related properties: I, V and G.
17B. The TCR according to para 16B, comprising:
a CDR3α amino acid sequence having at least about 95% sequence identity with a member selected from: SEQ ID NOS: 19; 21; and 22; in combination with:
a TCRβ chain variable domain comprising a CDR3β with an amino acid sequence having at least about 95% sequence identity with a member selected from: SEQ ID NOS: 48; 50; and 51.
18B. The TCR according to para 17B consisting of a variable domain comprising the CDR3 a and CDR3β of amino acid pairs selected from:
SEQ ID NOS: 19 and 48; SEQ ID NOS: 21 and 50; SEQ ID NOS: 22 and 50; and SEQ ID NOS: 21 and 51.
19B. The TCR according to para 16B, comprising:
a TCRα chain amino acid sequence having at least about 80%, about 85, about 90%, or about 95% sequence identity with a member selected from: SEQ ID NOS: 59; 61; and 62; in combination with:
a TCRβ chain amino acid sequence having at least about 80%, about 85, about 90%, or about 95% sequence identity with a member selected from: SEQ ID NOS: 72; 74; and 75.
20B. The TCR according to para 19B comprising a TCRα chain and TCRβ chain of polypeptide SEQ ID pairs selected from the group consisting of:
SEQ ID NOS: 59 and 72; SEQ ID NOS: 61 and 74; SEQ ID NO:62 and 74; and SEQ ID NOS: 61 and 75.
21B. The TCR according to para 16B, comprising:
a TCRα chain encoded by a nucleic acid sharing at least about 80%, about 85%, about 90%, or about 95% sequence identity with a member selected from: SEQ ID NOS: 83; 85; 86; 87; 113; 115; 116; and 117; in combination with:
a TCRβ chain encoded by a nucleic acid sharing at least about 80%, about 85%, about 90%, or about 95% sequence identity with a member selected from: SEQ ID NOS: 97; 99; 100; 101; 128; 129; and 130.
22B. The TCR according to para 21B comprising a TCRα chain and TCRβ chain of nucleotide SEQ ID pairs selected from the group consisting of:
SEQ ID NOS: 83 and 97; SEQ ID NOS: 85 and 99; SEQ ID NOS: 86 and 102; SEQ ID NOS: 87 and 101; SEQ ID NOS: 115 and 128; SEQ ID NOS: 116 and 129; SEQ ID NOS: 117 and 130.
23B. A TCR according to para 2B, wherein the HLA-A*02-restricted EBV LMP2-derived antigenic peptide has a sequence according to SEQ ID NO:146, comprising:
a CDR3α of amino acid sequence having at least about 95% sequence identity with SEQ ID NO:18; in combination with:
a CDR3β of amino acid sequence sharing at least about 95% sequence identity with SEQ ID NO:47.
24B. The TCR according to para 23B, comprising:
a TCRα chain of amino acid sequence having at least about 80%, about 85%, about 90%, or about 95% sequence identity with SEQ ID NO:58; in combination with:
a TCRβ chain of amino acid sequence having at least about 80%, about 85%, about 90%, or about 95% sequence identity with SEQ ID NO:71.
25B. The TCR according to para 23B, comprising:
a TCRα chain encoded by a nucleotide sequence sharing at least about 80%, about 85%, about 90%, or about 95% sequence identity with a member selected from: SEQ ID NOS: 82; and 112; in combination with:
a TCRβ chain encoded by a nucleotide sequence having at least about 80%, about 85%, about 90%, or about 95% sequence identity with a member selected from: SEQ ID NOS: 96; and 125.
26B. The TCR according to para 25B comprising a TCRα chain and TCRβ chain of nucleotide SEQ ID pairs selected from:
SEQ ID NOS: 82 and 96; and SEQ ID NOS: 112 and 125.
27B. A TCR according to para 2B, wherein the HLA-B*35-restricted EBV BZLF1-derived antigenic peptide has a sequence according to SEQ ID NO:145, comprising:
a CDR3α of amino acid sequence having at least about 95% sequence identity with SEQ ID NO:138; in combination with:
a CDR3β of amino acid sequence having at least about 95% sequence identity with SEQ ID NO:139.
28B. The TCR according to para 27B, comprising:
a TCRα chain of amino acid sequence having at least about 80%, about 85%, about 90%, or about 95% sequence identity with SEQ ID NO:140; in combination with:
a TCRβ chain of amino acid sequence SEQ ID NO:141.
29B. The TCR according to para 27B, comprising:
a TCRα chain encoded by a nucleotide sequence sharing at least about 80%, about 85%, about 90%, or about 95% sequence identity with a member selected from: SEQ ID NO: 142; and SEQ ID NO:134; in combination with:
a TCRβ chain encoded by a nucleotide sequence sharing at least about 80%, about 85%, about 90%, or about 95% sequence identity with a member selected from: SEQ ID NO:143; and SEQ ID NO:135.
30B. The TCR according to para 29B comprising a TCRα chain and TCRβ chain of nucleotide SEQ ID pairs selected from:
SEQ ID NOS: 142 and 143; and SEQ ID NOS: 134 and 135.
31B. An isolated T cell receptor (TCR) comprising a TCRα chain and a TCRβ chain binding to a mutated splice factor-induced splice variant-derived antigenic peptide when presented by a major histocompatibility complex (MHC) molecule, wherein said TCRα chain and said TCRβ chain each comprise one, two or three complementarity determining regions selected from CDR1, CDR2, and CDR3, each respectively comprising an amino acid sequence having at least about 95% sequence identity with an amino acid sequence selected from Table 3A.
32B. The TCR according to paras 31B, said TCR binding to an HLA-A*02-restricted Mutant splice factor-induced MAPK8IP2 splice variant-derived peptide having the amino acid sequence SEQ ID NO:147.
33B. A TCR according to para 32B, comprising:
a CDR3β of amino acid sequence selected from SEQ ID NOS: 42; 159; and 160, in combination with
a CDR3α of the following sequence: C-A-F-M-X¹-X²-D-S-X³-X⁴-Y-X⁵-X⁶-I-X⁷ (SEQ ID NO:185), wherein:
X¹ is L or I or E, or any of the following amino acids with related properties V or D;
X² is P or I or A, or any of the following amino acids with related properties: V, L or G;
X³ is G or N, or any of the following amino acids with related properties: Q, A, C or S;
X⁴ is T or no AA at this position, or S as an amino acid with related properties;
X⁵ is K or Q, or any of the following amino acids with related properties: R, H or N;
X⁶ is L or Y, or any of the following amino acids with related properties: I, V, F, W or H;
X⁷ is For W.
34B. The TCR according to para 33B, comprising:
a CDR3α amino acid sequence sharing at least 95% sequence identity with a member selected from SEQ ID NOS: 14; 151; or 152, in combination with:
a CDR3β amino acid sequence sharing at least 95% sequence identity with a member selected from SEQ ID NOS: 42; 159; or 160.
35B. The TCR according to para 34B comprising a variable domain comprising the CDR3α and CDR3β of polypeptide SEQ ID pairs selected from:
SEQ ID NOS: 14 and 42; SEQ ID NOS: 151 and 159; and SEQ ID NOS: 152 and 160.
36B. The TCR according to para 33B, comprising:
a TCRα chain variable domain comprising an amino acid sequence having at least about 80%, about 85%, about 90%, or about 95% sequence identity with a member selected from SEQ ID NOS: 66; 162; or 163, in combination with:
a TCRβ chain with the variable region amino acid sequence sharing at least about 80%, about 85%, about 90%, or about 95% sequence identity with a member selected from SEQ ID NOS: 91; 166; or 167.
37B. The TCR according to para 36B comprising a variable domain comprising a TCRα chain and a TCRβ chain of polypeptide SEQ ID pairs selected from:
SEQ ID NOS: 66 and 91; SEQ ID NOS: 162 and 166; and SEQ ID NOS: 163 and 167.
38B. The TCR according to para 32B, comprising:
a TCRα chain encoded by a nucleotide sequence selected from the group consisting of SEQ ID NOS: 169; 170; 175; 176; 186; or 188, in combination with:
a TCRβ chain encoded by a nucleotide sequence selected from the group consisting of SEQ ID NOS: 172; 173; 178; 179; 187; or 189.
39B. The TCR according to para 38B comprising a variable domain comprising a TCRα chain and a TCRβ chain of nucleotide SEQ ID pairs selected from the group consisting of:
SEQ ID NOS: 169 and 172; SEQ ID NOS: 170 and 173; SEQ ID NOS: 175 and 178; SEQ ID NOS: 176 and 179; SEQ ID NOS: 186 and 187; and SEQ ID NOS: 188 and 189.
40B. An isolated T cell receptor (TCR) comprising a TCRα chain and a TCRβ chain binding to a Human Endogenous Retrovirus (HERV)-derived antigenic peptide when presented by a major histocompatibility complex (MHC) molecule, wherein said TCRα chain and said TCRβ chain each comprise one, two or three complementarity determining regions selected from CDR1, CDR2, and CDR3, each respectively comprising an amino acid sequence having at least about 95% sequence identity with an amino acid sequence selected from Table 3A.
41B. The TCR according to para 40B, wherein the TCR binds to an HLA-A*02-restricted HERV-K-derived-antigenic peptide has a sequence according to SEQ ID NO:148, comprising:
a CDR3α amino acid sequence sharing at least about 95% sequence identity with SEQ ID NO:153; in combination with:
a CDR3β amino acid sequence sharing at least about 95% sequence identity with SEQ ID NO:161.
42B. A TCR according to para 41B, comprising a TCRα chain with the variable region amino acid sequence SEQ ID NO:164, in combination with a TCRβ chain with the variable region amino acid sequence SEQ ID NO: 168.
43B. The TCR according to para 41B, comprising:
a TCRα chain encoded by a nucleotide sequence sharing at least about 80%, about 85%, about 90%, or about 95% sequence identity with a member selected from: SEQ ID NOS: 171 and 177; in combination with:
a TCRβ chain encoded by a nucleotide sequence sharing at least about 80%, about 85%, about 90%, or about 95% sequence identity with a member selected from: SEQ ID NOS: 174 and 180.
44B. The TCR according to para 43B comprising a TCRα chain and a TCRβ chain of nucleotide SEQ ID pairs selected from the group consisting of: SEQ ID NOS: 171 and 174; and SEQ ID NOS: 177 and 180.
45B. An expression vector comprising a nucleotide sequence pair according to any one of paras 8B, 15B, 21B, 26B, 30B, 39B, or 44B.
46B. A host cell comprising the expression vector according to para 45B.
47B. The host cell according to para 46B wherein the host cell is an isolated host cell.
48B. The host cell according to para 46B or 47B expressing the TCR.
49B. Use of the TCR-expressing T-cells according to para 48B for T-cell-based adoptive cell transfer (ACT) as a therapeutic treatment in a subject suffering a splice-variant of MAPK8IP2-, or HERV-K-, or EBV-associated cancer.
50B. The use according to para 49B, wherein the ACT is used in combination with immune modulating agents, selected from the group consisting of cytokines, TLR agonists, RIG-I like receptor (RLR) agonists, immune checkpoint inhibitors, chemotherapeutic agents, antibodies, radiotherapy, and a combination thereof.
51B. Use of the host cell according to para 48B for T-cell-based adoptive cell transfer (ACT) as a therapeutic treatment in a subject suffering a cancer, condition, disease, disorder, or pathology associated with expression of SF3B1mut- or other genetically altered splice factors.
52B. The use according to para 51B wherein said SF3B1mut- or other genetically altered splice factor-associated condition, disease, disorder, or pathology includes:
SF3B1mut expressing cancers including but not limited to: myelodysplastic syndrome (MDS), non-small cell lung cancer (NSCLC), chronic lymphocytic leukemia, pancreatic cancer, acute myeloid leukemia and chronic myelomonocytic leukemia.
53B. Use of the host cell according to para 48B for T-cell-based adoptive cell transfer (ACT) as a therapeutic treatment in a subject suffering a cancer, condition, disease, disorder, or pathology associated with expression of human endogenous retrovirus protein HERV-K.
54B. Use of the TCRα chain and TCRβ chain pairs comprising a nucleotide sequence selected from the combinations set forth in Table 5 or Table 6 as part of a fusion construct, whereby said fusion construct consists of a TCR and a single-chain fragment that binds to a molecule specifically expressed on T cells, including but not limited to CD3.
55B. An expression vector comprising a nucleotide sequence pair encoding mouse constant regions SEQ ID NO: 190 or SEQ ID NO:191, and human variable regions of TCR pairs with SEQ ID NOs: 162 and 166; SEQ ID NOS: 163 and 167; SEQ ID NOS: 164 and 168; and SEQ ID NOS: 66 and 91.
56B. A host cell comprising the expression vector according to para 55B.
57B. Use of the TCR-expressing T-cells according to para 56B for T-cell-based adoptive cell transfer (ACT) as a therapeutic treatment in a subject suffering a splice-variant of MAPK8IP2 or HERV-K-associated cancer.
58B. A T cell that expresses a TCR according to any one of paras 8B, 15B, 21B, 26B, 30B, 39B, or 44B, wherein the heterologous TCR of desired antigen specificity is inserted into the genome of said T cell.
59B. The T cell according to para 58B, wherein the T cell is a human T cell.
60B. The TCR according to any one of paras 8B, 15B, 21B, 26B, 30B, 39B, or 44B, wherein said TCR is expressed by a T cell, and both the T cell and the cell expressing the selected antigen are present in a single subject.

### IV. Examples

### Example 1: TCRs Binding to EBV Protein BRLF1

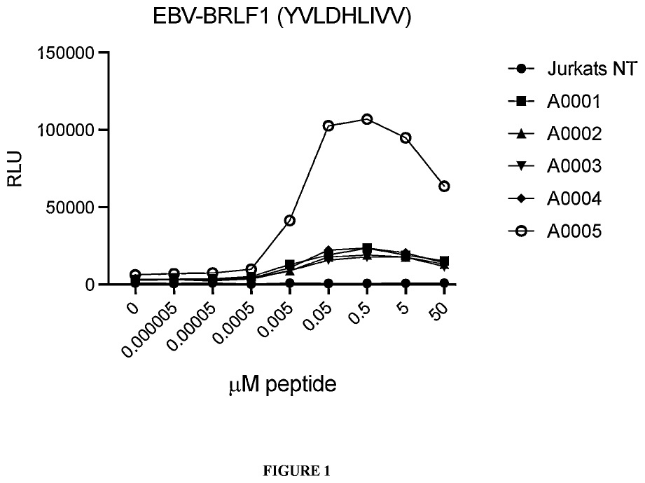

FIG. 1 shows the binding of Jurkat cells expressing EBV-BRLF1-specific TCRs A0001, A0002, A0003, A0004 and A0005 to T2 cells, used as antigen-presenting cells, presenting BRLF1 peptide YVLDHLIVV (SEQ ID NO: 105). TCRs binding to BRLF1109-117 peptide YVLDHLIVV (SEQ ID NO:105) were isolated from human donors. Modified TCR sequences were cloned into plasmids for lentivirus production. Lentiviral vectors were produced for each TCR by transfection of the transgene plasmid along with packaging plasmids encoding the additional lentiviral components into HEK293 cells. Lentiviral vectors were then used to transduce each TCR into Jurkat cells. Successful expression of the TCR was validated by flow cytometry. Specific binding of successfully engineered luciferase reporter T cells was validated by a dose-response experiment whereby increasing amount of YVLDHLIVV (SEQ ID NO:105) peptide was presented to engineered T cells on APCs. Activation of engineered reporter T cells that bound to the peptide was detected via luciferase expression. A TCRα-driven selection mechanism has been reported for BRLF1 whereby a TCRα chain comprising CDR3α CAVKDTDKLIF (SEQ ID NO:15) was found in several human donors (Kamga et al., 2019). In the current studies, TCRα variable regions comprising CDR3α CAVKDTDKLIF (SEQ ID NO:15) were identified in TCR_A0002 and TCR_A0004, but these TCRα chains pair with different TCRβ chains, representing novel TCRs that are distinct from those reported in Kamga et al. TCR sequence TCR_A0001, which has been reported previously (Kamga et al., 2019), was included for comparison.

TCR_A0002 (SEQ ID NO:55 and 67), TCR_A0003 (SEQ ID NO:56 and 68), TCR_A0004 (SEQ ID NO:55 and 69) and TCR_A0005 (SEQ ID NO:57 and 70) were expressed successfully and recognized BRLF1 peptide YVLDHLIVV presented on HLA-A*02:01-expressing APCs.

### Example 2: TCRs Binding to EBV Protein LMP2

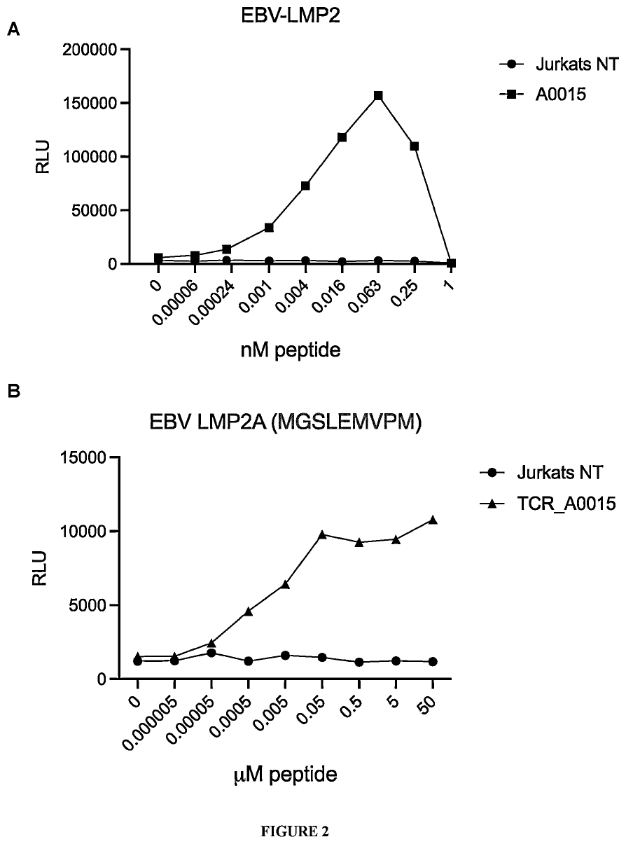

FIG. 2 shows binding of EBV LMP2-specific TCR_A0015 to an LMP2 peptide pool and to LMP2 peptide MGSLEMVPM (SEQ ID NO:146) presented on T2 cells used as antigen-presenting cells. TCR_A0015 was predicted in silico to bind to an EBV protein. A modified TCR sequence was cloned into a plasmid for lentivirus production. Lentiviral vectors were produced for this TCR by transfection of this transgene plasmid along with packaging plasmids encoding the additional lentiviral components into HEK293 cells. Lentiviral vectors were then used to for transduce this TCR into Jurkat cells. Binding to EBV protein was then tested with peptide pools. TCR_A0015 (SEQ ID NO:58 and 71) was expressed successfully and the TCR expressing Jurkat cells were activated by an LMP2 peptide pool and by peptide MGSLEMVPM (SEQ ID NO:146) presented on HLA-A*02:01-expressing APCs.

### Example 3: TCRs Binding to EBV Protein BZLF1

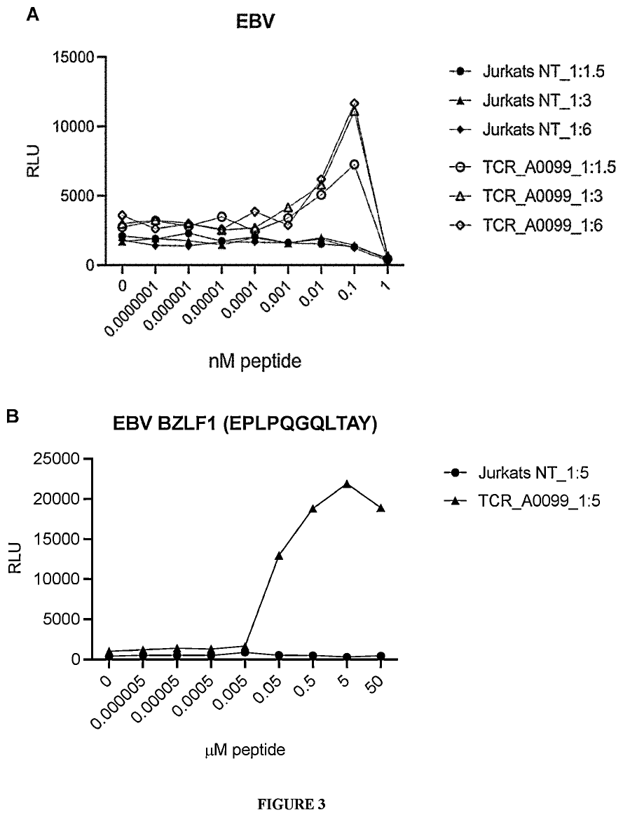

FIG. 3 shows binding of BZLF1-specific TCR_A0099 to an EBV peptide pool and to BZLF1-derived peptide EPLPQGQLTAY (SEQ ID NO:145) presented on PBMCs used as antigen-presenting cells. TCR_A0099 was predicted in silico to bind to an unknown EBV protein. A modified TCR sequence was cloned into a plasmid for lentivirus production. Lentiviral vectors were produced for this TCR by transfection of this transgene plasmid along with packaging plasmids encoding the additional lentiviral components into HEK293 cells. Lentiviral vectors were then used to transduce this TCR into Jurkat cells. Binding to EBV-derived epitopes was then tested with peptide pools of different peptides derived from multiple EBV proteins. Based on possible HLA restriction and peptides reported in the literature, a selected number of individual EBV peptides, including EPLPQGOLTAY (SEQ ID NO:145), were tested. TCR_A0099 was expressed successfully and the TCR expressing Jurkat cells were activated by an EBV peptide pool presented on PBMCs expressing HLA-A alleles 02:01 and 03:01 and HLA-B alleles 07:02 and 35:01, and by peptide EPLPQGQLTAY presented on PBMCs expressing HLA-A alleles 01:01 and 11:01 and HLA-B alleles 08:01 and 35:01 and HLA-C alleles 04:01 and 07:01.

TCR_A0099 (SEQ ID NO:140 and 141) was expressed successfully and recognized EBV peptide EPLPQGQLTAY (SEQ ID NO:145) presented on HLA-B*35:01-expressing PBMCs.

### Example 4: TCRs Binding to EBV Protein LMP2, Specific for Peptide CLGGLLTMV

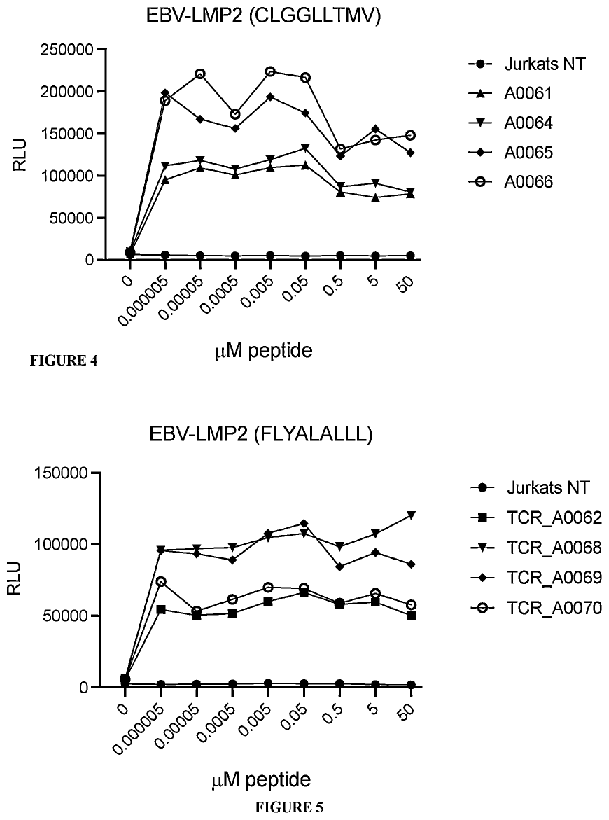

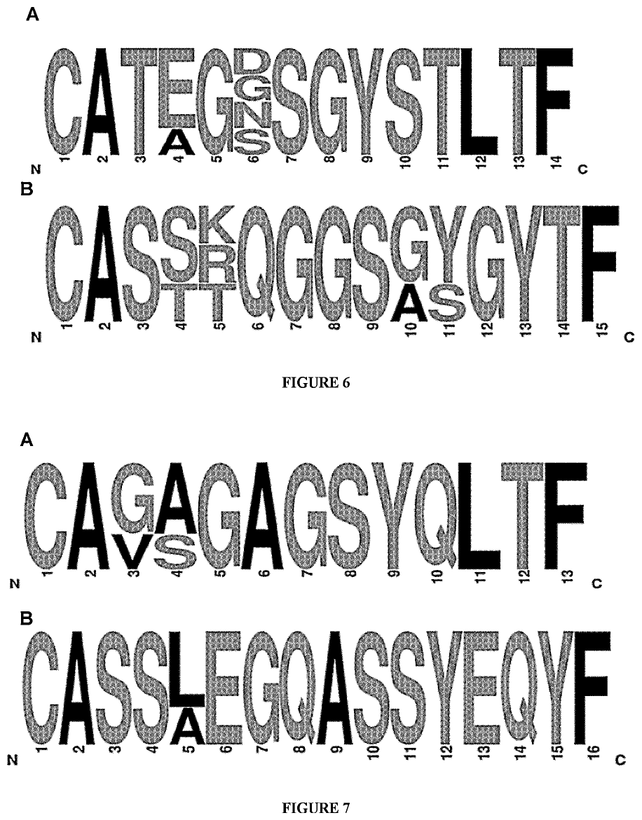

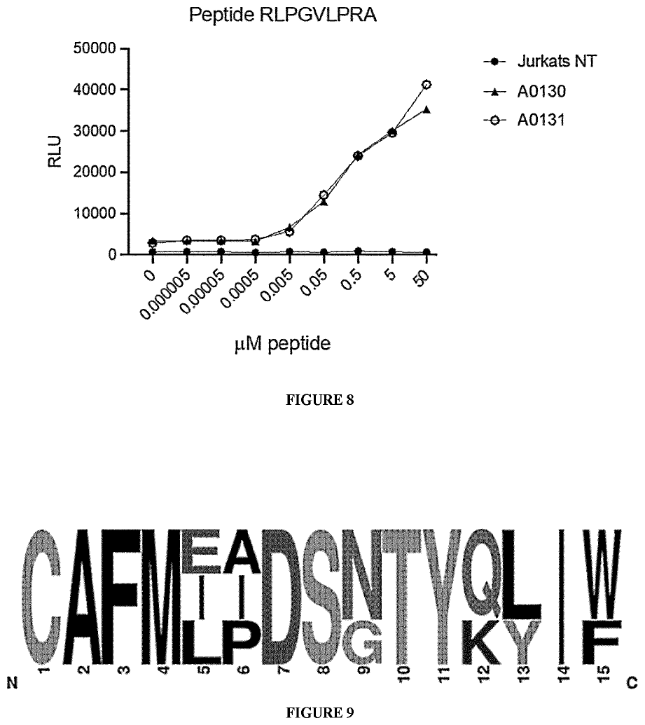

FIG. 4 shows binding of EBV-LMP2 specific TCRs to LMP2 peptide CLGGLLTMV (SEQ ID NO:106) presented on T2 cells used as antigen-presenting cells. TCRs binding to LMP2426-434 peptide CLGGLLTMV (SEQ ID NO:106) were isolated from human donors. Modified TCR sequences were cloned into plasmids for lentivirus production. Lentiviral vectors were produced for these TCRs by transfection of the transgene plasmid along with packaging plasmids encoding the additional lentiviral components into HEK293 cells. Lentiviral vectors were then used to transduce each TCR into Jurkat cells. Successful expression of TCRs was validated by flow cytometry. Specific binding of successfully engineered luciferase reporter T cells was validated by a dose-response experiment whereby increasing amount of CLGGLLTMV (SEQ ID NO:106) peptide was presented to engineered T cells on APCs. Activation of engineered reporter T cells that bound to the peptide was detected via luciferase expression.

TCR_A0061 (SEQ ID NO:59 and 72), TCR_A0064 (SEQ ID NO:61 and 74), TCR_A0065 (SEQ ID NO:62 and 74) and TCR_A0066 (SEQ ID NO:61 and 75) were expressed successfully and recognized LMP2 peptide CLGGLLTMV (SEQ ID NO:106) presented on HLA-A*02:01-expressing APCs.

### Example 5: TCRs Binding to EBV Protein LMP2, Specific for Peptide FLYALALLL

FIG. 5 shows binding of EBV-specific TCRs to LMP2 peptide FLYALALLL (SEQ ID NO:107) presented on T2 cells used as antigen-presenting cells. TCRs binding to LMP2356-364 peptide FLYALALLL (SEQ ID NO: 107) were isolated from human donors. Modified TCR sequences were cloned into plasmids for lentivirus production. Lentiviral vectors were produced for these TCRs by transfection of the transgene plasmid along with packaging plasmids encoding the additional lentiviral components into HEK293 cells. Lentiviral vectors were then used to transduce each TCR into Jurkat cells. Successful expression of TCRs was validated by flow cytometry. Specific binding of successfully engineered luciferase reporter T cells was validated by a dose-response experiment whereby increasing amount of FLYALALLL (SEQ ID NO:107) peptide was presented to engineered T cells on APCs. Activation of engineered reporter T cells that bound to the peptide was detected via luciferase expression.

TCR_0062 (SEQ ID NO:60 and 73), TCR_A0068 (SEQ ID NO:63 and 76), TCR_A0069 (SEQ ID NO:64 and 77) and TCR_A0070 (SEQ ID NO:65 and 78) were expressed successfully and recognized LMP2 peptide FLYALALLL (SEQ ID NO:107) presented on HLA-A*02:01-expressing APCs.

### Example 6: TCRs Binding to Mutated Splice Factor-Induced Peptide RLPGVLPRA

TCRs specific for mutated splice factor-induced peptide RLPGVLPRA (SEQ ID NO:147) were identified, modified and cloned into a lentivirus vector for expression in Jurkat luciferase reporter cells. Jurkat cells transduced with the lentiviral vector and successfully expressing the novel TCRs TCR_A0130 (SEQ ID NO: 162 and 166) and TCR_A0131 (SEQ ID NOs 163 and 167) were further tested in a specificity assay. Therefore, antigen presenting cells (APCs) expressing HLA-A*02:01 were incubated with peptide RLPGVLPRA (SEQ ID NO: 147) and mixed with the said Jurkat cells. Jurkat cells that are specifically activated by peptide RLPGVLPRA (SEQ ID NO:147) via the TCR produce luciferase. Luciferin, the substrate for luciferase, is then added along with additional reagents enabling a chemical reaction producing light. Expression of luciferase following TCR activation can thus be quantified as relative light units (RLU). An increasing response with increasing amount of peptide added to the cells is expected until reaching saturation in the system.

In FIG. 8 is described how T cells specifically bind to peptide RLPGVLPRA (SEQ ID NO:147) that can be presented on cells that express a mutated form of SF3B1 (SF3B1mut) and potentially other genetically altered splice factors including SUGP1. Since mutated forms of splice factors are expressed in various types of cancers including myelodysplastic syndrome, peptide RLPGVLPRA (SEQ ID NO:147) is a potential target for TCR-based immunotherapies. TCRs binding to peptide RLPGVLPRA (SEQ ID NO:147) were isolated from human donors. Modified TCR sequences were cloned into plasmids for lentivirus production. Lentiviral vectors were produced for each TCR by transfection of the transgene plasmid along with packaging plasmids encoding the additional lentiviral components into HEK293 cells. Lentiviral vectors were then used to for transduce each TCR into Jurkat cells. Successful expression was validated by flow cytometry. Specific binding of successfully engineered luciferase reporter T cells was validated by a dose-response experiment whereby increasing amount of peptide RLPGVLPRA (SEQ ID NO:147) was presented to engineered T cells on APCs. Activation of engineered reporter T cells that bound to the peptide was detected via luciferase expression. TCR_A0130 and TCR_A0131 were expressed successfully and recognized peptide RLPGVLPRA presented on HLA-A*02:01-expressing APCs.

### Example 7: TCRs Binding Human HERV-K-Derived Peptide FLQFKTWWI

TCRs specific for human endogenous retrovirus group K gag protein-derived peptide FLQFKTWWI (SEQ ID NO: 148) were identified, modified and cloned into a lentivirus vector for expression in Jurkat luciferase reporter cells. Jurkat cells transduced with the lentiviral vector and successfully expressing the novel TCR TCR_A0100 were further tested in a specificity assay. Therefore, antigen presenting cells (APCs) expressing HLA-A*02:01 were incubated with peptide FLQFKTWWI (SEQ ID NO:148) and mixed with the said Jurkat cells. Jurkat cells that are specifically activated by peptide FLQFKTWWI (SEQ ID NO:148) via the TCR produce luciferase. Luciferin, the substrate for luciferase, is then added along with additional reagents enabling a chemical reaction producing light. Expression of luciferase following TCR activation can thus be quantified as relative light units (RLU). An increasing response with increasing amount of peptide added to the cells is expected until reaching saturation in the system. TCR sequences suitable for expression of TCR protein in the form of exogenous receptor on the surface of T cells or as part of a fusion construct, are presented.

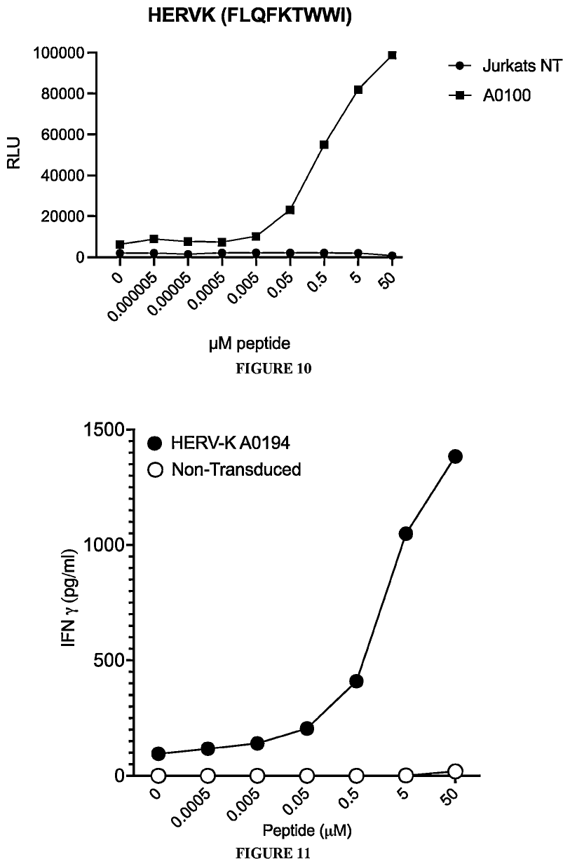

FIG. 10 describes the binding of HERV-K-specific T cells to HERV-K-derived peptide FLQFKTWWI. TCRs binding to peptide FLQFKTWWI (SEQ ID NO:148) were isolated from human donors. Modified TCR sequences were cloned into plasmids for lentivirus production. Lentiviral vectors were produced for each TCR by transfection of the transgene plasmid along with packaging plasmids encoding the additional lentiviral components into HEK293 cells. Lentiviral vectors were then used to for transduce each TCR into Jurkat cells. Successful expression was validated by flow cytometry. Specific binding of successfully engineered luciferase reporter T cells was validated by a dose-response experiment whereby increasing amount of peptide FLQFKTWWI (SEQ ID NO:148) was presented to engineered T cells on APCs. Activation of engineered reporter T cells that bound to the peptide was detected via luciferase expression.

TCR_A0100 (SEQ ID NO:164 and 168) were expressed successfully and recognized peptide FLQFKTWWI presented on HLA-A*02:01-expressing APCs.

### Example 8: Cytotoxicity Measurement of TCR-Transduced T Cells Specific for HERV-K

TCR_A0100 was expressed with a modified mouse constant region for functional testing. TCR_A0100 with a mouse constant domain is named A0194. Mouse/human hybrid TCRs have been shown previously to express more efficiently in human cells compared to fully human TCRs, and hybrid TCR constructs are now being used in the clinic for adoptive T cell therapy (Cohen et al. (2006) Cancer Res 1; 66(17):8878-8886; Leidner et al. (2022) New Engl J of Med 386:2112-2119; Yin et al. (2018) JCI Insight 3(8):e99488). As an additional advantage, tracing of the transferred TCR-transfected T cells is facilitated when they express a hybrid TCR, since an antibody binding specifically to the mouse constant region can be used to monitor and quantify the TCR-transfected T cells in the patient after adoptive transfer. The binding of the hybrid TCRs to the target antigen is anticipated to be comparable to the fully human TCR since the TCR variable region, which is interacting with its target, is not modified in the hybrid constructs.

IFNγ secreted by T cells into the culture medium during the cytotoxicity assay was quantified by ELISA methodology. The OptEIA IFNγ Kit from Becton Dickinson (BD) was used.

FIG. 11 describes the reactivity of TCR-expressing primary T cells to peptide-pulsed HLA-A*02-positive (+) target cells (T2 cell line) 4 hours after co-culture; Interferon Gamma (IFN-γ) secreted by the cells was quantified by ELISA.

Primary T cells transduced with TCR_A0194, which is TCR_A0100 with a mouse constant region, were used as effector cells. Non-TCR transduced T cells were used as control.

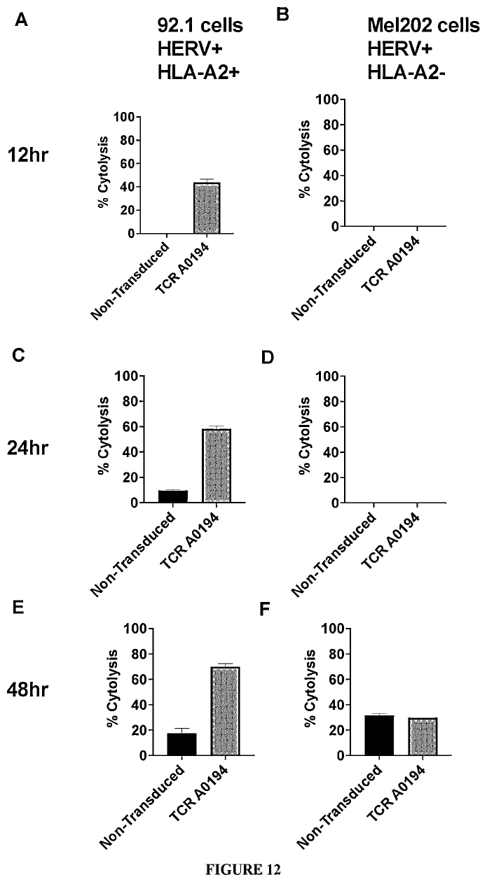

FIG. 12 shows cytolysis induced by TCR-expressing Primary T cells co-cultured with target cells in a real-time cell analyzer (Agilent xCelligence). Target cell index, measuring survival and growth of adherent target cells over time was used to calculate cytotoxicity of target cells using standard protocols and xCelligence Immunotherapy Software. Effectors were added at an effector to target ratio of 1:1. Timepoints indicate time elapsed after adding effectors.

### Example 9: Cytotoxicity Measurement of TCR Transduced T Cells Specific for Mutant Splice Factor-Induced Peptide of MAPK8IP2

Three TCRs targeting mutant splice factor-induced peptide of MAPK8IP2, known to be shared across patients with multiple types of cancer, were successfully isolated from a renal cell carcinoma patient. The TCR SEQ ID NOs are A0130 (A0130 (SEQ ID NO:162 and 166) with modified mouse constant region is named A0191), A0131 (A0131 (SEQ ID NO:163 and 167) with modified mouse constant region is named A0192) and A0132 (A0132 (SEQ ID NO:66 and 91) with modified mouse constant region is named A0193).

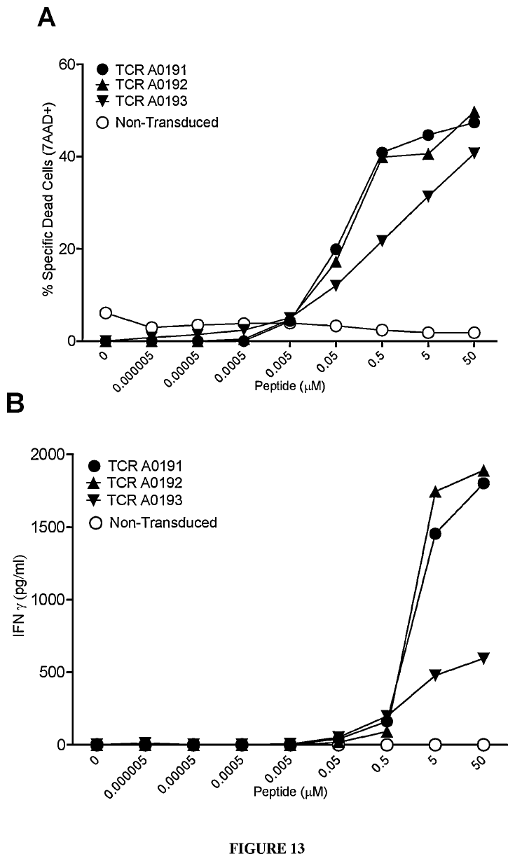

TCR-expressing T cells were used as effector cells and were labelled with Cell trace Violet (CTV) for 30 mins and resuspended in assay buffer (99% RPMI, 1% FBS). T2 cells were used as target cells and seeded in 96 well plates. Effector cells were added at the ratio of effector:target cells indicated in the brief description of the figures. Target peptide of the tested TCR was added to the wells at a range of concentrations as indicated in the figures, and cells were incubated for 20-24 hours. To analyze the cytotoxicity of effectors, cells were collected after centrifugation in a 96 well round bottom plate. Cells were stained with 7AAD for 15 mins and analyzed on an Attune flow cytometer (Thermo Fisher Scientific) (FIG. 13A). The percentage of killing for each peptide concentration and effector cell tested was assessed by quantifying the % of 7AAD+CTV-(target) cells per condition, using FlowJo software. Cell culture supernatant was kept for the analysis of secreted cytokines (FIG. 13B).

### Example 10: Further TCRs Binding to Mutated Splice Factor-Induced Peptide RLPGVLPRA

Further TCRs were identified that bind to peptide RLPGVLPRA (SEQ ID NO:147).

T cells from PBMC samples from human cancer patients were screened with hundreds of potential cancer-associated CD8+ T-cell peptides.

Samples identified as containing T cell specific for splice variant MAPK8IP2 peptide RLPGVLPRA were expanded in vitro for the isolation of TCR sequences. Human PBMCs were stimulated with peptide RLPGVLPRA (SEQ ID NO:147) and anti-CD28 antibody, and enriched based on CD137 expression. These cells were incubated with peptide RLPGVLPRA, activated monocytes and cytokines for 9-14 days.

In vitro expanded cells were then analysed using single cell RNA PCR technology to extract the TCR nucleotide sequence. Specific T cells were sorted into individual wells for single cell PCR (scPCR). RT reaction was performed, followed by a nested PCR protocol to amplify the variable TCR regions of the TCR alpha and -beta chain using primers binding in the V gene. The protocol was described previously in Dash P, Wang G C, Thomas P G. Methods Mol Biol. 2015; 1343:181-97. doi: 10.1007/978-1-4939-2963-4_15. TCR sequences were obtained by Sanger sequencing of PCR products.

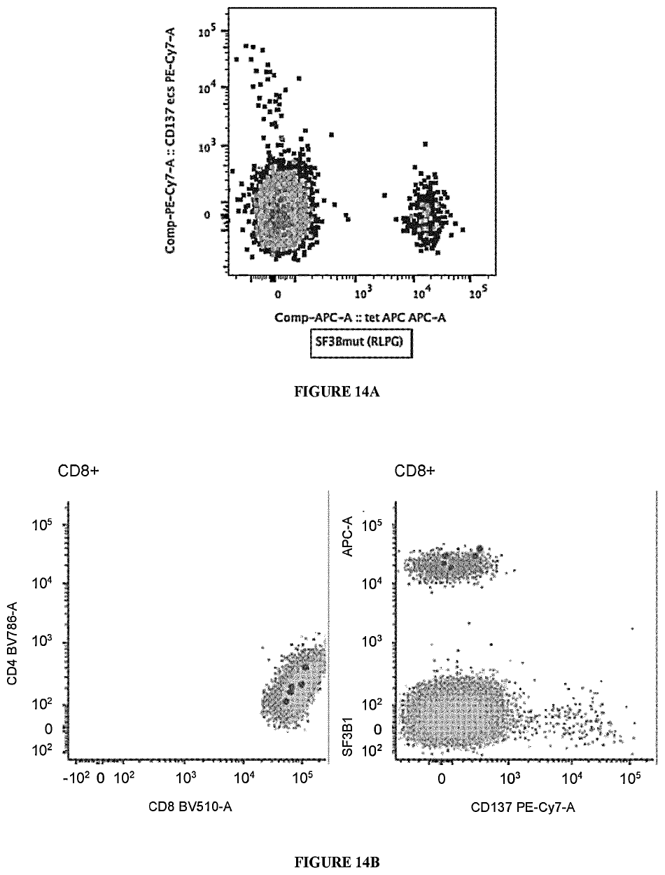

FIG. 14A shows the expansion of RLPGVLPRA-specific T cells, in order to isolate TCRs A0358 (SEQ ID NOS 200 and 201) and A0359 (SEQ ID NOs 202 and 203).

FIG. 14B shows the flow cytometry based sorting of RLPGVLPRA-loaded-tetramer binding T cells that led to the isolation of TCR_A0359.

The functionality of TCR_A0358 was assessed using a Jurkat reporter cell line transfected with TCR_A0358. Upon specific binding of the TCR to the target peptide RLPGVLPRA in the context of HLA-A*02 presentation, the TCR downstream signal results in the expression of luciferase, which can be quantified as relative light units (RLU). TCRs A0362 and A0363 were also assessed. A0362 is TCR_A0130 (SEQ ID NOs 162 and 166) expressed with human constant regions including Cys mutations (SEQ ID NOs 211 and 212). A0363 is TCR_A0131 (SEQ ID NOs 163 and 167) expressed with human constant regions including Cys mutations (SEQ ID NOs 211 and 212).

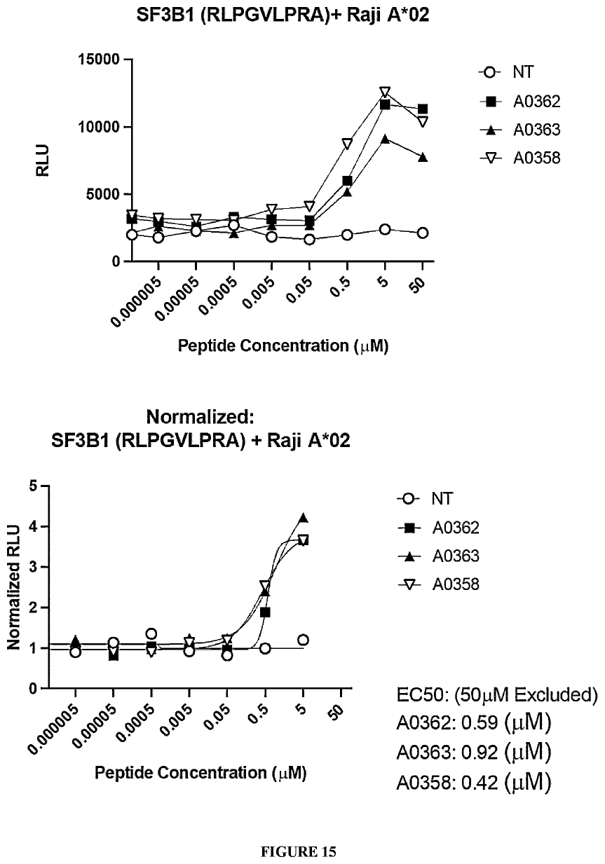

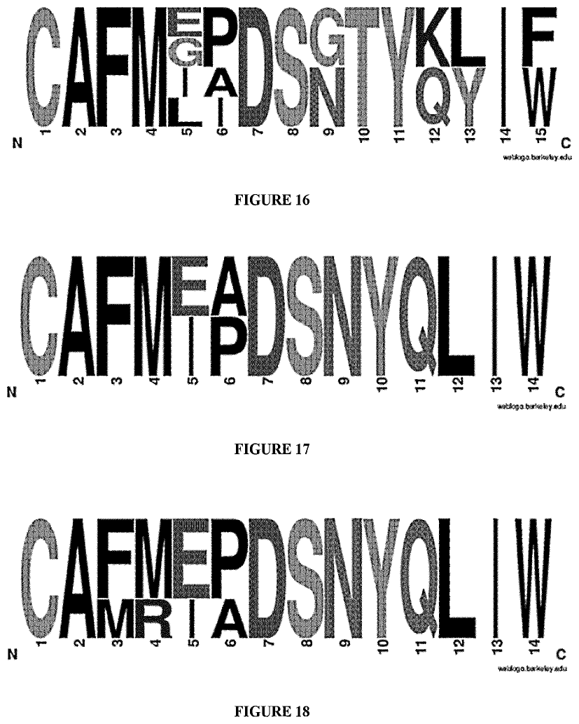

FIG. 15 shows the specific reactivity of TCRs A0358, A0130 and A0131 for peptide RLPGVLPRA.

### V. References

1. Abelson, J. N. and Simon, M. I. (2013) Methods in enzymology, eds.-in-chief, Academic Press, Inc., New York, Vols. 154-155, Wu et al. eds.
2. Alsafadi, S., Dayot, S., Tarin, M., Houy, A., Bellanger, D., Cornella, M., Wassef, M., Waterfall, J. J., Lehnert, E., Roman-Roman, S., et al. (2020). Genetic alterations of SUGP1 mimic mutant-SF3B1 splice pattern in lung adenocarcinoma and other cancers. Oncogene 2020 40:1 40, 85-96. https://doi.org/10.1038/s41388-020-01507-5.
3. Altschul, S. F., Gish, W., Miller, W., and Lipman, D. J. (1990) Basic local alignment search tool, J. Mol. Biol. 215:403-410
4. Ausubel, M., Brent, R., Kingston, R. E., Moore, D. D., Seidman, J. G., Smith, J. A., and Struhl, K. (1989) Current Protocols in Molecular Biology. Wiley and son Inc, Library of Congress, USA.
5. Bhat P, Leggatt G, Waterhouse N, Frazer I H. Interferon-γ derived from cytotoxic lymphocytes directly enhances their motility and cytotoxicity. Cell Death Dis. 2017 Jun. 1; 8(6):e2836. doi: 10.1038/cddis.2017.67. PMID: 28569770; PMCID: PMC5520949.
6. Bhati, M., Coloe, D. K., McCLuskey, J., Sewell, A. K., Rossjohn, J. (2013) The versatility of the αβ T-cell antigen receptor. Protein Science 23:260-272 (2014)
7. Bigot, J., Lalanne, A. I., Lucibello, F., Gueguen, P., Houy, A., Dayot, S., Ganier, O., Gilet, J., Tosello, J., Nemati, F., et al. (2021). Splicing patterns in sf3b 1mutated uveal melanoma generate shared immunogenic tumor-specific neoepitopes. Cancer Discovery 11, 1938-1951. https://doi.org/10.1158/2159-8290.CD-20-0555.
8. Birch, J. R. (1987) Culture of animal cells: a manual of basic technique. Freshney, R. I., Liss, A. R., Inc., New York, pp. xviii+397.
9. Bishop E L, Gudgeon N, Dimeloe S. Control of T Cell Metabolism by Cytokines and Hormones. Front Immunol. 2021 Apr. 13; 12:653605. doi: 10.3389/fimmu.2021.653605. PMID: 33927722; PMCID: PMC8076900.
10. Bollard, C. M., Gottschalk, S., Torrano, V., Diouf, O., Ku, S., Hazrat, Y., Carrum, G., Ramos, C., Fayad, L., Shpall, E. J., et al. (2014). Sustained Complete Responses in Patients With Lymphoma Receiving Autologous Cytotoxic T Lymphocytes Targeting Epstein-Barr Virus Latent Membrane Proteins. Journal of Clinical Oncology 32, 798.
11. Chandran, S. S. and Klebanoff, C. A. (2019) T cell receptor-based cancer immunotherapy: emerging efficacy and pathways of resistance. Immunol. Rev. 290:127-147.
12. Cheruiyot, A., Li, S., Srivatsan, S. N., Ahmed, T., Chen, Y., Lemacon, D. S., Li, Y., Yang, Z., Wadugu, B. A., Warner, W. A., et al. (2021). Nonsense-mediated RNA decay is a unique vulnerability of cancer cells harboring sf3b1 or u2af1 mutations. Cancer Research 81, 4499-4513. https://doi.org/10.1158/0008-5472.CAN-20-4016.
13. Cho, S. G., Kim, N., Sohn, H. J., Lee, S. K., Oh, S. T., Lee, H. J., Cho, H. il, Yim, H. W., Jung, S. E., Park, G., et al. (2015). Long-term Outcome of Extranodal NK/T Cell Lymphoma Patients Treated With Postremission Therapy Using EBV LMP1 and LMP2a-specific CTLs. Molecular Therapy 23, 1401-1409.
14. Behringer, R., Gertsenstein, M. Nagy, K., Nagy, A. (1986) Manipulating the Mouse Embryo. Cold Spring Harbor Laboratory Press, Cold Spring Harbor, N.Y.
15. Cohen et al. (2006) Enhanced antitumor activity of T cells engineered to express T-cell receptors with a second disulfide bond. Cancer Res 1; 66(17): 8878-8886
16. Davis, M. M. and Bjorkman, P. J. (1988) T-cell antigen receptor genes and T-cell recognition. Nature, 334, 395-402
17. Davis, M. M., Boniface, J. J., Reich, Z., Lyons, D. Hampl, J., Arden, B. and Chien, Y. (1998) Ligand recognition by αβ T cell receptors. Annu Rev Immunol, 16, 523-544.
18. Murphy, K. M. (2012) Janeway's immunobiology. 8^{th} ed., York, Garland Science xix, 868 p.
19. Ellis, G. I., Sheppard, N.C., and Riley, J. L. (2021). Genetic engineering of T cells for immunotherapy. Nature Reviews Genetics 22, 427-447. https://doi.org/10.1038/s41576-021-00329-9.
20. Feng, P., Ren, E. C., Liu, D., Chan, S. H., and Hu, H. (2000). Expression of Epstein-Barr virus lytic gene BRLF1 in nasopharyngeal carcinoma: Potential use in diagnosis. Journal of General Virology 81, 2417-2423.
21. Gait, M. J. (1984) Oligonucleotide synthesis: a practical approach. M. J. Gait (Editor)
22. Gao, Y., Yu, X. F., and Chen, T. (2021). Human endogenous retroviruses in cancer: Expression, regulation and function (Review). Oncology Letters 21, 1-1. https://doi.org/10.3892/OL.2020.12382/HTML.
23. Garber, K. (2018). Driving T-cell immunotherapy to solid tumors. Nature Biotechnology 36, 215-219.
24. Deininger, P. (1990) Methods in enzymology, Vol. 185, ‘Gene Expression Technology’ (D. Goeddel, ed.)
25. Gottschalk, S., Heslop, H. E., and Rooney, C. M. (2009). Adoptive Immunotherapy for EBV-associated Malignancies. Https://Doi.org/10.1080/10428190400002202 46, 1-10.
26. Henikoff, S. and Henikoff, J. G. (1992) Amino acid substitution matrices from protein blocks. Proc. Natl. Acad. Sci. USA 89:10915.
27. Hames, B. D. and Higgins, S. J. (1984) Nucleic acid hybridization: a practical approach. 245 S., 32 Abb., 49 Tab. Oxford-Washington DC, 1985, IRL Press
28. Houen, G., Trier, N. H. (2021). Epstein-Barr Virus and Systemic Autoimmune Diseases. Front. Immunol. January 2021, Vol. 11, art. 587380
29. Jansz, N., and Faulkner, G. J. (2021). Endogenous retroviruses in the origins and treatment of cancer. Genome Biology 2021 22:1 22, 1-22. https://doi.org/10.1186/S13059-021-02357-4.
30. Jin et al. (2018) Engineered T cells targeting E7 mediate regression of human papillomavirus cancers in a murine model. JCI Insight 3 (8):e99488
31. June, C. H., ed., 2001, In: Cancer Chemotherapy and Biotherapy: Principles and Practice, Lippincott Williams & Wilkins, Baltimore
32. Juo, P.-S. (2002) Concise Dictionary of Biomedicine and Molecular Biology. Second Edition By Pei-Show Juo (State University of New York, Potsdam). CRC Press LLC: Boca Raton. 2002. viii+1154 pp.
33. Kamga, L., Gil, A., Song, I., Brody, R., Ghersi, D., Aslan, N., Stern, L. J., Selin, L. K., and Luzuriaga, K. (2019). CDR3α drives selection of the immunodominant Epstein Barr virus (EBV) BRLF1-specific CD8 T cell receptor repertoire in primary infection. PLOS Pathogens 15. https://doi.org/10.1371/journal.ppat. 1008122.
34. Khan, G., and Hashim, M. J. (2014). Global burden of deaths from Epstein-Barr virus attributable malignancies 1990-2010. Infect Agent Cancer 9.
35. Lacky, J. M. (1999) The Dictionary of Cell and Molecular Biology, 3rd ed., Academic Press
36. Leeksma, A. C., Derks, I. A. M., Kasem, M. H., Kilic, E., de Klein, A., Jager, M. J., van de Loosdrecht, A. A., Jansen, J. H., Navrkalova, V., Faber, L. M., et al. (2021). The Effect of SF3B1 Mutation on the DNA Damage Response and Nonsense-Mediated mRNA Decay in Cancer. Frontiers in Oncology 10. https://doi.org/10.3389/fonc.2020.609409.
37. Leidner et al. (2022) Neoantigen T-Cell Receptor Gene Therapy in Pancreatic Cancer. New Engl J of Med 386:2112-2119
38. Mauro, V. P. (2018) Codon optimization in the production of recombinant biotherapeutics: potential risks and considerations. BioDrugs 32; 69-81.
39. Mayer, R. J. and Walker, J. H. (1987) Immunochemical methods in cell and molecular biology. eds., Academic Press, London, 1987
40. Miller, J. H. and Calos, M. P. (editors) (1987) Current Communications in Molecular Biology, Gene Transfer Vectors for Mammalian Cells. Cold Spring Harbor Laboratory
41. Munz, C. (2019) Latency and lytic replication in Epstein-Barr virus-associated oncogenesis. Nature Rev 17:691-700.
42. Munz, C. (2020) Redirecting T cells against Epstein-Barr virus infection and associated oncogenesis. Cells 9:1400.
43. Murphy, K., and Weaver Casey Janeway's Immunobiology 9.
44. Needleman, S. B. and Wunsch, C. D. (1970) A general method applicable to the search for similarities in the amino acid sequence of two proteins. J. Mol. Biol. 48:443.
45. Nguyen, J. Q. N., Drabarek, W., Yavuzyigitoglu, S., Salsench, E. M., Verdijk, R. M., Naus, N. C., de Klein, A., Kiliç, E., and Brosens, E. (2020). Spliceosome mutations in uveal melanoma. International Journal of Molecular Sciences 21, 1-15. https://doi.org/10.3390/ijms21249546.
46. Smith, D. A., Smith D. A. and Bernder, D. A. (2000) Oxford Dictionary Of Biochemistry And Molecular Biology, Revised, 2000, Oxford University Press, 738pp.
47. Springer, I., Besser, H., Tickotsky-Moskovitz, N., Dvorkin, S., Louzoun, Y. (2020) Prediction of Specific TCR-Peptide Binding From Large Dictionaries of TCR-Peptide Pairs. Front. Immunol. 25 August https://doi.org/10.3389/fimmu.2020.01803.
48. Oka, M., Xu, L., Suzuki, T., Yoshikawa, T., Sakamoto, H., Uemura, H., Yoshizawa, A. C., Suzuki, Y., Nakatsura, T., Ishihama, Y., et al. (2021). Aberrant splicing isoforms detected by full-length transcriptome sequencing as transcripts of potential neoantigens in non-small cell lung cancer. Genome Biology 22. https://doi.org/10.1186/s13059-020-02240-8.
49. Pastor, F., Kolonias, D., Giangrande, P. H., and Gilboa, E. (2010). Induction of tumour immunity by targeted inhibition of nonsense-mediated mRNA decay. Nature 465, 227. https://doi.org/10.1038/NATURE08999.
50. Perbal, P. (1984) A Practical Guide To Molecular Cloning. John Wiley & Sons. 554 pp.
51. Pearson & Lipman, Proc. Nat'l. Acad. Sci. USA 85:2444 (1988)
52. Rakoff-Nahoum, S., Kuebler, P. J., Heymann, J. J., Sheehy, M. E., Ortiz, G. M., Ogg, G. S., Barbour, J. D., Lenz, J., Steinfeld, A. D., and Nixon, D. F. (2006). Detection of T Lymphocytes Specific for Human Endogenous Retrovirus K (HERV-K) in Patients with Seminoma. Https://Home.Liebertpub.Com/Aid 22, 52-56. https://doi.org/10.1089/AID.2006.22.52.
53. Rosenberg, S. A., and Restifo, N. P. (2015). Adoptive cell transfer as personalized immunotherapy for human cancer. Science (1979) 348, 62-68.
54. Saini, S. K., Ørskov, A. D., Bjerregaard, A. M., Unnikrishnan, A., Holmberg-Thydén, S., Borch, A., Jensen, K. V., Anande, G., Bentzen, A. K., Marquard, A. M., et al. (2020). Human endogenous retroviruses form a reservoir of T cell targets in hematological cancers. Nature Communications 2020 11:1 11, 1-14. https://doi.org/10.1038/s41467-020-19464-8.
55. Sambrook, J. and Russel, D. W. (2001) Molecular Cloning: A Laboratory Manual. 3rd Edition, Vol. 1, Cold Spring Harbor Laboratory Press, New York.
56. Schroeder, H. W. and Cavacini, L. (2010) Structure and function of immunoglobulins. J Allergy Clin Immunol 125 (202): S41-S52.
57. Shafer, P., Kelly, L. M., and Hoyos, V. (2022). Cancer Therapy With TCR-Engineered T Cells: Current Strategies, Challenges, and Prospects. Frontiers in Immunology 0, 781. https://doi.org/10.3389/FIMMU.2022.835762.
58. Sidney, J., Peters, B., Frahm, N., Brander, C., and Sette, A. (2008). HLA class I supertypes: A revised and updated classification. BMC Immunology 9.
59. Smith, T. F. and Waterman, M. S. (1981) Comparison of biosequences. Adv. Appl. Math. 2:482-489.
60. Strobel (2022). Strobel-ImmTAC-Cancers2022. Cancers (Basel).
61. Thompson, M. P., and Kurzrock, R. (2004). Epstein-Barr virus and cancer. Clin Cancer Res 10, 803-821. https://doi.org/10.1158/1078-0432.CCR-0670-3.
62. Topalian, S. L., Horn, S. S., Kawakami, Y., Mancini, M., Schwartzentruber, D. J., Zakut, R., and Rosenberg, S. A. (1992). Recognition of shared melanoma antigens by human tumor-infiltrating lymphocytes. J Immunother (1991) 12, 203-206. https://doi.org/10.1097/00002371-199210000-00013.
63. Upadhaya, S., Hubbard-Lucey, V. M., and Yu, J. X. (2020). Immuno-oncology drug development forges on despite COVID-19. Nat Rev Drug Discov 19, 751-752.
64. Vignali and Kallikourdis, (2017) T cell costimulation blockade blunts pressure overload-induced heart failure. Cytokine Growth Factor Rev 36:107-116
65. Vonderheide et al., 2003, Immun. Research 27:1-15
66. Miller, S. D. and Jenkins, M. K. (1986) Detection of suppressor cells and suppressor factors for delayed-type hypersensitivity responses. In: Handbook of Experimental Immunology, Fourth Edition. D. M. Weir and C. C. Blackwell, eds.
67. Wang-Johanning, F., Radvanyi, L., Rycaj, K., Plummer, J. B., Yan, P., Sastry, K. J., Piyathilake, C. J., Hunt, K. K., and Johanning, G. L. (2008). Human Endogenous Retrovirus K Triggers an Antigen-Specific Immune Response in Breast Cancer Patients. Cancer Research 68, 5869-5877. https://doi.org/10.1158/0008-5472.CAN-07-6838.

### PATENT LITERATURE

1. Kuzushima, K., Ito, Y., Okamura, A., Akatsuka, Y. and Morishima, Y. (2006) CYTOTOXIC T-CELL EPITOPE PEPTIDES THAT SPECIFICALLY ATTACK EPSTEIN-BARR VIRUS-INFECTED CELLS AND USES THEREOF. US Patent Application No. 20090305324.
2. Mullis, K. B., Erlich, H. A., Arnheim, N., Horn, G. T., Saiki, R. K. and Scharf, S. J. (1986) Process for amplifying, detecting, and/or-cloning nucleic acid sequences. U.S. Pat. No. 4,683,195.
3. WO2021108500-T CELL RECEPTORS TARGETING MUTATIONS IN RNA SPLICING FACTORS; https://patentscope.wipo.int/search/en/detail.jsf?docId=WO2021108500&_cid=P10-L3CH84-52356-1 (this application seems to claim TCRs for peptides that are different from the one that is the target of the TCRs claimed in our application)
4. WO2021228999-NEOANTIGENIC EPITOPES ASSOCIATED WITH SF3B1 MUTATIONS; https://patentscope.wipo.int/search/en/detail.jsf?docId=WO2021228999&_cid=P10-L3CHT1-57676-1 (this application claims a large list of peptides including the one that is targeted by our TCRs, for application in vaccination, TCR therapy. No TCR sequences seem to be claimed)
5. WO2020049169A1 Herv-k-derived antigens as shared tumor antigens for Anti-cancer vaccine
6. June, C. H., Levine, B. L., Porter, D. L., Kalos, M. D. and Milone, M. C. (2011) Use of Chimeric Antigen Receptor-Modified T-Cells to Treat Cancer. US Patent Application No. 20130287748.
7. Long, F. (2019) Codon optimization. WIPO Pat. Appl. No. WO2020024917A1.

What is claimed is:

1. A T cell receptor (TCR) comprising a TCRα chain and a TCRβ chain, optionally isolated, that binds to a mutant splice factor-induced MAPK8IP2 splice variant-derived peptide having the amino acid sequence of SEQ ID NO:147, when presented by a major histocompatibility complex (MHC) molecule comprising an MHC class I α chain polypeptide encoded by a HLA-A*02 allele.

2. The TCR according to claim 1, wherein the TCR comprises:
(a)
a TCRβ chain variable domain comprising a CDR3β having an amino acid sequence selected from: SEQ ID NOs: 42, 159, 160 and 195; in combination with
a TCRα chain variable domain comprising a CDR3α having the following amino acid sequence: C-A-F-M-X¹-X²-D-S-X³-X⁴-Y-X³-X⁶-I-X⁷ (SEQ ID NO:304), wherein:
X¹ is L, I, E or G; or V or D;
X² is P, I or A; or V, L or G;
X³ is G or N; or Q, A, C or S;
X⁴ is T or no AA at this position; or S;
X⁵ is K or Q; or R, H or N;
X⁶ is L or Y; or I, V, F, W or H;
X⁷ is F or W; or
(b)
a TCRβ chain variable domain comprising a CDR3β having an amino acid sequence selected from: SEQ ID NOs: 42, 159 and 199; in combination with
a TCRα chain variable domain comprising a CDR3α having the following amino acid sequence: C-A-X¹-X²-X³-X⁴-D-S-N-Y-Q-L-I-W (SEQ ID NO:306), wherein:
X¹ is F or M; or Y or W;
X² is M or R; K or H;
X³ is I or E; or V, L or D;
X⁴ is P or A; or G; or
(c)
a TCRβ chain variable domain comprising a CDR3β having an amino acid sequence selected from: SEQ ID NOs: 42, 159 and 160; in combination with
a TCRα chain variable domain comprising a CDR3α having the following amino acid sequence: C-A-F-M-X¹-X²-D-S-X³-X⁴-Y-X⁵-X⁶-I-X⁷ (SEQ ID NO:185), wherein:
X¹ is L or I or E; or V or D;
X² is P or I or A; or V, L or G;
X³ is G or N; or Q, A, C or S;
X⁴ is T or no AA at this position; or S;
X⁵ is K or Q; or R, H or N;
X⁶ is L or Y; or I, V, F, W or H;
X⁷ is F or W; or
(d)
a TCRβ chain variable domain comprising a CDR3β having an amino acid sequence selected from: SEQ ID NO:42 and 159; in combination with
a TCRα chain variable domain comprising a CDR3α having the following sequence: C-A-F-M-X¹-X²-D-S-N-Y-Q-L-I-W (SEQ ID NO:305), wherein:
X¹ is I or E; or V or D;
X² is P or A; or V, L or G.

3. The TCR according to claim 1 or claim 2, wherein the TCR comprises:
(a)
a TCRα chain variable domain comprising a CDR3α with an amino acid sequence having at least 95% sequence identity to an amino acid sequence selected from: SEQ ID NOs: 14, 151, 152 and 194; in combination with:
a TCRβ chain variable domain comprising a CDR3β with an amino acid sequence having at least 95% sequence identity to an amino acid sequence selected from: SEQ ID NOs: 42, 159, 160 and 195; or
(b)
a TCRα chain variable domain comprising a CDR3α with an amino acid sequence having at least 95% sequence identity to an amino acid sequence selected from: SEQ ID NOs: 14, 151 and 196; in combination with:
a TCRβ chain variable domain comprising a CDR3β with an amino acid sequence having at least 95% sequence identity to an amino acid sequence selected from: SEQ ID NOs: 42, 159, and 199; or
(c)
a TCRα chain variable domain comprising a CDR3α with an amino acid sequence having at least 95% sequence identity to an amino acid sequence selected from: SEQ ID NOs: 14, 151, and 152; in combination with:
a TCRβ chain variable domain comprising a CDR3β with an amino acid sequence having at least 95% sequence identity to an amino acid sequence selected from: SEQ ID NOs: 42, 159 and 160; or
(d)
a TCRα chain variable domain comprising a CDR3α with an amino acid sequence having at least 95% sequence identity to an amino acid sequence selected from: SEQ ID NO:14 and 151; in combination with:
a TCRβ chain variable domain comprising a CDR3β with an amino acid sequence having at least 95% sequence identity to an amino acid sequence selected from: SEQ ID NO:42 and 159.

4. The TCR according to any one of claims 1 to 3, wherein the TCR comprises:
(a)
(i) a TCRα chain variable domain comprising a CDR3α having the amino acid sequence of SEQ ID NO: 14, and a TCRβ chain variable domain comprising a CDR3β having the amino acid sequence of SEQ ID NO:42; or (ii) a TCRα chain variable domain comprising a CDR3α having the amino acid sequence of SEQ ID NO:151, and a TCRβ chain variable domain comprising a CDR3β having the amino acid sequence of SEQ ID NO:159; or (iii) a TCRα chain variable domain comprising a CDR3α having the amino acid sequence of SEQ ID NO:152, and a TCRβ chain variable domain comprising a CDR3β having the amino acid sequence of SEQ ID NO:160; or (iv) a TCRα chain variable domain comprising a CDR3α having the amino acid sequence of SEQ ID NO:194, and a TCRβ chain variable domain comprising a CDR3β having the amino acid sequence of SEQ ID NO:195; or
(b)
(i) a TCRα chain variable domain comprising a CDR3α having the amino acid sequence of SEQ ID NO: 14, and a TCRβ chain variable domain comprising a CDR3β having the amino acid sequence of SEQ ID NO:42; or (ii) a TCRα chain variable domain comprising a CDR3α having the amino acid sequence of SEQ ID NO: 151, and a TCRβ chain variable domain comprising a CDR3β having the amino acid sequence of SEQ ID NO:159; or (iii) a TCRα chain variable domain comprising a CDR3α having the amino acid sequence of SEQ ID NO:196, and a TCRβ chain variable domain comprising a CDR3β having the amino acid sequence of SEQ ID NO:199; or
(c)
(i) a TCRα chain variable domain comprising a CDR3α having the amino acid sequence of SEQ ID NO: 14, and a TCRβ chain variable domain comprising a CDR3β having the amino acid sequence of SEQ ID NO:42; or (ii) a TCRα chain variable domain comprising a CDR3α having the amino acid sequence of SEQ ID NO:151, and a TCRβ chain variable domain comprising a CDR3β having the amino acid sequence of SEQ ID NO:159; or (iii) a TCRα chain variable domain comprising a CDR3α having the amino acid sequence of SEQ ID NO:152, and a TCRβ chain variable domain comprising a CDR3β having the amino acid sequence of SEQ ID NO:160; or
(d)
(i) a TCRα chain variable domain comprising a CDR3α having the amino acid sequence of SEQ ID NO: 14, and a TCRβ chain variable domain comprising a CDR3β having the amino acid sequence of SEQ ID NO:42; or (ii) a TCRα chain variable domain comprising a CDR3α having the amino acid sequence of SEQ ID NO:151, and a TCRβ chain variable domain comprising a CDR3β having the amino acid sequence of SEQ ID NO:159.

5. The TCR according to any one of claims 1 to 4, wherein the TCR comprises:
(a) (i) a TCRα chain variable domain incorporating the following CDRs:
CDR1α having the amino acid sequence of SEQ ID NO:149, 165 or 2
CDR2α having the amino acid sequence of SEQ ID NO:150 or 8
CDR3α having the amino acid sequence of SEQ ID NO:185, 304, 305 or 306; and
(ii) a TCRβ chain variable domain incorporating the following CDRs:
CDR1β having the amino acid sequence of SEQ ID NO: 154, 31, 32, 25 or 197
CDR2β having the amino acid sequence of SEQ ID NO:156, 157, 40, 33 or 198
CDR3β having the amino acid sequence of SEQ ID NO:159, 160, 42, 195 or 199; or
(b) (i) a TCRα chain variable domain incorporating the following CDRs:
CDR1α having the amino acid sequence of SEQ ID NO:149
CDR2α having the amino acid sequence of SEQ ID NO:150
CDR3α having the amino acid sequence of SEQ ID NO:151; and
(ii) a TCRβ chain variable domain incorporating the following CDRs:
CDR1β having the amino acid sequence of SEQ ID NO:154
CDR2β having the amino acid sequence of SEQ ID NO:156
CDR3β having the amino acid sequence of SEQ ID NO:159; or
(c) (i) a TCRα chain variable domain incorporating the following CDRs:
CDR1α having the amino acid sequence of SEQ ID NO:165
CDR2α having the amino acid sequence of SEQ ID NO:150
CDR3α having the amino acid sequence of SEQ ID NO:152; and
(ii) a TCRβ chain variable domain incorporating the following CDRs:
CDR1β having the amino acid sequence of SEQ ID NO:31
CDR2β having the amino acid sequence of SEQ ID NO:157
CDR3β having the amino acid sequence of SEQ ID NO:160; or
(d) (i) a TCRα chain variable domain incorporating the following CDRs:
CDR1α having the amino acid sequence of SEQ ID NO:165
CDR2α having the amino acid sequence of SEQ ID NO:150
CDR3α having the amino acid sequence of SEQ ID NO:194; and
(ii) a TCRβ chain variable domain incorporating the following CDRs:
CDR1β having the amino acid sequence of SEQ ID NO:25
CDR2β having the amino acid sequence of SEQ ID NO:33
CDR3β having the amino acid sequence of SEQ ID NO:195; or
(e) (i) a TCRα chain variable domain incorporating the following CDRs:
CDR1α having the amino acid sequence of SEQ ID NO:2
CDR2α having the amino acid sequence of SEQ ID NO:8
CDR3α having the amino acid sequence of SEQ ID NO:196; and
(ii) a TCRβ chain variable domain incorporating the following CDRs:
CDR1β having the amino acid sequence of SEQ ID NO:197
CDR2β having the amino acid sequence of SEQ ID NO:198
CDR3β having the amino acid sequence of SEQ ID NO:199.
(f) (i) a TCRα chain variable domain incorporating the following CDRs:
CDR1α having the amino acid sequence of SEQ ID NO:149
CDR2α having the amino acid sequence of SEQ ID NO:150
CDR3α having the amino acid sequence of SEQ ID NO:14; and
(ii) a TCRβ chain variable domain incorporating the following CDRs:
CDR1β having the amino acid sequence of SEQ ID NO:32
CDR2β having the amino acid sequence of SEQ ID NO:40
CDR3β having the amino acid sequence of SEQ ID NO:42.

6. The TCR according to any one of claims 1 to 5, wherein the TCR comprises:
(a)
a TCRα chain variable domain comprising an amino acid sequence having at least 80%, 85%, 90%, or 95% sequence identity to an amino acid sequence selected from: SEQ ID NOs: 66, 162, 163 and 200; in combination with:
a TCRβ chain variable domain comprising an amino acid sequence having at least 80%, 85%, 90%, or 95% sequence identity to an amino acid sequence selected from: SEQ ID NOs: 91, 166, 167 and 201; or
(b)
a TCRα chain variable domain comprising an amino acid sequence having at least 80%, 85%, 90%, or 95% sequence identity to an amino acid sequence selected from: SEQ ID NOs: 66, 162 and 202; in combination with:
a TCRβ chain variable domain comprising an amino acid sequence having at least 80%, 85%, 90%, or 95% sequence identity to an amino acid sequence selected from: SEQ ID NOs: 91, 166 and 203; or
(c)
a TCRα chain variable domain comprising an amino acid sequence having at least about 80%, about 85%, about 90%, or about 95% sequence identity to an amino acid sequence selected from: SEQ ID NOs: 66, 162 and 163; in combination with:
a TCRβ chain variable domain comprising an amino acid sequence having at least 80%, 85%, 90%, or 95% sequence identity to an amino acid sequence selected from: SEQ ID NOs: 91, 166 and 167 or;
(d)
a TCRα chain variable domain comprising an amino acid sequence having at least about 80%, about 85%, about 90%, or about 95% sequence identity to an amino acid sequence selected from: SEQ ID NOs: 66 and 162; in combination with:
a TCRβ chain variable domain comprising an amino acid sequence having at least 80%, 85%, 90%, or 95% sequence identity to an amino acid sequence selected from: SEQ ID NOs: 91 and 166.

7. The TCR according to any one of claims 1 to 6, wherein the TCR comprises:
(a)
(i) a TCRα chain having the amino acid sequence of SEQ ID NO:66, and a TCRβ chain having the amino acid sequence of SEQ ID NO:91; or (ii) a TCRα chain having the amino acid sequence of SEQ ID NO: 162, and a TCRβ chain having the amino acid sequence of SEQ ID NO: 166; or (iii) a TCRα chain having the amino acid sequence of SEQ ID NO:163, and a TCRβ chain having the amino acid sequence of SEQ ID NO:167; or (iv) a TCRα chain having the amino acid sequence of SEQ ID NO: 200, and a TCRβ chain having the amino acid sequence of SEQ ID NO:201; or
(b)
(i) a TCRα chain having the amino acid sequence of SEQ ID NO:66, and a TCRβ chain having the amino acid sequence of SEQ ID NO:91; or (ii) a TCRα chain having the amino acid sequence of SEQ ID NO: 162, and a TCRβ chain having the amino acid sequence of SEQ ID NO: 166; or (iii) a TCRα chain having the amino acid sequence of SEQ ID NO:202, and a TCRβ chain having the amino acid sequence of SEQ ID NO:203; or
(c)
(i) a TCRα chain having the amino acid sequence of SEQ ID NO:66, and a TCRβ chain having the amino acid sequence of SEQ ID NO:91; or (ii) a TCRα chain having the amino acid sequence of SEQ ID NO: 162, and a TCRβ chain having the amino acid sequence of SEQ ID NO: 166; or (iii) a TCRα chain having the amino acid sequence of SEQ ID NO:163, and a TCRβ chain having the amino acid sequence of SEQ ID NO: 167; or
(d)
(i) a TCRα chain having the amino acid sequence of SEQ ID NO:66, and a TCRβ chain having the amino acid sequence of SEQ ID NO:91; or (ii) a TCRα chain having the amino acid sequence of SEQ ID NO: 162, and a TCRβ chain having the amino acid sequence of SEQ ID NO:166.

8. The TCR according to any one of claims 1 to 7, wherein the TCR comprises:
(a)
a TCRα chain variable domain encoded by a nucleotide sequence having at least 80%, 85%, 90%, or 95% sequence identity to a nucleotide sequence selected from: SEQ ID NOs: 169, 170, 175, 219, 176, 221, 186, 188, 204, 213 and 215; in combination with:
a TCRβ chain variable domain encoded by a nucleotide sequence having at least 80%, 85%, 90%, or 95% sequence identity to a nucleotide sequence selected from: SEQ ID NOs: 172, 173, 178, 220, 179, 222, 187, 189, 205, 214 and 216; or
(b)
a TCRα chain variable domain encoded by a nucleotide sequence having at least 80%, 85%, 90%, or 95% sequence identity to a nucleotide sequence selected from: SEQ ID NOs: 169, 175, 219, 186, 188, 206, and 217; in combination with:
a TCRβ chain variable domain encoded by a nucleotide sequence having at least 80%, 85%, 90%, or 95% sequence identity to a nucleotide sequence selected from: SEQ ID NOs: 172, 178, 220, 187, 189, 207 and 218; or
(c)
a TCRα chain variable domain encoded by a nucleotide sequence having at least 80%, 85%, 90%, or 95% sequence identity to a nucleotide sequence selected from: SEQ ID NOs: 169, 170, 175, 219, 176, 221, 186 and 188; in combination with:
a TCRβ chain variable domain encoded by a nucleotide sequence having at least 80%, 85%, 90%, or 95% sequence identity to a nucleotide sequence selected from: SEQ ID NOs: 172, 173, 178, 220, 179, 222, 187 and 189; or
(d)
a TCRα chain variable domain encoded by a nucleotide sequence having at least 80%, 85%, 90%, or 95% sequence identity to a nucleotide sequence selected from: SEQ ID NOs: 169, 175, 219, 186 and 188; in combination with:
a TCRβ chain variable domain encoded by a nucleotide sequence having at least 80%, 85%, 90%, or 95% sequence identity to a nucleotide sequence selected from: SEQ ID NOs: 172, 178, 220, 187 and 189.

9. The TCR according to any one of claims 1 to 8, wherein the TCR comprises:
(a)
(i) a TCRα chain variable domain encoded by the nucleotide sequence of SEQ ID NO: 169, and a TCRβ chain variable domain encoded by the nucleotide sequence of SEQ ID NO:172; or (ii) a TCRα chain variable domain encoded by the nucleotide sequence of SEQ ID NO:170, and a TCRβ chain variable domain encoded by the nucleotide sequence of SEQ ID NO: 173; or (iii) a TCRα chain variable domain encoded by the nucleotide sequence of SEQ ID NO:175, and a TCRβ chain variable domain encoded by the nucleotide sequence of SEQ ID NO:178; or (iv) a TCRα chain variable domain encoded by the nucleotide sequence of SEQ ID NO:219, and a TCRβ chain variable domain encoded by the nucleotide sequence of SEQ ID NO:220; or (v) a TCRα chain variable domain encoded by the nucleotide sequence of SEQ ID NO:176, and a TCRβ chain variable domain encoded by the nucleotide sequence of SEQ ID NO:179; or (vi) a TCRα chain variable domain encoded by the nucleotide sequence of SEQ ID NO: 221, and a TCRβ chain variable domain encoded by the nucleotide sequence of SEQ ID NO: 222; or (vii) a TCRα chain variable domain encoded by the nucleotide sequence of SEQ ID NO: 186, and a TCRβ chain variable domain encoded by the nucleotide sequence of SEQ ID NO:187; or (viii) a TCRα chain variable domain encoded by the nucleotide sequence of SEQ ID NO:188, and a TCRβ chain variable domain encoded by the nucleotide sequence of SEQ ID NO:189; or (ix) a TCRα chain variable domain encoded by the nucleotide sequence of SEQ ID NO:204, and a TCRβ chain variable domain encoded by the nucleotide sequence of SEQ ID NO:205; or (x) a TCRα chain variable domain encoded by the nucleotide sequence of SEQ ID NO:213, and a TCRβ chain variable domain encoded by the nucleotide sequence of SEQ ID NO:214; or (xi) a TCRα chain variable domain encoded by the nucleotide sequence of SEQ ID NO:215, and a TCRβ chain variable domain encoded by the nucleotide sequence of SEQ ID NO:216.
(b)
(i) a TCRα chain variable domain encoded by the nucleotide sequence of SEQ ID NO:169, and a TCRβ chain variable domain encoded by the nucleotide sequence of SEQ ID NO:172; or (ii) a TCRα chain variable domain encoded by the nucleotide sequence of SEQ ID NO:175, and a TCRβ chain variable domain encoded by the nucleotide sequence of SEQ ID NO:178; or (iii) a TCRα chain variable domain encoded by the nucleotide sequence of SEQ ID NO:219, and a TCRβ chain variable domain encoded by the nucleotide sequence of SEQ ID NO:220; or (iv) a TCRα chain variable domain encoded by the nucleotide sequence of SEQ ID NO:186, and a TCRβ chain variable domain encoded by the nucleotide sequence of SEQ ID NO: 187; or (v) a TCRα chain variable domain encoded by the nucleotide sequence of SEQ ID NO:188, and a TCRβ chain variable domain encoded by the nucleotide sequence of SEQ ID NO:189; or (vi) a TCRα chain variable domain encoded by the nucleotide sequence of SEQ ID NO: 206, and a TCRβ chain variable domain encoded by the nucleotide sequence of SEQ ID NO: 207; or (vii) a TCRα chain variable domain encoded by the nucleotide sequence of SEQ ID NO: 217, and a TCRβ chain variable domain encoded by the nucleotide sequence of SEQ ID NO:218.
(c)
(i) a TCRα chain variable domain encoded by the nucleotide sequence of SEQ ID NO: 169, and a TCRβ chain variable domain encoded by the nucleotide sequence of SEQ ID NO:172; or (ii) a TCRα chain variable domain encoded by the nucleotide sequence of SEQ ID NO:170, and a TCRβ chain variable domain encoded by the nucleotide sequence of SEQ ID NO:173; or (iii) a TCRα chain variable domain encoded by the nucleotide sequence of SEQ ID NO:175, and a TCRβ chain variable domain encoded by the nucleotide sequence of SEQ ID NO:178; or (iv) a TCRα chain variable domain encoded by the nucleotide sequence of SEQ ID NO:219, and a TCRβ chain variable domain encoded by the nucleotide sequence of SEQ ID NO:220; (v) a TCRα chain variable domain encoded by the nucleotide sequence of SEQ ID NO:176, and a TCRβ chain variable domain encoded by the nucleotide sequence of SEQ ID NO: 179; or (vi) a TCRα chain variable domain encoded by the nucleotide sequence of SEQ ID NO: 221, and a TCRβ chain variable domain encoded by the nucleotide sequence of SEQ ID NO:222; or (vii) a TCRα chain variable domain encoded by the nucleotide sequence of SEQ ID NO:186, and a TCRβ chain variable domain encoded by the nucleotide sequence of SEQ ID NO:187; or (viii) a TCRα chain variable domain encoded by the nucleotide sequence of SEQ ID NO:188, and a TCRβ chain variable domain encoded by the nucleotide sequence of SEQ ID NO:189; or
(d)
(i) a TCRα chain variable domain encoded by the nucleotide sequence of SEQ ID NO: 169, and a TCRβ chain variable domain encoded by the nucleotide sequence of SEQ ID NO:172; or (ii) a TCRα chain variable domain encoded by the nucleotide sequence of SEQ ID NO:175, and a TCRβ chain variable domain encoded by the nucleotide sequence of SEQ ID NO: 178; or (iii) a TCRα chain variable domain encoded by the nucleotide sequence of SEQ ID NO:219, and a TCRβ chain variable domain encoded by the nucleotide sequence of SEQ ID NO:220; (iv) a TCRα chain variable domain encoded by the nucleotide sequence of SEQ ID NO:186, and a TCRβ chain variable domain encoded by the nucleotide sequence of SEQ ID NO:187; or (v) a TCRα chain variable domain encoded by the nucleotide sequence of SEQ ID NO:188, and a TCRβ chain variable domain encoded by the nucleotide sequence of SEQ ID NO:189.

10. A T cell receptor (TCR) comprising a TCRα chain and a TCRβ chain, optionally isolated, that binds to an Epstein-Barr Virus (EBV)-derived antigenic peptide having: (i) an amino acid sequence selected from SEQ ID NO: 105, SEQ ID NO: 106, SEQ ID NO: 107 and SEQ ID NO: 146, when presented by a major histocompatibility complex (MHC) molecule comprising an MHC class I α chain polypeptide encoded by a HLA-A*02 allele; or (ii) the amino acid sequence of SEQ ID NO:145, when presented by a MHC molecule comprising an MHC class I α chain polypeptide encoded by a HLA-B*35 allele.

11. The TCR according to claim 10, wherein the TCR binds to the EBV BRLF1-derived antigenic peptide having the amino acid sequence of SEQ ID NO:105, when presented by a MHC molecule comprising an MHC class I α chain polypeptide encoded by a HLA-A*02 allele, and comprises:
a TCRα chain variable domain comprising a CDR3α with an amino acid sequence having at least 95% sequence identity to an amino acid sequence selected from: SEQ ID NOs: 15, 16, and 17; in combination with:
a TCRβ chain variable domain comprising a CDR3β with an amino acid sequence having at least 95% sequence identity to an amino acid sequence selected from: SEQ ID NOs: 43, 44, 45, and 46.

12. The TCR according to claim 10 or claim 11, wherein the TCR comprises: (i) a TCRα chain variable domain comprising a CDR3α having the amino acid sequence of SEQ ID NO:15, and a TCRβ chain variable domain comprising a CDR3β having the amino acid sequence of SEQ ID NO:43; or (ii) a TCRα chain variable domain comprising a CDR3α having the amino acid sequence of SEQ ID NO:16, and a TCRβ chain variable domain comprising a CDR3β having the amino acid sequence of SEQ ID NO:44; or (iii) a TCRα chain variable domain comprising a CDR3α having the amino acid sequence of SEQ ID NO:15, and a TCRβ chain variable domain comprising a CDR3β having the amino acid sequence of SEQ ID NO:45; or (iv) a TCRα chain variable domain comprising a CDR3α having the amino acid sequence of SEQ ID NO:17, and a TCRβ chain variable domain comprising a CDR3β having the amino acid sequence of SEQ ID NO:46.

13. The TCR according to any one of claims 10 to 12, wherein the TCR comprises:
(a) (i) a TCRα chain variable domain incorporating the following CDRs:
CDR1α having the amino acid sequence of SEQ ID NO:1
CDR2α having the amino acid sequence of SEQ ID NO:7
CDR3α having the amino acid sequence of SEQ ID NO:15; and
(ii) a TCRβ chain variable domain incorporating the following CDRs:
CDR1β having the amino acid sequence of SEQ ID NO:26
CDR2β having the amino acid sequence of SEQ ID NO:34
CDR3β having the amino acid sequence of SEQ ID NO:43; or
(b) (i) a TCRα chain variable domain incorporating the following CDRs:
CDR1α having the amino acid sequence of SEQ ID NO:1
CDR2α having the amino acid sequence of SEQ ID NO:7
CDR3α having the amino acid sequence of SEQ ID NO:16; and
(ii) a TCRβ chain variable domain incorporating the following CDRs:
CDR1β having the amino acid sequence of SEQ ID NO:27
CDR2β having the amino acid sequence of SEQ ID NO:35
CDR3β having the amino acid sequence of SEQ ID NO:44; or
(c) (i) a TCRα chain variable domain incorporating the following CDRs:
CDR1α having the amino acid sequence of SEQ ID NO:1
CDR2α having the amino acid sequence of SEQ ID NO:7
CDR3α having the amino acid sequence of SEQ ID NO:15; and
(ii) a TCRβ chain variable domain incorporating the following CDRs:
CDR1β having the amino acid sequence of SEQ ID NO:25
CDR2β having the amino acid sequence of SEQ ID NO:33
CDR3β having the amino acid sequence of SEQ ID NO:45; or
(d) (i) a TCRα chain variable domain incorporating the following CDRs:
CDR1α having the amino acid sequence of SEQ ID NO:2
CDR2α having the amino acid sequence of SEQ ID NO:8
CDR3α having the amino acid sequence of SEQ ID NO:17; and
(ii) a TCRβ chain variable domain incorporating the following CDRs:
CDR1β having the amino acid sequence of SEQ ID NO:28
CDR2β having the amino acid sequence of SEQ ID NO:36
CDR3β having the amino acid sequence of SEQ ID NO:46.

14. The TCR according to any one of claims 10 to 13, wherein the TCR comprises:
a TCRα chain variable domain comprising an amino acid sequence having at least 80%, 85%, 90%, or 95% sequence identity to an amino acid sequence selected from: SEQ ID NOs: 55, 56, and 57; in combination with:
a TCRβ chain variable domain comprising an amino acid sequence having at least 80%, 85%, 90%, or 95% sequence identity to an amino acid sequence selected from: SEQ ID NOs: 67, 68, 69 and 70.

15. The TCR according to any one of claims 10 to 14, wherein the TCR comprises: (i) a TCRα chain having the amino acid sequence of SEQ ID NO:55, and a TCRβ chain having the amino acid sequence of SEQ ID NO: 67; or (ii) a TCRα chain having the amino acid sequence of SEQ ID NO:56, and a TCRβ chain having the amino acid sequence of SEQ ID NO:68; or (iii) a TCRα chain having the amino acid sequence of SEQ ID NO: 55, and a TCRβ chain having the amino acid sequence of SEQ ID NO:69; or (iv) a TCRα chain having the amino acid sequence of SEQ ID NO:57, and a TCRβ chain having the amino acid sequence of SEQ ID NO:70.

16. The TCR according to any one of claims 10 to 15, wherein the TCR comprises:
a TCRα chain variable domain encoded by a nucleotide sequence having at least 80%, 85%, 90%, or 95% sequence identity to a nucleotide sequence selected from: SEQ ID NOs: 79, 80, 81, 108, 109, 110 and 111; in combination with:
a TCRβ chain variable domain encoded by a nucleotide sequence having at least 80%, 85%, 90%, or 95% sequence identity to a nucleotide sequence selected from: SEQ ID NOs: 92, 93, 94, 95, 121, 122, 123 and 124.

17. The TCR according to any one of claims 10 to 16, wherein the TCR comprises: (i) a TCRα chain variable domain encoded by the nucleotide sequence of SEQ ID NO:79, and a TCRβ chain variable domain encoded by the nucleotide sequence of SEQ ID NO:92; or (ii) a TCRα chain variable domain encoded by the nucleotide sequence of SEQ ID NO:80, and a TCRβ chain variable domain encoded by the nucleotide sequence of SEQ ID NO:93; or (iii) a TCRα chain variable domain encoded by the nucleotide sequence of SEQ ID NO:79, and a TCRβ chain variable domain encoded by the nucleotide sequence of SEQ ID NO:94; or (iv) a TCRα chain variable domain encoded by the nucleotide sequence of SEQ ID NO:81, and a TCRβ chain variable domain encoded by the nucleotide sequence of SEQ ID NO:95; or (v) a TCRα chain variable domain encoded by the nucleotide sequence of SEQ ID NO:108, and a TCRβ chain variable domain encoded by the nucleotide sequence of SEQ ID NO:121; or (vi) a TCRα chain variable domain encoded by the nucleotide sequence of SEQ ID NO:109, and a TCRβ chain variable domain encoded by the nucleotide sequence of SEQ ID NO: 122; or (vii) a TCRα chain variable domain encoded by the nucleotide sequence of SEQ ID NO:110, and a TCRβ chain variable domain encoded by the nucleotide sequence of SEQ ID NO:123; or (viii) a TCRα chain variable domain encoded by the nucleotide sequence of SEQ ID NO:111, and a TCRβ chain variable domain encoded by the nucleotide sequence of SEQ ID NO:124.

18. The TCR according to claim 10, wherein the TCR binds to the EBV LMP2-derived antigenic peptide having the amino acid sequence of SEQ ID NO:107, when presented by a MHC molecule comprising an MHC class I α chain polypeptide encoded by a HLA-A*02 allele, and comprises:
a TCRα chain variable domain comprising a CDR3α having the following amino acid sequence: C-A-T-X¹-G-X²-S-G-Y-S-T-L-T-F (SEQ ID NO:181); in combination with:
a TCRβ chain variable domain comprising a CDR3β having the following amino acid sequence: C-A-S-X³-X⁴-Q-G-G-(S)-X⁵-X⁶-G-Y-T-F (SEQ ID NO:182), wherein(S) is optional, and wherein:
X¹ is E or A;
X² is D, G, N or S; or E, A, Q or T;
X³ is S or T; or N or Q;
X⁴ is K, R or T; or H, or S;
X⁵ is Gor A;
X⁶ is Y or S; or F, W, H or T.

19. The TCR according to claim 10 or claim 18, wherein the TCR comprises:
a TCRα chain variable domain comprising a CDR3α with an amino acid sequence having at least 95% sequence identity to an amino acid sequence selected from SEQ ID NOs: 20, 23 and 24; in combination with:
a TCRβ chain variable domain comprising a CDR3β with an amino acid sequence having at least 95% sequence identity to an amino acid sequence selected from SEQ ID NOs: 49, 52, 53 and 54.

20. The TCR according to any one of claim 10, 18 or 19, wherein the TCR comprises: (i) a TCRα chain variable domain comprising a CDR3α having the amino acid sequence of SEQ ID NO:20, and a TCRβ chain variable domain comprising a CDR3β having the amino acid sequence of SEQ ID NO:49; or (ii) a TCRα chain variable domain comprising a CDR3α having the amino acid sequence of SEQ ID NO:23, and a TCRβ chain variable domain comprising a CDR3β having the amino acid sequence of SEQ ID NO:52; or (iii) a TCRα chain variable domain comprising a CDR3α having the amino acid sequence of SEQ ID NO:23, and a TCRβ chain variable domain comprising a CDR3β having the amino acid sequence of SEQ ID NO:53; or (iv) a TCRα chain variable domain comprising a CDR3α having the amino acid sequence of SEQ ID NO:24, and a TCRβ chain variable domain comprising a CDR3β having the amino acid sequence of SEQ ID NO:54.

21. The TCR according to any one of claims 10, or 18 to 20, wherein the TCR comprises:
(a) (i) a TCRα chain variable domain incorporating the following CDRs:
CDR1α having the amino acid sequence of SEQ ID NO:5
CDR2α having the amino acid sequence of SEQ ID NO:11 or 13
CDR3α having the amino acid sequence of SEQ ID NO:181, 20, 23 or 24; and
(ii) a TCRβ chain variable domain incorporating the following CDRs:
CDR1β having the amino acid sequence of SEQ ID NO:31
CDR2β having the amino acid sequence of SEQ ID NO:39 or 41
CDR3β having the amino acid sequence of SEQ ID NO:182, 49, 52, 53 or 54; or
(b) (i) a TCRα chain variable domain incorporating the following CDRs:
CDR1α having the amino acid sequence of SEQ ID NO:5
CDR2α having the amino acid sequence of SEQ ID NO:11
CDR3α having the amino acid sequence of SEQ ID NO:20; and
(ii) a TCRβ chain variable domain incorporating the following CDRs:
CDR1β having the amino acid sequence of SEQ ID NO:31
CDR2β having the amino acid sequence of SEQ ID NO:39
CDR3β having the amino acid sequence of SEQ ID NO:49; or
(c) (i) a TCRα chain variable domain incorporating the following CDRs:
CDR1α having the amino acid sequence of SEQ ID NO:5
CDR2α having the amino acid sequence of SEQ ID NO:13
CDR3α having the amino acid sequence of SEQ ID NO:23; and
(ii) a TCRβ chain variable domain incorporating the following CDRs:
CDR1β having the amino acid sequence of SEQ ID NO:31
CDR2β having the amino acid sequence of SEQ ID NO:41
CDR3β having the amino acid sequence of SEQ ID NO:52; or
(d) (i) a TCRα chain variable domain incorporating the following CDRs:
CDR1α having the amino acid sequence of SEQ ID NO:5
CDR2α having the amino acid sequence of SEQ ID NO:13
CDR3α having the amino acid sequence of SEQ ID NO:23; and
(ii) a TCRβ chain variable domain incorporating the following CDRs:
CDR1β having the amino acid sequence of SEQ ID NO:31
CDR2β having the amino acid sequence of SEQ ID NO:41
CDR3β having the amino acid sequence of SEQ ID NO:53; or
(e) (i) a TCRα chain variable domain incorporating the following CDRs:
CDR1α having the amino acid sequence of SEQ ID NO:5
CDR2α having the amino acid sequence of SEQ ID NO:13
CDR3α having the amino acid sequence of SEQ ID NO:24; and
(ii) a TCRβ chain variable domain incorporating the following CDRs:
CDR1β having the amino acid sequence of SEQ ID NO:31
CDR2β having the amino acid sequence of SEQ ID NO:41
CDR3β having the amino acid sequence of SEQ ID NO:54.

22. The TCR according to any one of claims 10, or 18 to 21, wherein the TCR comprises:
a TCRα chain variable domain comprising an amino acid sequence having at least 80%, 85%, 90%, or 95% sequence identity to an amino acid sequence selected from SEQ ID NOs: 60, 63, 64 and 65; in combination with:
a TCRβ chain variable domain comprising an amino acid sequence shares at least 80%, 85%, 90%, or 95% sequence identity to an amino acid sequence selected from SEQ ID NOs: 73, 76, 77 and 78.

23. The TCR according to any one of claims 10, or 18 to 22, wherein the TCR comprises: (i) a TCRα chain having the amino acid sequence of SEQ ID NO:60, and a TCRβ chain having the amino acid sequence of SEQ ID NO: 73; or (ii) a TCRα chain having the amino acid sequence of SEQ ID NO:63, and a TCRβ chain having the amino acid sequence of SEQ ID NO:76; or (iii) a TCRα chain having the amino acid sequence of SEQ ID NO: 64, and a TCRβ chain having the amino acid sequence of SEQ ID NO:77; or (iv) a TCRα chain having the amino acid sequence of SEQ ID NO:65, and a TCRβ chain having the amino acid sequence of SEQ ID NO:78.

24. The TCR according to any one of claims 10, or 18 to 23, wherein the TCR comprises:
a TCRα chain variable domain encoded by a nucleotide sequence having at least 80%, 85%, 90%, or 95% sequence identity to a nucleotide sequence selected from SEQ ID NOs: 84, 88, 89, 90, 114, 118, 119 and 120; in combination with:
a TCRβ chain variable domain encoded by a nucleotide sequence having at least 80%, 85%, 90%, or 95% sequence identity to a nucleotide sequence selected from SEQ ID NOs: 98, 102, 103, 104, 127, 131, 132 and 133.

25. The TCR according to any one of claims 10, or 18 to 24, wherein the TCR comprises: (i) a TCRα chain variable domain encoded by the nucleotide sequence of SEQ ID NO:84, and a TCRβ chain variable domain encoded by the nucleotide sequence of SEQ ID NO:98; or (ii) a TCRα chain variable domain encoded by the nucleotide sequence of SEQ ID NO:88, and a TCRβ chain variable domain encoded by the nucleotide sequence of SEQ ID NO:102; or (iii) a TCRα chain variable domain encoded by the nucleotide sequence of SEQ ID NO:89, and a TCRβ chain variable domain encoded by the nucleotide sequence of SEQ ID NO: 103; or (iv) a TCRα chain variable domain encoded by the nucleotide sequence of SEQ ID NO:90, and a TCRβ chain variable domain encoded by the nucleotide sequence of SEQ ID NO: 104; or (v) a TCRα chain variable domain encoded by the nucleotide sequence of SEQ ID NO:114, and a TCRβ chain variable domain encoded by the nucleotide sequence of SEQ ID NO:127; or (vi) a TCRα chain variable domain encoded by the nucleotide sequence of SEQ ID NO:118, and a TCRβ chain variable domain encoded by the nucleotide sequence of SEQ ID NO:131; or (vii) a TCRα chain variable domain encoded by the nucleotide sequence of SEQ ID NO:119, and a TCRβ chain variable domain encoded by the nucleotide sequence of SEQ ID NO: 132; or (viii) a TCRα chain variable domain encoded by the nucleotide sequence of SEQ ID NO:120, and a TCRβ chain variable domain encoded by the nucleotide sequence of SEQ ID NO:133.

26. The TCR according to claim 10, wherein the TCR binds to the EBV LMP2-derived antigenic peptide having the amino acid sequence of SEQ ID NO:106, when presented by a MHC molecule comprising an MHC class I α chain polypeptide encoded by a HLA-A*02 allele, and comprises:
a TCRα chain variable domain comprising a CDR3α having the following amino acid sequence: C-A-X¹-X²-G-A-G-S-Y-Q-L-T-F (SEQ ID NO:183); in combination with:
a TCRβ chain variable domain comprising a CDR3β having the following amino acid sequence: C-A-S-S-X³-E-G-Q-A-S-S-Y-E-Q-Y-F (SEQ ID NO:184), wherein:
X¹ is G or V; or A, I or L;
X² is A or S; or G or T;
X³ is L or A; or I, V or G.

27. The TCR according to claim 10 or claim 26, wherein the TCR comprises:
a TCRα chain variable domain comprising a CDR3α with an amino acid sequence amino acid sequence having at least 95% sequence identity to an amino acid sequence selected from SEQ ID NOs: 19, 21 and 22; in combination with:
a TCRβ chain variable domain comprising a CDR3β with an amino acid sequence having at least 95% sequence identity to an amino acid sequence selected from SEQ ID NOs: 48, 50 and 51.

28. The TCR according to any one of claim 10, 26 or 27, wherein the TCR comprises: (i) a TCRα chain variable domain comprising a CDR3α having the amino acid sequence of SEQ ID NO:19, and a TCRβ chain variable domain comprising a CDR3β having the amino acid sequence of SEQ ID NO:48; or (ii) a TCRα chain variable domain comprising a CDR3α having the amino acid sequence of SEQ ID NO:21, and a TCRβ chain variable domain comprising a CDR3β having the amino acid sequence of SEQ ID NO:50; or (iii) a TCRα chain variable domain comprising a CDR3α having the amino acid sequence of SEQ ID NO:22, and a TCRβ chain variable domain comprising a CDR3β having the amino acid sequence of SEQ ID NO:50; or (iv) a TCRα chain variable domain comprising a CDR3α having the amino acid sequence of SEQ ID NO:21, and a TCRβ chain variable domain comprising a CDR3β having the amino acid sequence of SEQ ID NO:51.

29. The TCR according to any one of claims 10, or 26 to 28, wherein the TCR comprises:
(a) (i) a TCRα chain variable domain incorporating the following CDRs:
CDR1α having the amino acid sequence of SEQ ID NO:4 or 6
CDR2α having the amino acid sequence of SEQ ID NO:10 or 12
CDR3α having the amino acid sequence of SEQ ID NO:183, 19, 21 or 22; and
(ii) a TCRβ chain variable domain incorporating the following CDRs:
CDR1β having the amino acid sequence of SEQ ID NO:30 or 32
CDR2β having the amino acid sequence of SEQ ID NO:38 or 40
CDR3β having the amino acid sequence of SEQ ID NO:184, 48, 50 or 51; or
(b) (i) a TCRα chain variable domain incorporating the following CDRs:
CDR1α having the amino acid sequence of SEQ ID NO:4
CDR2α having the amino acid sequence of SEQ ID NO:10
CDR3α having the amino acid sequence of SEQ ID NO:19; and
(ii) a TCRβ chain variable domain incorporating the following CDRs:
CDR1β having the amino acid sequence of SEQ ID NO:30
CDR2β having the amino acid sequence of SEQ ID NO:38
CDR3β having the amino acid sequence of SEQ ID NO:48; or
(c) (i) a TCRα chain variable domain incorporating the following CDRs:
CDR1α having the amino acid sequence of SEQ ID NO:6
CDR2α having the amino acid sequence of SEQ ID NO:12
CDR3α having the amino acid sequence of SEQ ID NO:21; and
(ii) a TCRβ chain variable domain incorporating the following CDRs:
CDR1β having the amino acid sequence of SEQ ID NO:32
CDR2β having the amino acid sequence of SEQ ID NO:40
CDR3β having the amino acid sequence of SEQ ID NO:50; or
(d) (i) a TCRα chain variable domain incorporating the following CDRs:
CDR1α having the amino acid sequence of SEQ ID NO:6
CDR2α having the amino acid sequence of SEQ ID NO:12
CDR3α having the amino acid sequence of SEQ ID NO:22; and
(ii) a TCRβ chain variable domain incorporating the following CDRs:
CDR1β having the amino acid sequence of SEQ ID NO:32
CDR2β having the amino acid sequence of SEQ ID NO:40
CDR3β having the amino acid sequence of SEQ ID NO:50; or
(e) (i) a TCRα chain variable domain incorporating the following CDRs:
CDR1α having the amino acid sequence of SEQ ID NO:6
CDR2α having the amino acid sequence of SEQ ID NO:12
CDR3α having the amino acid sequence of SEQ ID NO:21; and
(ii) a TCRβ chain variable domain incorporating the following CDRs:
CDR1β having the amino acid sequence of SEQ ID NO:32
CDR2β having the amino acid sequence of SEQ ID NO:40
CDR3β having the amino acid sequence of SEQ ID NO:51.

30. The TCR according to any one of claims 10, or 26 to 29, wherein the TCR comprises:
a TCRα chain variable domain comprising an amino acid sequence having at least 80%, 85%, 90%, or 95% sequence identity to an amino acid sequence selected from: SEQ ID NOs: 59, 61 and 62; in combination with:
a TCRβ chain variable domain comprising an amino acid sequence having at least 80%, 85%, 90%, or 95% sequence identity to an amino acid sequence selected from: SEQ ID NOs: 72, 74 and 75.

31. The TCR according to any one of claims 10, or 26 to 30, wherein the TCR comprises: (i) a TCRα chain having the amino acid sequence of SEQ ID NO:59, and a TCRβ chain having the amino acid sequence of SEQ ID NO: 72; or (ii) a TCRα chain having the amino acid sequence of SEQ ID NO:61, and a TCRβ chain having the amino acid sequence of SEQ ID NO:74; or (iii) a TCRα chain having the amino acid sequence of SEQ ID NO: 62, and a TCRβ chain having the amino acid sequence of SEQ ID NO:74; or (iv) a TCRα chain having the amino acid sequence of SEQ ID NO:61, and a TCRβ chain having the amino acid sequence of SEQ ID NO:75.

32. The TCR according to any one of claims 10, or 26 to 31, wherein the TCR comprises:
a TCRα chain variable domain encoded by a nucleotide sequence having at least 80%, 85%, 90%, or 95% sequence identity to a nucleotide sequence selected from: SEQ ID NOs: 83, 85, 86, 87, 113, 115, 116 and 117; in combination with:
a TCRβ chain variable domain encoded by a nucleotide sequence having at least 80%, 85%, 90%, or 95% sequence identity to a nucleotide sequence selected from: SEQ ID NOs: 97, 99, 100, 101, 126, 128, 129 and 130.

33. The TCR according to any one of claims 10, or 26 to 32, wherein the TCR comprises: (i) a TCRα chain variable domain encoded by the nucleotide sequence of SEQ ID NO:83, and a TCRβ chain variable domain encoded by the nucleotide sequence of SEQ ID NO:97; or (ii) a TCRα chain variable domain encoded by the nucleotide sequence of SEQ ID NO:85, and a TCRβ chain variable domain encoded by the nucleotide sequence of SEQ ID NO:99; or (iii) a TCRα chain variable domain encoded by the nucleotide sequence of SEQ ID NO:86, and a TCRβ chain variable domain encoded by the nucleotide sequence of SEQ ID NO:100; or (iv) a TCRα chain variable domain encoded by the nucleotide sequence of SEQ ID NO:87, and a TCRβ chain variable domain encoded by the nucleotide sequence of SEQ ID NO: 101; or (v) a TCRα chain variable domain encoded by the nucleotide sequence of SEQ ID NO:113, and a TCRβ chain variable domain encoded by the nucleotide sequence of SEQ ID NO:126; or (vi) a TCRα chain variable domain encoded by the nucleotide sequence of SEQ ID NO:115, and a TCRβ chain variable domain encoded by the nucleotide sequence of SEQ ID NO:128; or (vii) a TCRα chain variable domain encoded by the nucleotide sequence of SEQ ID NO:116, and a TCRβ chain variable domain encoded by the nucleotide sequence of SEQ ID NO:129; or (viii) a TCRα chain variable domain encoded by the nucleotide sequence of SEQ ID NO:117, and a TCRβ chain variable domain encoded by the nucleotide sequence of SEQ ID NO:130.

34. The TCR according to claim 10, wherein the TCR binds to the EBV LMP2-derived antigenic peptide having the amino acid sequence of SEQ ID NO:146, when presented by a MHC molecule comprising an MHC class I α chain polypeptide encoded by a HLA-A*02 allele, and comprises:
a TCRα chain variable domain comprising a CDR3α with an amino acid sequence having at least 95% sequence identity to SEQ ID NO:18; in combination with:
a TCRβ chain variable domain comprising a CDR3β with an amino acid sequence having at least 95% sequence identity to SEQ ID NO:47.

35. The TCR according to claim 10 or claim 34, wherein said TCR comprises:
(i) a TCRα chain variable domain incorporating the following CDRs:
CDR1α having the amino acid sequence of SEQ ID NO:3
CDR2α having the amino acid sequence of SEQ ID NO:9
CDR3α having the amino acid sequence of SEQ ID NO:18; and
(ii) a TCRβ chain variable domain incorporating the following CDRs:
CDR1β having the amino acid sequence of SEQ ID NO:29
CDR2β having the amino acid sequence of SEQ ID NO:37
CDR3β having the amino acid sequence of SEQ ID NO:47.

36. The TCR according to any one of claim 10, 34 or 35, wherein the TCR comprises:
a TCRα chain variable domain comprising an amino acid sequence having at least 80%, 85%, 90%, or 95% sequence identity to SEQ ID NO:58; in combination with:
a TCRβ chain variable domain comprising an amino acid sequence having at least 80%, 85%, 90%, or 95% sequence identity to SEQ ID NO:71.

37. The TCR according to any one of claims 10, or 34 to 36, wherein the TCR comprises:
a TCRα chain variable domain encoded by a nucleotide sequence having at least 80%, 85%, 90%, or 95% sequence identity to a nucleotide sequence selected from: SEQ ID NOs: 82 and 112; in combination with:
a TCRβ chain variable domain encoded by a nucleotide sequence having at least 80%, 85%, 90%, or 95% sequence identity to a nucleotide sequence selected from: SEQ ID NOs: 96 and 125.

38. The TCR according to any one of claims 10, or 34 to 37, wherein the TCR comprises: (i) a TCRα chain variable domain encoded by the nucleotide sequence of SEQ ID NO:82, and a TCRβ chain variable domain encoded by the nucleotide sequence of SEQ ID NO:96; or (ii) a TCRα chain variable domain encoded by the nucleotide sequence of SEQ ID NO:112, and a TCRβ chain variable domain encoded by the nucleotide sequence of SEQ ID NO:125.

39. The TCR according to claim 10, wherein the TCR binds to the EBV BZLF1-derived antigenic peptide having the amino acid sequence of SEQ ID NO:145, when presented by a MHC molecule comprising an MHC class I α chain polypeptide encoded by a HLA-B*35 allele, and comprises:
a TCRα chain variable domain comprising a CDR3α with an amino acid sequence having at least 95% sequence identity to SEQ ID NO:138; in combination with:
a TCRβ chain variable domain comprising a CDR3β with an amino acid sequence having at least 95% sequence identity to SEQ ID NO: 139.

40. The TCR according to claim 10 or claim 39, wherein said TCR comprises:
(i) a TCRα chain variable domain incorporating the following CDRs:
CDR1α having the amino acid sequence of SEQ ID NO:136
CDR2α having the amino acid sequence of SEQ ID NO:137
CDR3α having the amino acid sequence of SEQ ID NO:138; and
(ii) a TCRβ chain variable domain incorporating the following CDRs:
CDR1β having the amino acid sequence of SEQ ID NO:27
CDR2β having the amino acid sequence of SEQ ID NO:35
CDR3β having the amino acid sequence of SEQ ID NO:139.

41. The TCR according to any one of claim 10, 39 or 40, wherein the TCR comprises:
a TCRα chain variable domain comprising an amino acid sequence having at least 80%, 85%, 90%, or 95% sequence identity to SEQ ID NO:140; in combination with:
a TCRβ chain variable domain comprising an amino acid sequence having at least 80%, 85%, 90%, or 95% sequence identity to SEQ ID NO:141.

42. The TCR according to any one of claims 10, or 39 to 41, wherein the TCR comprises:
a TCRα chain variable domain encoded by a nucleotide sequence having at least 80%, 85%, 90%, or 95% sequence identity to a nucleotide sequence selected from: SEQ ID NO:142 and 134; in combination with:
a TCRβ chain variable domain encoded by a nucleotide sequence having at least 80%, 85%, 90%, or 95% sequence identity to a nucleotide sequence selected from: SEQ ID NO:143 and 135.

43. The TCR according to any one of claims 10, or 39 to 42, wherein the TCR comprises: (i) a TCRα chain variable domain encoded by the nucleotide sequence of SEQ ID NO:142, and a TCRβ chain variable domain encoded by the nucleotide sequence of SEQ ID NO:143; or (ii) a TCRα chain variable domain encoded by the nucleotide sequence of SEQ ID NO:134, and a TCRβ chain variable domain encoded by the nucleotide sequence of SEQ ID NO:135.

44. A T cell receptor (TCR) comprising a TCRα chain and a TCRβ chain, optionally isolated, that binds to a Human Endogenous Retrovirus (HERV)-derived antigenic peptide having the amino acid sequence of SEQ ID NO: 148, when presented by a major histocompatibility complex (MHC) molecule comprising an MHC class I α chain polypeptide encoded by a HLA-A*02 allele.

45. The TCR according to claim 44, wherein the TCR comprises:
a TCRα chain variable domain comprising a CDR3α amino acid sequence having at least 95% sequence identity to SEQ ID NO:153; in combination with:
a TCRβ chain variable domain comprising a CDR3β amino acid sequence having at least 95% sequence identity to SEQ ID NO:161.

46. The TCR according to claim 44 or claim 45, wherein the TCR comprises:
(i) a TCRα chain variable domain incorporating the following CDRs:
CDR1α having the amino acid sequence of SEQ ID NO:4
CDR2α having the amino acid sequence of SEQ ID NO:10
CDR3α having the amino acid sequence of SEQ ID NO:153; and
(ii) a TCRβ chain variable domain incorporating the following CDRs:
CDR1β having the amino acid sequence of SEQ ID NO:155
CDR2β having the amino acid sequence of SEQ ID NO:158
CDR3β having the amino acid sequence of SEQ ID NO:161.

47. The TCR according to any one of claims 44 to 46, wherein the TCR comprises: a TCRα chain variable domain comprising an amino acid sequence having at least 80%, 85%, 90%, or 95% sequence identity to SEQ ID NO: 164; in combination with a TCRβ chain variable domain comprising an amino acid sequence having at least 80%, 85%, 90%, or 95% sequence identity to SEQ ID NO:168.

48. The TCR according to any one of claims 44 to 47, wherein the TCR comprises:
a TCRα chain variable domain encoded by a nucleotide sequence having at least 80%, 85%, 90%, or 95% sequence identity to a nucleotide sequence selected from: SEQ ID NOs: 171 and 177; in combination with:
a TCRβ chain variable domain encoded by a nucleotide sequence having at least 80%, 85%, 90%, or 95% sequence identity to a nucleotide sequence selected from: SEQ ID NOs: 174 and 180.

49. The TCR according to any one of claims 44 to 48, wherein the TCR comprises: (i) a TCRα chain variable domain encoded by the nucleotide sequence of SEQ ID NO:171, and a TCRβ chain variable domain encoded by the nucleotide sequence of SEQ ID NO:174; or (ii) a TCRα chain variable domain encoded by the nucleotide sequence of SEQ ID NO:177, and a TCRβ chain variable domain encoded by the nucleotide sequence of SEQ ID NO:180.

50. A T cell receptor (TCR), optionally isolated, comprising: a TCRα chain comprising a TCRα constant region having at least 80%, 85%, 90%, or 95% sequence identity to an amino acid sequence selected from: SEQ ID NO: 211, 208 and 190; and a TCRβ chain comprising a TCRβ constant region having at least 80%, 85%, 90%, or 95% sequence identity to an amino acid sequence selected from: SEQ ID NO:212, 210, 209 and 191; wherein the TCR comprises:
(i) a TCRα chain variable domain and a TCRβ chain variable domain as defined in any one of claims 1 to 9; or
(ii) a TCRα chain variable domain and a TCRβ chain variable domain as defined in any one of claims 10 to 43; or
(iii) a TCRα chain variable domain and a TCRβ chain variable domain as defined in any one of claims 44 to 49.

51. A T cell receptor (TCR), optionally isolated, comprising:
(i) a TCRα chain and a TCRβ chain as defined in any one of claims 1 to 9, or claim 50 part (i); or
(ii) a TCRα chain and a TCRβ chain as defined in any one of claims 10 to 43, or claim 50 part (ii); or
(iii) a TCRα chain and a TCRβ chain as defined in any one of claims 44 to 49, or claim 50 part (iii);
wherein the TCR further comprises an antigen-binding moiety specific for an immune cell surface molecule, optionally wherein the immune cell surface molecule is a CD3 polypeptide.

52. An expression vector comprising a nucleotide sequence encoding a TCRα chain and a nucleotide sequence encoding a TCRβ chain, wherein the expression vector comprises:
(i) (a) a nucleotide sequence having at least 80%, 85%, 90%, or 95% sequence identity to a nucleotide sequence selected from: SEQ ID NOs: 169, 170, 175, 219, 176, 221, 186, 188, 204, 213 and 215; and a nucleotide sequence having at least 80%, 85%, 90%, or 95% sequence identity to a nucleotide sequence selected from: SEQ ID NOs: 172, 173, 178, 220, 179, 222, 187, 189, 205, 214 and 216; or
(b) a nucleotide sequence having at least 80%, 85%, 90%, or 95% sequence identity to a nucleotide sequence selected from: SEQ ID NOs: 169, 175, 219, 186, 188, 206, and 217; and a nucleotide sequence having at least 80%, 85%, 90%, or 95% sequence identity to a nucleotide sequence selected from: SEQ ID NOs: 172, 178, 220, 187, 189, 207 and 218; or
(c) a nucleotide sequence having at least 80%, 85%, 90%, or 95% sequence identity to a nucleotide sequence selected from: SEQ ID NOs: 169, 170, 175, 219, 176, 221, 186 and 188; and a nucleotide sequence having at least 80%, 85%, 90%, or 95% sequence identity to a nucleotide sequence selected from: SEQ ID NOs: 172, 173, 178, 220, 179, 222, 187 and 189; or
(d) a nucleotide sequence having at least 80%, 85%, 90%, or 95% sequence identity to a nucleotide sequence selected from: SEQ ID NOs: 169, 175, 219, 186 and 188; and a nucleotide sequence having at least 80%, 85%, 90%, or 95% sequence identity to a nucleotide sequence selected from: SEQ ID NOs: 172, 178, 220, 187 and 189; or
(ii) a nucleotide sequence having at least 80%, 85%, 90%, or 95% sequence identity to a nucleotide sequence selected from: SEQ ID NOs: 79, 80, 81, 108, 109, 110 and 111; and a nucleotide sequence having at least 80%, 85%, 90%, or 95% sequence identity to a nucleotide sequence selected from: SEQ ID NOs: 92, 93, 94, 95, 121, 122, 123 and 124; or
(iii) a nucleotide sequence having at least 80%, 85%, 90%, or 95% sequence identity to a nucleotide sequence selected from: SEQ ID NOs: 84, 88, 89, 90, 114, 118, 119 and 120; and a nucleotide sequence having at least 80%, 85%, 90%, or 95% sequence identity to a nucleotide sequence selected from: SEQ ID NOs: 98, 102, 103, 104, 127, 131, 132 and 133; or
(iv) a nucleotide sequence having at least 80%, 85%, 90%, or 95% sequence identity to a nucleotide sequence selected from: SEQ ID NOs: 83, 85, 86, 87, 113, 115, 116 and 117; and a nucleotide sequence having at least 80%, 85%, 90%, or 95% sequence identity to a nucleotide sequence selected from: SEQ ID NOs: 97, 99, 100, 101, 126, 128, 129 and 130; or
(v) a nucleotide sequence having at least 80%, 85%, 90%, or 95% sequence identity to a nucleotide sequence selected from: SEQ ID NOs: 82 and 112; and a nucleotide sequence having at least 80%, 85%, 90%, or 95% sequence identity to a nucleotide sequence selected from: SEQ ID NOs: 96 and 125; or
(vi) a nucleotide sequence having at least 80%, 85%, 90%, or 95% sequence identity to a nucleotide sequence selected from: SEQ ID NOs: 142 and 134; and a nucleotide sequence having at least 80%, 85%, 90%, or 95% sequence identity to a nucleotide sequence selected from: SEQ ID NOs: 143 and 135; or
(vii) a nucleotide sequence having at least 80%, 85%, 90%, or 95% sequence identity to a nucleotide sequence selected from: SEQ ID NOs: 171 and 177; and a nucleotide sequence having at least 80%, 85%, 90%, or 95% sequence identity to a nucleotide sequence selected from: SEQ ID NOs: 174 and 180.

53. An immune cell, optionally isolated, comprising:
(i) a TCR according to any one of claims 1 to 9 claim 50 part (i), or claim 51 part (i), or
(ii) the expression vector according to claim 52 part (i), wherein the T cell expresses a TCR comprising the TCRα chain and the TCRβ chain encoded by the nucleotide sequences of the expression vector.

54. An immune cell, optionally isolated, comprising:
(i) a TCR according to any one of claims 10 to 43 claim 50 part (ii), or claim 51 part (ii), or
(ii) the expression vector according to claim 52 part (ii), (iii), (iv), (v) or (vi), wherein the T cell expresses a TCR comprising the TCRα chain and the TCRβ chain encoded by the nucleotide sequences of the expression vector.

55. An immune cell, optionally isolated, comprising:
(i) a TCR according to any one of claims 44 to 49 claim 50 part (iii), or claim 51 part (iii), or
(ii) the expression vector according to claim 52 part (vii), wherein the T cell expresses a TCR comprising the TCRα chain and the TCRβ chain encoded by the nucleotide sequences of the expression vector.

56. The immune cell according to any one of claims 53 to 55, for use in the treatment of a subject suffering from a splice-variant of MAPK8IP2-, or HERV-K-, or EBV-associated disease/condition.

57. The immune cell for use according to claim 56, wherein the treatment further comprises administering one or more immune modulating agents to the subject, wherein the one or more immune modulating agents are selected from the group consisting of: cytokines, TLR agonists, RIG-I like receptor (RLR) agonists, immune checkpoint inhibitors, chemotherapeutic agents, antibodies, radiotherapy, and a combination thereof.

58. The immune cell according to claim 53, for use in the treatment of a disease/condition selected from: a cancer associated with mutation to SF3B1, a cancer associated with mutation to SUGP1, a cancer comprising cells comprising a mutant splice-factor-induced peptide of MAPK8IP2, a cancer comprising cells comprising the peptide of SEQ ID NO:147, a hematological cancer, a myeloid hematologic malignancy, myelodysplastic syndrome, leukemia, chronic lymphocytic leukemia, pancreatic cancer, acute myeloid leukemia and chronic myelomonocytic leukemia, melanoma, uveal melanoma, lung cancer, non-small cell lung cancer and pancreatic cancer.

59. The immune cell according to claim 54, for use in the treatment of a disease/condition selected from: an EBV-associated cancer, a cancer comprising cells comprising the peptide of SEQ ID NO: 105, a cancer comprising cells comprising the peptide of SEQ ID NO: 106, a cancer comprising cells comprising the peptide of SEQ ID NO:107, a cancer comprising cells comprising the peptide of SEQ ID NO:145, a cancer comprising cells comprising the peptide of SEQ ID NO: 146, a hematological cancer, a myeloid hematologic malignancy, a hematopoietic malignancy, a lymphoblastic hematologic malignancy, myelodysplastic syndrome, leukemia, T cell leukemia, acute myeloid leukemia, chronic myeloid leukemia, acute lymphoblastic leukemia, lymphoma, Hodgkin's lymphoma, non-Hodgkin's lymphoma, B cell non-Hodgkin's lymphoma, diffuse large B cell lymphoma, primary mediastinal B cell lymphoma, EBV-associated lymphoma, EBV-positive B cell lymphoma, EBV-positive diffuse large B cell lymphoma, EBV-positive lymphoma associated with X-linked lymphoproliferative disorder, EBV-positive lymphoma associated with HIV infection/AIDS, oral hairy leukoplakia, Burkitt's lymphoma, post-transplant lymphoproliferative disease, central nervous system lymphoma, anaplastic large cell lymphoma, T cell lymphoma, ALK-positive anaplastic T cell lymphoma, ALK-negative anaplastic T cell lymphoma, peripheral T cell lymphoma, cutaneous T cell lymphoma, NK-T cell lymphoma, extra-nodal NK-T cell lymphoma, thymoma, multiple myeloma, a solid cancer, epithelial cell cancer, gastric cancer, gastric carcinoma, gastric adenocarcinoma, gastrointestinal adenocarcinoma, liver cancer, hepatocellular carcinoma, cholangiocarcinoma, head and neck cancer, head and neck squamous cell carcinoma, oral cavity cancer, oropharyngeal cancer, oropharyngeal carcinoma, oral cancer, laryngeal cancer, nasopharyngeal carcinoma, oesophageal cancer, colorectal cancer, colorectal carcinoma, colon cancer, colon carcinoma, cervical carcinoma, prostate cancer, lung cancer, non-small cell lung cancer, small cell lung cancer, lung adenocarcinoma, squamous lung cell carcinoma, bladder cancer, urothelial carcinoma, skin cancer, melanoma, advanced melanoma, renal cell cancer, renal cell carcinoma, ovarian cancer, ovarian carcinoma, mesothelioma, breast cancer, brain cancer, glioblastoma, prostate cancer, pancreatic cancer, mastocytosis, advanced systemic mastocytosis, germ cell tumor, testicular embryonal carcinoma, an autoimmune disease, SLE, systemic scleroderma, multiple sclerosis, Sjögren's syndrome, arthritis, rheumatoid arthritis, juvenile idiopathic arthritis, inflammatory bowel disease, Crohn's disease, ulcerative colitis, diabetes, type 1 diabetes, and celiac disease.

60. The immune cell according to claim 55, for use in the treatment of a disease/condition selected from: a cancer comprising cells expressing a HERV protein, a cancer comprising cells expressing a HERV-K protein, a cancer comprising cells comprising a HERV-K gag protein-derived peptide, a cancer comprising cells comprising the peptide of SEQ ID NO:148, breast cancer, pancreatic cancer, germ cell tumor, a hematological cancer, leukemia, prostate cancer, bladder cancer, ovarian cancer, lung cancer, liver cancer, hepatocellular carcinoma, lymphoma, uterine cancer, choriocarcinoma, colorectal cancer, colorectal carcinoma, sarcoma, soft tissue sarcoma and Kaposi's sarcoma.


SEQUENCE LISTING (309 sequences)

SEQ ID NO: 1 - protein, 6 aa, synthetic construct
YGGTVN

SEQ ID NO: 2 - protein, 7 aa, synthetic construct
TSDQSYG

SEQ ID NO: 3 - protein, 6 aa, synthetic construct
SSVSVY

SEQ ID NO: 4 - protein, 6 aa, synthetic construct
DSAIYN

SEQ ID NO: 5 - protein, 5 aa, synthetic construct
TSINN

SEQ ID NO: 6 - protein, 5 aa, synthetic construct
TTLSN

SEQ ID NO: 7 - protein, 8 aa, synthetic construct
YFSGDPLV

SEQ ID NO: 8 - protein, 8 aa, synthetic construct
QGSYDEQN

SEQ ID NO: 9 - protein, 8 aa, synthetic construct
YLSGSTLV

SEQ ID NO: 10 - protein, 7 aa, synthetic construct
IQSSQRE

SEQ ID NO: 11 - protein, 7 aa, synthetic construct
IRSNERE

SEQ ID NO: 12 - protein, 7 aa, synthetic construct
LVKSGEV

SEQ ID NO: 13 - protein, 7 aa, synthetic construct
IRSNERE

SEQ ID NO: 14 - protein, 14 aa, synthetic construct
CAFMEADSNYQLIW

SEQ ID NO: 15 - protein, 11 aa, synthetic construct
CAVKDTDKLIF

SEQ ID NO: 16 - protein, 12 aa, synthetic construct
CAGGAAGNKLTF

SEQ ID NO: 17 - protein, 14 aa, synthetic construct
CAMREGGNFNKFYF

SEQ ID NO: 18 - protein, 16 aa, synthetic construct
CAVSALSYNQGGKLIF

SEQ ID NO: 19 - protein, 13 aa, synthetic construct
CAVLMDSNYQLIW

SEQ ID NO: 20 - protein, 14 aa, synthetic construct
CATEGSSGYSTLTF

SEQ ID NO: 21 - protein, 13 aa, synthetic construct
CAGAGAGSYQLTF

SEQ ID NO: 22 - protein, 13 aa, synthetic construct
CAVSGAGSYQLTF

SEQ ID NO: 23 - protein, 14 aa, synthetic construct
CATEGGSGYSTLTF

SEQ ID NO: 24 - protein, 14 aa, synthetic construct
CATAGNSGYSTLTF

SEQ ID NO: 25 - protein, 5 aa, synthetic construct
KGHDR

SEQ ID NO: 26 - protein, 5 aa, synthetic construct
LGHDT

SEQ ID NO: 27 - protein, 5 aa, synthetic construct
SGHAT

SEQ ID NO: 28 - protein, 5 aa, synthetic construct
SQVTM

SEQ ID NO: 29 - protein, 5 aa, synthetic construct
SGHNS

SEQ ID NO: 30 - protein, 5 aa, synthetic construct
WSHSY

SEQ ID NO: 31 - protein, 5 aa, synthetic construct
MNHEY

SEQ ID NO: 32 - protein, 5 aa, synthetic construct
SGHRS

SEQ ID NO: 33 - protein, 6 aa, synthetic construct
SFDVKD

SEQ ID NO: 34 - protein, 6 aa, synthetic construct
YNNKEL

SEQ ID NO: 35 - protein, 6 aa, synthetic construct
FQNNGV

SEQ ID NO: 36 - protein, 7 aa, synthetic construct
ANQGSEA

SEQ ID NO: 37 - protein, 6 aa, synthetic construct
FNNNVP

SEQ ID NO: 38 - protein, 6 aa, synthetic construct
SAAADI

SEQ ID NO: 39 - protein, 6 aa, synthetic construct
SVGAGI

SEQ ID NO: 40 - protein, 6 aa, synthetic construct
YFSETQ

SEQ ID NO: 41 - protein, 6 aa, synthetic construct
SVGAGI

SEQ ID NO: 42 - protein, 17 aa, synthetic construct
CASKGRRGPDYNSPLHF

SEQ ID NO: 43 - protein, 12 aa, synthetic construct
CASSPDFNEQFF

SEQ ID NO: 44 - protein, 18 aa, synthetic construct
CASSSPLGGFAGANVLTF

SEQ ID NO: 45 - protein, 13 aa, synthetic construct
CATSDFISDTQYF

SEQ ID NO: 46 - protein, 17 aa, synthetic construct
CSVGGTSGTLPANEQFF

SEQ ID NO: 47 - protein, 12 aa, synthetic construct
CASSWTGNEQYF

SEQ ID NO: 48 - protein, 14 aa, synthetic construct
CASSSDGMNTEAFF

SEQ ID NO: 49 - protein, 14 aa, synthetic construct
CASSKQGGGYGYTF

SEQ ID NO: 50 - protein, 16 aa, synthetic construct
CASSLEGQASSYEQYF

SEQ ID NO: 51 - protein, 16 aa, synthetic construct
CASSAEGQASSYEQYF

SEQ ID NO: 52 - protein, 15 aa, synthetic construct
CASSRQGGSGSGYTF

SEQ ID NO: 53 - protein, 14 aa, synthetic construct
CASTTQGGAYGYTF

SEQ ID NO: 54 - protein, 13 aa, synthetic construct
CASTPQGGNEAFF

SEQ ID NO: 55 - protein, 111 aa, synthetic construct
AQSVSQHNHHVILSEAASLELGCNYSYGGTVNLFWYVQYPGQHLQLLLKYFSGDPLVKGIKGFEAEFIKSKFSFNLRKPSVQWSDTAEYFCAVKDTDKLIFGTGTRLQVFP

SEQ ID NO: 56 - protein, 112 aa, synthetic construct
AQSVSQHNHHVILSEAASLELGCNYSYGGTVNLFWYVQYPGQHLQLLLKYFSGDPLVKGIKGFEAEFIKSKFSFNLRKPSVQWSDTAEYFCAGGAAGNKLTFGGGTRVLVKP

SEQ ID NO: 57 - protein, 115 aa, synthetic construct
AQKITQTQPGMFVQEKEAVTLDCTYDTSDQSYGLFWYKQPSSGEMIFLIYQGSYDEQNATEGRYSLNFQKARKSANLVISASQLGDSAMYFCAMREGGNFNKFYFGSGTKLNVKP

SEQ ID NO: 58 - protein, 116 aa, synthetic construct
AQSVTQLDSQVPVFEEAPVELRCNYSSSVSVYLFWYVQYPNQGLQLLLKYLSGSTLVKGINGFEAEFNKSQTSFHLRKPSVHISDTAEYFCAVSALSYNQGGKLIFGQGTELSVKP

SEQ ID NO: 59 - protein, 112 aa, synthetic construct
KQEVTQIPAALSVPEGENLVLNCSFTDSAIYNLQWFRQDPGKGLTSLLLIQSSQREQTSGRLNASLDKSSGRSTLYIAASQPGDSATYLCAVLMDSNYQLIWGAGTKLIIKP

SEQ ID NO: 60 - protein, 112 aa, synthetic construct
SQQGEEDPQALSIQEGENATMNCSYKTSINNLQWYRQNSGRGLVHLILIRSNEREKHSGRLRVTLDTSKKSSSLLITASRAADTASYFCATEGSSGYSTLTFGKGTMLLVSP

SEQ ID NO: 61 - protein, 111 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAGAGAGSYQLTFGKGTKLSVIP

SEQ ID NO: 62 - protein, 111 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAVSGAGSYQLTFGKGTKLSVIP

SEQ ID NO: 63 - protein, 112 aa, synthetic construct
SQQGEEDPQALSIQEGENATMNCSYKTSINNLQWYRQNSGRGLVHLILIRSNEREKHSGRLRVTLDTSKKSSSLLITASRAADTASYFCATEGGSGYSTLTFGKGTMLLVSP

SEQ ID NO: 64 - protein, 112 aa, synthetic construct
SQQGEEDPQALSIQEGENATMNCSYKTSINNLQWYRQNSGRGLVHLILIRSNEREKHSGRLRVTLDTSKKSSSLLITASRAADTASYFCATEGDSGYSTLTFGKGTMLLVSP

SEQ ID NO: 65 - protein, 112 aa, synthetic construct
SQQGEEDPQALSIQEGENATMNCSYKTSINNLQWYRQNSGRGLVHLILIRSNEREKHSGRLRVTLDTSKKSSSLLITASRAADTASYFCATAGNSGYSTLTFGKGTMLLVSP

SEQ ID NO: 66 - protein, 115 aa, synthetic construct
AQTVTQSQPEMSVQEAETVTLSCTYDTSESNYYLFWYKQPPSRQMILVIRQEAYKQQNATENRFSVNFQKAAKSFSLKISDSQLGDTAMYFCAFMEADSNYQLIWGAGTKLIIKP

SEQ ID NO: 67 - protein, 111 aa, synthetic construct
DTAVSQTPKYLVTQMGNDKSIKCEQNLGHDTMYWYKQDSKKFLKIMFSYNNKELIINETVPNRFSPKSPDKAHLNLHINSLELGDSAVYFCASSPDFNEQFFGPGTRLTVL

SEQ ID NO: 68 - protein, 118 aa, synthetic construct
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSSPLGGFAGANVLTFGAGSRLTVL

SEQ ID NO: 69 - protein, 112 aa, synthetic construct
DADVTQTPRNRITKTGKRIMLECSQTKGHDRMYWYRQDPGLGLRLIYYSFDVKDINKGEISDGYSVSRQAQAKFSLSLESAIPNQTALYFCATSDFISDTQYFGPGTRLTVL

SEQ ID NO: 70 - protein, 118 aa, synthetic construct
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGGTSGTLPANEQFFGPGTRLTVL

SEQ ID NO: 71 - protein, 112 aa, synthetic construct
DAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSWTGNEQYFGPGTRLTVT

SEQ ID NO: 72 - protein, 113 aa, synthetic construct
DAGITQSPRYKITETGRQVTLMCHQTWSHSYMFWYRQDLGHGLRLIYYSAAADITDKGEVPDGYVVSRSKTENFPLTLESATRSQTSVYFCASSSDGMNTEAFFGQGTRLTVV

SEQ ID NO: 73 - protein, 113 aa, synthetic construct
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSKQGGGYGYTFGSGTRLTVV

SEQ ID NO: 74 - protein, 115 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLEGQASSYEQYFGPGTRLTVT

SEQ ID NO: 75 - protein, 115 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSAEGQASSYEQYFGPGTRLTVT

SEQ ID NO: 76 - protein, 114 aa, synthetic construct
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSRQGGSGSGYTFGSGTRLTVV

SEQ ID NO: 77 - protein, 113 aa, synthetic construct
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASTTQGGAYGYTFGSGTRLTVV

SEQ ID NO: 78 - protein, 112 aa, synthetic construct
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASTPQGGNEAFFGQGTRLTVV

SEQ ID NO: 79 - DNA, 333 bp, synthetic construct
  source 1..333: mol_type = other DNA
gcccagtctgtgagccagcataaccaccacgtaattctctctgaagcagcctcactggagttgggatgcaactattcctatggtggaactgttaatctcttctggtatgtccagtaccctggtcaacaccttcagcttctcctcaagtacttttcaggggatccactggttaaaggcatcaagggctttgaggctgaatttataaagagtaaattctcctttaatctgaggaaaccctctgtgcagtggagtgacacagctgagtacttctgtgccgtgaaggacaccgacaagctcatctttgggactgggaccagattacaagtctttcca

SEQ ID NO: 80 - DNA, 336 bp, synthetic construct
  source 1..336: mol_type = other DNA
gcccagtctgtgagccagcataaccaccacgtaattctctctgaagcagcctcactggagttgggatgcaactattcctatggtggaactgttaatctcttctggtatgtccagtaccctggtcaacaccttcagcttctcctcaagtacttttcaggggatccactggttaaaggcatcaagggctttgaggctgaatttataaagagtaaattctcctttaatctgaggaaaccctctgtgcagtggagtgacacagctgagtacttctgtgccgggggagctgcaggcaacaagctaacttttggaggaggaaccagggtgctagttaaacca

SEQ ID NO: 81 - DNA, 345 bp, synthetic construct
  source 1..345: mol_type = other DNA
gcccagaagataactcaaacccaaccaggaatgttcgtgcaggaaaaggaggctgtgactctggactgcacatatgacaccagtgatcaaagttatggtctattctggtacaagcagcccagcagtggggaaatgatttttcttatttatcaggggtcttatgacgagcaaaatgcaacagaaggtcgctactcattgaatttccagaaggcaagaaaatccgccaaccttgtcatctccgcttcacaactgggggactcagcaatgtatttctgtgcaatgagagagggcgggaacttcaacaaattttactttggatctgggaccaaactcaatgtaaaacca

SEQ ID NO: 82 - DNA, 348 bp, synthetic construct
  source 1..348: mol_type = other DNA
gcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttaaaggcatcaacggttttgaggctgaatttaacaagagtcaaacttccttccacttgaggaaaccctcagtccatataagcgacacggctgagtacttctgtgctgtgagtgccctttcttataaccagggaggaaagcttatcttcggacagggaacggagttatctgtgaaaccc

SEQ ID NO: 83 - DNA, 336 bp, synthetic construct
  source 1..336: mol_type = other DNA
aaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtccttatggatagcaactatcagttaatctggggcgctgggaccaagctaattataaagcca

SEQ ID NO: 84 - DNA, 336 bp, synthetic construct
  source 1..336: mol_type = other DNA
agtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggagggctcttcaggatacagcaccctcacctttgggaaggggactatgcttctagtctctcca

SEQ ID NO: 85 - DNA, 333 bp, synthetic construct
  source 1..333: mol_type = other DNA
ggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcaggagctggggctgggagttaccaactcactttcgggaaggggaccaaactctcggtcatacca

SEQ ID NO: 86 - DNA, 333 bp, synthetic construct
  source 1..333: mol_type = other DNA
ggacaacaggtaatgcaaattcctcagtaccagcatgtacaagaaggagaagacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcagtctctggggctgggagttaccaactcactttcgggaaggggaccaaactctcggtcatacca

SEQ ID NO: 87 - DNA, 333 bp, synthetic construct
  source 1..333: mol_type = other DNA
ggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcaggggctggggctgggagttaccaactcactttcgggaaggggaccaaactctcggtcatacca

SEQ ID NO: 88 - DNA, 336 bp, synthetic construct
  source 1..336: mol_type = other DNA
agtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctactgagggcggttcaggatacagcaccctcacctttgggaaggggactatgcttctagtctctcca

SEQ ID NO: 89 - DNA, 336 bp, synthetic construct
  source 1..336: mol_type = other DNA
agtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggagggggattcaggatacagcaccctcacctttgggaaggggactatgcttctagtctctcca

SEQ ID NO: 90 - DNA, 336 bp, synthetic construct
  source 1..336: mol_type = other DNA
agtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggccggtaattcaggatacagcaccctcacctttgggaaggggactatgcttctagtctctcca

SEQ ID NO: 91 - protein, 116 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASKGRRGPDYNSPLHFGNGTRLTVT

SEQ ID NO: 92 - DNA, 333 bp, synthetic construct
  source 1..333: mol_type = other DNA
gacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagcccagacttcaatgagcagttcttcgggccagggacacggctcaccgtgcta

SEQ ID NO: 93 - DNA, 354 bp, synthetic construct
  source 1..354: mol_type = other DNA
gaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccaacctgcaaagcttgaggactcggccgtgtatctctgtgccagcagttcaccattgggggggttcgcgggggccaacgtcctgactttcggggccggcagcaggctgaccgtgctg

SEQ ID NO: 94 - DNA, 336 bp, synthetic construct
  source 1..336: mol_type = other DNA
gatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgatttcatctcagatacgcagtattttggcccaggcacccggctgacagtgctc

SEQ ID NO: 95 - DNA, 354 bp, synthetic construct
  source 1..354: mol_type = other DNA
agtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttggtgggactagcgggactctccctgccaatgagcagttcttcgggccagggacacggctcaccgtgcta

SEQ ID NO: 96 - DNA, 336 bp, synthetic construct
  source 1..336: mol_type = other DNA
gatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagctggacagggaacgagcagtacttcgggccgggcaccaggctcacggtcaca

SEQ ID NO: 97 - DNA, 339 bp, synthetic construct
  source 1..339: mol_type = other DNA
gatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctatgttgtctccagatccaagacagagaatttccccctcactctggagtcagctacccgctcccagacatctgtgtatttctgcgccagcagctcggacgggatgaacactgaagctttctttggacaaggcaccagactcacagttgta

SEQ ID NO: 98 - DNA, 339 bp, synthetic construct
  source 1..339: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagtaaacagggagggggctatggctacaccttcggttcggggaccaggttaaccgttgta

SEQ ID NO: 99 - DNA, 345 bp, synthetic construct
  source 1..345: mol_type = other DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcctcgagggacaggcgagctcctacgagcagtacttcgggccgggcaccaggctcacggtcaca

SEQ ID NO: 100 - DNA, 345 bp, synthetic construct
  source 1..345: mol_type = other DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcttggaggggcaggcctcctcctacgagcagtacttcgggccgggcaccaggctcacggtcaca

SEQ ID NO: 101 - DNA, 345 bp, synthetic construct
  source 1..345: mol_type = other DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcgcggagggacaggcttcctcctacgagcagtacttcgggccgggcaccaggctcacggtcaca

SEQ ID NO: 102 - DNA, 342 bp, synthetic construct
  source 1..342: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagtcgacaagggggttccgggagtggctacaccttcggttcggggaccaggttaaccgttgta

SEQ ID NO: 103 - DNA, 339 bp, synthetic construct
  source 1..339: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcaccacccaggggggggcctatggctacaccttcggttcggggaccaggttaaccgttgta

SEQ ID NO: 104 - DNA, 336 bp, synthetic construct
  source 1..336: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcaccccccaggggggcaacgaagctttctttggacaaggcaccagactcacagttgta

SEQ ID NO: 105 - protein, 9 aa, synthetic construct
YVLDHLIVV

SEQ ID NO: 106 - protein, 9 aa, synthetic construct
CLGGLLTMV

SEQ ID NO: 107 - protein, 9 aa, synthetic construct
FLYALALLL

SEQ ID NO: 108 - DNA, 333 bp, synthetic construct
  source 1..333: mol_type = other DNA
gcgcagagcgtttcgcaacacaaccaccacgtcatcctgtccgaggctgcttctctggagctggggtgcaactacagctacggtggcacggtcaatctattttggtacgtgcagtatccaggacagcatctccagctgctgctcaagtacttttcaggggacccgttggtgaaaggcatcaagggcttcgaagcagagttcattaagtcgaaattttccttcaacctgcgtaagccttccgtgcagtggtctgatactgccgagtacttctgtgccgtgaaggacaccgacaagcttatcttcggtaccggcacccgcctgcaggtgttcccc

SEQ ID NO: 109 - DNA, 336 bp, synthetic construct
  source 1..336: mol_type = other DNA
gcgcagagcgtgtcccaacacaaccaccacgtcatcctgtccgaggctgcttccctggagctggggtgcaactacagctacggcggcaccgtcaatttgttctggtacgtgcagtatccgggacagcatctccagctgctgctcaagtactttagtggtgatccacttgttaaaggcatcaagggcttcgaagccgagttcattaagtcgaaattttcattcaacctgcgcaagccctctgtgcagtggtctgacaccgcagagtacttctgtgccgggggcgcggccggcaacaagctgaccttcggaggcggtactcgtgtgctggtgaagcct

SEQ ID NO: 110 - DNA, 333 bp, synthetic construct
  source 1..333: mol_type = other DNA
gcgcagagcgtgtcccaacacaaccaccacgttatcctgtccgaggctgcatccctggagctggggtgcaactacagctacggtggcaccgtcaatctattttggtacgtgcagtatcctggacagcatttgcagctgctgctcaagtacttcagtggggacccgctggtgaaaggcatcaagggcttcgaagctgagttcattaagtcgaaattttcattcaacctgcgcaagccctctgtgcagtggtctgatacggccgagtacttttgtgccgtgaaggacaccgacaagcttatcttcggtaccggcactcgtctccaggtcttcccc

SEQ ID NO: 111 - DNA, 345 bp, synthetic construct
  source 1..345: mol_type = other DNA
gcacagaagatcacccagactcaacctggtatgtttgtgcaggagaaggaggctgtcacattggactgtacctacgacacctccgaccagagctacggccttttctggtacaagcagccgagttcgggggagatgatcttcctgatctatcagggctcctacgacgaacagaacgcgacggagggccgctactccctcaacttccagaaggcccgcaaaagcgcgaacctggtgatttctgcttcccagctgggtgattctgccatgtatttctgcgccatgcgtgagggcggcaacttcaacaagttctacttcggatcagggaccaagctgaatgtgaagccc

SEQ ID NO: 112 - DNA, 348 bp, synthetic construct
  source 1..348: mol_type = other DNA
gcgcagagcgtcacccagttggattctcaggtcccagtgttcgaggaggctccggtggagcttcgttgtaactactcctcgtcagtatccgtgtacctcttttggtacgtgcagtatcccaaccagggtctgcagctgctgctcaagtacctgtctggctccactctggtgaagggcattaatggcttcgaagcagagttcaacaagtcgcagacctccttccatctgcgcaagccctctgtccacatctccgacaccgccgagtacttttgcgccgtgtccgccctgagctacaaccaagggggtaagctaatcttcggacagggcaccgagctgagtgttaaacct

SEQ ID NO: 113 - DNA, 336 bp, synthetic construct
  source 1..336: mol_type = other DNA
aagcaggaggtgacacaaattcccgccgccctgtccgtccccgagggcgagaatctggtgctcaactgctcttttaccgacagtgccatctacaacctgcagtggttccgccaggacccgggcaagggtctgacctccctgctgctcatccagagctcacagcgggaacagacttccggccgcctgaacgcgtctttggacaaaagctccgggcgctcgaccctgtacatcgccgcttcccagccaggtgattctgctacctacctgtgcgccgtgctgatggacagcaactatcagcttatttggggcgccggcaccaagctgatcatcaagcct

SEQ ID NO: 114 - DNA, 336 bp, synthetic construct
  source 1..336: mol_type = other DNA
tcacaacagggcgaggaagatcctcaggccctcagcatccaggagggggagaatgcaacaatgaactgctcttacaagaccagcattaacaacctgcagtggtaccgccagaactccggtcgtggtttggtgcatttgatcctgatccgcagcaacgagagggagaagcacagtggacgcctgcgggtcaccctggacacctccaagaagtcgtcctctctgctcatcaccgcttcccgcgccgcggacactgctagctatttttgtgccaccgagggctcctctggctactccactcttaccttcggcaaaggcaccatgctgctggtgtcgccc

SEQ ID NO: 115 - DNA, 333 bp, synthetic construct
  source 1..333: mol_type = other DNA
ggacagcaggtcatgcaaattcctcagtaccagcatgtccaggagggcgaggacttcaccacttactgcaatagctcgaccactttgagcaacatccagtggtacaagcagcgtccgggcggccaccccgtgttcctgatccagctggtgaagtctggggaagttaagaaacagaagcgcctgaccttccagtttggagaggccaagaagaactcctctcttcacatcaccgccacccagacaaccgatgtggggacctacttttgtgctggtgcgggtgcaggctcctatcagctcaccttcggcaaaggcaccaagctgtccgtgatccca

SEQ ID NO: 116 - DNA, 333 bp, synthetic construct
  source 1..333: mol_type = other DNA
ggacagcaggtcatgcaaattcctcagtatcagcatgtgcaggagggcgaagacttcaccacttactgtaattcctcgaccaccttgagcaacatccagtggtacaagcagcgtcccggcggccaccccgtgttcctgatccagctggtgaaatctggcgaggtgaagaaacagaagcgcctgaccttccagtttggggaggccaagaagaactccagccttcacatcaccgccacccagactacagatgtggggacctacttttgcgctgtttctggtgcgggctcctaccagctcaccttcggcaagggcaccaagctgagtgtcatcccg

SEQ ID NO: 117 - DNA, 333 bp, synthetic construct
  source 1..333: mol_type = other DNA
ggacagcaggtcatgcaaattcctcagtaccagcatgtgcaggagggcgaagacttcaccacttactgtaattcctcgaccacactcagcaacatccagtggtacaagcagcgtcccggcggccaccccgtgttcctgatccagctggtgaagtctggggaggttaagaaacagaagcgcctgacctttcagttcggagaggccaagaagaactcctctttgcacatcaccgccacccagacgactgatgtggggacctacttttgcgctggtgcaggtgcgggctcctatcagcttaccttcggcaaaggcaccaagctgagcgtcatcccg

SEQ ID NO: 118 - DNA, 336 bp, synthetic construct
  source 1..336: mol_type = other DNA
tcacagcaaggcgaggaagatcctcaggccctcagcatccaggagggggagaatgcaactatgaactgcagctacaagaccagtattaacaacctgcagtggtaccgccagaactccggacgtggtctagtgcatttgatcctgatccgcagcaacgagagggagaagcactcgggtcgcctgcgggtcaccctggacacctccaagaagtcttcttctctgctcatcactgcttcccgcgccgcggacacagctagctatttttgtgccaccgagggcggctccggctactccacccttaccttcggcaaaggcaccatgctgctggtgtcgccc

SEQ ID NO: 119 - DNA, 336 bp, synthetic construct
  source 1..336: mol_type = other DNA
tcacaacagggcgaggaggaccctcaggccctctctatccaggagggcgagaatgcaacaatgaactgcagctacaagaccagcattaacaacctgcagtggtaccggcagaactccggccgtggtttggtgcatctaatcctgatccgcagcaacgagagggagaagcacagtgggcgcctgcgcgtcaccctggacacctccaagaagtcgtcctctctgctcatcaccgcttcccgcgccgcggacactgctagctatttttgtgccactgaaggtgattctggctactccacccttaccttcggcaaaggcaccatgctgctggtgtcgccc

SEQ ID NO: 120 - DNA, 336 bp, synthetic construct
  source 1..336: mol_type = other DNA
tcacaacagggagaggaggaccctcaggccctcagcatccaggagggcgagaatgccactatgaactgctcttacaagaccagcattaacaacctgcagtggtaccgccagaacagtgggcgtggtttggtgcatctcatcctgatccgcagcaacgagcgcgaaaagcactcgggtcgcctgcgggtcaccttggatacctccaagaagtcctcttctctgctgatcactgcttccagggcggcggacaccgcaagctatttttgtgctacagccggcaactccggctactccaccctgaccttcggcaaaggcaccatgcttctggtgtcgccc

SEQ ID NO: 121 - DNA, 333 bp, synthetic construct
  source 1..333: mol_type = other DNA
gacaccgccgtttcccagacaccgaagtacctggtgacccagatgggcaacgacaagagcatcaagtgcgagcagaacctgggccatgacacgatgtattggtacaagcaggactccaagaaatttctgaagatcatgtttagctacaacaacaaggagctcatcattaacgagaccgtgcccaaccgcttctcaccaaagtcgcccgacaaagcgcacttgaatctacacatcaattctctggagctgggtgattctgccgtgtacttctgtgcttcctcgccggatttcaacgaacagttcttcggccctgggactcgtctgaccgtcctt

SEQ ID NO: 122 - DNA, 354 bp, synthetic construct
  source 1..354: mol_type = other DNA
gaagcaggggtggctcagagcccgcgctacaagattattgagaagcgccagtccgtggcgttctggtgcaatcccatctctggccacgccactctttattggtaccaacagatcctgggacagggccctaaattgctcatccagttccagaacaacggtgtggtcgatgacagccagctgcccaaggacaggttttcagccgagcgcctgaagggcgtcgactcgaccctcaaaatccagccagccaagctggaggacagtgcggtgtacctgtgcgcctcctcctctcctctgggcggcttcgcgggggccaacgtgctgaccttcggtgctggctcccgtctgaccgttcta

SEQ ID NO: 123 - DNA, 336 bp, synthetic construct
  source 1..336: mol_type = other DNA
gatgcggacgtgacccagactccccgcaaccgcatcaccaagaccggcaagcgcatcatgttggagtgctctcaaacaaagggccacgacaggatgtactggtaccggcaggacccggggctgggcctccgtcttatctactactccttcgacgtgaaggacatcaataagggtgagatcagcgatggctactccgtgtcgcgacaggctcaggccaaattttcactatctctggagtccgccatccccaaccagacggcactgtacttctgtgccacctccgacttcattagtgacacccagtatttcggacctggtactcgcctgaccgtgctg

SEQ ID NO: 124 - DNA, 354 bp, synthetic construct
  source 1..354: mol_type = other DNA
tcggcagtgattagccagaagccctctcgggacatctgccagcgtggtacaagtttgaccatccagtgccaagttgattctcaggtcaccatgatgttttggtaccgccagcagccgggacagagcctaactcttatcgcgacggccaaccagggctccgaggctacctacgagagcggcttcgtcattgacaagtttcccatctcccgccctaacctgaccttctcgactctcaccgtgtccaatatgtctcctgaagacagctccatctatctgtgctccgtgggcggcacctccggcaccctgccagccaacgagcagttcttcggtcccgggacccgcctgaccgtgctg

SEQ ID NO: 125 - DNA, 336 bp, synthetic construct
  source 1..336: mol_type = other DNA
gacgcaggggtgatccagagcccgcgccatgaagtcaccgagatgggccaggaggtgactcttaggtgtaaacccatctctggccacaactccctcttttggtaccgccagactatgatgcgtggtctggagctgctgatttacttcaacaacaatgtgcccatcgatgactctggtatgcctgaggaccgcttttcagccaagatgcccaacgcgtccttctcgaccctgaagatccagccgtccgagccacgggacagcgccgtgtacttctgcgcttccagttggaccggcaacgagcagtatttcggacctggcacccgcttgacggttaca

SEQ ID NO: 126 - DNA, 339 bp, synthetic construct
  source 1..339: mol_type = other DNA
gatgcaggcatcacccagagcccgcgctacaagattacagagaccggccgccaggtcaccctgatgtgccaccagacctggtctcatagttacatgttttggtacaggcaggacctgggccacggtctccgtcttatctactactccgctgctgccgacatcaccgacaaaggggaggtgcccgacggctacgtggtgtcgcggtccaagactgagaacttccctttgactctggagagcgccactcgctcgcaaacgtccgtttatttctgcgcctcctctagcgatggtatgaacacggaagcgttcttcggacagggcacccgcctgaccgtggtc

SEQ ID NO: 127 - DNA, 339 bp, synthetic construct
  source 1..339: mol_type = other DNA
aatgcaggtgtcacccagactccgaaatttcaggtcctgaagaccggtcagagcatgactttgcagtgcgcccaggacatgaaccatgagtacatgagttggtaccgccaggatccaggaatgggccttcggctcattcactactccgtgggggccggcatcaccgaccagggggaggtgcctaacggctacaacgtgtcccgctcgaccacagaggacttccccctgcgtctgctgtccgccgccccctctcaaacgagcgtgtacttctgtgcttcttccaagcagggcggtggatacggctataccttcggctccggcacccgcctgaccgtggtt

SEQ ID NO: 128 - DNA, 345 bp, synthetic construct
  source 1..345: mol_type = other DNA
aaagccggcgtgacccagactccgcgctacctaattaagactcgtggtcaacaggtcaccctgagctgttcgcccatctctggccaccggtccgttagttggtaccagcagacgccaggacagggcctccagttcctgttcgagtacttctccgagacccagcgcaacaagggcaactttcctgggcgattctctggtcgccagttttcaaattccaggtcggagatgaacgtgtccacccttgagctgggggacagcgcgctgtacctgtgcgcttcctctttggagggccaggccagcagctacgaacagtatttcggacccggcacccgcctgaccgtgaca

SEQ ID NO: 129 - DNA, 345 bp, synthetic construct
  source 1..345: mol_type = other DNA
aaagccggcgtgacccagacgccacgatacctaattaagacccgtggtcaacaggtcaccctttcatgctctcccatctcgggccaccggtccgtgagttggtaccagcagactccgggacagggcctccagttcctgttcgagtacttctccgagacccagcgcaacaagggcaactttcccgggcgcttctctggacgccagttttccaattccaggtcggagatgaacgtgtccactctggagctgggggacagcgcgctgtacctgtgcgcctcctctttggagggccaggccagcagctacgaacagtatttcggtcctggcacccgcctgaccgttaca

SEQ ID NO: 130 - DNA, 345 bp, synthetic construct
  source 1..345: mol_type = other DNA
aaagccggtgttacccagactccgcgctacctcattaagaccagaggacaacaggtcactctttcatgctctcccatctctggccaccggtccgtgagttggtaccagcagacgccaggacagggcttgcagttcctgttcgagtacttctccgagacccagcgcaacaagggcaactttcccgggcgtttctctggtcgccagttttcaaattccaggtcggagatgaacgtgtcgaccctggagctaggggacagcgcgctgtacctgtgcgcctccagcgcagagggccaggcctccagctacgaacagtatttcggccctggcacccgcctgaccgtgaca

SEQ ID NO: 131 - DNA, 342 bp, synthetic construct
  source 1..342: mol_type = other DNA
aacgcgggtgtcacccagactccgaagtttcaggtcctgaagaccggtcagagcatgactttgcagtgcgcccaggacatgaatcatgagtacatgagttggtacaggcaggatccaggaatgggcctccgtcttattcactactccgttggggccggcatcaccgaccagggggaggtgcctaacggctacaacgtgtcccgctcgaccacagaggacttccccctgcggctgctgagcgcagctccctctcaaacgtccgtgtacttctgtgcttctagccgccagggcggttcaggctccggctataccttcggctcgggcacccgcctgaccgtggtg

SEQ ID NO: 132 - DNA, 339 bp, synthetic construct
  source 1..339: mol_type = other DNA
aacgcgggtgtcacccagactcctaagtttcaggtcttgaagactggacagagcatgacactgcagtgtgcccaggacatgaaccatgagtacatgagttggtaccgccaggacccgggaatgggcctccgtcttattcactactccgtgggtgctggcatcaccgaccagggggaggtgccaaatggctacaacgtgtcccgctcaacgaccgaggatttccccctgcggctgctgtctgcagctccctctcaaactagcgtgtacttctgcgcctcgaccacccagggcggggcctacggctataccttcggctccggcacccgcctgaccgtggtt

SEQ ID NO: 133 - DNA, 336 bp, synthetic construct
  source 1..336: mol_type = other DNA
aacgcaggtgtcacccagactccgaagtttcaggtcttgaagaccggccagagcatgacgctgcagtgcgcccaggacatgaatcatgagtacatgagttggtaccgccaggatccaggtatgggccttcgtctcattcactactccgtgggggccggcatcaccgaccagggggaggtgcctaacggctacaacgtgtcccggtcgaccacagaagacttccccctgcgcctgctgtccgccgccccctctcaaacgtctgtttatttctgtgcttccactcctcagggaggcaacgaggcgttcttcggacagggcacccgcctgaccgtggtg

SEQ ID NO: 134 - DNA, 342 bp, synthetic construct
  source 1..342: mol_type = other DNA
atactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgccgttaatgctggtggtactagctatggaaagctgacatttggacaagggaccatcttgactgtccatcca

SEQ ID NO: 135 - DNA, 345 bp, synthetic construct
  source 1..345: mol_type = other DNA
gaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccaacctgcaaagcttgaggactcggccgtgtatctctgtgccagcagctccgattggacagcgaacaatgagcagttcttcgggccagggacacggctcaccgtgcta

SEQ ID NO: 136 - protein, 6 aa, synthetic construct
SSNFYA

SEQ ID NO: 137 - protein, 7 aa, synthetic construct
MTLNGDE

SEQ ID NO: 138 - protein, 15 aa, synthetic construct
CAVNAGGTSYGKLTF

SEQ ID NO: 139 - protein, 15 aa, synthetic construct
CASSSDWTANNEQFF

SEQ ID NO: 140 - protein, 114 aa, synthetic construct
ILNVEQSPQSLHVQEGDSTNFTCSFPSSNFYALHWYRWETAKSPEALFVMTLNGDEKKKGRISATLNTKEGYSYLYIKGSQPEDSATYLCAVNAGGTSYGKLTFGQGTILTVHP

SEQ ID NO: 141 - protein, 115 aa, synthetic construct
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSSDWTANNEQFFGPGTRLTVL

SEQ ID NO: 142 - DNA, 342 bp, synthetic construct; identical to SEQ ID NO: 134
  source 1..342: mol_type = other DNA

SEQ ID NO: 143 - DNA, 345 bp, synthetic construct; identical to SEQ ID NO: 135
  source 1..345: mol_type = other DNA

SEQ ID NO: 144 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 145 - protein, 11 aa, synthetic construct
EPLPQGQLTAY

SEQ ID NO: 146 - protein, 9 aa, synthetic construct
MGSLEMVPM

SEQ ID NO: 147 - protein, 9 aa, synthetic construct
RLPGVLPRA

SEQ ID NO: 148 - protein, 9 aa, synthetic construct
FLQFKTWWI

SEQ ID NO: 149 - protein, 7 aa, synthetic construct
TSESNYY

SEQ ID NO: 150 - protein, 8 aa, synthetic construct
QEAYKQQN

SEQ ID NO: 151 - protein, 14 aa, synthetic construct
CAFMIPDSNYQLIW

SEQ ID NO: 152 - protein, 15 aa, synthetic construct
CAFMLIDSGTYKYIF

SEQ ID NO: 153 - protein, 12 aa, synthetic construct
CAVGGNNNDMRF

SEQ ID NO: 154 - protein, 5 aa, synthetic construct
LGHNA

SEQ ID NO: 155 - protein, 5 aa, synthetic construct
PRHDT

SEQ ID NO: 156 - protein, 6 aa, synthetic construct
YNFKEQ

SEQ ID NO: 157 - protein, 6 aa, synthetic construct
SMNVEV

SEQ ID NO: 158 - protein, 6 aa, synthetic construct
FYEKMQ

SEQ ID NO: 159 - protein, 17 aa, synthetic construct
CASSQVGTSGRGGELFF

SEQ ID NO: 160 - protein, 14 aa, synthetic construct
CASSLGQGTETQYF

SEQ ID NO: 161 - protein, 12 aa, synthetic construct
CASSLINTEAFF

SEQ ID NO: 162 - protein, 115 aa, synthetic construct
AQTVTQSQPEMSVQEAETVTLSCTYDTSESNYYLFWYKQPPSRQMILVIRQEAYKQQNATENRFSVNFQKAAKSFSLKISDSQLGDTAMYFCAFMIPDSNYQLIWGAGTKLIIKP

SEQ ID NO: 163 - protein, 116 aa, synthetic construct
AQTVTQSQPEMSVQEAETVTLSCTYDTSENNYYLFWYKQPPSRQMILVIRQEAYKQQNATENRFSVNFQKAAKSFSLKISDSQLGDTAMYFCAFMLIDSGTYKYIFGTGTRLKVLA

SEQ ID NO: 164 - protein, 111 aa, synthetic construct
KQEVTQIPAALSVPEGENLVLNCSFTDSAIYNLQWFRQDPGKGLTSLLLIQSSQREQTSGRLNASLDKSSGRSTLYIAASQPGDSATYLCAVGGNNNDMRFGAGTRLTVKP

SEQ ID NO: 165 - protein, 7 aa, synthetic construct
TSENNYY

SEQ ID NO: 166 - protein, 116 aa, synthetic construct
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSQVGTSGRGGELFFGEGSRLTVL

SEQ ID NO: 167 - protein, 113 aa, synthetic construct
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGQGTETQYFGPGTRLLVL

SEQ ID NO: 168 - protein, 111 aa, synthetic construct
AAGVIQSPRHLIKEKRETATLKCYPIPRHDTVYWYQQGPGQDPQFLISFYEKMQSDKGSIPDRFSAQQFSDYHSELNMSSLELGDSALYFCASSLINTEAFFGQGTRLTVV

SEQ ID NO: 169 - DNA, 345 bp, synthetic construct
  source 1..345: mol_type = other DNA
gcccagacagtcactcagtctcaaccagagatgtctgtgcaggaggcagagactgtgaccctgagttgcacatatgacaccagtgagagtaattattatttgttctggtacaaacagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcatgataccggatagcaactatcagttaatctggggcgctgggaccaagctaattataaagcca

SEQ ID NO: 170 - DNA, 348 bp, synthetic construct
  source 1..348: mol_type = other DNA
gcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcatgttaatagactcaggaacctacaaatacatctttggaacaggcaccaggctgaaggttttagca

SEQ ID NO: 171 - DNA, 333 bp, synthetic construct
  source 1..333: mol_type = other DNA
aaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgggaggcaataacaatgacatgcgctttggagcagggaccagactgacagtaaaacca

SEQ ID NO: 172 - DNA, 348 bp, synthetic construct
  source 1..348: mol_type = other DNA
gaaacgggagttacgcagacaccaagacacctggtcatgggaatgacaaataagaagtctttgaaatgtgaacaacatctggggcataacgctatgtattggtacaagcaaagtgctaagaagccactggagctcatgtttgtctacaactttaaagaacagactgaaaacaacagtgtgccaagtcgcttctcacctgaatgccccaacagctctcacttattccttcacctacacaccctgcagccagaagactcggccctgtatctctgtgccagcagccaagttgggactagcgggaggggcggggagctgttttttggagaaggctctaggctgaccgtactg

SEQ ID NO: 173 - DNA, 339 bp, synthetic construct
  source 1..339: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtcttggacagggaacagagacccagtacttcgggccaggcacgcggctcctggtgctc

SEQ ID NO: 174 - DNA, 333 bp, synthetic construct
  source 1..333: mol_type = other DNA
gctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagcttaattaacactgaagctttctttggacaaggcaccagactcacagttgta

SEQ ID NO: 175 - DNA, 345 bp, synthetic construct
  source 1..345: mol_type = other DNA
gcacagacggtcacccagagccagccggagatgtctgtgcaggaggctgaaaccgtgaccttgtcatgcacttacgacacctccgagagcaactactacctgttttggtacaagcagccaccctctcgtcagatgatcctggtgattcgccaggaggcctacaagcaacagaacgcgactgagaaccgcttctccgttaatttccagaaggccgccaaatcgttttccctcaaaatctccgacagtcagctgggtgatacagccatgtacttctgtgcgttcatgatccccgacagcaactatcagcttatttggggcgctggcaccaagctgatcatcaagcct

SEQ ID NO: 176 - DNA, 348 bp, synthetic construct
  source 1..348: mol_type = other DNA
gcgcagacggttacccagagccaacctgagatgtccgtgcaggaggctgaaaccgtgaccttgtcatgcacttacgacacctccgagaacaactattacctgttttggtacaagcagccgccctctcgtcagatgatcctggtgatccgccaggaggcctacaaacagcagaacgcaaccgagaatcggttttcggtcaacttccagaaggctgccaaatccttctccctcaagatcagcgattctcagctgggcgacacggccatgtatttctgtgcgttcatgcttattgacagtggcacctacaagtacatcttcgggacaggtactcgcctgaaggtgctggcc

SEQ ID NO: 177 - DNA, 333 bp, synthetic construct; identical to SEQ ID NO: 171
  source 1..333: mol_type = other DNA

SEQ ID NO: 178 - DNA, 348 bp, synthetic construct
  source 1..348: mol_type = other DNA
gagactggcgtcacccagactccgcgccacctggtgatgggaatgaccaacaagaaatctcttaaatgcgagcaacatctaggccacaacgccatgtattggtacaagcagagcgccaagaagcccctggagctgatgttcgtgtacaacttcaaggagcagacggagaacaactccgtgccctctcggttcagccctgaatgcccaaattcgagtcacttgttcctgcacttgcatacactccagccggaggacagcgcgctgtacctgtgcgcctcctcacaggttggcacctccggtcgtggtggggagctctttttcggcgagggctcccgcctgaccgtgctg

SEQ ID NO: 179 - DNA, 339 bp, synthetic construct
  source 1..339: mol_type = other DNA
gaagctcaggtcacccagaatccacgttatctcatcacagtcaccggcaagaagctcacggttacctgctctcagaacatgaaccacgagtacatgagttggtacaggcaggacccgggccttggcttgcggcaaatttactactccatgaacgtggaggtgaccgacaaaggtgatgtgcctgagggctacaaggtgtcccgcaaggagaagcgcaactttcccctgatcctggagagcccttcccccaaccagacttctctgtacttctgtgccagctcgctaggacagggcaccgagacccagtatttcggtcccgggactcgcctgctggtgctg

SEQ ID NO: 180 - DNA, 333 bp, synthetic construct; identical to SEQ ID NO: 174
  source 1..333: mol_type = other DNA

SEQ ID NO: 181 - protein, 14 aa, synthetic construct
  VARIANT 4: note = X is E or A
  VARIANT 6: note = X is D, G, N or S, or any of the following amino acids with related properties: E, A, Q or T
CATXGXSGYSTLTF

SEQ ID NO: 182 - protein, 15 aa, synthetic construct
  VARIANT 4: note = X is S or T, or any of the following amino acids with related properties: N or Q
  VARIANT 5: note = X is K, R or T, or any of the following amino acids with related properties: H, S
  VARIANT 10: note = X is G or A
  VARIANT 11: note = X is Y or S, or any of the following amino acids with related properties: F, W, H or T
CASXXQGGSXXGYTF

SEQ ID NO: 183 - protein, 13 aa, synthetic construct
  VARIANT 3: note = X is G or V, or any of the following amino acids with related properties: A, I or L
  VARIANT 4: note = X is A or S, or any of the following amino acids with related properties: G or T
CAXXGAGSYQLTF

SEQ ID NO: 184 - protein, 16 aa, synthetic construct
  VARIANT 5: note = X is L or A, or any of the following amino acids with related properties: I, V or G
CASSXEGQASSYEQYF

SEQ ID NO: 185 - protein, 15 aa, synthetic construct
  VARIANT 5: note = X is L or I or E, or any of the following amino acids with related properties: V or D
  VARIANT 6: note = X is P or I or A, or any of the following amino acids with related properties: V, L or G
  VARIANT 9: note = X is G or N, or any of the following amino acids with related properties: Q, A, C or S
  VARIANT 10: note = X is T or no AA at this position, or S as an amino acid with related properties
  VARIANT 12: note = X is K or Q, or any of the following amino acids with related properties: R, H or N
  VARIANT 13: note = X is L or Y, or any of the following amino acids with related properties: I, V, F, W or H
  VARIANT 15: note = X is F or W
CAFMXXDSXXYXXIX

SEQ ID NO: 186 - DNA, 345 bp, synthetic construct
  source 1..345: mol_type = other DNA
gcccagacagtcactcagtctcaaccagagatgtctgtgcaggaggcagagactgtgaccctgagttgcacatatgacaccagtgagagtaattattatttgttctggtacaaacagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcatggaggcggatagcaactatcagttaatctggggcgctgggaccaagctaattataaagcca

SEQ ID NO: 187 - DNA, 348 bp, synthetic construct
  source 1..348: mol_type = other DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcaagggcaggcgggggccggactataattcacccctccactttgggaacgggaccaggctcactgtgaca

SEQ ID NO: 188 - DNA, 345 bp, synthetic construct
  source 1..345: mol_type = other DNA
gcgcagacggtgacccagagccagccggagatgtccgtgcaggaggctgagaccgtcaccctgtcgtgcacttacgacacctccgagagcaactactacctgttttggtacaagcagccaccctctcgccagatgatcctggtgattcgtcaggaggcctacaaacagcagaacgcgacagagaaccgcttctcggttaatttccagaaggcagccaagtccttctccctcaaaattagcgattctcaattgggtgacactgccatgtacttctgtgcttttatggaagcggacagtaactatcagcttatctggggcgccggcaccaagctgatcatcaagcct

SEQ ID NO: 189 - DNA, 348 bp, synthetic construct
  source 1..348: mol_type = other DNA
aaggccggcgttacccagacgcctcgttatcttattaagacccgaggacagcaggtcacactatcttgctctcccatctctggccaccgctccgtgagttggtaccaacagactccgggtcagggcctccagttcctgttcgagtacttcagcgaaacccagcgcaacaagggcaacttcccagggcgcttcagcggacgccagttttcaaattccaggtcggagatgaacgtgtcgaccctggagctgggtgatagcgcgctgtacctgtgcgcctccaaaggccggcgtgggcccgactacaactcccctttgcattttggcaacggcacccgcctgaccgtgact

SEQ ID NO: 190 - protein, 137 aa, Mus musculus
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 191 - protein, 173 aa, Mus musculus
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 192 - protein, 15 aa, synthetic construct
CATSDWDDSTGELFF

SEQ ID NO: 193 - protein, 114 aa, synthetic construct
DADVTQTPRNRITKTGKRIMLECSQTKGHDRMYWYRQDPGLGLRLIYYSFDVKDINKGEISDGYSVSRQAQAKFSLSLESAIPNQTALYFCATSDWDDSTGELFFGEGSRLTVL

SEQ ID NO: 194 - protein, 15 aa, synthetic construct
CAFMGPDSGTYKYIF

SEQ ID NO: 195 - protein, 14 aa, synthetic construct
CATSDSDRIYGYTF

SEQ ID NO: 196 - protein, 14 aa, synthetic construct
CAMREPDSNYQLIW

SEQ ID NO: 197 - protein, 5 aa, synthetic construct
SNHLY

SEQ ID NO: 198 - protein, 6 aa, synthetic construct
FYNNEI

SEQ ID NO: 199 - protein, 13 aa, synthetic construct
CASQKGLEYEQYF

SEQ ID NO: 200 - protein, 116 aa, synthetic construct
AQTVTQSQPEMSVQEAETVTLSCTYDTSENNYYLFWYKQPPSRQMILVIRQEAYKQQNATENRFSVNFQKAAKSFSLKISDSQLGDTAMYFCAFMGPDSGTYKYIFGTGTRLKVLA

SEQ ID NO: 201 - protein, 113 aa, synthetic construct
DADVTQTPRNRITKTGKRIMLECSQTKGHDRMYWYRQDPGLGLQLIYYSFDVKDINKGEISDGYSVSRQAQAKFSLSLESAIPNQTALYFCATSDSDRIYGYTFGSGTRLTVV

SEQ ID NO: 202 - protein, 115 aa, synthetic construct
AQKITQTQPGMFVQEKEAVTLDCTYDTSDQSYGLFWYKQPSSGEMIFLIYQGSYDEQNATEGRYSLNFQKARKSANLVISASQLGDSAMYFCAMREPDSNYQLIWGAGTKLIIKP

SEQ ID NO: 203 - protein, 113 aa, synthetic construct
EPEVTQTPSHQVTQMGQEVILRCVPISNHLYFYWYRQILGQKVEFLVSFYNNEISEKSEIFDDQFSVERPDGSNFTLKIRSTKLEDSAMYFCASQKGLEYEQYFGPGTRLTVT

SEQ ID NO: 204 - DNA, 348 bp, synthetic construct
  source 1..348: mol_type = other DNA
gcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcatgggacctgactcaggaacctacaaatacatctttggaacaggcaccaggctgaaggttttagca

SEQ ID NO: 205 - DNA, 339 bp, synthetic construct
  source 1..339: mol_type = other DNA
gatgctgatgttacccagaccccaaggaataggatcacaaagacaggaaagaggattatgctggaatgttctcagactaagggtcatgatagaatgtactggtatcgacaagacccaggactgggcctacagttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgattccgacagaatctatggctacaccttcggttcggggaccaggttaaccgttgta

SEQ ID NO: 206 - DNA, 345 bp, synthetic construct
  source 1..345: mol_type = other DNA
gcccagaagataactcaaacccaaccaggaatgttcgtgcaggaaaaggaggctgtgactctggactgcacatatgacaccagtgatcaaagttatggtctattctggtacaagcagcccagcagtggggaaatgatttttcttatttatcaggggtcttatgacgagcaaaatgcaacagaaggtcgctactcattgaatttccagaaggcaagaaaatccgccaaccttgtcatctccgcttcacaactgggggactcagcaatgtatttctgtgcaatgagagagcccgatagcaactatcagttaatctggggcgctgggaccaagctaattataaagcca

SEQ ID NO: 207 - DNA, 340 bp, synthetic construct
  source 1..340: mol_type = other DNA
gaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagccaaaagggactagagtacgagcagtacttcgggccgggcaccaggctcacggtcacag

SEQ ID NO: 208 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 209 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 210 - protein, 178 aa, Homo sapiens
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 211 - protein, 141 aa, synthetic construct
NIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 212 - protein, 179 aa, synthetic construct
EDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 213 - DNA, 348 bp, synthetic construct
  source 1..348: mol_type = other DNA
gcacagacggtcacccagagccagccggagatgtccgtgcaggaggccgagaccgtgactctttcatgcacttacgacacctccgagaacaactactacctcttttggtacaagcaacctccctctcggcagatgatcctggtgatccgtcaggaggcttataaacagcagaacgcgacagaaaaccgcttctcggtcaatttccagaaggctgccaagtccttttctttgaaaattagtgacagccagctgggagatacggccatgtatttctgtgcgttcatggggcccgactccggcacctacaagtacatcttcggtaccggcacccgcctgaaggtgctggcc

SEQ ID NO: 214 - DNA, 339 bp, synthetic construct
  source 1..339: mol_type = other DNA
gacgcggacgtgacccagacaccccgcaaccgcatcaccaagaccggcaagcgtatcatgcttgagtgctctcaaactaagggccacgatcgaatgtattggtacaggcaggacccgggtctgggtctccagctgatttactactccttcgacgtgaaggacattaataagggagagatctcggacggctattccgtgtcccgccaggctcaggcaaaattttcattgagcctggagagcgccatccctaaccagactgctctgtacttctgtgccaccagcgattctgatcggatctacggctacaccttcggctccgggacccgcctgaccgtggtt

SEQ ID NO: 215 - DNA, 348 bp, synthetic construct
  source 1..348: mol_type = other DNA
gctcaaacagtgacccagagccagcccgagatgagcgtgcaggaagctgaaaccgtcaccctgtcttgtacctacgacaccagcgagaacaactactacctgttttggtataagcagccacctagcagacagatgatcctggtgatccggcaggaggcctacaaacagcagaacgccacagagaatagattctctgtgaacttccagaaggccgccaagtccttcagcctgaagatcagcgacagccaactgggcgacaccgccatgtacttctgcgcctttatgggacctgattccggcacatacaagtacatcttcggcacaggcaccagactgaaagtgctggcc

SEQ ID NO: 216 - DNA, 339 bp, synthetic construct
  source 1..339: mol_type = other DNA
gatgccgacgtgacccagacccctagaaatagaattacaaagaccggcaagcggatcatgctggaatgtagccagaccaaaggccacgaccggatgtactggtaccggcaggaccccggactgggcctccagctgatctactactcttttgatgtcaaggacatcaacaagggcgagatcagcgacggctactccgtgtccagacaagctcaggccaagttcagcctgtctctggagagcgccatccctaaccagaccgccctgtacttctgcgccaccagcgacagcgatagaatctacggctatacattcggcagcggaacaagactgaccgtggtg

SEQ ID NO: 217 - DNA, 345 bp, synthetic construct
  source 1..345: mol_type = other DNA
gcccagaaaatcacacagacccagcccggcatgttcgtgcaggagaaggaagccgtgaccctggactgtacctacgacaccagcgaccagagctacggcctgttttggtacaaacagcctagcagcggcgagatgatcttcctgatctaccaaggatcttatgatgagcagaacgccacagagggaagatacagcctgaacttccagaaggccagaaagtccgctaatctggtgatcagcgcttctcagctgggcgactccgccatgtacttctgcgccatgcgggaacctgatagcaactaccaactgatctggggcgccggcaccaagctcattatcaagcca

SEQ ID NO: 218 - DNA, 339 bp, synthetic construct
  source 1..339: mol_type = other DNA
gagcctgaggtgacccagacccctagccaccaggtgacccaaatgggccaggaggtcatcctcagatgtgtgcccatcagcaaccacctgtacttttactggtatagacagatcctgggccagaaagtggaattcctggtgtccttctacaacaacgagattagcgagaagtccgagatcttcgacgaccagttcagcgtggaacggcctgacggatctaatttcaccctgaagatccggagcacaaagctggaagatagcgccatgtacttctgcgcctctcagaagggcctggaatacgagcagtactttggccccggcaccagactgacagtgaca

SEQ ID NO: 219 - DNA, 348 bp, synthetic construct
  source 1..348: mol_type = other DNA
gcccagaccgtcacccagtcccagcctgagatgagcgtgcaggaggccgagacagtgaccctgagctgtacctacgacacatctgaaaacaactactatctcttctggtacaaacaacctcccagccggcagatgatcctggtgatcagacaagaagcctacaagcagcagaacgccacagagaatagattctccgtgaacttccagaaagccgctaagagctttagcctgaagatctctgatagccagctgggcgacaccgccatgtacttctgcgccttcatgctgatcgacagcggcacctacaagtacatctttggaaccggcacaagactgaaggtgctggct

SEQ ID NO: 220 - DNA, 348 bp, synthetic construct
  source 1..348: mol_type = other DNA
gaaaccggcgtgacccagacccctagacacctggtcatgggcatgaccaacaaaaagtccctgaagtgcgagcagcacctgggccacaacgccatgtactggtataagcagagcgccaagaaaccactggaactgatgttcgtgtacaacttcaaggaacaaacagagaacaacagcgtgcccagccggttcagccccgagtgtcctaatagctcccacctgttcctgcacctccatacactgcagcctgaggacagcgctctgtacctgtgcgcctctagccaggtgggcacaagcggcagaggcggagagctgtttttcggcgagggatctagactgaccgtgctg

SEQ ID NO: 221 - DNA, 348 bp, synthetic construct; identical to SEQ ID NO: 219
  source 1..348: mol_type = other DNA

SEQ ID NO: 222 - DNA, 339 bp, synthetic construct
  source 1..339: mol_type = other DNA
gaggcccaggtgacccaaaatcctagatacctgatcaccgtcacaggcaagaaactgaccgtgacatgtagccagaacatgaaccacgagtacatgagctggtatagacaggaccccggcctgggactgcggcagatctactacagcatgaacgtggaagtgaccgataagggcgacgtgccagagggctacaaggtgtccagaaaggaaaagcggaacttccctctgatcctggaatctcctagccccaaccagaccagcctctacttctgcgcctctagcctgggccagggcaccgagacacagtactttggccctggaaccagactgctggtgctg

SEQ ID NO: 223 - DNA, 426 bp, synthetic construct
  source 1..426: mol_type = other DNA
aatatccagaatcctgatcctgctgtgtaccagctgagagattctaaaagcagcgacaaatctgtgtgcctgttcaccgacttcgacagccaaacaaacgtgtcccagagcaaggacagcgacgtgtacatcaccgacaagtgcgtgctggacatgagaagcatggacttcaagagcaacagcgccgtcgcttggtccaacaagtctgattttgcctgcgccaacgccttcaacaacagcatcattccagaggacaccttcttccccagccctgaaagctcttgtgatgtgaagctggtggaaaagtccttcgagacagatacaaatctgaacttccagaacctgagcgtgatcggctttagaatcctgctgctcaaggtggccggcttcaacctgctgatgaccctgcggctgtggagcagctga

SEQ ID NO: 224 - DNA, 537 bp, synthetic construct
  source 1..537: mol_type = other DNA
gaagatctgaaaaacgtgtttccacctgaggtggccgtgttcgagccttccgaggccgagatcagccacacccagaaggccacactggtgtgtctggccacaggcttttaccccgaccacgtggaactgagctggtgggttaacggcaaagaagtgcattctggagtgtgcaccgacccccagcctctgaaggaacagcctgccctgaatgatagcagatactgcctgagcagccgcctgagagtgtccgccaccttctggcagaaccccagaaaccacttccggtgccaggtgcaattctacggcctgagcgagaacgacgagtggacccaagatagagctaagcctgtgacccagatcgtgtctgctgaagcctggggaagagccgactgcggcttcaccagcgagagctaccagcagggcgtgctgagcgctacaatcctgtacgagatcctgctgggcaaggccaccctgtatgccgtgctggtgtctgccctggtcctcatggccatggtgaagcggaaggacagccggggc

SEQ ID NO: 225 - protein, 26 aa, synthetic construct
AQSVSQHNHHVILSEAASLELGCNYS

SEQ ID NO: 226 - protein, 17 aa, synthetic construct
LFWYVQYPGQHLQLLLK

SEQ ID NO: 227 - protein, 33 aa, synthetic construct
KGIKGFEAEFIKSKFSFNLRKPSVQWSDTAEYF

SEQ ID NO: 228 - protein, 10 aa, synthetic construct
GTGTRLQVFP

SEQ ID NO: 229 - protein, 10 aa, synthetic construct
GGGTRVLVKP

SEQ ID NO: 230 - protein, 26 aa, synthetic construct
AQKITQTQPGMFVQEKEAVTLDCTYD

SEQ ID NO: 231 - protein, 17 aa, synthetic construct
LFWYKQPSSGEMIFLIY

SEQ ID NO: 232 - protein, 33 aa, synthetic construct
ATEGRYSLNFQKARKSANLVISASQLGDSAMYF

SEQ ID NO: 233 - protein, 10 aa, synthetic construct
GSGTKLNVKP

SEQ ID NO: 234 - protein, 26 aa, synthetic construct
AQSVTQLDSQVPVFEEAPVELRCNYS

SEQ ID NO: 235 - protein, 17 aa, synthetic construct
LFWYVQYPNQGLQLLLK

SEQ ID NO: 236 - protein, 33 aa, synthetic construct
KGINGFEAEFNKSQTSFHLRKPSVHISDTAEYF

SEQ ID NO: 237 - protein, 10 aa, synthetic construct
GQGTELSVKP

SEQ ID NO: 238 - protein, 26 aa, synthetic construct
KQEVTQIPAALSVPEGENLVLNCSFT

SEQ ID NO: 239 - protein, 17 aa, synthetic construct
LQWFRQDPGKGLTSLLL

SEQ ID NO: 240 - protein, 33 aa, synthetic construct
QTSGRLNASLDKSSGRSTLYIAASQPGDSATYL

SEQ ID NO: 241 - protein, 10 aa, synthetic construct
GAGTKLIIKP

SEQ ID NO: 242 - protein, 26 aa, synthetic construct
SQQGEEDPQALSIQEGENATMNCSYK

SEQ ID NO: 243 - protein, 17 aa, synthetic construct
LQWYRQNSGRGLVHLIL

SEQ ID NO: 244 - protein, 33 aa, synthetic construct
KHSGRLRVTLDTSKKSSSLLITASRAADTASYF

SEQ ID NO: 245 - protein, 10 aa, synthetic construct
GKGTMLLVSP

SEQ ID NO: 246 - protein, 26 aa, synthetic construct
GQQVMQIPQYQHVQEGEDFTTYCNSS

SEQ ID NO: 247 - protein, 17 aa, synthetic construct
IQWYKQRPGGHPVFLIQ

SEQ ID NO: 248 - protein, 33 aa, synthetic construct
KKQKRLTFQFGEAKKNSSLHITATQTTDVGTYF

SEQ ID NO: 249 - protein, 10 aa, synthetic construct
GKGTKLSVIP

SEQ ID NO: 250 - protein, 26 aa, synthetic construct
ILNVEQSPQSLHVQEGDSTNFTCSFP

SEQ ID NO: 251 - protein, 17 aa, synthetic construct
LHWYRWETAKSPEALFV

SEQ ID NO: 252 - protein, 33 aa, synthetic construct
KKKGRISATLNTKEGYSYLYIKGSQPEDSATYL

SEQ ID NO: 253 - protein, 10 aa, synthetic construct
GQGTILTVHP

SEQ ID NO: 254 - protein, 26 aa, synthetic construct
AQTVTQSQPEMSVQEAETVTLSCTYD

SEQ ID NO: 255 - protein, 17 aa, synthetic construct
LFWYKQPPSRQMILVIR

SEQ ID NO: 256 - protein, 33 aa, synthetic construct
ATENRFSVNFQKAAKSFSLKISDSQLGDTAMYF

SEQ ID NO: 257 - protein, 10 aa, synthetic construct
GTGTRLKVLA

SEQ ID NO: 258 - protein, 10 aa, synthetic construct
GAGTRLTVKP

SEQ ID NO: 259 - protein, 26 aa, synthetic construct
DTAVSQTPKYLVTQMGNDKSIKCEQN

SEQ ID NO: 260 - protein, 17 aa, synthetic construct
MYWYKQDSKKFLKIMFS

SEQ ID NO: 261 - protein, 36 aa, synthetic construct
IINETVPNRFSPKSPDKAHLNLHINSLELGDSAVYF

SEQ ID NO: 262 - protein, 9 aa, synthetic construct
GPGTRLTVL

SEQ ID NO: 263 - protein, 26 aa, synthetic construct
EAGVAQSPRYKIIEKRQSVAFWCNPI

SEQ ID NO: 264 - protein, 17 aa, synthetic construct
LYWYQQILGQGPKLLIQ

SEQ ID NO: 265 - protein, 37 aa, synthetic construct
VDDSQLPKDRFSAERLKGVDSTLKIQPAKLEDSAVYL

SEQ ID NO: 266 - protein, 9 aa, synthetic construct
GAGSRLTVL

SEQ ID NO: 267 - protein, 26 aa, synthetic construct
DADVTQTPRNRITKTGKRIMLECSQT

SEQ ID NO: 268 - protein, 17 aa, synthetic construct
MYWYRQDPGLGLRLIYY

SEQ ID NO: 269 - protein, 36 aa, synthetic construct
INKGEISDGYSVSRQAQAKFSLSLESAIPNQTALYF

SEQ ID NO: 270 - protein, 26 aa, synthetic construct
SAVISQKPSRDICQRGTSLTIQCQVD

SEQ ID NO: 271 - protein, 17 aa, synthetic construct
MFWYRQQPGQSLTLIAT

SEQ ID NO: 272 - protein, 37 aa, synthetic construct
TYESGFVIDKFPISRPNLTFSTLTVSNMSPEDSSIYL

SEQ ID NO: 273 - protein, 26 aa, synthetic construct
DAGVIQSPRHEVTEMGQEVTLRCKPI

SEQ ID NO: 274 - protein, 17 aa, synthetic construct
LFWYRQTMMRGLELLIY

SEQ ID NO: 275 - protein, 37 aa, synthetic construct
IDDSGMPEDRFSAKMPNASFSTLKIQPSEPRDSAVYF

SEQ ID NO: 276 - protein, 9 aa, synthetic construct
GPGTRLTVT

SEQ ID NO: 277 - protein, 26 aa, synthetic construct
DAGITQSPRYKITETGRQVTLMCHQT

SEQ ID NO: 278 - protein, 17 aa, synthetic construct
MFWYRQDLGHGLRLIYY

SEQ ID NO: 279 - protein, 36 aa, synthetic construct
TDKGEVPDGYVVSRSKTENFPLTLESATRSQTSVYF

SEQ ID NO: 280 - protein, 9 aa, synthetic construct
GQGTRLTVV

SEQ ID NO: 281 - protein, 26 aa, synthetic construct
NAGVTQTPKFQVLKTGQSMTLQCAQD

SEQ ID NO: 282 - protein, 17 aa, synthetic construct
MSWYRQDPGMGLRLIHY

SEQ ID NO: 283 - protein, 36 aa, synthetic construct
TDQGEVPNGYNVSRSTTEDFPLRLLSAAPSQTSVYF

SEQ ID NO: 284 - protein, 9 aa, synthetic construct
GSGTRLTVV

SEQ ID NO: 285 - protein, 26 aa, synthetic construct
KAGVTQTPRYLIKTRGQQVTLSCSPI

SEQ ID NO: 286 - protein, 17 aa, synthetic construct
VSWYQQTPGQGLQFLFE

SEQ ID NO: 287 - protein, 36 aa, synthetic construct
RNKGNFPGRFSGRQFSNSRSEMNVSTLELGDSALYL

SEQ ID NO: 288 - protein, 26 aa, synthetic construct
ETGVTQTPRHLVMGMTNKKSLKCEQH

SEQ ID NO: 289 - protein, 17 aa, synthetic construct
MYWYKQSAKKPLELMFV

SEQ ID NO: 290 - protein, 36 aa, synthetic construct
TENNSVPSRFSPECPNSSHLFLHLHTLQPEDSALYL

SEQ ID NO: 291 - protein, 9 aa, synthetic construct
GEGSRLTVL

SEQ ID NO: 292 - protein, 26 aa, synthetic construct
EAQVTQNPRYLITVTGKKLTVTCSQN

SEQ ID NO: 293 - protein, 17 aa, synthetic construct
MSWYRQDPGLGLRQIYY

SEQ ID NO: 294 - protein, 36 aa, synthetic construct
TDKGDVPEGYKVSRKEKRNFPLILESPSPNQTSLYF

SEQ ID NO: 295 - protein, 9 aa, synthetic construct
GPGTRLLVL

SEQ ID NO: 296 - protein, 26 aa, synthetic construct
AAGVIQSPRHLIKEKRETATLKCYPI

SEQ ID NO: 297 - protein, 17 aa, synthetic construct
VYWYQQGPGQDPQFLIS

SEQ ID NO: 298 - protein, 36 aa, synthetic construct
SDKGSIPDRFSAQQFSDYHSELNMSSLELGDSALYF

SEQ ID NO: 299 - protein, 9 aa, synthetic construct
GNGTRLTVT

SEQ ID NO: 300 - protein, 17 aa, synthetic construct
MYWYRQDPGLGLQLIYY

SEQ ID NO: 301 - protein, 26 aa, synthetic construct
EPEVTQTPSHQVTQMGQEVILRCVPI

SEQ ID NO: 302 - protein, 17 aa, synthetic construct
FYWYRQILGQKVEFLVS

SEQ ID NO: 303 - protein, 37 aa, synthetic construct
SEKSEIFDDQFSVERPDGSNFTLKIRSTKLEDSAMYF

SEQ ID NO: 304 - protein, 15 aa, synthetic construct
  VARIANT 5: note = X is L or I or E or G, or any of the following amino acids with related properties: V or D
  VARIANT 6: note = X is P or I or A, or any of the following amino acids with related properties: V, L or G
  VARIANT 9: note = X is G or N, or any of the following amino acids with related properties: Q, A, C or S
  VARIANT 10: note = X is T or no AA at this position, or S as an amino acid with related properties
  VARIANT 12: note = X is K or Q, or any of the following amino acids with related properties: R, H or N
  VARIANT 13: note = X is L or Y, or any of the following amino acids with related properties: I, V, F, W or H
  VARIANT 15: note = X is F or W
CAFMXXDSXXYXXIX

SEQ ID NO: 305 - protein, 14 aa, synthetic construct
  VARIANT 5: note = X is I or E, or any of the following amino acids with related properties: V or D
  VARIANT 6: note = X is P or A, or any of the following amino acids with related properties: V, L or G
CAFMXXDSNYQLIW

SEQ ID NO: 306 - protein, 14 aa, synthetic construct
  VARIANT 3: note = X is F or M, or any of the following amino acids with related properties: Y or W
  VARIANT 4: note = X is M or R, or any of the following amino acids with related properties: K or H
  VARIANT 5: note = X is I or E, or any of the following amino acids with related properties: V, L or D.
  VARIANT 6: note = X is P or A, or G as an amino acid with related properties
CAXXXXDSNYQLIW

SEQ ID NO: 307 - DNA, 334 bp, synthetic construct
  source 1..334: mol_type = other DNA
gcccagtctgtgagccagcataaccaccacgtaattctctctgaagcagcctcactggagttgggatgcaactattcctatggtggaactgttaatctcttctggtatgtccagtaccctggtcaacaccttcagcttctcctcaagtacttttcaggggatccactggttaaaggcatcaagggctttgaggctgaatttataaagagtaaattctcctttaatctgaggaaaccctctgtgcagtggagtgacacagctgagtacttctgtgccgtgaaagacaccgacaagctcatctttgggactgggaccagattacaagtctttccaa

SEQ ID NO: 308 - DNA, 343 bp, synthetic construct
  source 1..343: mol_type = other DNA
gatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgattgggacgacagcaccggggagctgttttttggagaaggctctaggctgaccgtactgg

SEQ ID NO: 309 - protein, 15 aa, synthetic construct
  VARIANT 5: note = X is L or I, or V as an amino acid with related properties
  VARIANT 6: note = X is P or I, or any of the following amino acids with related properties: V and L
  VARIANT 9: note = X is G or N, or any of the following amino acids with related properties: Q, A, C or S
  VARIANT 10: note = X is T or no AA at this position, or S as an amino acid with related properties
  VARIANT 12: note = X is K or Q, or any of the following amino acids with related properties: R, H or N
  VARIANT 13: note = X is L or Y, or any of the following amino acids with related properties: I, V, F, W or H
  VARIANT 15: note = X is F or W
CAFMXXDSXXYXXIX